(19) United States
(12) Patent Application Publication
(10) Pub. No.: US 2021/0395346 A1
(43) Pub. Date: Dec. 23, 2021

(54) STEREOTYPIC NEUTRALIZING VH CLONOTYPES AGAINST SARS-COV-2 RBD IN COVID-19 PATIENTS AND THE HEALTHY POPULATION

(71) Applicant: Seoul National University R&DB Foundation, Seoul (KR)

(72) Inventors: Junho CHUNG, Seoul (KR); Sang iL Kim, Seoul (KR); Sujeong Kim, Seoul (KR); Jinsung Noh, Seoul (KR); Younggeun Choi, Suwon (KR); Duck Kyun Yoo, Seoul (KR); Yonghee Lee, Seoul (KR); Hyunho Lee, Seoul (KR); Wan Beom Park, Seoul (KR); Myoung-don oh, Seoul (KR); Sunghoon Kwon, Seoul (KR)

(21) Appl. No.: 17/353,437

(22) Filed: Jun. 21, 2021

Related U.S. Application Data
(60) Provisional application No. 63/042,396, filed on Jun. 22, 2020.
(60) Provisional application No. 63/044,707, filed on Jun. 26, 2020.
(60) Provisional application No. 63/042,901, filed on Jun. 23, 2020.
(60) Provisional application No. 63/119,207, filed on Nov. 30, 2020.

(51) Int. Cl. C07K 16/10

(52) U.S. Cl. CPC C07K 16/10; C07K 2317/21; C07K 2317/522; C07K 2317/92; C07K 2317/565; C07K 2317/732; C07K 2317/526

(57) ABSTRACT

Described are stereotypic-naïve SARS-CoV-2 neutralizing antibodies that inhibit that SARS-CoV-2 virus replication. The antibodies comprise variable heavy chain (VH) clonotypes, encoded by either immunoglobulin heavy variable (IGHV)3-53 or IGHV3-66 and immunoglobulin heavy joining (IGHJ)6, and were identified in IgM, IgG3, IgG1, IgA1, IgG2, and IgA2 subtypes, with minimal somatic mutations, and could be paired with diverse light chains, resulting in binding to the SARS-CoV-2 receptor-binding domain (RBD). One of these clonotypes potently inhibited viral replication. Interestingly, these VH clonotypes pre-existed in six of 10 healthy individuals, predominantly as IgM isotypes, which could explain the expeditious and stereotypic development of these clonotypes among SARS-CoV-2 patients

### CROSS-REFERENCES TO RELATED APPLICATIONS

This application claims priority to and benefit of U.S. Provisional Patent Application Nos. 63/042,396, filed Jun. 22, 2020, 63/042,901, filed Jun. 23, 2020, 63/044,707, filed Jun. 26, 2020, and 63/119,207, filed Nov. 23, 2020, which are all incorporated by reference in their entirety.

This research was funded by the National Research Foundation of Korea [NRF-2016M3A9B6918973] and the Ministry of Science and ICT(MSIT) of the Republic of Korea and the National Research Foundation of Korea [NRF-2020R1A3B3079653]. This research was supported by the Global Research Development Center Program, through the NRF, funded by the MSIT [2015K1A4A3047345]. This work was supported by the Brain Korea 21 Plus Project in 2020.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing which has been submitted electronically in ASCII format and is hereby incorporated by reference in its entirety. Said ASCII copy, created on Jun. 18, 2021, is named 106094-1255889-USNP101_SL.txt and is 218,628 bytes in size.

### BACKGROUND

The coronavirus SARS-CoV-2 is responsible for the disease Covid-19. SARS-CoV-2 uses the spike (S) protein for receptor binding and membrane fusion. The S protein interacts with the cellular receptor angiotensin-converting enzyme II (ACE2) to gain entry into the host cell.

Stereotypic neutralizing antibodies (nAbs) that are identified in convalescent patients can be valuable, providing critical information regarding the epitopes that should be targeted during the development of a vaccine. Those antibodies with naïve sequences, little to no somatic mutations, and IgM or IgD isotypes are especially precious (1, 2) because these characteristics effectively exclude the possibility that these nAbs evolved from pre-existing clonotypes that are reactive to similar viruses. This critical phenomenon is referred to as original antigenic sin (OAS), and predisposed antibody-dependent enhancement (ADE) enhancing the severity of viral infections, which can sometimes be fatal, as in the case of the dengue virus vaccine (3-6). Several groups have identified nAbs for severe acute respiratory syndrome coronavirus 2 (SARS-CoV-2) (7-11), and one report suggested the possibility that stereotypic nAbs utilizing germline immunoglobulin heavy variable (IGHV)3-53 and IGHV3-66 segments may exist among convalescent patients (7). Furthermore, the structural basis of the stereotypic nAb reaction to SARS-CoV-2 was clarified using the co-crystal structure of two IGHV3-53 nAbs in complex with SARS-CoV-2 receptor-binding domain (RBD) by defining critical germline-encoded residues in the binding site of angiotensin-converting enzyme II (ACE2) (12). However, the prevalence of these stereotypic nAb clonotypes among SARS-CoV-2 patients and their characteristics, such as frequency in immunoglobulin (IG) repertoires, somatic mutations, isotypes, and chronological changes remain to be elucidated. Described herein are neutralizing antibodies that bind SARS-CoV-2 and methods of using same.

### BRIEF SUMMARY

Described herein are neutralizing antibodies that bind to a coronavirus, pharmaceutical compositions comprising the antibodies, methods for producing and using the antibodies to induce an immune response in a subject infected with a coronavirus or recovering from a coronavirus infection, and methods for treating a subject infected with a coronavirus. In some embodiments, the coronavirus is SARS-CoV-2, and the subject is suffering from Covid-19.

Thus, in one aspect, an isolated neutralizing antibody that binds SARS-CoV-2 is provided. In some embodiments, the antibody is an IgG, IgA, IgA or IgM class antibody. In some embodiments, the antibody is an IgG1, IgA1, or IgA2 subclass antibody.

In some embodiments, the antibody binds to the S1, S2, RBD and/or N proteins of SARS-CoV-2.

In some embodiments, the antibody comprises an amino acid sequence having at least 60%, 65%, 70%, 75%, 80%, 85%, 90%, or 95% sequence identity to one or more sequences shown in FIG. 1B-1D, Table 1, Table 3, Table 4, or Table 8, or Table 10, or a functional variant thereof.

In some embodiments, the antibody comprises an amino acid sequence having at least 60%, 65%, 70%, 75%, 80%, 85%, 90%, or 95% sequence identity to a light chain and/or a heavy chain variable region amino acid sequence shown in Table 10. In some embodiments, the antibody comprises a light chain variable region (VL) having an amino acid sequence selected from SEQ ID NOs: 1, 3, 5, 7, 9, 11, 13, 15, 17, 19, 21, 23, or 25, or an amino acid sequence having at least 60%, 65%, 70%, 75%, 80%, 85%, 90%, or 95% sequence identity to SEQ ID NOs: 1, 3, 5, 7, 9, 11, 13, 15, 17, 19, 21, 23, or 25. In some embodiments, the antibody comprises a heavy chain variable region (VH) having an amino acid sequence selected from SEQ ID NOs: 2, 4, 6, 8, 10, 12, 14, 16, 18, 20, 22, 24, or 26, or an amino acid sequence having at least 60%, 65%, 70%, 75%, 80%, 85%, 90%, or 95% sequence identity to SEQ ID NOs: 2, 4, 6, 8, 10, 12, 14, 16, 18, 20, 22, 24, or 26. In some embodiments, the antibody comprises:

i) a VL amino acid sequence of SEQ ID NO:1 and a VH amino acid sequence of SEQ ID NO:2;

ii) a VL amino acid sequence of SEQ ID NO:3 and a VH amino acid sequence of SEQ ID NO:4;

iii) a VL amino acid sequence of SEQ ID NO:5 and a VH amino acid sequence of SEQ ID NO:6;

iv) a VL amino acid sequence of SEQ ID NO:7 and a VH amino acid sequence of SEQ ID NO:8;

v) a VL amino acid sequence of SEQ ID NO:9 and a VH amino acid sequence of SEQ ID NO:10;

vi) a VL amino acid sequence of SEQ ID NO:11 and a VH amino acid sequence of SEQ ID NO:12;

vii) a VL amino acid sequence of SEQ ID NO:13 and a VH amino acid sequence of SEQ ID NO:14;

viii) a VL amino acid sequence of SEQ ID NO:15 and a VH amino acid sequence of SEQ ID NO:16;

ix) a VL amino acid sequence of SEQ ID NO:17 and a VH amino acid sequence of SEQ ID NO:18;

x) a VL amino acid sequence of SEQ ID NO:19 and a VH amino acid sequence of SEQ ID NO:20;

xi) a VL amino acid sequence of SEQ ID NO:21 and a VH amino acid sequence of SEQ ID NO:22,

xii) a VL amino acid sequence of SEQ ID NO:23 and a VH amino acid sequence of SEQ ID NO:24; or

xiii) a VL amino acid sequence of SEQ ID NO:25 and a VH amino acid sequence of SEQ ID NO:26;

or an amino acid sequence having at least 60%, 65%, 70%, 75%, 80%, 85%, 90%. or 95% sequence identity thereto.

In some embodiments, the antibody comprises a V gene and/or a J gene in FIG. 1B, FIG. 1D, Table 1, Table 3, Table 4, Table 5, or Table 8, or a functional variant thereof.

In some embodiments, the antibody comprises a HCDR3 amino acid sequence in FIG. 1B (SEQ ID NOS 685, 51, 113, 49, 118, 121, 82, 43, 89, 110, 107, respectively), or a functional variant thereof. In some embodiments, the antibody comprises a HCDR3 amino acid sequence in Table 1, or a functional variant thereof. In some embodiments, the antibody comprises a heavy chain variable region amino acid sequence having at least 80%, 85%, 90%, or 95% sequence identity to one or more sequences shown in FIG. 1C (SEQ ID NOS 686-700 respectively). In some embodiments, the antibody comprises a light chain CDR3 (LCDR3) sequence shown in FIG. 1D (SEQ ID NOS 701-708 respectively), or a functional variant thereof. In some embodiments, the antibody comprises a HCDR1, HCDR2 or HCDR3 sequence shown in Table 3, or a functional variant thereof. In some embodiments, the antibody comprises a HCDR1, HCDR2 or HCDR3 sequence shown in Table 4, or a functional variant thereof. In some embodiments, the antibody comprises a HCDR1, HCDR2 or HCDR3 sequence shown in Table 8, or a functional variant thereof.

In some embodiments, the antibody inhibits binding of SARS-CoV-2 S glycoprotein to ACE2.

In some embodiments, the antibody binds to a mutant RBD comprising one or more amino acid substitutions selected from V341I; F342L; N354D; D364Y; N354D and D364Y; V367F; A435S; W436R; G476S; V483A; G476S and V483A; N501Y; N439K; K417V; K417V and N439K; K417N; E484K; K417N, E484K, and N501Y; K417T; K417T, E484K, and N501Y; L452R; S477N; E484K; E484Q; or E484Q and L452R, or combinations thereof.

In some embodiments, the clonotype is IGHV3-53/IGHV3-66 and IGHJ6. In some embodiments, the antibody is a naïve stereotypic IGHV3-53/IGHV3-66 and IGHJ6 clone.

In some embodiments, the antibody is an scFv, Fab, or other antigen binding fragment or format thereof.

In another aspect, a pharmaceutical composition comprising an antibody described herein is provided.

In another aspect, a nucleic acid encoding a heavy chain variable region and/or a light chain variable region of an antibody described herein is provided.

In another aspect, a vector comprising a nucleic acid encoding a heavy chain variable region and/or a light chain variable region of an antibody described herein is provided. In some embodiments, the vector further comprises a nucleic acid encoding a hIgG₁ Fc region (hFc) or hCκ region operably linked to the nucleic acid encoding the heavy chain variable region or the nucleic acid encoding the light chain variable region.

In another aspect, a host cell comprising a vector described herein is provided.

In another aspect, a method for producing an antibody is described. In some embodiments, the method comprises culturing a host cell described herein under conditions in which the nucleic acids encoding the heavy and light chain variable regions are expressed.

In another aspect, an in vitro method for detecting binding of an antibody to SARS-CoV-2 antigens is described. In some embodiments, the method comprises contacting a cell infected with SARS-CoV-2 with an antibody described herein, and detecting binding of the antibody to the cell. In some embodiments, the method comprises contacting a recombinant SARS-CoV-2 antigen with an antibody described herein, and detecting binding of the antibody to the antigen. In some embodiments, the recombinant SARS-CoV-2 antigen comprises the SARS-CoV-2 spike, S1, S2, or N protein, or a recomdinant RBD domain of the S protein. In some embodiments, the recombinant SARS-CoV-2 antigen is fused to a molecular tag, such as a HIS tag, or fused to an antibody domain, such as a human Cκ domain.

In another aspect, a method of inducing an immune response in a subject is described. In some embodiments, the method comprises administering an antibody or pharmaceutical composition described herein to a subject.

In another aspect, a method of treating a patient infected with SARS-CoV-2 or suffering from COVID-19 is described. In some embodiments, the method comprises administering a therapeutically effective amount of an antibody or pharmaceutical composition described herein to the patient.

In another aspect, provided is an antibody or pharmaceutical composition described herein for use in the treatment of one or more symptoms of SARS-CoV-2 infection or COVID-19 disease in a subject. In some embodiments, provided is a pharmaceutical composition comprising an antibody described herein for the treatment of one or more symptoms of SARS-CoV-2 infection or COVID-19 disease in a subject.

### BRIEF DESCRIPTION OF THE DRAWINGS

FIG. 1A-1F. Characteristics of nAbs, derived from Patients A and E, stereotypic IGH clonotypes that are highly homologous to E-3B1, and the predicted RBD-binding clones that were enriched through biopanning. (A) Serially diluted IgG2/4 was mixed with an equal volume of SARS-CoV-2 containing 100 TCID₅₀ and the IgG2/4-virus mixture was added to Vero cells with 8 repeats and incubated for 5 days. Cells infected with 100 TCID₅₀ of SARS-CoV-2, isotype IgG2/4 control, or without the virus, were applied as positive, negative, and uninfected controls, respectively. CPE in each well was observed 5 days post-infection. (B) Characteristics of nAbs discovered in Patients A and E. FIG. 1B discloses SEQ ID NOS 685, 51, 113, 49, 118, 121, 82, 43, 89, 110, 107, respectively, in order of appearance. (C) IGH clonotypes that are highly homologous to E-3B1 and reactive against recombinant SARS-CoV-2 S and RBD proteins. The right column shows the results of the phage ELISA. All experiments were performed in quadruplicate, and the data are presented as the mean±SD. FIG. 1C discloses SEQ ID NOS 686-700, respectively, in order of appearance. (D) List of diverse IGL clonotypes that can be paired with the IGH clonotypes from (B) to achieve reactivity. FIG. 1D discloses SEQ ID NOS 701-708, respectively, in order of appearance. (E) J and (F) VJ gene usage in the IGH repertoire of patients (upper) and the binding-predicted IGH clones (bottom). For the VJ gene usage heatmap, the frequency values for the IGH repertoire of all 17 patients were averaged and are displayed (upper) along with those of the predicted RBD-binding IGH clones (bottom). N/A: not applicable.

FIG. 2A-2D. Deep profiling of the IGH repertoires of Patients A and E. (A and B) IGH repertoires of (A) Patient A and (B) Patient E were analyzed 11, 17, and 45 (A_d11, A_d17, A_d45) days and 23, 44, and 99 (E_d23, E_d44, E_d99) days after symptom onset, respectively. IGH repertoires were examined according to divergence from the germline and the isotype composition of the sequences. Values for divergence from the germline were calculated separately for each isotype and are presented as violin plots, ordered by the class-switch event. The bar graphs on the top of the violin plots represent the proportion of each isotype in the repertoire. (C and D) Mapping of three types of RBD-binding IGH sequences (neutralize, bind, and predicted), derived from either (C) Patient A or (D) Patient E, against the corresponding IGH repertoire. The positions of the RBD-binding IGH sequences in the divergence value were annotated as dot plots, on the same scale used for (A) and (B). Bar graphs on the top of the dot plots indicate the isotype compositions of the sequences in the repertoire.

FIG. 3. Titrations of serum IgG in ELISA. Plasma samples from 17 SARS-CoV-2 patients were diluted (1:100) and added to plates coated with recombinant SARS-CoV-2 spike, S1, S2, or N proteins, fused to HIS tag, or RBD protein, fused to human Cκ domain. The amount of bound IgG was determined using anti-human IgG (Fc-specific) antibody. ABTS was used as the substrate. All experiments were performed in duplicate, and the data are presented as the mean±SD.

FIG. 4. Titrations of serum IgG in ELISA. Plasma samples of 17 SARS-CoV-2 patients were serially diluted and added to plates coated with recombinant MERS-CoV spike, RBD, and S2 proteins, fused to HIS. The amount of bound IgG was determined using anti-human IgG (Fc-specific) antibody. ABTS was used as the substrate. All experiments were performed in duplicate, and the data are presented as the mean±SD.

FIG. 5. Reactivity of anti-SARS-CoV-2 scFv antibodies against recombinant SARS-CoV-2 RBD. Recombinant SARS-CoV-2 RBD-coated microtiter plates were incubated with varying concentrations of scFv-hCκ fusion proteins. HRP-conjugated anti-human Ig kappa light chain antibody was used as the probe, and TMB was used as the substrate. All experiments were performed in duplicate, and the data are presented as the mean±SD.

FIG. 6. Inhibition of recombinant SARS-CoV-2 S glycoprotein binding to ACE2-expressing cells, by flow cytometry. The recombinant scFv-hFc fusion proteins (200 nM or 600 nM) were mixed and incubated with recombinant SARS-CoV-2 S glycoprotein (200 nM) fused with a HIS tag at the C-terminus. After incubation with Vero E6 (ACE2⁺) cells, the relative amount of bound, recombinant SARS-CoV-2 S glycoprotein was measured using a FITC-conjugated anti-HIS antibody. For each sample, 10,000 cells were monitored. All experiments were performed in duplicate and the data are presented as the mean±SD. P-values from t-tests between the irrelevant scFv-hFc fusion protein 4 and each scFv fusion protein is annotated at the top of the bar plot.

FIG. 7. Neutralization of SARS-COV-2 in an in vitro experiment. The recombinant scFv-hCκ fusion proteins were mixed with 2,500 TCID₅₀ of SARS-CoV-2 (BetaCoV/Korea/SNU01/2020, accession number MT039890), and the mixture was added to the Vero cells. After 0, 24, 48, and 72 h of infection, the culture supernatant was collected to measure the viral titers.

FIGS. 8A and 8B. Mapping of the 11 nAbs to the overlapping IGH repertoire. (FIG. 8A) The number class-switched IGH sequences in the overlapping repertoire, mapped to nAbs. The allowed number of HCDR3 amino acid sequence substitutions during the mapping process is represented on the x-axis of the plot, after normalizing against the sequence length. The number of mapped sequences was normalized against the total number of IGH sequences in each patient, and their sum is represented in the y-axis of the plot. (FIG. 8B) The number of patients expressing the overlapping class-switched IGH sequences, which were mapped to the nAbs. The x-axis is the same as described for (A), and the y-axis indicates the number of patients.

FIG. 9A-9G. Existence of VL that can be paired with the stereotypic V_{H}. V_{L} was mapped according to identical VJ gene usage and perfectly matched LCDR3 sequences at the amino acid level, which were identified in the IGL repertoires of seven patients (FIG. 9A-G). The frequency values of the mapped sequences in the repertoires of all sampling points were summed. Patient identification can be found above each bar graph.

FIG. 10A-10G. VJ gene usage among the IG kappa light chain repertoire of patients. The frequency values of all sampling points were averaged and represented for each patient. Patient identification can be found at the top left corner of each heatmap.

FIG. 11A-11G. VJ gene usage among the IG lambda light chain repertoire of patients. The frequency values of all sampling points were averaged and are represented for each patient. Patient identification can be found at the left top corner of each heatmap.

FIG. 12. Reactivity of phage-displayed scFv clones in phage ELISA. Recombinant SARS-CoV-2, SARS-CoV, or MERS-CoV RBD protein-coated microtiter plates were incubated with phage clones. HRP-conjugated anti-M13 antibody was used as the probe, and ABTS was used as the substrate. All experiments were performed in quadruplicate, and the data are presented as the mean±SD.

FIG. 13A-130. Deep profiling of the IGH repertoire of Patients B, C, D, F, and G. (A to O) IGH repertoires of (A) Patient B, (B) Patient C, (C) Patient D, (D) Patient F, (E) Patient G, (F) Patient H, (G) Patient I, (H) Patient J, (I) Patient K, (J) Patient L, (K) Patient M, (L) Patient N, (M) Patient O, (N) Patient P, and (O) Patient Q were examined according to divergence from the germline and the isotype composition of the sequences. Values of divergence from the germline were calculated separately, for each isotype, and are presented as violin plots, class-switching event order. The bar graphs above the violin plots represent the proportions of each isotype.

FIG. 14. Reactivity of nAbs against recombinant SARS-CoV-2 spike mutants. Recombinant wild-type or mutant (V341I, F342L, N354D, V367F, R408I, A435S, G476S, V483A, and D614G) SARS-CoV-2 S, S1, or RBD protein-coated microtiter plates were incubated with varying concentrations of scFv-hFc fusion proteins. HRP-conjugated anti-human IgG antibody was used as the probe, and ABTS was used as the substrate. All experiments were performed in triplicate, and the data are presented as the mean±SD.

FIG. 15A-15Q. The nearest-neighbor distance histogram for HCDR3 amino acid sequences in the IGH repertoires of patients. The frequency values of the histograms were approximated by the binned kernel estimation method, in the Gaussian kernel setting (black line). The threshold value for each patient was set as the x value of the points with a minimum frequency value between two peaks of the bimodal distribution (red vertical line). The x and y values of the threshold-setting point are indicated in the upper right corner of each histogram.

FIG. 16. Frequency scatter plots for the NGS data of the four libraries, after each round of biopanning. The x- and y-axes represent the frequency values for the NGS data in each biopanning round. The line on the scatter plots indicates the identity line (y=x). Input and output virus titer values are also presented, above the matched scatter plots.

FIG. 17. The results of principal component analysis, applied to the NGS data of four libraries, after each round of biopanning. Information regarding the PC weight vectors, and the cumulative proportion of variance explained by the PCs are listed on the left side of the plots. PCA plots for PC1 and PC2 on shown on the right side of the plots. The binding-predicted clones were defined based on the value of PC1 and the ratio between PC1 and PC2, by setting a constant threshold value for each. The population of clones defined as predicted clones is marked in pink. The clones known bind to SARS-CoV-2 RBD are marked in red.

FIG. 18-A-E. Binding of antibodies to RBD variants. Binding of antibodies A-1H4 (A), A-2F1 (B), A-2H4 (C), E-3B1 (D), and E-3G9 (E) to SARS-CoV-2 to the indicated RBD variants was determined by ELISA.

### TERMINOLOGY

The term “stereotypic” refers to a characteristic shared between many or most individuals, or a non-heterogeneous characteristic.

The term “clonotype” refers to a collection of B cell receptor sequences sharing identical or similar functions expected to be derived from the same progenitor cells, and includes stereotypic antibodies comprising a VH clonotype encoding the same VH and JH genes and perfectly matched HCDR3 sequences, at the amino acid level.

The term “antibody” refers to an immunoglobulin (Ig) molecule or fragment or format thereof that specifically binds to a target antigen. The term includes monoclonal antibodies and the IgA, IgD, IgE, IgG, and IgM isotypes and subtypes. The term also includes antigen-binding fragments or formats thereof, such as Fab (fragment, antigen binding), Fv (variable domain), scFv (single chain fragment variable), disulfide-bond stabilized scFv (ds-scFv), single chain Fab (scFab), dimeric and multimeric antibody formats like dia-, tria- and tetra-bodies, minibodies (miniAbs) comprising scFvs linked to oligomerization domains, VHH/VH of camelid heavy chain Abs and single domain Abs (sdAb). The term also includes fusion proteins of that antibodies or antigen-binding fragments thereof, such as scFv-light chain fusion proteins, or scFv-Fc fusion proteins. The term also includes antibodies or antigen-binding fragments thereof that include an Fc domain to provide effector functions such as Antibody-Dependent Cell-Mediated Cytotoxicity (ADCC) and Complement Dependent Cytotoxicity (CDC).

The term “neutralizing antibody” refers to an antibody or fragment thereof that prevents infection of a host cell by a virus, or blocks attachment to the cell and/or entry of the virus into the cell.

The term “subject” refers to an animal, for example a mammal, including but not limited to a human, a rodent such as a mouse or rat, a companion animal such as a dog or cat, and livestock such as cows, horses, and sheep. The term subject can also be used interchangeably with the term “patient.”

The term “sequence identity” refers to two or more amino acid or nucleic acid sequences, or subsequences thereof, that are the same. Sequences can also have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity over a specified region), when compared and aligned for maximum correspondence over a comparison window, or designated region as measured using a sequence comparison algorithm or by manual alignment and visual inspection. Two or more amino acid or nucleic acid sequences can also have 90%, 91%, 92%, 93%, 94%, 95%, 96%, 97%, 98% or 99% sequence identity, thereby excluding sequences that are 100% identical (for example, a variant sequence is less than 100% identical to a wild-type or reference sequence). Two amino acid sequences can also be similar, i.e., they have a specified percentage of amino acid residues that are either the same or similar as defined by a conservative amino acid substitutions (e.g., at least 60%, 65%, 70%, 75%, 80%, 85%, 90%, 91%, 92%, 93%, 94%, 95%, 96%, 97%, 98%, or 99% similar over a specified region), when compared and aligned for maximum correspondence over a comparison window, or designated region as measured using a sequence comparison algorithm described herein or by manual alignment and visual inspection. The above definitions also refer to the complement of a nucleotice sequence. For sequence comparison, one sequence typically acts as a reference sequence, to which test sequences are compared. When using a sequence comparison algorithm, test and reference sequences are entered into a computer, subsequence coordinates are designated, if necessary, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters.

The “percentage of sequence identity” can determined by comparing two optimally aligned sequences over a comparison window, wherein the portion of the sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison can be determined, for example, by the local homology algorithm of Smith and Waterman (Adv. Appl. Math. 2:482, 1970), by the homology alignment algorithm of Needleman and Wunsch Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (see the internet at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

The term “host cell” refers to both single-cell prokaryote and eukaryote organisms (e.g., bacteria, yeast, and actinomycetes) and single cells derived from multicellular plants or animals. Host cells are typically isolated and grown in cell culture.

The term “vector” refers to a nucleic acid sequence, typically double-stranded DNA, which can comprise a fragment of heterologous nucleic acid sequence (e.g., a heterologous DNA sequence) inserted into the vector sequence. The vector can be derived from a bacterial plasmid. Vectors can contain polynucleotide sequences that facilitate the autonomous replication of the vector in a host cell. The term “heterologous” refers to nucleic acid sequences not naturally found in the host cell, for example, sequences that function to replicate the vector molecule, or sequences that encode a selectable or screenable marker, or encode a transgene. A vector can used to transport the heterologous nucleic acid sequence into a suitable host cell. Once in the host cell, the vector can replicate independently of or coincidental with the host chromosomal DNA, and multiple copies of the vector and its inserted DNA can be generated. In addition, the vector can also contain the necessary elements that permit transcription of the heterologous DNA into an mRNA molecule or otherwise cause replication of the heterologous DNA into multiple copies of RNA. Expression vectors can contain additional sequence elements adjacent to the inserted DNA that increase the half-life of the expressed mRNA and/or allow translation of the mRNA into a protein molecule.

### DETAILED DESCRIPTION

Neutralizing Antibodies

Described herein are stereotypic neutralizing antibodies (nAbs) that bind SARS-CoV-2 antigens. The antibodies can comprise naïve immunoglobulin (Ig) sequences having few or no somatic mutations. For example, described herein are stereotypic-naïve SARS-CoV-2 neutralizing antibody clonotypes that are present in the majority of patients with few somatic mutations and class-switched isotypes, and also pre-exist in the majority of individuals in the healthy human population, predominantly as an IgM isotype.

The inventors have unexpectedly found that the stereotypic-naïve nAbs described herein can rapidly initiate virus neutralization upon SARS-CoV-2 infection. The stereotypic-naïve SARS-CoV-2 nAbs described herein also provide the unexpected advantage of allowing a naïve heavy chain variable region sequence to pair with multiple light chain variable region sequences (referred to herein as light chain plasticity), and the resulting antibodies can bind the RBD and neutralize virus infection of host cells. The naïve heavy chain clonotypes described herein further provide the advantage of potentially providing near-immediate protection to subjects exposed to SARS-CoV-2 and thereby improve clinical outcomes. The nAbs described herein also provide the unexpected advantage of binding to known mutations within the RBD, therefore potentially providing protection against many SARS-CoV-2 mutants. Thus, the nAbs described herein may prevent “escape” of viral mutants in patients administered an antibody described herein, or prevent a reduction in the secondary immune response due to subsequent exposure to variant strains of SARS-CoV-2 (referred to as original antigenic sin).

In some embodiments, the nAbs described herein do not activate effector functions in response to closely related viruses. In some embodiments, the nAbs described herein do not trigger antibody-dependent enhancement (ADE) when administered to a subject.

In some embodiments, the stereotypic nAb is perfectly naïve and comprises a variable region encoded by a germline variable region gene (i.e., a genomic nucleic acid sequence). In some embodiments, the stereotypic nAb comprises a germline heavy chain variable region sequence joined to a germline J region sequence. In some embodiments, the stereotypic nAb has a low frequency of somatic mutations, for example, less than 2.695%+/−0.700%.

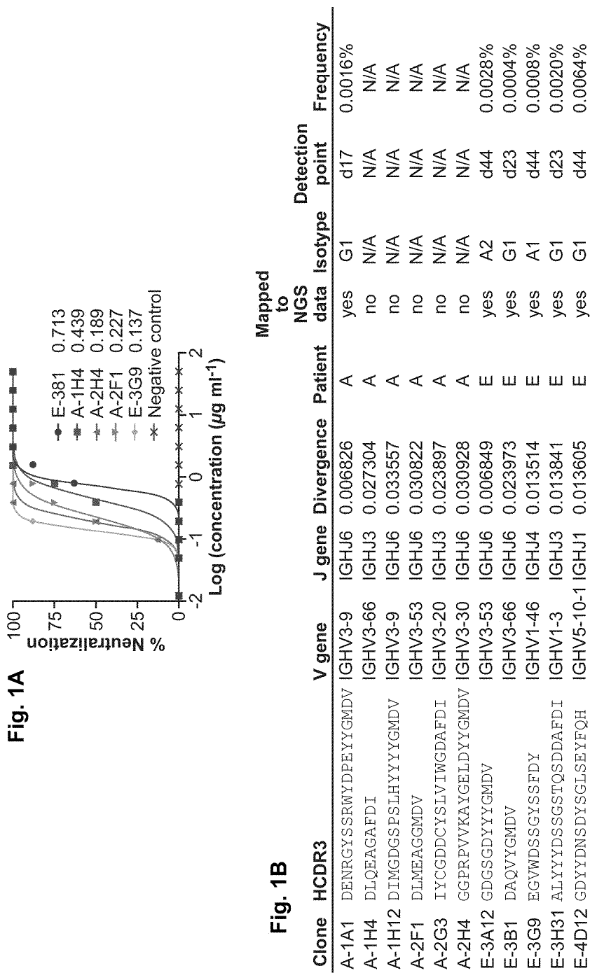

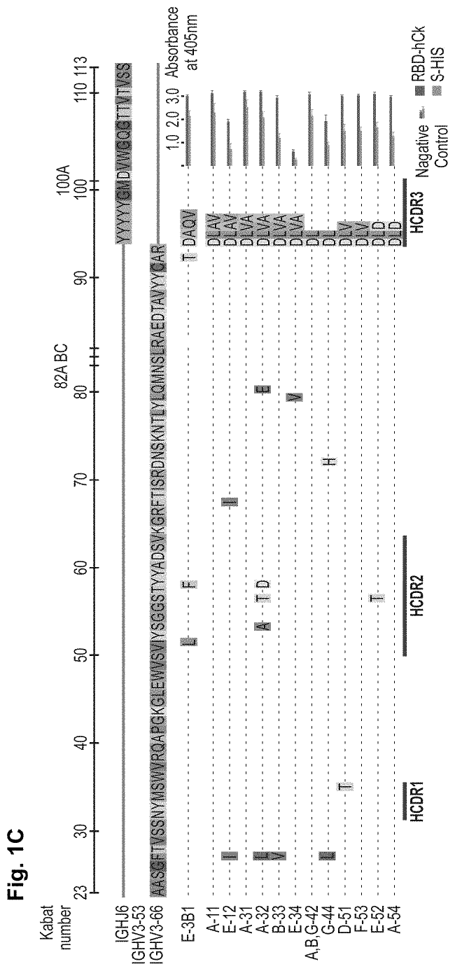

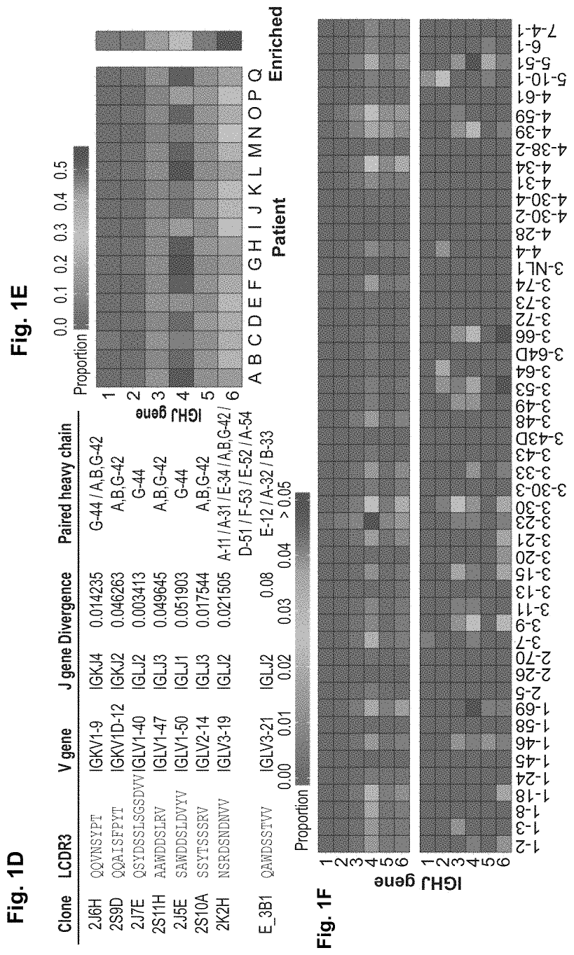

In some embodiments, the heavy chain of the stereotypic nAb is encoded by immunoglobulin heavy variable gene IGHV3-53. In some embodiments, the heavy chain of the stereotypic nAb is encoded by immunoglobulin heavy variable gene IGHV3-66. In some embodiments, the stereotypic nAb comprises a heavy chain variable region (VH) amino acid sequence having at least 80%, 85%, 90%, or 95% sequence identity to one or more sequences shown in Table 10. In some embodiments, the VH sequence comprises an HCDR3 having the amino acid sequence shown in FIG. 1B, Tables 1, 3, 4, or 8, or a variant thereof having one or more amino acid substitutions therein. In some embodiments, the VH sequence comprises an HCDR3 having the amino acid sequence DLYYYGMDV (SEQ ID NO: 27). In some embodiments, the heavy chain variable region comprises a V gene or J gene shown in FIG. 1B or FIG. 1F.

In some embodiments, the joining region of the stereotypic nAb is encoded by the immunoglobulin heavy joining 6 gene IGHJ6.

In some embodiments, the stereotypic nAb is an IgM isotype. In some embodiments, the stereotypic nAb is an IgG (e.g., IgG1, IgG2, IgG3) isotype, IgA (e.g. IgA1, IgA2) isotype, or IgD isotype.

In some aspects, the stereotypic nAb comprises a common heavy chain paired with different light chains (referred to as “light chain plasticity”). For example, In some embodiments, the stereotypic nAb comprises a heavy chain encoded by IGHV3-53 or IGHV3-66, and a light chain encoded by one of five different Vκ/Vλ genes. Representative examples of a common heavy chain paired with different light chains are shown in FIG. 1D (e.g. heavy chain “A,B,G-42” pairs with light chain clones 2J6H, 2S9D, 2S11H, 2S10A, and 2K2H) and described in the Examples.

In some embodiments, the stereotypic nAb comprises a heavy chain variable region (VH) paired with a light chain variable region (VL) clone shown in FIG. 1D, where the VH is selected from i) clone shown in FIG. 1B; ii) a clone or amino acid sequence shown in FIG. 1C; iii) an IGHV gene or IGHJ gene shown in FIG. 1F; iv) a CDR3 amino acid sequence, V gene and/or J gene shown in Table 1; v) a clone, HCDR1, HCDR2, HCDR3 amino acid sequence, V gene and/or J gene shown in Table 3; vi) a HCDR1, HCDR2, HCDR3 amino acid sequence, V gene and/or J gene shown in Table 4; or vii) a HCDR1, HCDR2, HCDR3 amino acid sequence, V gene and/or J gene shown in Table 8. In some embodiments, the stereotypic nAb comprises a heavy chain variable region paired with a light chain variable region, where the VH is selected from i) a HCDR3 amino acid sequence shown in FIG. 1B, ii) an amino acid sequence shown in FIG. 1C, iii) a CDR3 amino acid sequence shown in Table 1, iv) a HCDR1, HCDR2 and/or HCDR3 amino acid sequence shown in Table 3, v) a HCDR1, HCDR2 and/or HCDR3 amino acid sequence shown in Table 4, or vi) a HCDR1, HCDR2 and/or HCDR3 amino acid sequence shown in Table 8, wherein the VL comprises the LCDR3 amino acid sequence, V gene and/or J gene shown in FIG. 1D.

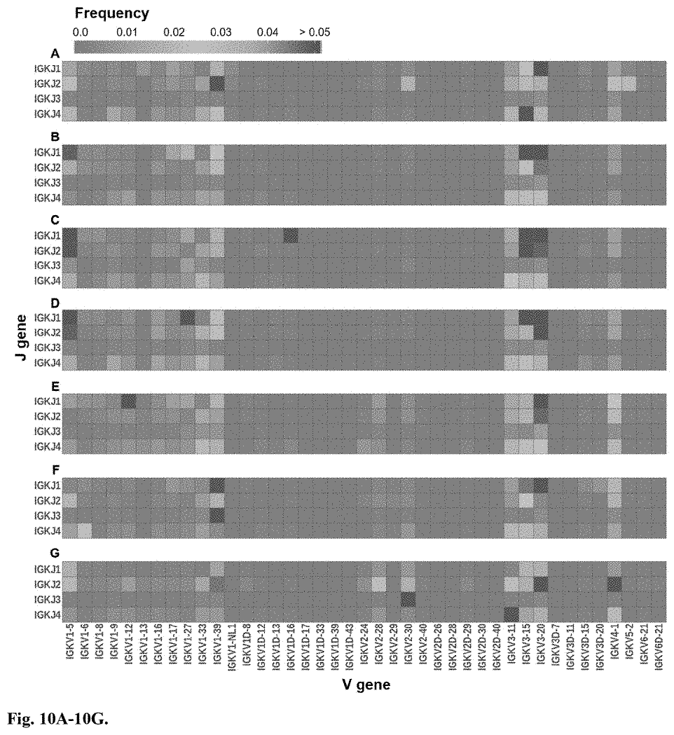

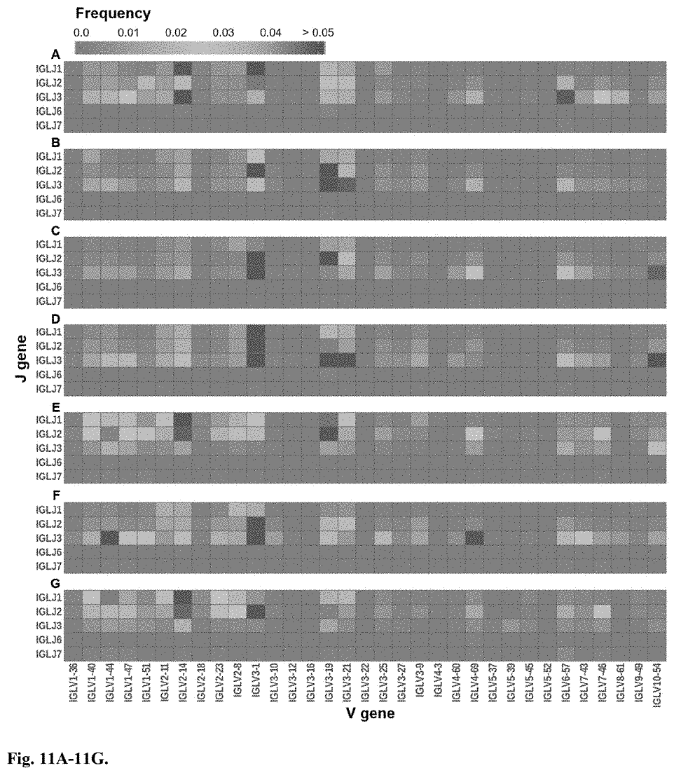

In some embodiments, the stereotypic nAb comprises a heavy chain variable region comprising a HCDR3 amino acid sequence shown in FIG. 1B paired with a light chain variable region comprising a LCDR3 amino acid sequence shown in FIG. 1D. In some embodiments, the stereotypic nAb comprises a heavy chain variable region comprising an amino acid sequence shown in FIG. 1C paired with a light chain variable region comprising a LCDR3 amino acid sequence shown in FIG. 1D. In some embodiments, the heavy chain variable region is paired with a light chain V gene or J gene shown in FIG. 1D, FIG. 10, or FIG. 11. In some embodiments, the heavy chain variable region is paired with IGLV2-14/IGLJ3, IGLV3-19/IGLJ2, and IGLV3-21/IGLJ2 (V gene/J gene).

In some embodiments, the stereotypic nAb comprises a light chain variable region (VL) amino acid sequence having at least 80%, 85%, 90%, or 95% sequence identity to one or more sequences shown in Table 10. In some embodiments, the stereotypic nAb comprises a light chain variable region LCDR3 amino acid sequence shown in FIG. 1D. In some embodiments, the stereotypic nAb comprises a light chain variable region V gene or J gene in FIG. 1D.

In some aspects, the clonotypes described herein comprise substantially identical heavy chain variable region (VH) amino acid sequences, for example the VH amino acid sequences are at least 90%, 91%, 92%, 93%, 94%, 95%, 96%, 97%, 98%, or 99% identical. In some embodiments, the clonotypes comprise VH sequences having a low frequency of somatic mutations, for example, a frequency of less than 5%, 4%, 3%, 2%, or 1% somatic mutations.

In some embodiments, the nAbs inhibit the binding of the coronavirus spike (S) protein to angiotensin-converting enzyme II (ACE2). ACE2 is the cellular receptor for SARS-CoV-2 in humans, which allows the virus to gain entry into a host cell. In some embodiments, the nAbs bind recombinant S protein. In some embodiments, the nAbs bind recombinant receptor-binding domain (RBD) protein. The RBD is located within the S1 region of the S protein. In some embodiments, the nAbs bind recombinant SARS-CoV-2 nucleocapsid (NP), S, S1 subunit, S2 subunit, and/or RBD proteins. The S1 subunit of the spike protein contains the receptor binding domain and is responsible for recognition and binding to the host cell receptor. The S2 domain is thought to be responsible for fusion between the viral envelope and the host cell membrane.

In some embodiments, the nAb binds to a mutant RBD comprising an amino acid substitution selected from one or more of the following: V341I, F342L, N354D, D364Y; N354D and D364Y, V367F, A435S, W436R, G476S, V483A; G476S and V483A; N501Y; N439K; K417V; K417V and N439K; K417N; E484K; K417N, E484K, and N501Y; K417T; K417T, E484K, and N501Y; L452R; S477N; E484K; E484Q; or E484Q and L452R, or combinations thereof.

Neutralization Assays

Neutralizing antibodies can be identified using a suitable neutralization assay. In some embodiments, the neutralization assay comprises inoculating or infecting cells or a cell line with SARS-CoV-2 virus, culturing the cells under conditions whereby the cells produce the virus, isolating the virus from the cells, mixing an amount (e.g., a predetermined amount) of the isolated virus with the antibody, contacting the mixture of virus and antibodies with non-infected cells or a non-infected cell line, and culturing the cells or cell line for an amount of time (for example, 24, 48 or 72 hours, or 1 to 5 days). In some embodiments, culture supernatant is collected, the viral titer is determined, for example by using a TCID50 assay, and the amount of viral RNA in the supernatant is quantified, for example, based on a standard curve using in vitro transcribed RNA. In some embodiments, the cell line is a Vero cells (ATCC CCL-81). In some embodiments, the cells or cell line are incubated with 100 to 2,500 TCID50 of SARS-CoV-2 virus.

Another example of a neutralization assay comprises determining the cytopathic effect (CPE) of cells infected with SARS-CoV-2 virus in the presence and absence of an antibody described herein. In some embodiments, a cell or cell line is incubated with a mixture of SARS-CoV-2 virus and the antibody, cultured for an amount of time (for example, 24, 48 or 72 hours, or 1 to 5 days), and the CPE determined, for example by calculating an IC50. In some embodiments, the cell line is a Vero cells (ATCC CCL-81). In some embodiments, the cells or cell line are incubated with 100 to 2,500 TCID₅₀ of SARS-CoV-2 virus.

In some embodiments, the antibodies described herein inhibit binding of the SARS-CoV-2 virus to a target cell. Thus, antibodies that inhibit binding of the SARS-CoV-2 virus to a target cell can be identified using an assay that measures inhibition of binding between a SARS-CoV-2 virus and a cells. In some embodiments, the assay detects inhibition of binding between recombinant SARS-CoV-2 S protein and cells expressing the ACE2 receptor. In some embodiments, the assay comprises mixing recombinant SARS-CoV-2 S protein with an antibody described herein, and determining the binding of the S protein to a cell or cell line expressing the ACE2 receptor. The binding can be measured by flow cytometry using an labeled antibody that binds to the recombinant SARS-CoV-2 S protein, where a decrease in signal from the label compared to a positive control indicates that the antibody inhibited binding of the S protein to the ACE2 receptor. In some embodiments, the recombinant SARS-CoV-2 S protein is fused with a polyhistidine (HIS)-tag, and the relative amount of bound, recombinant SARS-875 CoV-2 S glycoprotein is measured using a fluorescein isothiocyanate (FITC)-conjugated anti-HIS antibody. In some embodiments, the antibodies described herein inhibited binding between recombinant S protein and cells expressing the ACE2 receptor at an equimolar (1:1) ratio of recombinant S protein to antibody concentration, or up to a molar ration of 1:3 recombinant S protein to antibody concentration. In some embodiments, the antibodies described herein exhibit a half-maximal inhibitory concentration (IC₅₀) from 0.1 to 0.8 μg/mL. In some embodiments, the cell expressing the ACE2 receptor is a Vero E6 cell.

Antibody Formats

In some embodiments, the nAbs described herein are monoclonal antibodies comprising two Fab arms and one Fc region. In some embodiments, the two Fab arms bind to the same epitope of SARS-CoV-2. In some embodiments, the antibody comprises a single-chain variable fragment (scFv). In some embodiments, the antibody comprises a scFv fusion protein. In some embodiments, the scFv fusion protein comprises a scFv-light chain fusion protein. In some embodiments, the scFv fusion protein comprises a scFv-human kappa light chain fragment (hCκ) fusion protein (scFv-hCκ fusion proteins). In some embodiments, the scFv fusion protein comprises a scFv-human Fc region fusion protein (scFv-hFc fusion proteins).

Variants

Also described herein are variants of the neutralizing antibodies described herein. In some embodiments, the variant antibodies comprise one or more amino acid substitutions in the heavy or light chain sequence of an antibody described herein. In some embodiments, the variant antibodies comprise one or more amino acid substitutions in the heavy chain variable region (VH) or light chain variable region (VL) sequence of an antibody described herein. In some embodiments, the variant antibodies comprise one or more amino acid substitutions in the complementarity-determining regions (CDRs) of an antibody described herein, for example one or more amino acid substitutions in the heavy chain CDR1 (HCDR1), HCDR2 or HCDR3 sequence, or one or more amino acid substitutions in the light chain CDR1 (LCDR1), LCDR2 or LCDR3 sequence.

Antibody Libraries

Also provided are libraries comprising the antibodies described herein. The libraries can be prepared from biological samples from subjects infected by SARS-CoV-2. In some embodiments, the biological sample is a blood sample. Peripheral blood mononuclear cells (PBMCs) present in the blood sample are then isolated, and total RNA is prepared from the PBMCs. cDNA is synthesized from the RNA using primers that bind to the poly A tail of mRNA, or using gene specific primers. In some embodiments, the gene specific primers bind to sequences in the constant region (CH1 domain) of each isotype (IgM, IgD, IgG, IgA, and IgE). Following second strand cDNA synthesis, the double stranded DNA is purified, and the IgG genes are amplified, for example by PCR. For example, the VH and VL (V_{K} and V_{λ}) encoding genes can be amplified by PCR. In some embodiments, overlap extension PCR is used to link the amplified VH and VK/Vλ encoding fragments. In some embodiments, the VH and VK/Vλ encoding fragments are linked to produce scFv fusion constructs, that are then cloned into a phagemid vector. The synthesized VH and VL (V_{K} and V_{λ}) encoding genes can be amplified to produce scFv libraries, for example by PCR. The amplified scFV fragments can be cloned into phagemid vectors to produce a phage library. In some embodiments, the library can contain V_{K}/V_{λ} shuffled libraries.

Methods for Identifying Antibodies that Bind SARS-Cov-2 Antigens

The antibody libraries described herein can be used to identify antibodies that bind recombinant SARS-CoV-2 antigens, for example recombinant SARS-CoV-2 S and RBD proteins. In some embodiments, the recombinant SARS-CoV-2 antigenic proteins are fused to an Fc region or an antibody constant region, as described in the Examples. Methods for identifying antibodies that bind recombinant SARS-CoV-2 antigens include phage display followed by contacting the expressed antibodies to recombinant SARS-CoV-2 antigens, and eluting the bound antibodies. The recombinant SARS-CoV-2 antigens can be bound or conjugated to beads or magnetic beads. The bind and elute steps can be repeated multiple time, e.g., by biopanning, to identify high affinity antibodies.

In some embodiments, antibodies that bind SARS-CoV-2 antigens can be identified using an enzyme-linked immunosorbent assay (ELISA).

In some embodiments, neutralizing antibodies that bind SARS-CoV-2 antigens can be identified using a neutralization assay described herein. In some embodiments, neutralizing antibodies that bind SARS-CoV-2 antigens can be identified using an inhibition assay described herein.

In some embodiments, neutralizing antibodies that bind SARS-CoV-2 antigens can be identified using a phage ELISA. For example, antibodies can be selected that bind to SARS-CoV-2 S protein using recombinant S and RBD protein-coated microtiter plates, as described previously (45). In some embodiments, the antibody is an scFv. Antibodies can be sequenced to determine their nucleotide sequences.

Also provided are methods for identifying antibodies that have neutralizing activity against the SARS-CoV-2 virus. The method can comprise mutagenizing a polynucleotide encoding a heavy chain variable region or a light chain variable region of an antibody; expressing the antibody comprising the mutagenized heavy chain and/or light chain variable region; and selecting an antibody with neutralizing activity. The antibody with neutralizing activity can be selected using an assay described herein.

Representative antibody libraries and methods for producing same are described in the Examples.

Samples

To obtain antibodies against SARS-CoV-2, biological samples are typically obtained from the subject or patient. Samples include blood samples, plasma samples, and/or serum samples. In some embodiments, the sample comprises PBMCs. In some embodiments, the subject or patient is or has been infected by SARS-CoV-2. In some embodiments, the subject or patient is a human.

Pharmaceutical Compositions

Also described herein are pharmaceutical compositions comprising an antibody described herein. The pharmaceutical compositions can include additives such as a filler, bulking agent, buffer, stabilizer, or excipient. Standard pharmaceutical formulation techniques are well known to persons skilled in the art (see, e.g., 2005 Physicians' Desk Reference©, Thomson Healthcare: Montvale, N.J., 2004; Remington: The Science and Practice of Pharmacy, 20th ed., Gennado et al., Eds. Lippincott Williams & Wilkins: Philadelphia, Pa., 2000). In some embodiments, the pharmaceutical compositions contain pH buffering reagents, wetting or emulsifying agents, preservatives or stabilizers.

The pharmaceutical composition can also be formulated based on the intended route of administrations and other parameters (see, e.g., Rowe et al., Handbook of Pharmaceutical Excipients, 4th ed., APhA Publications, 2003). For example, the pharmaceutical composition be formulated for parental administration by intravenous, subcutaneous, intramuscular, or intra-articular administration. In some embodiments, the pharmaceutical composition is provided as a liquid or lyophilized form. In some embodiments, the pharmaceutical composition is a sterile, non-pyrogenic solution.

Nucleic Acids

Also provided are nucleic acid molecules such as polynucleotides that comprise a sequence encoding the amino acid sequence of an antibody described herein. In some embodiments, the nucleic acid molecule encodes a heavy chain and/or a light chain of an antibody described herein. In some embodiments, the nucleic acid molecule encodes a heavy chain variable region or a light chain variable region of an antibody described herein. In some embodiments, the nucleic acid molecule comprises sequences encoding both a heavy chain, or heavy chain variable region, and a light chain, or light chain variable region, of an antibody described herein. In some embodiments, the heavy and light chain variable regions are linked together. Methods for linking together the heavy and light chain variable regions, include but are not limited to ligation and overlap extension PCR. In some embodiments, the nucleic acid molecule is a DNA molecule. In some embodiments, the nucleic acid molecule is an RNA molecule.

In some embodiments, the nucleic acid molecule encodes an amino acid sequence shown in FIG. 1B-1D, or an amino acid sequence having at least, 80%, 85%, 90%, 91%, 92%, 93%, 94%, 95%, 96%, 97%, 98% or 99% sequence identity to an amino acid sequence shown in FIG. 1B-1D, or an amino acid sequence having 80%, 85%, 90%, 91%, 92%, 93%, 94%, 95%, 96%, 97%, 98% or 99% sequence identity to an amino acid sequence shown in FIG. 1B-1D, or a SARS-CoV-2 antigen binding variant thereof. In some embodiments, the nucleic acid molecule encodes a VH CDR3 amino acid sequence in Table 1, or SARS-CoV-2 RBD binding variant thereof. In some embodiments, the nucleic acid molecule encodes a HCDR1, HCDR2, and/or HCDR3 sequence in Table 3, or SARS-CoV-2 RBD binding variants thereof. In some embodiments, the nucleic acid molecule encodes a HCDR1, HCDR2, and/or HCDR3 sequence in Table 4, or SARS-CoV-2 antigen binding variants thereof. In some embodiments, the nucleic acid molecule encodes a HCDR1, HCDR2, and/or HCDR3 sequence in Table 8, or SARS-CoV-2 RBD binding variants thereof. In some embodiments, the nucleic acid molecule encodes a VH amino acid sequence and/or a VL amino acid sequence shown in Table 10. In some embodiments, the nucleic acid molecule encodes a light chain variable region (VL) having an amino acid sequence selected from SEQ ID NOs: 1, 3, 5, 7, 9, 11, 13, 15, 17, 19, 21, 23, or 25, or an amino acid sequence having at least 60%, 65%, 70%, 75%, 80%, 85%, 90%, or 95% sequence identity to SEQ ID NOs: 1, 3, 5, 7, 9, 11, 13, 15, 17, 19, 21, 23, or 25, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 1, 3, 5, 7, 9, 11, 13, 15, 17, 19, 21, 23, or 25. In some embodiments, the nucleic acid molecule encodes a heavy chain variable region (VH) having an amino acid sequence selected from SEQ ID NOs: 2, 4, 6, 8, 10, 12, 14, 16, 18, 20, 22, 24, or 26, or an amino acid sequence having at least 60%, 65%, 70%, 75%, 80%, 85%, 90%, or 95% sequence identity to SEQ ID NOs: 2, 4, 6, 8, 10, 12, 14, 16, 18, 20, 22, 24, or 26, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 2, 4, 6, 8, 10, 12, 14, 16, 18, 20, 22, 24, or 26.

Vectors

Also described herein are vectors comprising one or more nucleic acid sequences, for example, one or more nucleic acid sequences encoding an antibody described herein. In some embodiments, the vector comprises one or more nucleic acid sequences encoding a light chain variable region and/or a heavy chain variable region described herein. In some embodiments, the vector comprises one or more nucleic acid sequences encoding an amino acid sequence shown in FIG. 1B-1D, or an amino acid sequence having at least, 80%, 85%, 90%, 91%, 92%, 93%, 94%, 95%, 96%, 97%, 98% or 99% sequence identity to an amino acid sequence shown in FIG. 1B-1D, or an amino acid sequence having 80%, 85%, 90%, 91%, 92%, 93%, 94%, 95%, 96%, 97%, 98% or 99% sequence identity to an amino acid sequence shown in FIG. 1B-1D, or a SARS-CoV-2 antigen binding variant thereof. In some embodiments, the vector comprises one or more nucleic acid sequences encoding a VH CDR3 amino acid sequence in Table 1, or SARS-CoV-2 RBD binding variant thereof. In some embodiments, the vector comprises one or more nucleic acid sequences encoding a HCDR1, HCDR2, and/or HCDR3 amino acid sequence in Table 4, or SARS-CoV-2 antigen binding variants thereof. In some embodiments, the vector comprises one or more nucleic acid sequences encoding a HCDR1, HCDR2, and/or HCDR3 amino acid sequences in Table 8, or SARS-CoV-2 RBD binding variants thereof.

In some embodiments, the vector comprises one or more nucleic acid sequences encoding a light chain variable region (VL) having an amino acid sequence selected from SEQ ID NOs: 1, 3, 5, 7, 9, 11, 13, 15, 17, 19, 21, 23, or 25, or an amino acid sequence having at least 60%, 65%, 70%, 75%, 80%, 85%, 90%, or 95% sequence identity to SEQ ID NOs: 1, 3, 5, 7, 9, 11, 13, 15, 17, 19, 21, 23, or 25, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 1, 3, 5, 7, 9, 11, 13, 15, 17, 19, 21, 23, or 25. In some embodiments, the vector comprises one or more nucleic acid sequences encoding a heavy chain variable region (VH) having an amino acid sequence selected from SEQ ID NOs: 2, 4, 6, 8, 10, 12, 14, 16, 18, 20, 22, 24, or 26, or an amino acid sequence having at least 60%, 65%, 70%, 75%, 80%, 85%, 90%, or 95% sequence identity to SEQ ID NOs: 2, 4, 6, 8, 10, 12, 14, 16, 18, 20, 22, 24, or 26, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 2, 4, 6, 8, 10, 12, 14, 16, 18, 20, 22, 24, or 26. In some embodiments, the vector comprises one or more nucleic acid sequences encoding a light chain variable region (VL) having an amino acid sequence selected from SEQ ID NOs: 1, 3, 5, 7, 9, 11, 13, 15, 17, 19, 21, 23, or 25, and one or more nucleic acid sequences encoding a heavy chain variable region (VH) having an amino acid sequence selected from SEQ ID NOs: 2, 4, 6, 8, 10, 12, 14, 16, 18, 20, 22, 24, or 26. In some embodiments, the vector comprises one or more nucleic acid sequences encoding a light chain variable region (VL) comprising an amino acid sequence having at least 60%, 65%, 70%, 75%, 80%, 85%, 90%, or 95% sequence identity to SEQ ID NOs: 1, 3, 5, 7, 9, 11, 13, 15, 17, 19, 21, 23, or 25, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 1, 3, 5, 7, 9, 11, 13, 15, 17, 19, 21, 23, or 25, and one or more nucleic acid sequences encoding a heavy chain variable region (VH) comprising an amino acid sequence having at least 60%, 65%, 70%, 75%, 80%, 85%, 90%, or 95% sequence identity to SEQ ID NOs: 2, 4, 6, 8, 10, 12, 14, 16, 18, 20, 22, 24, or 26, or an amino acid sequence having 60%, 65%, 70%, 75%, 80%, 85%, 90%, 91%, 92%, 93%, 94%, 95%, 96%, 97%, 98% or 99% sequence identity to SEQ ID NOs: 2, 4, 6, 8, 10, 12, 14, 16, 18, 20, 22, 24, or 26.

In some embodiments, vector is an expression vector, such as a mammalian expression vector. In some embodiments, the vector is a phagemid vector. The expression vector can further comprise a constitutive or inducible promoter sequence for regulating transcription of the one or more nucleic acids, and a terminator sequence for terminating transcription. The one or more nucleic acids can be separated by internal ribosome entry sites (IRESes) that allow expression of different proteins from the same transcription unit.

In some embodiments, the vector comprises a nucleotide sequence encoding an Fc region or an antibody constant region at the 3′ end. In some embodiments, the Fc region is a human IgG1 Fc region. In some embodiments, the constant region is a human kappa constant region (hCκ). In some embodiments, the vector comprises the CH1 and hinge regions of an antibody. In some embodiments, the vector comprises the CH1 and hinge regions of a human or humanized antibody. In some embodiments, the vector comprises the the CH2 and CH3 regions of an antibody. In some embodiments, the vector comprises the the CH2 and CH3 regions of a human or humanized antibody. In some embodiments, the vector comprises the CH1 and hinge regions of human IgG2 fused to the CH2 and CH3 regions of human IgG4.

Host Cells

Also described herein are host cells. The host cells can comprise a vector described herein, and/or can comprise a nucleic acid sequence encoding an antibody described herein. Examples of host cells include single celled prokaryotes and eukaryotes, such as bacteria or yeast, or cells derived from multicellular organisms such as plants or animals. In some embodiments, the host cell is from a mammalian cell line. In some embodiments, the host cell is an Expi293F cell (Invitrogen).

In some embodiments, the host cell is capable of being infected by SARS-CoV-2. In some embodiments, the host cell expresses the ACE2 receptor. In some embodiments, the host cell expressing the ACE2 receptor is a Vero cell, or aVero E6 cell.

Methods of Producing Antibodies

In some embodiments, the neutralizing antibodies to SARS-CoV-2 described herein can be produced by transfecting a host cell with a nucleic acid encoding a heavy chain variable region and/or a light chain variable region of the antibody, and culturing the host cell under conditions suitable for expressing the heavy and/or light chain variable region protein. In some embodiments, the host cell comprises one or more nucleic acids encoding both a heavy chain variable region and a light chain variable region of the antibody, and the heavy and light chains self-assemble to form a functional antibody that specifically binds a SARS-CoV-2 antigen.

In some embodiments, the host cell comprises an expression vector comprising one or more nucleic acid sequences encoding a heavy chain variable region and/or a light chain variable region of a neutralizing antibody to SARS-CoV-2 described herein.

In some embodiments, the method for producing an antibody comprises synthesizing the amino acid sequence of the heavy chain variable region and/or light chain variable region of an antibody described herein.

Neutralizing antibodies to SARS-CoV-2 can also be obtained from biological samples from subjects infected with SARS-CoV-2. In some embodiments, the biological sample is a blood sample. In some embodiments, the antibodies so obtained can be used to generate antibody libraries.

In Vitro Methods for Detecting Binding of an Antibody to SARS-Cov-2 Antigens

In another aspect, an in vitro method for detecting binding of an antibody to SARS-CoV-2 antigens is described. In some embodiments, the method comprises contacting a cell infected with SARS-CoV-2 with an antibody described herein in vitro, and detecting binding of the antibody to the cell.

In some embodiments, the method comprises contacting a SARS-CoV-2 antigen with an antibody described herein in vitro, and detecting binding of the antibody to the antigen. The binding of the antibody to the SARS-CoV-2 antigen can be detected using an enzyme-linked immunosorbent assay (ELISA), or by detecting the signal from a labeled antibody such as a fluorescein labeled antibody.

In some embodiments, binding of an antibody described herein to SARS-CoV-2 antigens is detected by inhibiting binding between the SARS-CoV-2 S protein and a cell that expresses the ACE2 receptor. In some embodiments, the SARS-CoV-2 S protein is recombinantly labeled with a poly-HIS tag, and the relative amount of bound, recombinant SARSCoV-2 S glycoprotein is measured using a FITC-conjugated anti-HIS antibody. A decrease in fluorescent signal indicates that the antibody inhibits binding between the S glycoprotein and the ACE2 receptor.

Method of Inducing an Immune Response in a Subject

In another aspect, a method of inducing an immune response in a subject is provided. In some embodiments, the method comprises administering an antibody described herein to a subject. Following administration of the antibody, induction of the immune response can be detected in a biological sample from the subject, such as a blood or serum sample. Induction of an immune response includes induction of cytokines such an Type I IFNs (IFN-α and IFN-β) and IFN-γ, and changes to the TCR and BCR repertoire.

Methods of Treatment

Also described are methods for treating a subject infected with SARS-CoV-2, or displaying the symptoms of Covid-19. In some embodiments, the method comprises administering a therapeutically effective amount of an antibody described herein to the subject or patient. In some embodiments, the method comprises administering a therapeutically effective amount of a pharmaceutical composition comprising an antibody described herein to the subject or patient.

The antibodies described herein can be administered to a subject using an route of administration, such as parenterally, intravenously, subcutaneously, or intramuscularly. The antibody can be administered daily, weekly, or monthly. The antibody can be administered in a body—size-based, for example in a range from 1 milligram/square meter to 500 mg/square meter of body surface, or from 1 mg/kg to 10 mg/kg of body weight. The total single dose can range from 400 to 10,000 milligrams (0.4 to 10 grams) for a human subject. The dose can be a single dose, or multiple doses, such as two or more weekly doses.

The treatment method may further comprise administering one or more additional treatments, such as therapeutic agents or medical procedures, to the subject. In some embodiments, the one or more additional treatments include antivirals, immune-based therapies, other neutralizing antibodies, and administering oxygen and/or mechanical ventilators for patients with respiratory conditions or failure. In some embodiments, the antiviral is selected from Remdesivir, Lopinavir/Ritonavir (Kaletra®), Favipiravir, azithromycin or Arbidol. In some embodiments, the additional treatment comprises administering hydroxychloroquine or chloroquine to the subject.

In some embodiments, the additional treatment comprises administering an immune-based therapy, such as convalescent plasma and/or SARS-CoV-2-specific immune globulins, to the subject. In some embodiments, the additional treatment comprises immune suppressant drugs to treat the so-called “cytokine storm” associated with Covid-19 infection in patients that develop acute respiratory distress syndrome (ARDS). The immunosuppressant drug can be selected from those currently being tested in clinical trials, including baricitinib, a drug for rheumatoid arthritis; CM4620-IE, a drug for pancreatic cancer; and Interleukin inhibitors such as IL-6 inhibitors (e.g., sarilumab, siltuximab, or tocilizumab). In some embodiments, the additional treatment comprises administering immunomodulators, such as alpha and beta interferons and kinase inhibitors, to the patient.

In some embodiments, the additional treatment comprises administering corticosteroids to the patient. In some embodiments, the additional treatment comprises administering antithrombotic therapy to the patient. Antithrombotic therapy can include anticoagulants and antiplatelet therapy. Thus, in some embodiments, the additional treatment comprises administering venous thromboembolism (VTE) prophylaxis per the standard of care.

In some embodiments, the additional treatment comprises filtering cytokines out of the blood of Covid-19 patients. Suitable filters include those granted emergency use authorization by the FDA, including the Spectra Optia Apheresis System (Terumo BCT Inc.) and Depuro D2000 Adsorption Cartridge (Marker Therapeutics AG) devices.

In some embodiments, the additional treatment comprises administering oxygen therapy to the patient. In some embodiments, the additional treatment comprises placing the patient on ventilator support if the patient presents acute hypoxemic respiratory failure despite conventional oxygen therapy. In one embodiment, the treatment comprises administering high-flow nasal cannula (HFNC) oxygen to the patient.

### EXAMPLES

### Example 1

This example describes the identification, cloning and expression of neutralizing antibodies that bind SARS-CoV-2.

Isolation and Characterization of Human nAbs

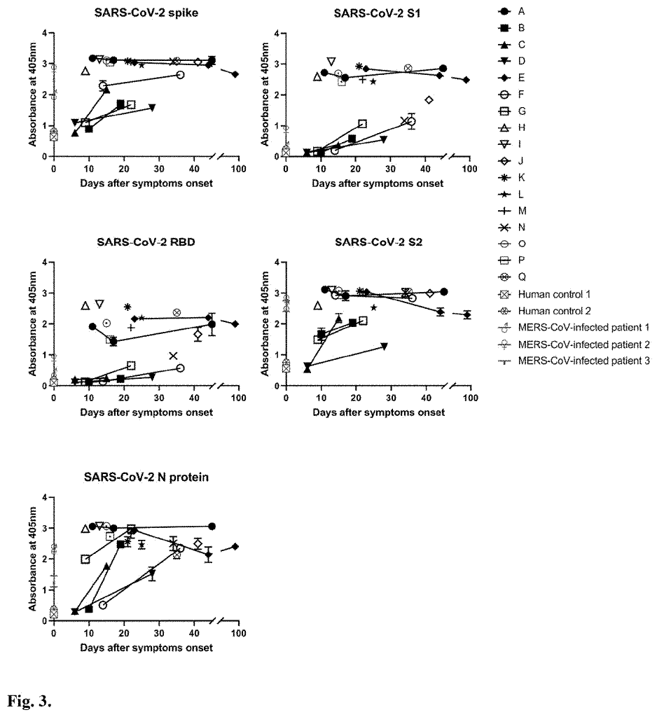

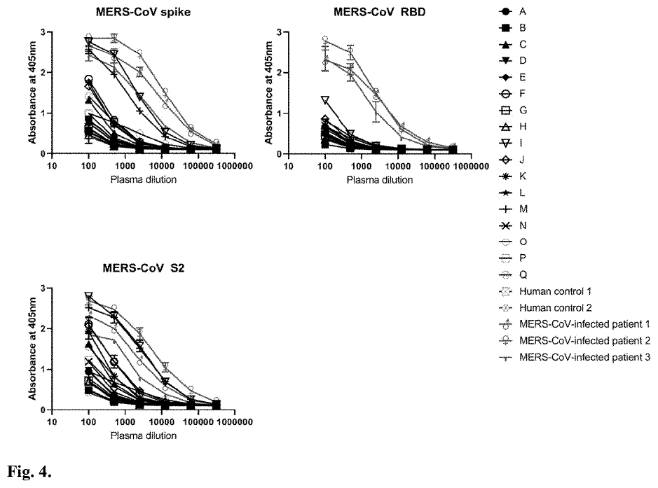

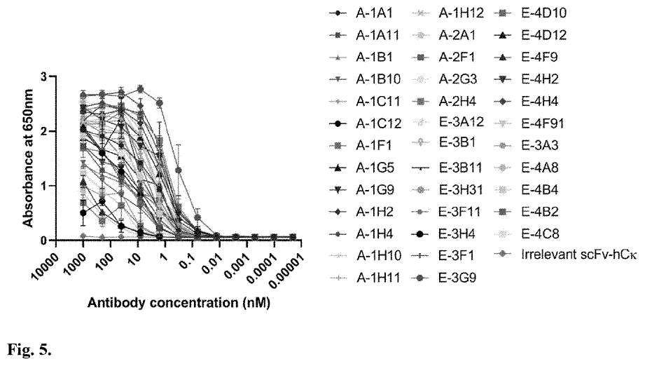

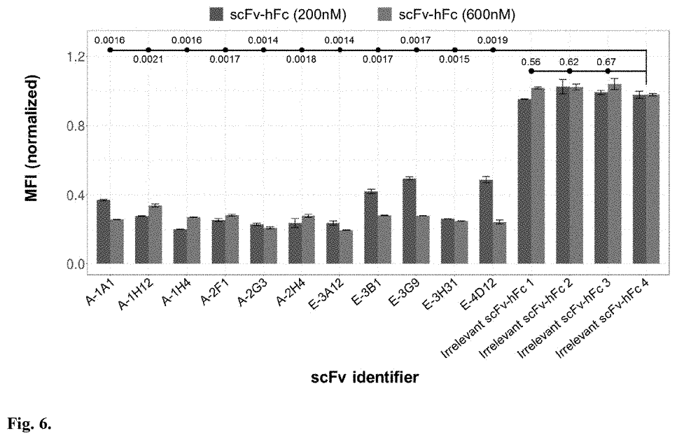

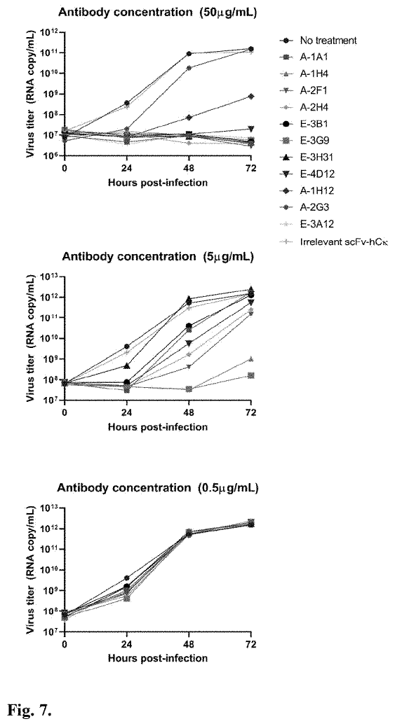

To obtain monoclonal nAbs against SARS-CoV-2, blood samples were collected from 17 SARS-CoV-2-infected patients (Patients A-Q) and used them to generate human antibody libraries. Similar to severe acute respiratory syndrome coronavirus (SARS-CoV), SARS-CoV-2 also uses a spike (S) protein for receptor binding and membrane fusion (13). This protein interacts with the cellular receptor ACE2 to gain entry into the host cell (14, 15). A previous report suggested that a human monoclonal antibody (mAb), which reacted with the RBD, within the S1 region of the S protein, could hinder the initial interaction between the virus and the cell, effectively neutralizing SARS-CoV-2 (11). The reactivity of the sera derived from patients against recombinant SARS-CoV-2 S and RBD proteins was confirmed. Patients A and E, who presented with extensive pneumonic infiltrates, also showed high plasma IgG titers against all recombinant SARS-CoV-2 nucleocapsid (NP), S, S1, S2, and RBD proteins, which could be detected 11, 17, and 45 days after symptom onset in Patient A and 23, 44, and 99 days after symptom onset in Patient E (Table 2 and FIG. 3). Notably, the sera samples from Middle East respiratory syndrome coronavirus (MERS-CoV) patients cross-reacted with the SARS-CoV-2 S protein, showing a higher titer against the S2 domain, and vice versa (FIGS. 3 and 4), suggesting the potential risk for ADE. Four human antibody libraries were generated, utilizing a phage-display system, based on the blood samples from Patient A, which were collected on days 17 and 45 (A_d17 and A_d45), and Patient E, which were collected on days 23 and 44 (E_d23 and E_d44). After biopanning, 38 single-chain variable fragment (scFv) clones were successfully isolated that were reactive against recombinant SARS-CoV-2 RBD using an enzyme-linked immunosorbent assay (ELISA) (FIG. 5 and Table 3). The half-maximal binding of these scFv-human kappa light chain fragment (hCκ) fusion proteins with the coated antigens occurred at concentrations ranging from 0.32 to 364 nM, which was compatible with the findings of previous reports that have described human mAbs against SARS-CoV-2 RBD (8, 11). These antibody clones were tested to determine if they could inhibit the binding between recombinant SARS-CoV-2 S protein and Vero E6 cells expressing the ACE2 receptor. When incubated with 1.5×105 Vero E6 cells, the recombinant polyhistidine (HIS)-tagged SARS-CoV-2 S protein showed saturated binding at 200 nM, according to flow cytometry analysis, using a fluorescein isothiocyanate (FITC)-labeled anti-HIS antibody. For the analysis, recombinant S protein (200 nM) was mixed with scFv-hFc fusion proteins, at a final concentration of either 200 nM (equimolar) or 600 nM (molar ratio of 1:3). Eleven clones (A-1A1, A-1H4, A-1H12, A-2F1, A-2H4, A-2G3, E-3A12, E-3B1, E-3G9, E-3H31, and E-4D12) almost completely inhibited the binding between recombinant S protein and Vero E6 cells at 600 nM, and some showed potent inhibition activity, even at 200 nM (FIG. 6). The neutralizing potency of these 11 clones for inhibition of viral replication was tested using an in vitro assay. Vero cells, in a T-25 flask, were infected with authentic SARS-CoV-2 encoding D614 in the viral S protein, at a medium tissue culture infectious dose (TCID50) of 2,500 and in the presence of scFv-hCκ fusion proteins, at concentrations of 0.5, 5, or 50 μg/mL. Viral RNA concentrations in the culture supernatant were determined 0, 24, 48, and 72 h after infection. Nine antibodies exhibited complete neutralizing activity, at 50 μg/mL (FIG. 7), and two antibodies (A-1H4 and E-3G9) showed potent neutralization, even at 5 μg/mL (FIG. 7). In IgG2/4 format, five nAbs (E-3B1, A-1H4, A-2H4, A-2F1, and E-3G9) exhibited potent neutralizing activity against authentic SARS-CoV-2, with half-maximal inhibitory concentration (IC50) ranging from 0.137 to 0.713 μg/mL (FIG. 1A).

Identification of Stereotypic Clonotypes from IGH Repertoire of SARS-CoV-2-Infected Patients

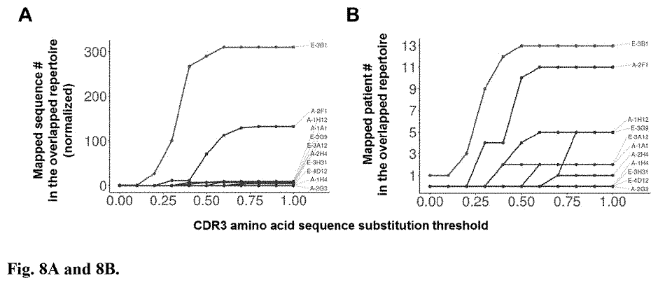

Deep profiling of the IG repertoire in three chronological blood samples each from Patients A and E, two chronological samples from each of Patients B, C, D, F, and G, and a single timepoint sample from each of the other ten patients (H-Q) was performed. nAb clonotypes that possessed identical variable (V) and joining (J) gene combinations and perfectly matched heavy chain complementarity-determining region 3 (HCDR3) amino acid sequences among the immunoglobulin heavy chain (IGH) repertoires of Patients A and E was determined. One and five nAb clonotypes were successfully identified in Patients A and E, respectively (FIG. 1B). Notably, three nAbs (A-2F1, E-3A12, and E-3B1) were encoded by IGHV3-53/IGHV3-66 and IGHJ6 (FIG. 1B). These two VH genes, IGHV3-53*01 and IGHV3-66*01 share an identical amino acid sequence, except for the H12 residue (isoleucine in IGHV3-53 and valine in IGHV3-66), and only five nucleotide differences exist between their sequences. Furthermore, four clonotypes were IgG1, and two clonotypes were class-switched to IgA1 and IgA2 when examined 44 days after symptom onset (FIG. 1B). These clonotypes had a very low frequency of somatic mutations (1.03%+/−0.51%), which was compatible with findings regarding other nAbs in previous reports (7, 8). Then, all VH sequences from the 17 patients were collected and the clonotypes of 11 nAbs that were encoded by the same VJ genes and showed 66.6% or higher identity in the amino acid sequence for HCDR3 (FIG. 8) was determined. Interestingly, clonotypes that were highly homologous to the E-3B1 nAb were found among 13 of 17 patients, with a total of 126 clonotypes having the isotype of IgG3 (Patients I, K, and P), IgG1 (Patients A, B, D-I, K, M, O, and P), IgA1 (Patients E, G, I, and J), IgG2 (Patients I-K) and IgA2 (Patient E) (Table 4). These clonotypes shared nearly identical VH sequences (92.45%+/−3.04% identity between amino acid sequences), with E-3B1 displaying an extremely low frequency of somatic mutations (0.98%+/−1.48%). Among these 126 clonotypes, 43 unique HCDR3s were identified, in amino acid sequence, and 12 unique HCDR3s existed in more than one patient (Table 1).

### Light Chain Plasticity of the Stereotypic VH Clonotypes for Binding to SARS-CoV-2 RBD

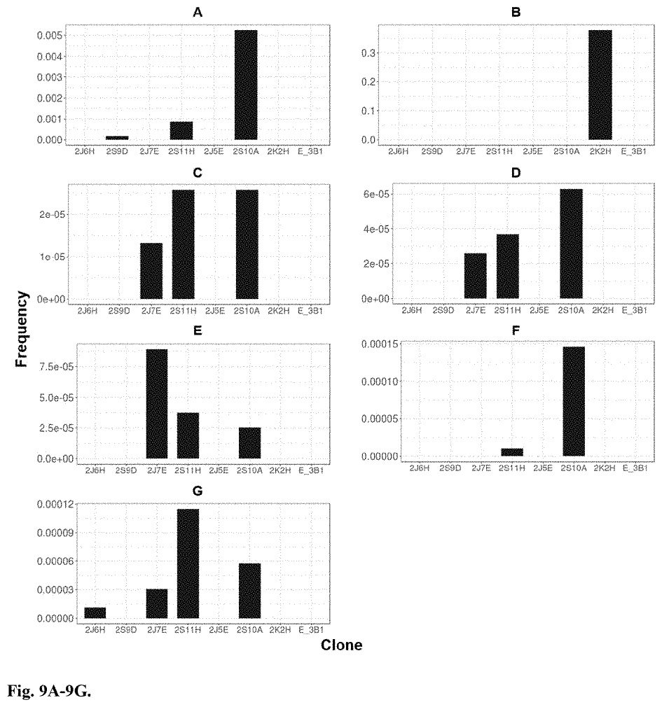

To test the reactivity of clonotypes homologous to E-3B1 against the SARS-CoV-2 S protein, 12 IGH clonotypes (FIG. 1C), containing five different HCDR3s, from the IGH repertoires of 13 patients were arbitrarily sampled. The genes encoding these IGH clonotypes were chemically synthesized and used to construct scFv genes, using the variable lambda chain (V2) gene from the E-3B1 clone. Then, the reactivities of these scFv clones were tested using an ELISA. Three clones (E-12, A-32, and B-33) reacted against the recombinant S and RBD proteins (FIG. 1C). Then, scFv libraries were constructed, using the A-11, A-31, E-34, A,B,G-42, G-44, D-51, F-53, E-52, and A-54 genes, and the variable kappa chain (Vκ)/Vλ genes amplified from Patients A, E, and G. All 12 IGH clonotypes were reactive against both recombinant S and RBD proteins when paired with eight different Vκ and Vλ genes (FIGS. 1C and 1D). Moreover, all seven light chain profiled patients (A-G) possessed these Vκ/Vλ clonotypes with identical VJ gene usage and perfectly matched light chain CDR3 (LCDR3) amino acid sequences (FIG. 9). In particular, immunoglobulin lambda variable (IGLV)2-14/immunoglobulin lambda joining (IGLJ)3, IGLV3-19/IGLJ2, and IGLV3-21/IGLJ2 were frequently used across all seven patients (FIGS. 10 and 11). Because E-3B1 effectively inhibited the replication of SARS-CoV-2 (FIG. 1A), these 126 clonotypes are likely to neutralize the virus when paired with an optimal light chain.

### Stereotypic-Naïve IGH Clonotype Against SARS-Cov-2 Pre-Existed in the Healthy Population

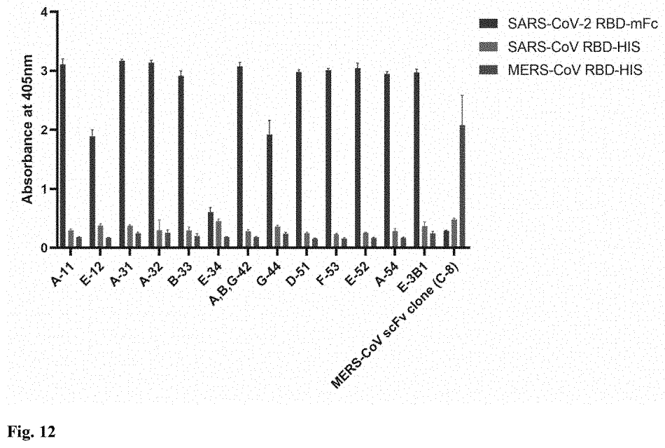

Among these IGH clonotypes, A,B,G-42 was quite unique, presenting little to no (0.6%+/−0.8%) somatic mutations and containing an HCDR3 (DLYYYGMDV (SEQ ID NO: 27)) formed by the simple joining of IGHV3-53 and IGHJ6. This naïve VH sequence existed in the IGH repertoire of five patients (Patients A, B, G, I, and K), as IgM and IgG1, IgM and IgG1, IgG1 and IgA1, IgM, or IgG1 subtypes, respectively (Table 1). More interestingly, the IGH clonotypes encoded by IGHV3-53/IGHV3-66 and IGHJ6 that possessed an HCDR3 (DLYYYGMDV (SEQ ID NO: 27)) with zero to one somatic mutation could be identified within the IGH repertoire of six of 10 healthy individuals, predominantly as an IgM isotype (16), based on publicly available IGH repertoires (Table 1). The A,B,G-42 clonotype showed light chain plasticity and paired with five Vκ/Vλ genes to achieve RBD binding. In particular, the Vκ gene (2J6H) accumulated only five somatic mutations (1.4% divergence). None of the 12 clones, including A,B,G-42, reacted against the recombinant RBD proteins from either SARS-CoV or MERS-CoV (FIG. 12). In prior experiments, none of the 37 identified MERS-RBD-binding human mAbs, from two patients, were encoded by IGHV3-53/IGHV3-66 and IGHJ6 (Table 5) (17). Therefore, the presence of these stereotypic-naïve IGH clonotypes in the healthy population, and their light chain plasticity to achieve SARS-CoV-2 RBD binding, may be unique to SARS-CoV-2, which might provide a rapid and effective humoral response to the virus among patients who express these clonotypes. These findings provide the majority of the population possess germline-precursor B cells, encoded by IGHV3-53/IGHV3-66 and IGHJ6, which can actively initiate virus neutralization upon SARS-CoV-2 infection.

### Distinctive V and J Gene Usage of the SARS-CoV-2 RBD-Binding Antibodies

To further elucidate the preferential use of IGHV3-53/IGHV3-66 and IGHJ6 genes during the generation of SARS-CoV-2 RBD-binding antibodies, 252 predicted RBD-binding clones were extracted from the biopanning data (See Methods). It was previously shown that antibody clones with binding properties can be predicted by employing next-generation sequencing (NGS) technology and analyzing the enrichment patterns of biopanned clones (18, 19). Although the IGHJ4 gene was more prominent in the IGH repertoires of 17 patients, similar to healthy human samples (16, 20), the predicted RBD-binding clones primarily used the IGHJ6 gene (FIG. 1E). Furthermore, the predicted RBD-binding clones showed the dominant usage of IGHV3-53/IGHJ6 and IGHV3-66/IGHJ6 pairs, which was not observed in the whole IGH repertoires of patients (FIG. 1F).

Chronological Follow-Up of IGH Repertoire and the SARS-CoV-2 RBD-Binding Antibodies from Patients

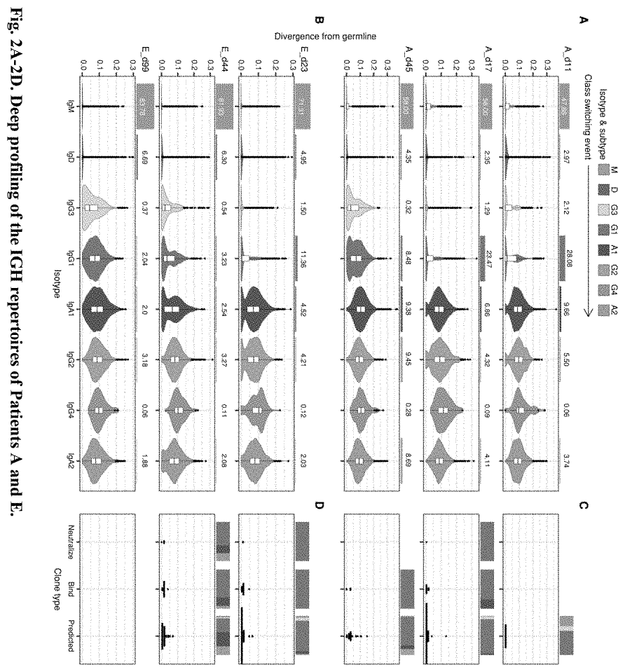

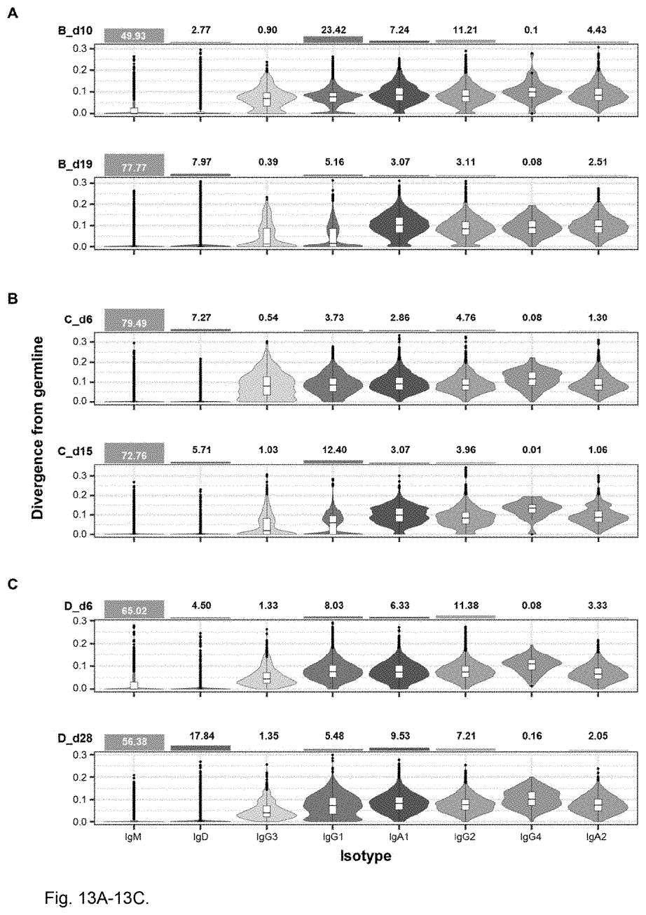

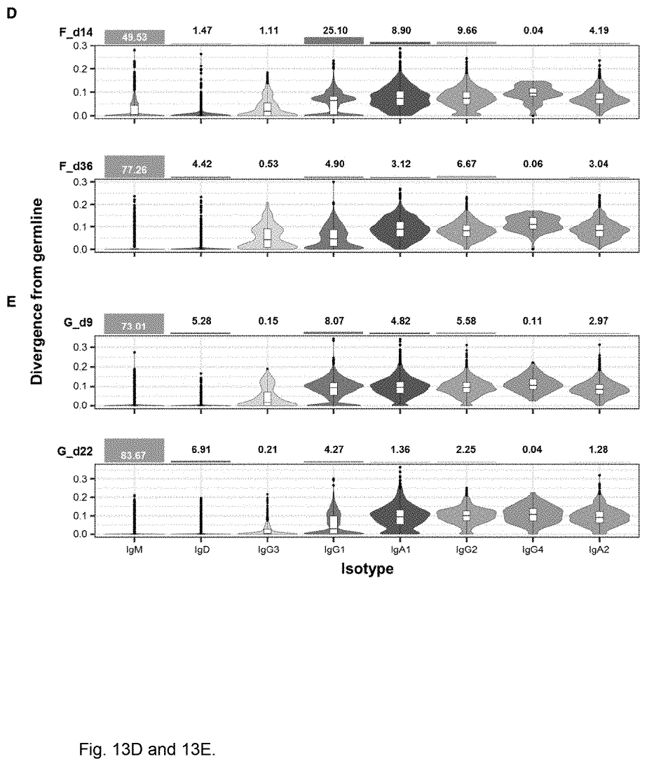

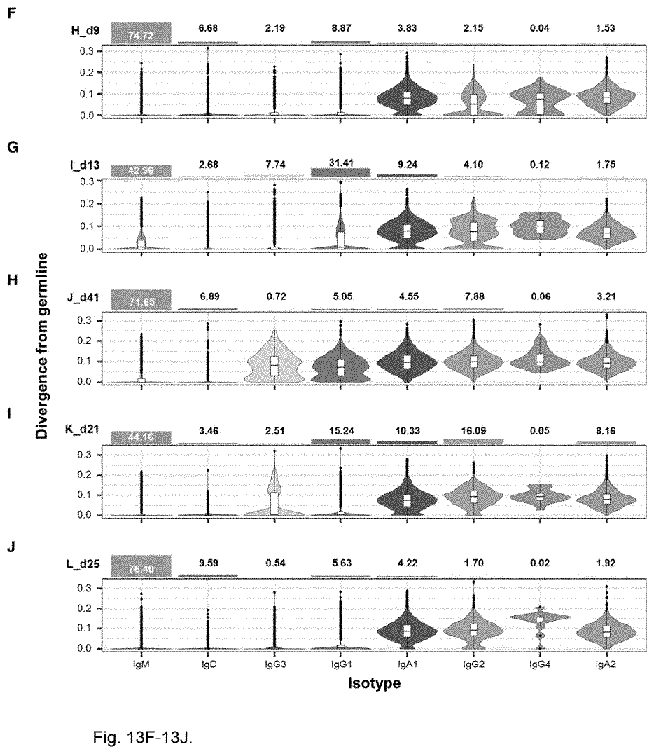

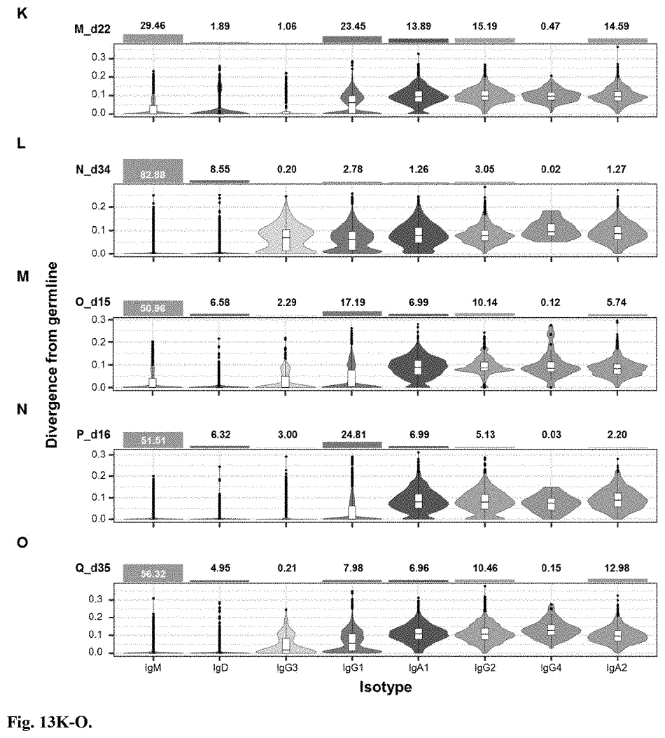

Naïve B cells typically undergo somatic hypermutations, clonal selection, and class-switching following antigen exposure. Thus, the chronological events that occurred in all IGH clonotypes identified in Patients A-G and those that were reactive against the SARS-CoV-2 RBD were examined. In the entire patient IGH repertoire, naïve-derived IGH clonotypes with minimal somatic mutations (<2.695%+/−0.700%) showed increased IgG3 and IgG1 subtypes, and the proportion of the IgG1 subtype was dramatically increased for a period (FIGS. 2A and 2B and FIG. 13). Furthermore, the naïve-derived IGH clonotypes were detected as minor populations as IgA1 and IgG2 subtypes in Patients A and E (FIGS. 2A and 2B), and as an IgA2 subtype in Patient E (FIG. 2B). RBD-reactive clones were categorized into three groups: 1) neutralizing antibodies (neutralize), 2) binding-confirmed antibodies (bind), and 3) binding-predicted antibodies (predicted). In all three groups, these IGH clonotypes appeared and disappeared throughout the disease course, showed a low frequency of somatic mutations (FIGS. 2C and 2D), and displayed rapid class-switching, especially to IgG1, IgA1, and IgA2. To summarize, RBD-reactive IGH clonotypes rapidly emerged and underwent class-switching, to IgG1, IgA1, and IgA2, without experiencing many somatic mutations. However, this dramatic temporal surge of naïve IGH clonotypes, with rapid class-switching, occurred across the entire IGH repertoire of the patients and was not confined to those reactive to the SARS-CoV-2 RBD.

Selected nAbs Retained the Ability to Bind to Most Current SARS-CoV-2 Mutants

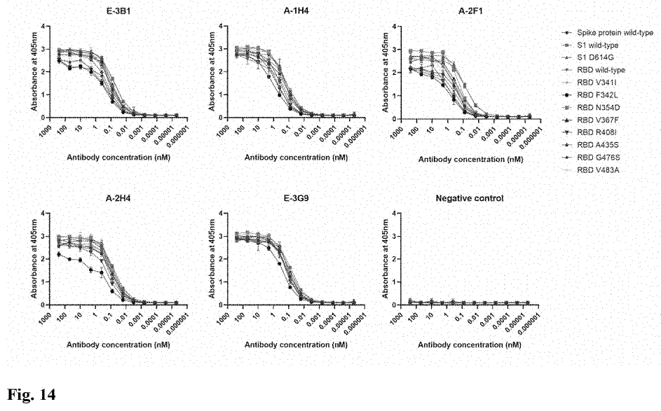

Because several mutations within the S1 have been identified along the course of the SARS-CoV-2 pandemic, worldwide (21), the probability of emerging escape mutants from the IGH repertoire induced by the wild-type virus infection was examined. The E-3B1, A-1H4, A-2F1, A-2H4, and E-3G9 nAbs successfully bound to recombinant mutant S1 proteins (V341I, F342L, N354D, V367F, R408I, A435S, G476S, V483A, and D614G) in a dose-dependent manner, with compatible reactivity against recombinant wild-type S1 and RBD protein (FIG. 14). Therefore, the human IGH immune repertoire may provide effective protection against most current SARS-CoV-2 mutants.

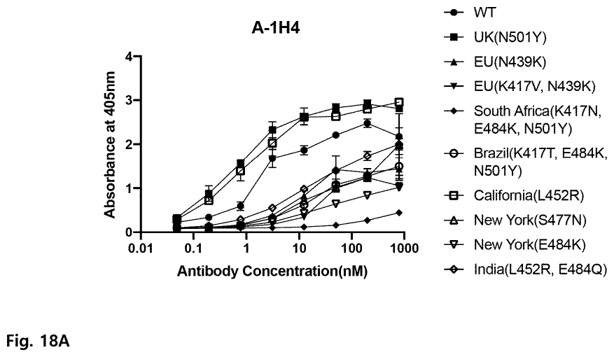

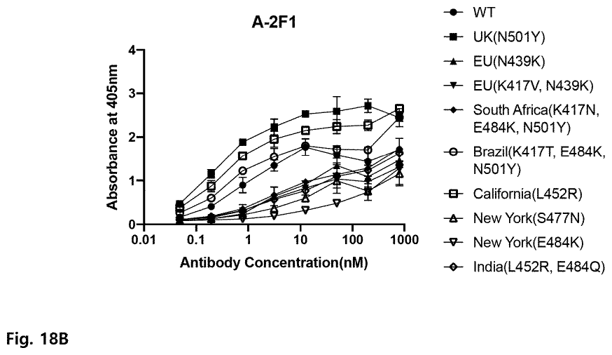

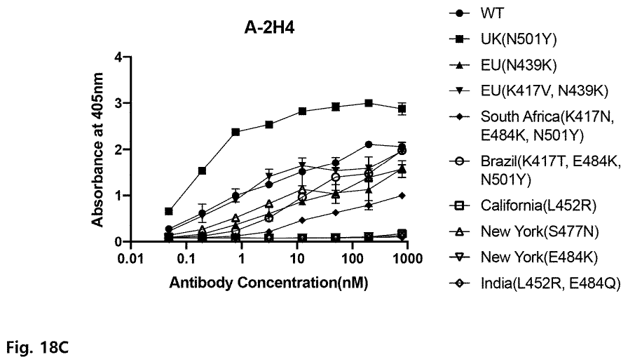

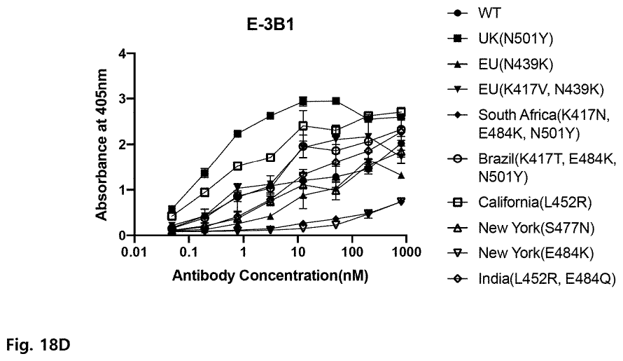

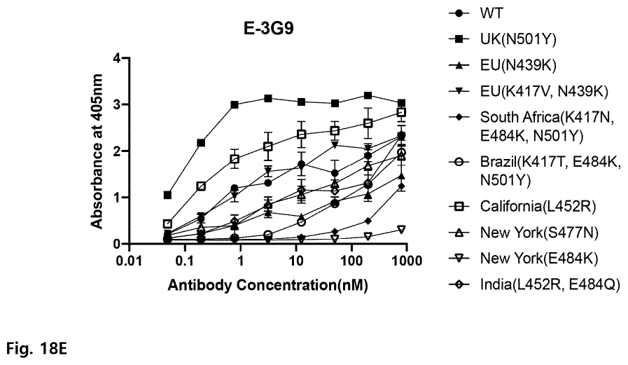

In addition, the ability of nABs to bind to receptor binding domain variants of the SARS-CoV-2 spike protein was also determined. The A-1H4, A-2F1, A-2H4, E-3B1, and E-3G9 antibodies bound to wild-type (WT) and 9 different variants of the SARS-CoV-2 spike protein RBD shown in the Table below in a dose dependent manner. See FIG. 18A-18E.

Table of SARS-CoV-2 RBD variants.

| | |
|---|---|
| WT | K417T, E484K, N501Y (Brazil) |
| N501Y (UK) | L452R (California) |
| N439K (Europe) | S477N (New York) |
| K417V, N439K (Europe) | E484K (New York) |
| K417N, E484K, N501Y (South Africa) | E484Q, L452R (India) |

### DISCUSSION

In response to SARS-CoV-2 infection, most human IGH repertoires efficiently generate clonotypes encoded by IGHV3-53/IGHV3-66 and IGHJ6, which can pair with diverse light chains, for both RBD binding and virus neutralization, with few to no somatic mutations. These clonotypes undergo swift class-switching to IgG1, IgA1, and even IgA2 subtypes. The expeditious development of these IGH clonotypes is possible because the naïve-stereotypic IGHV3-53/IGHV3-66 and IGHJ6 clonotypes pre-exist in the majority of the healthy population, predominantly as an IgM isotype. The data above show that IGHV3-53/IGHV3-66 and IGHJ6 are able to pair with diverse light chains to obtain reactivity to the RBD. It is expected that the extent of light chain plasticity is broad enough for virus-exposed people to successfully evolve nAbs because class-switched IGHV3-53/IGHV3-66 and IGHJ6 clonotypes were present in 13 of 17 patients from the current study.

Currently, it is not known whether the stereotypic nAbs are polyreactive or autoreactive. Rather, the selected stereotypic nAbs including A, B, G-42 do not cross-react with the recombinant RBD proteins of either SARS-CoV or MERS-CoV.

A possible correlation between clinical features and antibody response of 17 individuals who were infected with SARS-CoV-2 was analyzed. Of 17 laboratory-confirmed patients, two patients (Patients M and O) had a severe respiratory illness that required mechanical ventilation and six patients (Patients A, H, I, K, L, and P) with moderate illness required supplemental oxygenation. Together, these eight patients with relatively severe clinical courses had high titers of IgG antibody against SARS-CoV-2. However, some patients (Patients E, J, and Q) with mild/moderate symptoms also showed elevated titers of IgG antibody. Therefore, it is not clear whether antibody titer correlates with the clinical course of the patients.

In the humoral response to SAR-CoV-2, which elicits severe respiratory infection, it is beneficial for patients to produce both systemic and mucosal nAbs. The results presented herein showed that IGHV3-53/IGHV3-66 and IGHJ6 successfully class-switched to IgA1 in Patients G, I, and J, whereas they were class-switched to IgA1 and IgA2 in Patient E (Table 4). Furthermore, it deserves mention that after 99 days from the onset of symptoms, no RBD-reactive IGH clonotypes in the peripheral blood of Patient E were detected; however, the antibody titer to the RBD protein still remained high (FIG. 2D and FIG. 3). This observation is in line with the findings that nAb titers remained detectable among a fraction of SARS and MERS patients 1-2 years after infection (28, 29). Therefore, it can be inferred that nAb-producing plasmablast cells were mobilized from the peripheral blood and kept producing nAbs from within bone marrow niches in Patient E. In these niches, plasmablast cells are able to further differentiate into mature plasma cells and may survive for decades (30).

Meanwhile, in Patient A, only one of six nAbs was mapped to the IGH repertoire. It has been reported that the frequency of RBD-reactive B-cell clones is extremely low (0.07% to 0.005%) among circulating B cells (24). The frequency of isolated nAb clonotypes in the IGH repertoire was also extremely low (0.0004%-0.0064%) (FIG. 1B). As the complexity of scFv phage-display libraries exceeded 3.8×108 and 6.7×108 colony-forming units for Patient A, diverse RBD-binding clones could be enriched by biopanning. While only 199,561 unique IGH sequences were sampled by NGS in Patient A, S15,994 IGH sequences were obtained in Patient E at the sampling points when scFv phage-display libraries were constructed. This difference in NGS throughput might explain the discrepant allocation of nAb clonotypes in the IGH repertoire of Patients A and E. Consistent with this hypothesis, only 38.3% and 22.0% of “bind” and “predicted” clones were mapped for Patient A, respectively, while 77.8% and 32.1% of “bind” and “predicted” clones were individually mapped for Patient E.

In summary, it was found that stereotypic nAb clonotypes pre-existed in the majority of the naïve population, were prevalent among the patients who displayed rapid class-switching to IgG and IgA isotypes, and exhibited light chain plasticity among the SARS-CoV-2 RBD-binding antibodies. These results strongly suggest that stereotypic nAb clonotypes could contribute to the milder clinical course and lower mortality rate seen in patients with SARS-CoV-2 compared to patients with SARS-CoV (9.5%) or MERS-CoV (34.4%) (33) in which similar stereotypic nAb clonotypes have not been reported.

### Materials and Methods

### Study Design

To investigate stereotypic nAb clonotypes of SARS-CoV-2, 26 blood samples collected from 17 patients were subjected to NGS analysis of IG sequences. Human antibody libraries were prepared and subjected to biopanning against recombinant SARS-CoV-2 RBD proteins. RBD-binders were selected using ELISA and their neutralizing activity was tested using flow cytometry with ACE2-expressing cells and recombinant SARS-CoV-2 S protein and microneutralization assay. NGS analysis of the enrichment patterns of clones through biopanning was performed for in silico selection of RBD-binding clones. IG repertoire analyses were conducted to identify and characterize nAb clonotypes, including their prevalence among patients, frequency in IG repertoires, somatic mutations, isotypes, chronological changes, and existence in the naïve un-infected population.

### Human Samples

Three chronological blood samples were drawn from Patients A and E. From Patients B, C, D, F, and G, two chronological samples were obtained. Blood samples were collected once from Patients H-Q. All patients were confirmed to be infected by SARS-CoV-2 by a positive reverse transcriptase-quantitative polymerase chain reaction (RT-qPCR) result, and sample collection was performed at Seoul National University Hospital. PBMCs and plasma were isolated using Lymphoprep (Stemcell Technologies, Vancouver, BC, Canada), according to the manufacturer's protocol. The PBMCs were subjected to total RNA isolation, using the TRI Reagent (Invitrogen, Carlsbad, Calif., USA), according to the manufacturer's protocol. The study involving human sample collection was approved by the Institutional Ethics Review Board of Seoul National University Hospital (IRB approval number: 2004-230-1119).

### NGS

Genes encoding VH and part of the CH1 domain were amplified, using specific primers, as described previously (16, 34). All primers used are listed in Table 9. Briefly, total RNA was used as a template to synthesize cDNA, using the Superscript IV First-Strand Synthesis System (Invitrogen), with specific primers targeting the constant region (CH1 domain) of each isotype (IgM, IgD, IgG, IgA, and IgE) (34), according to the manufacturer's protocol. Following cDNA synthesis, 1.8 volumes of SPRI beads (AmpureXP, Beckman Coulter, Brea, Calif., USA) were used to purify cDNA, which was eluted in 40 μL water. The purified cDNA (18 μL) was subjected to second-strand synthesis in a 25-4 reaction volume, using V gene-specific primers (16) and KAPA Biosystems (KAPA HiFi HotStart, Roche, Basel, Switzerland). The PCR conditions were as follows: 95° C. for 3 min, 98° C. for 1 min, 55° C. for 1 min, and 72° C. for 5 min. Following the second-strand synthesis, double-strand DNA (dsDNA) was purified, using SPRI beads, as described above. VH genes were amplified using 15 μL eluted dsDNA and 2.5 pmol of the primers listed in Table 9, in a 50-μL total reaction volume (KAPA Biosystems), using the following thermal cycling program: 95° C. for 3 min; 17 cycles of 98° C. for 30 sec, 65° C. for 30 sec, and 72° C. for 1 min 10 sec; and 72° C. for 5 min. The number of PCR cycles was increased, from 17 to 19, for samples from Patients B (d10 and 19), C (d6), E (d23), and G (d9 and 22). PCR products were purified using SPRI beads and eluted in 30 μL water. Genes encoding Vκ and Vλ were amplified using specific primers, as described previously (20, 35). Briefly, total RNA was used as a template to synthesize cDNA, using the Superscript IV First-Strand Synthesis System (Invitrogen), with specific primers targeting the constant region, which are listed in Table 9, according to the manufacturer's protocol. Following cDNA synthesis, SPRI beads were used to purify cDNA, which was eluted in 40 μL water. Purified cDNA (18 μL) was used for the first amplification, in a 25-4 reaction volume, using VJ gene-specific primers, which are listed in Table 9, and KAPA Biosystems. The PCR conditions were as follows: 95° C. for 3 min, 4 cycles of 98° C. for 1 min, 55° C. for 1 min, and 72° C. for 1 min; and 72° C. for 10 min. Subsequently, DNA was purified using SPRI beads, and the Vκ and Vλ genes were amplified using 15 μL eluted dsDNA and 2.5 pmol of the primers listed in Table 9, in a 50-μL total reaction volume (KAPA Biosystems). The PCR conditions were as follows: 95° C. for 3 min; 17 cycles of 98° C. for 30 sec, 65° C. for 30 sec, and 72° C. for 1 min 10 sec; and 72° C. for 5 min. PCR products were purified using SPRI beads, as described above. For the amplification of VH from each round of biopanning (rounds 0-4), gene fragments were amplified from phagemid DNA, using the primers listed in Table 9. SPRI-purified sequencing libraries were quantified with a 4200 TapeStation System (Agilent Technologies), using a D1000 ScreenTape Assay, before performing sequencing on an Illumina MiSeq Platform.

### NGS Data Processing

### Pre-Processing of the NGS Data for the IG Repertoire

The raw NGS forward (R1) and reverse (R2) reads were merged by PEAR, v0.9.10, in default setting (36). The merged reads were q-filtered using the condition q20p95, which results in 95% of the base-pairs in a read having Phread scores higher than 20. The location of the primers was recognized from the q-filtered reads while allowing one substitution or deletion (Table 9). Then, primer regions that specifically bind to the molecules were trimmed in the reads, to eliminate the effects of primer synthesis errors. Based on the primer recognition results, unique molecular identifier (UMI) sequences were extracted, and the reads were clustered according to the UMI sequences. To eliminate the possibility that the same UMI sequences might be used for different read amplifications, the clustered reads were sub-clustered, according to the similarity of the reads (Five mismatches were allowed in each sub-cluster). The sub-clustered reads were aligned, using a multiple sequence alignment tool, Clustal Omega, v1.2.4, in default setting (37, 38). From the aligned reads, the frequency of each nucleotide was calculated, and a consensus sequence of each sub-cluster was defined using the frequency information. Then, the read count of the consensus sequence was re-defined as the number of UMI sub-clusters that belong to the consensus sequences.

### Sequence Annotation, Functionality Filtering, and Throughput Adjustment

Sequence annotation consisted of two parts, isotype annotation and VDJ annotation. For annotation, the consensus sequence was divided into two sections, a VDJ region and a constant region, in a location-based manner. For isotype annotation, the extracted constant region was aligned with the IMGT (international immunogenetics information system) constant gene database (39). Based on the alignment results, the isotypes of the consensus sequences were annotated. Then, the VDJ regions of the consensus sequences were annotated, using IgBLAST, v1.8.0 (40). Among the annotation results, V/D/J genes (V/J genes for VL), CDR1/2/3 sequences, and the number of mutations from the corresponding V genes were extracted, for further analysis. Divergence values were defined as the number of mutations identified in the aligned V gene, divided by the aligned length. Then, the non-functional consensus reads were defined using the following criteria and filtered-out: 1. sequence length shorter than 250 bp; 2. existence of stop-codon or frame-shift in the full amino acid sequence; 3. annotation failure in one or more of the CDR1/2/3 regions; and 4. isotype annotation failure. Then, the functional consensus reads were random-sampled, to adjust the throughput of the VH data (Table 6). Throughput adjustment was not conducted for VL data (Table 7).

### Pre-Processing of the Biopanning NGS Data

Pre-processing of the biopanning NGS data was performed as previously reported, except for the application of the q-filtering condition q20p95 instead of q20p100 (41).

### Overlapping IGH Repertoire Construction

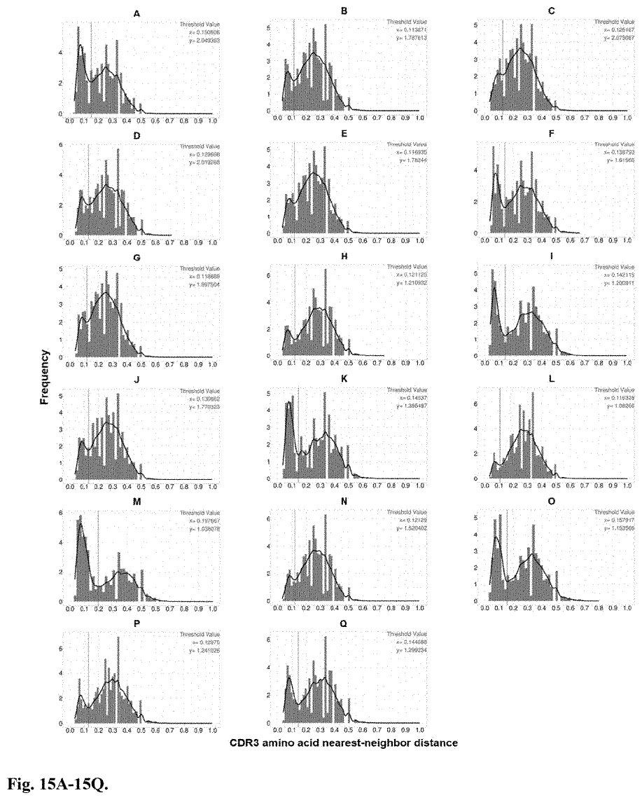

To investigate the shared IGH sequences among the patients, the overlapping IGH repertoire of the patients was defined. First, histograms for the nearest-neighbor distances of the HCDR3 amino acid sequences were calculated for the repertoire data. A hierarchical, distance-based analysis, which was reported previously (42), was applied to the HCDR3 amino acid sequences, to cluster functionally similar IGH sequences. The IGH sequences for all repertoire data could be approximated into a bimodal distribution, allowing the functionally similar IGH sequences to be extracted by capturing the first peak of the distribution (FIG. 15). Threshold values for each data set were defined as the nearest-neighbor distance value of those points with a minimum frequency between the two peaks of the distribution. Then, the minimum value among all threshold values, 0.113871, was used to construct the overlapping IGH repertoire, which means that 11.3871% of mismatches in the HCDR3 amino acid sequence were allowed in the overlapping IGH repertoire construction. To construct the overlapping IGH repertoire, the repertoire data sets of all patients were merged into one data set. The IGH sequences in the merged data set were then clustered, using the following conditions: 1. the same V and J gene usage; and 2. mismatch smaller than 11.3871% among the HCDR3 amino acid sequences. Subsequently, clusters containing IGH sequences from more than one patient were included in the overlapping IGH repertoire data set.

### Extraction of Binding-Predicted Clones

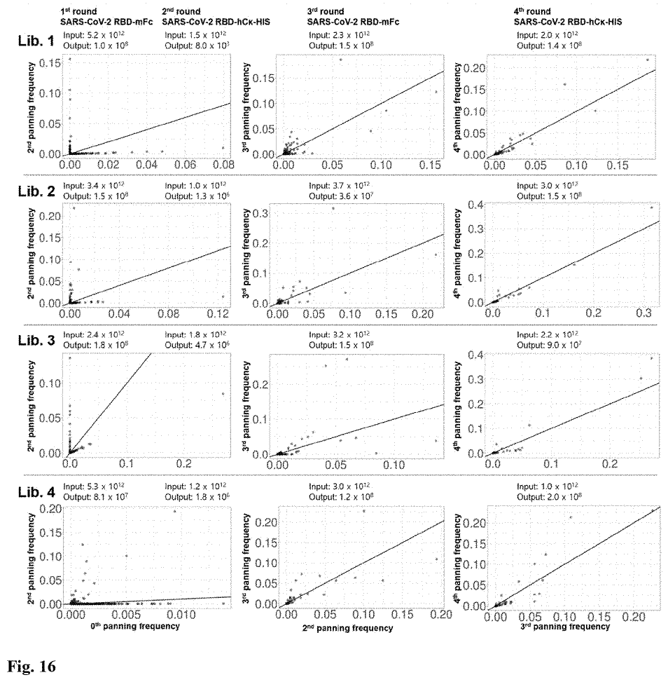

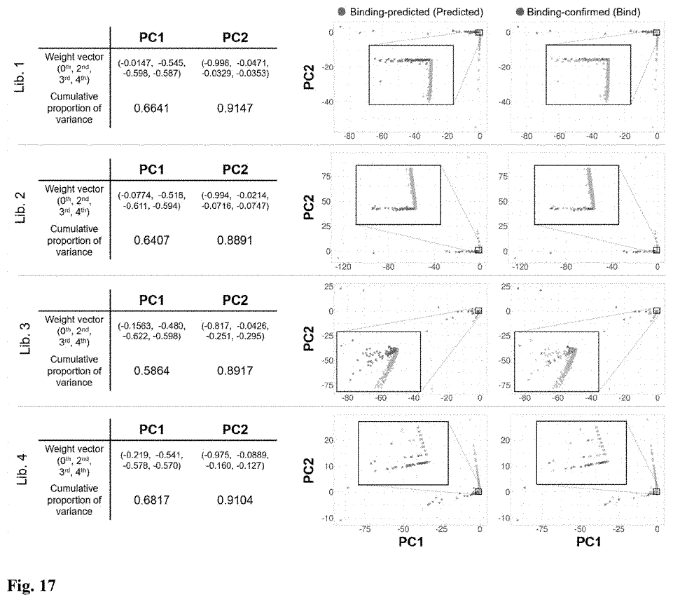

From each round of biopanning (rounds 0, 2, 3, and 4), the VH genes were amplified and subjected to NGS analysis, using the MiSeq platform, as described previously (19). Binding-predicted clones from biopanning were defined by employing frequency the values of the NGS data from four libraries, A_d17, A_d45, E_d23, and E_d44, at each round of biopanning. The enrichment of clones primarily occurred during the second round of biopanning, based on the input/output virus titer values for each round of biopanning and the frequencies of the clones in the NGS data (FIG. 16). Then, the frequency information in the NGS data sets for biopanning rounds 0, 2, 3, and 4 was subject to principal component analysis (PCA), for dimension reduction. Accordingly, principal component (PC)1 and PC2, which represented clone enrichment and clone depletion, respectively, were extracted. In the biopanning data, PC1 was primarily composed of the frequencies in rounds 2, 3, and 4, whereas PC2 was primarily composed of the frequency in round 0 (FIG. 17). Thus, PC1-major clones were defined as the predicted clones, by setting constant threshold values on the PC1 value and the ratio between PC1 and PC2 (Table 8). Subsequently, 94.74% of the RBD-binding clones were successfully mapped to the predicted clones (FIG. 17).

Construction of a Human scFv Phage-Display Library and VL Shuffled Libraries

For the VH gene, the cDNA prepared for the NGS analysis was used. For the Vκ and Vλ genes, total RNA was used to synthesize cDNA, using the Superscript IV First-Strand Synthesis System (Invitrogen), with oligo(dT) primers, according to the manufacturer's instructions. Then, the genes encoding VK/Vλ and VH were amplified, from the oligo(dT)-synthesized cDNA and the cDNA prepared for NGS analysis, respectively, using the primers listed in Table 9 and KAPA Biosystems. The PCR conditions were as follows: preliminary denaturation at 95° C. for 3 min; 4 cycles of 98° C. for 1 min, 55° C. for 1 min, and 72° C. for 1 min; and 72° C. for 10 min. Subsequently, DNA was purified using SPRI beads, as described above. The purified DNA was amplified using the primers listed in Table 9 and KAPA Biosystems. The PCR conditions were as follows: preliminary denaturation, at 95° C. for 3 min; 25 cycles of 98° C. for 30 sec, 58° C. for 30 sec, and 72° C. for 90 sec; and 72° C. for 10 min. Then, the VH and VK/Vλ fragments were subjected to electrophoresis, on a 1% agarose gel, and purified, using a QIAquick Gel Extraction Kit (Qiagen Inc., Valencia, Calif., USA), according to the manufacturer's instructions. The purified VH and VK/Vλ fragments were mixed, at equal ratios at 50 ng, and subjected to overlap extension, to generate scFv genes, using the primers listed in Table 9 and KAPA Biosystems. The PCR conditions were as follows: preliminary denaturation, at 94° C. for 5 min; 25 cycles of 98° C. for 15 sec, 56° C. for 15 sec, and 72° C. for 2 min; and 72° C. for 10 min. The amplified scFv fragment was purified and cloned into a phagemid vector, as described previously (43).

For the construction of VK/Vλ shuffled libraries, gBlocks Gene Fragments (Integrated DNA Technologies, Coralville, Iowa, USA), encoding A-11, E-12, A-31, A-32, B-33, E-34, A,B,G-42, G-44, D-51, F-53, E-52, and A-54, were synthesized. Synthesized VH and the VK/Vλ genes from Patients A, E, and G were used to synthesize the scFv libraries using PCR, as described previously (43). Then, the amplified scFv fragments were purified and cloned into the phagemid vector, as described above.

### Biopanning

Phage display of the human scFv libraries exceeded complexity of 3.8×108, 6.7×108, 2.0×108, and 7.2×108 colony-forming units for A_d17, A_d45, E_d23, and E_d44, respectively. These libraries were subjected to four rounds of biopanning against the recombinant SARS-CoV-2 RBD protein (Sino Biological Inc., Beijing, China), fused to mFc or hCκ, as described previously (44). Briefly, 3 μg of the recombinant SARS-CoV-2 RBD protein was conjugated to 1.0×107 magnetic beads (Dynabeads M-270 epoxy, Invitrogen) and incubated with the scFv phage-display libraries (approximately 1012 phages), for 2 h at 37° C. During the first round of biopanning, the beads were washed once with 500 μL of 0.05% (v/v) Tween-20 (Sigma-Aldrich, St. Louis, Mo., USA) in phosphate-buffered saline (PBST). For the other rounds of biopanning, 1.5 μg of recombinant SARS-CoV-2 RBD protein was conjugated to 5.0×106 magnetic beads, and the number of washes was increased to three. After each round of biopanning, the bound phages were eluted and rescued, as described previously (44).

### Phage ELISA

To select SARS-CoV-2 S reactive clones, phage ELISA was performed, using recombinant S and RBD protein-coated microtiter plates, as described previously (45). Reactive scFv clones were subjected to Sanger sequencing (Cosmogenetech, Seoul, Republic of Korea), to determine their nucleotide sequences.

### Expression of Recombinant Proteins

A human, codon-optimized, SARS-CoV-2 RBD (YP 009724390.1, amino acids 306-543) gene was synthesized (Integrated DNA Technologies). Using a synthesized wild-type RBD gene as a template, RBD mutants (V341I, F342L, N354D, N354D/D364Y, V367F, R408I, A435S, W436R, G476S, and V483A) were generated through two-step PCR, using the primers listed in Table 9. The genes encoding wild-type or mutant SARS-CoV-2 RBD were cloned into a modified mammalian expression vector, containing the hCκ gene (44), and transfected into Expi293F (Invitrogen) cells. The fusion proteins were purified by affinity chromatography, using KappaSelect Columns (GE Healthcare, Chicago, Ill., USA), as described previously (46). Due to low expression yields, two RBD mutants (N354D/D364Y, W436R) were excluded from further studies.

The genes encoding the selected scFv clones were cloned into a modified mammalian expression vector, containing the hIgG1 Fc regions (hFc) or hCκ at the C-terminus (44, 47), before being transfected and purified by affinity chromatography, as described above.

Genes encoding VH and VL were amplified, cloned into a mammalian expression vector containing the CH1 and hinge regions of human IgG2 fused to the CH2 and CH3 regions of human IgG4 (48, 49), and transfected into Expi293F cells (Invitrogen) as described previously (50). Then, IgG2/4 was purified by affinity chromatography using MabSelect columns with the AKTA Pure chromatography system (GE Healthcare) following the manufacturer's protocol.

### ELISA

First, 100 ng of each recombinant SARS-CoV-2 S (Sino Biological Inc.), S1 (Sino Biological Inc.), S1 D614G (Sino Biological Inc.), S2 (Sino Biological Inc.), NP (Sino Biological Inc.), RBD, RBD mutants, SARS-CoV RBD (Sino Biological Inc.), MERS-CoV S (Sino Biological Inc.), RBD (Sino Biological Inc.), S2 (Sino Biological Inc.) proteins were added to microtiter plates (Costar), in coating buffer (0.1 M sodium bicarbonate, pH 8.6). After incubation at 4° C., overnight, and blocking with 3% bovine serum albumin (BSA) in PBS, for 1 h at 37° C., serially diluted plasma (5-fold, 6 dilutions, starting from 1:100) or scFv-hFc (5-fold, 12 dilutions, starting from 1,000 or 500 nM) in blocking buffer was added to individual wells and incubated for 1, h at 37° C. Then, the plates were washed three times with 0.05% PBST. Horseradish peroxidase (HRP)-conjugated rabbit anti-human IgG antibody (Invitrogen) or anti-human Ig kappa light chain antibody (Millipore, Temecula, Calif., USA), in blocking buffer (1:5,000), was added into wells and incubated for 1 h at 37° C. After washing three times with PBST, 2,2′-azino-bis-3-ethylbenzothiazoline-6-sulfonic (ThermoFisher Scientific Inc., Waltham, Mass., USA) or 3,3′,5,5′-Tetramethylbenzidine liquid substrate system (ThermoFisher Scientific Inc.) was added to the wells. Absorbance was measured at 405 nm or 650 nm, using a microplate spectrophotometer (Multiskan GO; Thermo Scientific).

### Flow Cytometry

The recombinant SARS-CoV-2 S protein (200 nM), fused with a HIS-tag at the C-terminus (Sino Biological Inc.), was incubated with scFv-hFc fusion proteins at a final concentration of either 200 nM (equimolar) or 600 nM (molar ratio of 1:3), in 50 μL of 1% (w/v) BSA in PBS, containing 0.02% (w/v) sodium azide (FACS buffer), at 37° C. for 1 h. Irrelevant scFv-hFc or scFv-hCκ fusion proteins were used as negative controls. Vero E6 cells (ACE2+) were seeded into v-bottom 96-well plates (Corning, Corning, N.Y., USA), at a density of 1.5×105 cells per well. Then, the mixture was added to each well and incubated, at 37° C. for 1 h. After washing three times with FACS buffer, FITC-labeled rabbit anti-HIS Ab (Abcam, Cambridge, UK) was incubated, at 37° C. for 1 h. Then, the cells were washed three times with FACS buffer, resuspended in 150 μL of PBS, and subjected to analysis by flow cytometry, using a FACS Canto II instrument (BD Bioscience, San Jose, Calif., USA). For each sample, 10,000 cells were assessed.

### Microneutralization Assay

The virus (BetaCoV/Korea/SNU01/2020, accession number MT039890) was isolated at the Seoul National University Hospital and propagated in Vero cells (ATCC CCL-81), using Dulbecco's Modified Eagle's Medium (DMEM, Welgene, Gyeongsan, Republic of Korea) supplemented with 2% fetal bovine serum (Gibco) (51). The cells were grown in T-25 flasks, (ThermoFisher Scientific Inc.), inoculated with SARS-CoV-2, and incubated at 37° C., in a 5% CO2 environment. Then, 3 days after inoculation, the viruses were harvested and stored at −80° C. The virus titer was determined via a TCID50 assay (52).

Vero cells were seeded in T-25 flasks and grown for 24 h, at 37° C., in a 5% CO2 environment, to ensure 80% confluency on the day of inoculation. The recombinant scFv-hCκ fusion proteins (0.5, 5, or 50 μg/mL) were mixed with 2,500 TCID50 of SARS-CoV-2, and the mixture was incubated for 2 h, at 37° C. Then, the mixture (1 mL) was added to the Vero cells and incubated for 1 h, at 37° C., in a 5% CO2 environment. After incubation for 1 h, 6 mL of complete media was added to the flasks and incubated, at 37° C., in a 5% CO2 environment. After 0, 24, 48, and 72 h of infection, the culture supernatant was collected, to measure the virus titers. RNA was extracted, using the MagNA Pure 96 DNA and Viral NA small volume kit (Roche, Germany), according to the manufacturer's instructions. Viral RNA was detected using the PowerChek 2019-nCoV Real-time PCR Kit (Kogene Biotech, Seoul, Republic of Korea), for the amplification of the E gene, and quantified according to a standard curve, which was constructed using in vitro transcribed RNA, provided by the European Virus Archive (https://www.european-virus-archive.com). Another neutralization assay was performed as described previously (53). Briefly, Vero cells seeded in 96-well plates in DMEM medium were grown for 24 h at 37° C. in a 5% CO2 environment. 50 μl of two-fold serially diluted IgG2/4 were mixed with an equal volume of SARS-CoV-2 containing 100 TCID50 and the IgG2/4-virus mixture was incubated at 37° C. for 1 h. The mixture was then transferred into a 96-well microtiter plate containing Vero cells with 8 repeats and incubated for 5 days at 37° C. in a 5% CO2 environment. Cells infected with 100 TCID50 of SARS-CoV-2, isotype IgG2/4 control, or without the virus, were applied as positive, negative, and uninfected controls, respectively. The cytopathic effect (CPE) in each well was observed 5 days post-infection. The IC50 was calculated using GraphPad Prism 8 (GraphPad Software, San Diego, Calif., USA). All experiments using authentic SARS-CoV-2 were conducted in Biosafety Level 3 laboratory.

### Statistical Analyses

Data are represented as mean±standard deviation. Statistical analyses were performed using R software v.3.4.3. For the flow cytometry analysis using ACE2-expressing cells and recombinant SARS-CoV-2 S protein, results were analyzed by independent t-tests.

### REFERENCES AND NOTES

1. J. G. Jardine, T. Ota, D. Sok, M. Pauthner, D. W. Kulp, O. Kalyuzhniy, P. D. Skog, T. C. Thinnes, D. Bhullar, B. Briney, S. Menis, M. Jones, M. Kubitz, S. Spencer, Y. Adachi, D. R. Burton, W. R. Schief, D. Nemazee, HIV-1 VACCINES. Priming a broadly neutralizing antibody response to HIV-1 using a germline-targeting immunogen. Science 349, 156-161 (2015).
2. J. R. Willis, J. A. Finn, B. Briney, G. Sapparapu, V. Singh, H. King, C. C. LaBranche, D. C. Montefiori, J. Meiler, J. E. Crowe, Jr., Long antibody HCDR3s from HIV-naïve donors presented on a PG9 neutralizing antibody background mediate HIV neutralization Proc Natl Acad Sci USA 113, 4446-4451 (2016).
3. N. Eroshenko, T. Gill, M. K. Keaveney, G. M. Church, J. M. Trevejo, H. Rajaniemi, Implications of antibody-dependent enhancement of infection for SARS-CoV-2 countermeasures. Nat Biotechnol, (2020).
4. A. Iwasaki, Y. Yang, The potential danger of suboptimal antibody responses in COVID-19. Nat Rev Immunol, (2020).
5. M. K. Smatti, A. A. Al Thani, H. M. Yassine, Viral-Induced Enhanced Disease Illness. Front Microbiol 9, 2991 (2018).
6. A. Zhang, H. D. Stacey, C. E. Mullarkey, M. S. Miller, Original Antigenic Sin: How First Exposure Shapes Lifelong Anti-Influenza Virus Immune Responses. J Immunol 202, 335-340 (2019).
7. Y. Cao, B. Su, X. Guo, W. Sun, Y. Deng, L. Bao, Q. Zhu, X. Zhang, Y. Zheng, C. Geng, X. Chai, R. He, X. Li, Q. Lv, H. Zhu, W. Deng, Y. Xu, Y. Wang, L. Qiao, Y. Tan, L. Song, G. Wang, X. Du, N. Gao, J. Liu, J. Xiao, X. D. Su, Z. Du, Y. Feng, C. Qin, C. Qin, R. Jin, X. S. Xie, Potent neutralizing antibodies against SARS-CoV-2 identified by high-throughput single-cell sequencing of convalescent patients' B cells. Cell, (2020).
8. B. Ju, Q. Zhang, J. Ge, R. Wang, J. Sun, X. Ge, J. Yu, S. Shan, B. Zhou, S. Song, X. Tang, J. Yu, J. Lan, J. Yuan, H. Wang, J. Zhao, S. Zhang, Y. Wang, X. Shi, L. Liu, J. Zhao, X. Wang, Z. Zhang, L. Zhang, Human neutralizing antibodies elicited by SARS-CoV-2 infection. Nature, (2020).
9. T. F. Rogers, F. Zhao, D. Huang, N. Beutler, A. Burns, W. T. He, O. Limbo, C. Smith, G. Song, J.
Woehl, L. Yang, R. K. Abbott, S. Callaghan, E Garcia, J. Hurtado, M. Parren, L. Peng, S. Ramirez, J. Ricketts, M. J. Ricciardi, S. A. Rawlings, N. C. Wu, M. Yuan, D. M. Smith, D. Nemazee, J. R. Teijaro, J. E. Voss, I. A. Wilson, R. Andrabi, B. Briney, E. Landais, D. Sok, J. G. Jardine, D. R. Burton, Isolation of potent SARS-CoV-2 neutralizing antibodies and protection from disease in a small animal model. Science 369, 956-963 (2020).
10. R. Shi, C. Shan, X. Duan, Z. Chen, P. Liu, J. Song, T. Song, X. Bi, C. Han, L. Wu, G. Gao, X. Hu, Y. Zhang, Z. Tong, W. Huang, W. J. Liu, G. Wu, B. Zhang, L. Wang, J. Qi, H. Feng, F. S. Wang, Q. Wang, G. F. Gao, Z. Yuan, J. Yan, A human neutralizing antibody targets the receptor binding site of SARS-CoV-2. Nature, (2020).
11. Y. Wu, F. Wang, C. Shen, W. Peng, D. Li, C. Zhao, Z. Li, S. Li, Y. Bi, Y. Yang, Y. Gong, H. Xiao, Z. Fan, S. Tan, G. Wu, W. Tan, X. Lu, C. Fan, Q. Wang, Y. Liu, C. Zhang, J. Qi, G. F. Gao, F. Gao, L. Liu, A noncompeting pair of human neutralizing antibodies block COVID-19 virus binding to its receptor ACE2. Science, (2020).
12. M. Yuan, H. Liu, N. C. Wu, C. D. Lee, X. Zhu, F. Zhao, D. Huang, W. Yu, Y. Hua, H. Tien, T. F. Rogers, E. Landais, D. Sok, J. G. Jardine, D. R. Burton, I. A. Wilson, Structural basis of a shared antibody response to SARS-CoV-2. Science, (2020).
13. D. Wrapp, N. Wang, K. S. Corbett, J. A. Goldsmith, C. L. Hsieh, O. Abiona, B. S. Graham, J S McLellan, Cryo-EM structure of the 2019-nCoV spike in the prefusion conformation. Science 367, 1260-1263 (2020).
14. Q. Wang, Y. Zhang, L. Wu, S. Niu, C. Song, Z. Zhang, G. Lu, C. Qiao, Y. Hu, K. Y. Yuen, Q. Wang, H. Zhou, J. Yan, J. Qi, Structural and Functional Basis of SARS-CoV-2 Entry by Using Human ACE2. Cell 181, 894-904 e899 (2020).
15. P. Zhou, X. L. Yang, X. G. Wang, B. Hu, L. Zhang, W. Zhang, H. R. Si, Y. Zhu, B. Li, C. L. Huang, H. D. Chen, J. Chen, Y. Luo, H. Guo, R. D. Jiang, M. Q. Liu, Y. Chen, X. R. Shen, X. Wang, X. S. Zheng, K. Zhao, Q. J. Chen, F. Deng, L. L. Liu, B. Yan, F. X. Zhan, Y. Y. Wang, G. F. Xiao, Z. L. Shi, A pneumonia outbreak associated with a new coronavirus of probable bat origin. Nature 579, 270-273 (2020).
16. B. Briney, A. Inderbitzin, C. Joyce, D. R. Burton, Commonality despite exceptional diversity in the baseline human antibody repertoire. Nature 566, 393-397 (2019).
17. S. I. Kim, S. Kim, J. Kim, S. Y. Chang, J. M. Shim, J. Jin, C. Lim, S. Baek, J. Y. Min, W. B. Park, M. D. Oh, S. Kim, J. Chung, Generation of a Nebulizable CDR-Modified MERS-CoV Neutralizing Human Antibody. Int J Mot Sci 20, (2019).
18. W. Yang, A. Yoon, S. Lee, S. Kim, J. Han, J. Chung, Next-generation sequencing enables the discovery of more diverse positive clones from a phage-displayed antibody library. Exp Mol Med 49, e308 (2017).
19. D. K. Yoo, S. R. Lee, Y. Jung, H. Han, H. K. Lee, J. Han, S. Kim, J. Chae, T. Ryu, J. Chung, Machine Learning-Guided Prediction of Antigen-Reactive In Silico Clonotypes Based on Changes in Clonal Abundance through Bio-Panning. Biomolecules 10, (2020).
20. C. Soto, R. G. Bombardi, A. Branchizio, N. Kose, P. Matta, A. M. Sevy, R. S. Sinkovits, P. Gilchuk, J. A. Finn, J. E. Crowe, Jr., High frequency of shared clonotypes in human B cell receptor repertoires. Nature 566, 398-402 (2019).
21. J. Ou, Z. Zhou, R. Dai, J. Zhang, W. Lan, S. Zhao, J. Wu, D. Seto, L. Cui, G. Zhang, Q. Zhang, Emergence of RBD mutations in circulating SARS-CoV-2 strains enhancing the structural stability and human ACE2 receptor affinity of the spike protein. bioRxiv, 2020.2003.2015.991844 (2020).
22. J. Kreye, S. M. Reincke, H.-C. Kornau, E. Sanchez-Sendin, V. M. Corman, H. Liu, M. Yuan, N. C. Wu, X. Zhu, C.-C. D. Lee, J. Trimpert, M. Holtje, K. Dietert, L. Stoffler, N. von Wardenburg, S. van Hoof, M. A. Homeyer, J. Hoffmann, A Abdelgawad, A. D. Gruber, L. D. Bertzbach, D Vladimirova, L. Y. Li, P. C. Barthel, K. Skriner, A. C. Hocke, S. Hippenstiel, M. Witzenrath, N. Suttorp, F. Kurth, C. Franke, M. Endres, D. Schmitz, L. M. Jeworowski, A. Richter, M. L. Schmidt, T. Schwarz, M. A. Muller, C. Drosten, D. Wendisch, L. E. Sander, N. Osterrieder, I. A. Wilson, H. Priiss, A SARS-CoV-2 neutralizing antibody protects from lung pathology in a COVID-19 hamster model. bioRxiv, 2020.2008.2015.252320 (2020).
23. C. Kreer, M. Zehner, T. Weber, M. S. Ercanoglu, L. Gieselmann, C Rohde, S. Halwe, M. Korenkov, P. Schommers, K. Vanshylla, V. Di Cristanziano, H. Janicki, R. Brinker, A. Ashurov, V. Krahling, A. Kupke, H. Cohen-Dvashi, M. Koch, J. M. Eckert, S. Lederer, N. Pfeifer, T. Wolf, M. Vehreschild, C. Wendtner, R. Diskin, H. Gruell, S. Becker, F. Klein, Longitudinal Isolation of Potent Near-Germline SARS-CoV-2-Neutralizing Antibodies from COVID-19 Patients. Cell 182, 843-854 e812 (2020).
24. D. F. Robbiani, C. Gaebler, F. Muecksch, J. C. C. Lorenzi, Z. Wang, A. Cho, M. Agudelo, C. O. Barnes, A. Gazumyan, S. Finkin, T Hagglof, T. Y. Oliveira, C. Viant, A. Hurley, H. H. Hoffmann, K G. Millard, R. G. Kost, M. Cipolla, K. Gordon, F. Bianchini, S. T. Chen, V. Ramos, R. Patel, J. Dizon, I. Shimeliovich, P. Mendoza, H. Hartweger, L. Nogueira, M. Pack, J. Horowitz, F. Schmidt, Y. Weisblum, E. Michailidis, A. W. Ashbrook, E. Waltari, J. E. Pak, K. E. Huey-Tubman, N. Koranda, P. R. Hoffman, A. P. West, Jr., C. M. Rice, T. Hatziioannou, P. J. Bjorkman, P D Bieniasz, M. Caskey, M. C. Nussenzweig, Convergent antibody responses to SARS-CoV-2 in convalescent individuals. Nature 584, 437-442 (2020).
25. J. Zhao, Q. Yuan, H. Wang, W. Liu, X. Liao, Y. Su, X. Wang, J. Yuan, T. Li, J. Li, S. Qian, C. Hong, F. Wang, Y. Liu, Z. Wang, Q. He, Z. Li, B. He, T. Zhang, Y. Fu, S. Ge, L. Liu, J. Zhang, N. Xia, Z. Zhang, Antibody responses to SARS-CoV-2 in patients of novel coronavirus disease 2019. Clin Infect Dis, (2020).
26. W. H. Kong, R. Zhao, J. B. Zhou, F. Wang, D. G. Kong, J. B. Sun, Q. F. Ruan, M. Q. Liu, Serologic Response to SARS-CoV-2 in COVID-19 Patients with Different Severity. Virol Sin, (2020).
27. K. K. To, 0. T. Tsang, W. S. Leung, A. R. Tam, T. C. Wu, D. C. Lung, C. C. Yip, J. P. Cai, J. M. Chan, T. S. Chik, D. P. Lau, C. Y. Choi, L. L. Chen, W. M. Chan, K. H. Chan, J. D. Ip, A. C. Ng, R. W. Poon, C. T. Luo, V. C. Cheng, J. F. Chan, I. F. Hung, Z. Chen, H. Chen, K. Y. Yuen, Temporal profiles of viral load in posterior oropharyngeal saliva samples and serum antibody responses during infection by SARS-CoV-2: an observational cohort study. Lancet Infect Dis 20, 565-574 (2020).
28. P. G. Choe, R. Perera, W. B. Park, K. H. Song, J. H. Bang, E. S. Kim, H. B. Kim, L. W. R. Ko, S. W. Park, N. J. Kim, E. H. Y. Lau, L. L. M. Poon, M. Peiris, M. D. Oh, MERS-CoV Antibody Responses 1 Year after Symptom Onset, South Korea, 2015. Emerg Infect Dis 23, 1079-1084 (2017).
29. L. P. Wu, N. C. Wang, Y. H. Chang, X. Y. Tian, D. Y. Na, L. Y. Zhang, L. Zheng, T. Lan, L. F. Wang, G. D. Liang, Duration of antibody responses after severe acute respiratory syndrome. Emerg Infect Dis 13, 1562-1564 (2007).
30. D. Tarlinton, A. Radbruch, F. Hiepe, T. Dormer, Plasma cell differentiation and survival. Curr Opin Immunol 20, 162-169 (2008).
31. W. Deng, L. Bao, J. Liu, C. Xiao, J. Liu, J. Xue, Q. Lv, F. Qi, H. Gao, P. Yu, Y. Xu, Y. Qu, F. Li, Z. Xiang, H. Yu, S. Gong, M. Liu, G. Wang, S. Wang, Z. Song, Y. Liu, W. Zhao, Y. Han, L. Zhao, X. Liu, Q. Wei, C. Qin, Primary exposure to SARS-CoV-2 protects against reinfection in rhesus macaques. Science, (2020).
32. R. a. S. TILLETT, JOEL and HARTLEY, PAUL and KERWIN, HEATHER and CRAWFORD, NATALIE and GORZALSKI, ANDREW and LAVERDURE, CHRISTOPHER and VERMA, SUBHASH and ROSSETTO, CYPRIAN and FARRELL, MEGAN and JACKSON, DAVID and Pandori, Mark and VAN HOOSER, STEPHANIE, Genomic Evidence for a Case of Reinfection with SARS-CoV-2.
33. V. J. Munster, M. Koopmans, N. van Doremalen, D. van Riel, E. de Wit, A Novel Coronavirus Emerging in China—Key Questions for Impact Assessment. N Engl J Med 382, 692-694 (2020).
34. F. Horns, C. Vollmers, D. Croote, S. F. Mackey, G. E. Swan, C. L. Dekker, M. M. Davis, S. R. Quake, Lineage tracing of human B cells reveals the in vivo landscape of human antibody class switching. Elife 5, (2016).
35. H. Wardemann, S Yurasov, A. Schaefer, J. W. Young, E. Meffre, M. C. Nussenzweig, Predominant autoantibody production by early human B cell precursors. Science 301, 1374-1377 (2003).
36. J. Zhang, K. Kobert, T. Flouri, A. Stamatakis, PEAR: a fast and accurate Illumina Paired-End reAd mergeR. Bioinformatics 30, 614-620 (2014).
37. F. Sievers, D. G. Higgins, Clustal Omega for making accurate alignments of many protein sequences. Protein Sci 27, 135-145 (2018).
38. F. Sievers, A. Wilm, D. Dineen, T. J. Gibson, K. Karplus, W. Li, R. Lopez, H. McWilliam, M Remmert, J. Soding, J. D. Thompson, D. G. Higgins, Fast, scalable generation of high-quality protein multiple sequence alignments using Clustal Omega. Mol Syst Biol 7, 539 (2011).
39. M. P. Lefranc, IMGT databases, web resources and tools for immunoglobulin and T cell receptor sequence analysis, http://imgt.cines.fr. Leukemia 17, 260-266 (2003).
40. J. Ye, N. Ma, T. L. Madden, J. M. Ostell, IgBLAST: an immunoglobulin variable domain sequence analysis tool. Nucleic Acids Res 41, W34-40 (2013).
41. S. Kim, H. Lee, J. Noh, Y. Lee, H. Han, D. K. Yoo, H. Kim, S. Kwon, J. Chung, Efficient Selection of Antibodies Reactive to Homologous Epitopes on Human and Mouse Hepatocyte Growth Factors by Next-Generation Sequencing-Based Analysis of the B Cell Repertoire. Int J Mol Sci 20, (2019).
42. N. T. Gupta, K. D. Adams, A. W. Briggs, S. C. Timberlake, F. Vigneault, S. H. Kleinstein, Hierarchical Clustering Can Identify B Cell Clones with High Confidence in Ig Repertoire Sequencing Data. J Immunol 198, 2489-2499 (2017).
43. J. Andris-Widhopf, P. Steinberger, R. Fuller, C. Rader, C. F. Barbas, 3rd, Generation of human scFv antibody libraries: PCR amplification and assembly of light- and heavy-chain coding sequences. Cold Spring Harb Protoc 2011, (2011).
44. Y. Lee, H. Kim, J. Chung, An antibody reactive to the Gly63-Lys68 epitope of NT-proBNP exhibits 0-glycosylation-independent binding. Exp Mol Med 46, e114 (2014).
45. D. R. B. Carlos F. Barbas III, Jamie K. Scott, Gregg J. Silverman, Phage Display: A Laboratory Manual. Cold Spring Harbor Laboratory Press 76, 487-488 (2001).
46. S. Lee, I. H. Yoon, A. Yoon, J. M. Cook-Mills, C. G. Park, J. Chung, An antibody to the sixth Ig-like domain of VCAM-1 inhibits leukocyte transendothelial migration without affecting adhesion. J Immunol 189, 4592-4601 (2012).
47. S. Park, D. H. Lee, J. G. Park, Y. T. Lee, J. Chung, A sensitive enzyme immunoassay for measuring cotinine in passive smokers. Clin Chun Acta 411, 1238-1242 (2010).
48. J. P. Mueller, M. A. Giannoni, S. L. Hartman, E A Elliott, S. P. Squinto, L. A. Matis, M. J. Evans, Humanized porcine VCAM-specific monoclonal antibodies with chimeric IgG2/G4 constant regions block human leukocyte binding to porcine endothelial cells. Mol Immunol 34, 441-452 (1997).
49. R. P. Rother, S. A. Rollins, C. F. Mojcik, R. A. Brodsky, L. Bell, Discovery and development of the complement inhibitor eculizumab for the treatment of paroxysmal nocturnal hemoglobinuria. Nat Biotechnol 25, 1256-1264 (2007).
50. J. Jin, G. Park, J. B. Park, S. Kim, H. Kim, J. Chung, An anti-EGFR x cotinine bispecific antibody complexed with cotinine-conjugated duocarmycin inhibits growth of EGFR-positive cancer cells with KRAS mutations. Exp Mol Med 50, 1-14 (2018).
51. W. B. Park, N. J. Kwon, S. J. Choi, C. K. Kang, P. G. Choe, J. Y. Kim, J. Yun, G. W. Lee, M. W. Seong, N. J. Kim, J. S. Seo, M. D. Oh, Virus Isolation from the First Patient with SARS-CoV-2 in Korea. J Korean Med Sci 35, e84 (2020).
52. L. J. REED, H. MUENCH, A SIMPLE METHOD OF ESTIMATING FIFTY PERCENT ENDPOINTS12. American Journal of Epidemiology 27, 493-497 (1938).
53. R. Shi, C. Shan, X. Duan, Z. Chen, P. Liu, J. Song, T. Song, X. Bi, C. Han, L. Wu, G. Gao, X. Hu, Y. Zhang, Z. Tong, W. Huang, W. J. Liu, G. Wu, B. Zhang, L. Wang, J. Qi, H. Feng, F. S. Wang, Q. Wang, G. F. Gao, Z. Yuan, J. Yan, A human neutralizing antibody targets the receptor-binding site of SARS-CoV-2. Nature 584, 120-124 (2020).

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

TABLE 1
The stereotypic V_{H} clonotypes against SARS-CoV-2 RBD in the healthy population
and patients
Healthy population

| sample | V gene | J gene | CDR3 AA | Divergence | Isotype | Occurrence |
|---|---|---|---|---|---|---|
| 326650 | IGHV3-53 / 3-66 | IGHJ6 | DLYYYGMDV | 0.007 ± 0.003 | M (100%) | 12 |
| | | | (SEQ ID NO: 27) | | | |
| 326713 | IGHV3-53 / 3-66 | IGHJ6 | DLYYYGMDV | 0.005 ± 0.010 | M (92.3%), G (7.7%) | 13 |
| | | | (SEQ ID NO: 27) | | | |
| 326780 | IGHV3-53 / 3-66 | IGHJ6 | DLYYYGMDV | 0.014 ± 0.010 | M (97.4%), G (2.6%) | 38 |
| | | | (SEQ ID NO: 27) | | | |
| 326797 | IGHV3-53 | IGHJ6 | DLYYYGMDV | 0.004 | M (100%) | 1 |
| | | | (SEQ ID NO: 27) | | | |
| 327059 | IGHV3-53 / 3-66 | IGHJ6 | DLYYYGMDV | 0.003 ± 0.005 | M (100%) | 8 |
| | | | (SEQ ID NO: 27) | | | |
| D103 | IGHV3-53 | IGHJ6 | DLYYYGMDV | 0.008 ± 0.020 | M (100%) | 9 |
| | | | (SEQ ID NO: 27) | | | |
| 326650 | IGHV3-53 / 3-66 | IGHJ6 | DLDYYGMDV | 0.006 ± 0.002 | M (75%), G (25%) | 4 |
| | | | (SEQ ID NO: 28) | | | |
| 326713 | IGHV3-53 / 3-66 | IGHJ6 | DLDYYGMDV | 0.012 ± 0.018 | M (100%) | 4 |
| | | | (SEQ ID NO: 28) | | | |
| 326797 | IGHV3-66 | IGHJ6 | DLDYYGMDV | 0.055 | M (100%) | 1 |
| | | | (SEQ ID NO: 28) | | | |
| 327059 | IGHV3-53 / 3-66 | IGHJ6 | DLDYYGMDV | 0.001 ± 0.002 | M (100%) | 4 |
| | | | (SEQ ID NO: 28) | | | |
| D103 | IGHV3-53 | IGHJ6 | DLDYYGMDV | 0.053 | M (100%) | 1 |
| | | | (SEQ ID NO: 28) | | | |
| 326713 | IGHV3-53 / 3-66 | IGHJ6 | DLVAYGMDV | 0.008 ± 0.011 | M (100%) | 2 |
| | | | (SEQ ID NO: 29) | | | |
| 326713 | IGHV3-53 | IGHJ6 | DLVYYGDMV | 0.001 ± 0.002 | M (100%) | 3 |
| | | | (SEQ ID NO: 30) | | | |
| 326797 | IGHV3-53 | IGHJ6 | DLVYYGMDV | 0.089 ± 0.008 | M (100%) | 2 |
| | | | (SEQ ID NO: 31) | | | |
| 326713 | IGHV3-53 | IGHJ6 | DLVVYGMDV | 0.024 ± 0.052 | M (100%) | 5 |
| | | | (SEQ ID NO: 32) | | | |
| 326780 | IGHV3-53 / 3-66 | IGHJ6 | DLSYYGMDV | 0.024 ± 0.024 | M (98.44%), D (0.78%), | 128 |
| | | | (SEQ ID NO: 33) | | G (0.78%) | |
| D103 | IGHV3-53 | IGHJ6 | DLSYYGMDV | 0.022 ± 0.003 | M (100%) | 2 |
| | | | (SEQ ID NO: 33) | | | |
| 327059 | IGHV3-53 | IGHJ6 | DLGDYGMDV | 0.000 | M (100%) | 1 |
| | | | (SEQ ID NO: 34) | | | |
| 326713 | IGHV3-66 | IGHJ6 | DAVSYGMDV | 0.000 ± 0.000 | M (100%) | 2 |
| | | | (SEQ ID NO: 35) | | | |

SARS-CoV-2-infected patients

| sample | V gene | J gene | CDR3 AA | Divergence | Isotype | Occurrence |
|---|---|---|---|---|---|---|
| A | IGHV3-53 | IGHJ6 | DLYYYGMDV | 0.002 ± 0.004 | M (5.1%), G1 (94.9%) | 59 |
| | | | (SEQ ID NO: 27) | | | |
| B | IGHV3-53 | IGHJ6 | DLYYYGMDV | 0.000 ± 0.000 | M (33.3%), G1 (66.7%) | 3 |
| | | | (SEQ ID NO: 27) | | | |
| G | IGHV3-53 / 3-66 | IGHJ6 | DLYYYGMDV | 0.005 ± 0.003 | G1 (84.6%), Al (15.4%) | 14 |
| | | | (SEQ ID NO: 27) | | | |
| I | IGHV3-53 | IGHJ6 | DLYYYGMDV | 0.000 ± 0.000 | M (100%) | 4 |
| | | | (SEQ ID NO: 27) | | | |
| K | IGHV3-53 | IGHJ6 | DLYYYGMDV | 0.009 ± 0.000 | G1 (100%) | 2 |
| | | | (SEQ ID NO: 27) | | | |
| A | IGHV3-53 | IGHJ6 | DLAVYGMDV | 0.004 ± 0.000 | G1 (100%) | 2 |
| | | | (SEQ ID NO: 36) | | | |
| E | IGHV3-66 | IGHJ6 | DLAVYGMDV | 0.018 ± 0.000 | G1 (100%) | 6 |
| | | | (SEQ ID NO: 36) | | | |
| A | IGHV3-53 | IGHJ6 | DLDYYGMDV | 0.000 ± 0.000 | G1 (100%) | 3 |
| | | | (SEQ ID NO: 28) | | | |
| E | IGHV3-53 | IGHJ6 | DLDYYGMDV | 0.004 ± 0.000 | A1 (100%) | 4 |
| | | | (SEQ ID NO: 28) | | | |
| I | IGHV3-66 | IGHJ6 | DLDYYGMDV | 0.002 ± 0.003 | G1 (100%) | 5 |
| | | | (SEQ ID NO: 28) | | | |
| K | IGHV3-53 | IGHJ6 | DLDYYGMDV | 0.007 ± 0.005 | G1 (100%) | 107 |
| | | | (SEQ ID NO: 28) | | | |
| M | IGHV3-53 | IGHJ6 | DLDYYGMDV | 0.018 | G1 (100%) | 1 |
| | | | (SEQ ID NO: 28) | | | |
| A | IGHV3-53 | IGHJ6 | DLVAYGMDV | 0.008 ± 0.017 | G1 (100%) | 14 |
| | | | (SEQ ID NO: 29) | | | |
| B | IGHV3-53 | IGHJ6 | DLVAYGMDV | 0.009 | G1 (100%) | 1 |
| | | | (SEQ ID NO: 29) | | | |
| E | IGHV3-53 | IGHJ6 | DLVAYGMDV | 0.005 ± 0.002 | G1 (100%) | 6 |
| | | | (SEQ ID NO: 29) | | | |
| D | IGHV3-53 | IGHJ6 | DLVYYGMDV | 0.004 | G1 (100%) | 1 |
| | | | (SEQ ID NO: 31) | | | |
| E | IGHV3-53 | IGHJ6 | DLVYYGMDV | 0.013 | A1 (100%) | 1 |
| | | | (SEQ ID NO: 31) | | | |
| F | IGHV3-53 | IGHJ6 | DLVYYGDMV | 0.001 ± 0.003 | M (75%), G1 (25%) | 16 |
| | | | (SEQ ID NO: 30) | | | |
| B | IGHV3-53 | IGHJ6 | DLVVYGMDV | 0.002 ± 0.002 | M (27.3%), G1 (72.7%) | 11 |
| | | | (SEQ ID NO: 32) | | | |
| E | IGHV3-53 | IGHJ6 | DLVVYGMDV | 0.013 ± 0.000 | A2 (100%) | 4 |
| | | | (SEQ ID NO: 32) | | | |
| H | IGHV3-53 | IGHJ6 | DLVVYGMDV | 0.009 ± 0.000 | G1 (100%) | 7 |
| | | | (SEQ ID NO: 32) | | | |
| A | IGHV3-53 | IGHJ6 | DLSYYGMDV | 0.013 ± 0.016 | G1 (100%) | 5 |
| | | | (SEQ ID NO: 33) | | | |
| F | IGHV3-53 | IGHJ6 | DLSYYGMDV | 0.018 | G1 (100%) | 1 |
| | | | (SEQ ID NO: 33) | | | |
| O | IGHV3-53 | IGHJ6 | DLSYYGMDV | 0.000 | G1 (100%) | 1 |
| | | | (SEQ ID NO: 33) | | | |
| A | IGHV3-53 | IGHJ6 | DLGDYGMDV | 0.009 ± 0.000 | G1 (100%) | 3 |
| | | | (SEQ ID NO: 34) | | | |
| E | IGHV3-53 | IGHJ6 | DLGDYGMDV | 0.018 ± 0.019 | G1 (85.7%), Al (14.3%) | 7 |
| | | | (SEQ ID NO: 34) | | | |
| F | IGHV3-53 | IGHJ6 | DLGDYGMDV | 0.003 ± 0.002 | M (92.0%), G1 (8.0%) | 163 |
| | | | (SEQ ID NO: 34) | | | |
| H | IGHV3-53 | IGHJ6 | DLGDYGDMV | 0.004 ± 0.000 | G1 (100%) | 8 |
| | | | (SEQ ID NO: 37) | | | |
| G | IGHV3-53 | IGHJ6 | DAVSYGMDV | 0.004 ± 0.004 | M (7.0%), G1 (93.0%) | 57 |
| | | | (SEQ ID NO: 35) | | | |
| I | IGHV3-53 | IGHJ6 | DAVSYGMDV | 0.007 ± 0.003 | G1 (100%) | 9 |
| | | | (SEQ ID NO: 35) | | | |
| P | IGHV3-53 | IGHJ6 | DAVSYGMDV | 0.000 ± 0.000 | G1 (100%) | 3 |
| | | | (SEQ ID NO: 35) | | | |
| E | IGHV3-53 | IGHJ6 | DLGPYGMDV | 0.009 | G1 (100%) | 1 |
| | | | (SEQ ID NO: 38) | | | |
| I | IGHV3-53 / 3-66 | IGHJ6 | DLGPYGMDV | 0.010 ± 0.003 | G3 (40%), G1 (40%), | 4 |
| | | | (SEQ ID NO: 38) | | A1 (20%) | |
| A | IGHV3-53 | IGHJ6 | DLVIYGMDV (SEQ | 0.003 ± 0.004 | M (5.9%), G1 (94.1%) | 17 |
| | | | ID NO: 39) | | | |
| I | IGHV3-66 | IGHJ6 | DLVIYGMDV (SEQ | 0.007 ± 0.004 | G1 (100%) | 8 |
| | | | ID NO: 39) | | | |
| E | IGHV3-53 / 3-66 | IGHJ6 | DLVVLGMDV | 0.009 ± 0.000 | A2 (100%) | 20 |
| | | | (SEQ ID NO: 40) | | | |
| I | IGHV3-53 | IGHJ6 | DLVVLGMDV | 0.000 | G1 (100%) | 1 |
| | | | (SEQ ID NO: 40) | | | |

The healthy samples based on publicly available IGH repertoires or patient identification can be found in the sample column. Clonotypes were mapped according to identical VJ gene usage of IGHV3-53/IGHV3-66 and IGHJ6 and perfectly matched HCDR3 amino acid sequence. Read counts of the mapped sequences in the repertoires of each sample were annotated in the occurrence column. For clonotypes with multiple occurrences, mean and standard deviation of divergence were represented. The proportion of each isotype is indicated for all samples.

TABLE 2
Demographic and clinical characteristics

| | | | | | | Highest | |
|---|---|---|---|---|---|---|---|
| Patient | | | | BMI | Underlying | temperature | |
| no. | Age | Sex | Race | (kg/m²) | diseases | (° C.) | Symptoms |
| A | 55 | Male | Korean | 31.35 | — | 39.7 | Dyspnea, |
| | | | | | | | myalgia, |
| | | | | | | | diarrhea |
| B | 55 | Male | Korean | 24.09 | DM, HTN, | 38.4 | Sputum, |
| | | | | | DL | | myalgia |
| C | 53 | Female | Korean | 23.1 | — | 38 | Sputum, |
| | | | | | | | myalgia |
| D | 24 | Male | Korean | 21.51 | — | 37.8 | Myalgia |
| E | 48 | Male | Chinese | 27.02 | — | 37.8 | Cough, |
| | | | | | | | myalgia, |
| | | | | | | | diarrhea |
| F | 40 | Female | Chinese | 22.15 | — | 37.8 | Cough, |
| | | | | | | | sputum, |
| | | | | | | | myalgia, |
| | | | | | | | diarrhea |
| G | 59 | Female | Korean | 18 | DM, DL | 37 | — |
| H | 92 | Female | Korean | 25.9 | HTN | 38.1 | Cough, |
| | | | | | | | sputum, |
| | | | | | | | myalgia |
| I | 58 | Male | Korean | 17.03 | — | 39.6 | Cough, |
| | | | | | | | sputum |
| J | 48 | Male | Korean | 23.32 | HTN | 36.7 | Myalgia, |
| | | | | | | | diarrhea |
| K | 79 | Female | Korean | 20.21 | HTN | 38.6 | Cough, |
| | | | | | | | sputum, |
| | | | | | | | diarrhea |
| L | 67 | Female | Korean | 22.14 | DM, HTN | 37.6 | Dyspnea, |
| | | | | | | | sputum |
| M | 84 | Male | Korean | 21.09 | HTN | 38.1 | Cough, |
| | | | | | | | sputum |
| N | 50 | Male | Korean | 23.18 | — | 36.5 | Cough, |
| | | | | | | | sputum, |
| | | | | | | | diarrhea |
| O | 76 | Female | Korean | 35.29 | HTN | 37.7 | Dyspnea |
| P | 45 | Male | Korean | 23.43 | HTN | 37.3 | Dyspnea |
| Q | 48 | Male | Korean | 30.69 | HTN, | 37.2 | Cough, |
| | | | | | ankylosing | | sputum, |
| | | | | | spondylitis | | sore throat, |
| | | | | | | | myalgia, |
| | | | | | | | diarrhea |
| | | | | | | | Blood samples |
| | | | | | | | collected after |
| | Patient | Pneumonic | Oxygen | | Antiviral | Antibiotic | symptoms |
| | no. | infiltrates | therapy | Ventilator | Treatment | Treatment | onset (Days) |
| | A | Extensive | Yes | No | Lopinavir/ | Levofloxacin | 11, 17, 45 |
| | | | | | ritonavir | | |
| | B | Limited | No | No | — | — | 10, 19 |
| | C | Limited | No | No | — | — | 6, 15 |
| | D | Limited | No | No | — | — | 6, 28 |
| | E | Extensive | No | No | Lopinavir/ | — | 23, 44, 99 |
| | | | | | ritonavir | | |
| | F | Limited | No | No | Lopinavir/ | — | 14, 36 |
| | | | | | ritonavir | | |
| | G | Limited | No | No | — | — | 9, 22 |
| | H | Extensive | Yes | No | — | Levofloxacin, | 9 |
| | | | | | | piperacillin/ | |
| | | | | | | tazobactam | |
| | I | Extensive | Yes | No | Remdesivir | Levofloxacin, | 13 |
| | | | | | | piperacillin/ | |
| | | | | | | tazobactam | |
| | J | Limited | No | No | — | — | 41 |
| | K | Limited | Yes | No | Remdesivir | — | 21 |
| | L | Limited | Yes | No | — | — | 25 |
| | M | Extensive | Yes | Yes | Remdesivir | Piperacillin/ | 22 |
| | | | | | | tazobactam | |
| | N | Limited | No | No | — | — | 34 |
| | O | Limited | Yes | Yes | Remdesivir | Ceftriaxone, | 15 |
| | | | | | | levofloxacin | |
| | P | Limited | Yes | No | | — | 16 |
| | Q | Limited | No | No | — | — | 35 |

BMI, body mass index;
DM, diabetes mellitus;
HUN, hypertension;
DL, dyslipidemia

TABLE 3
SARS-CoV-2 RBD-reactive scFv clones

| | | | | | J | | Mapped | Mapped |
|---|---|---|---|---|---|---|---|---|
| Clone | HCDR1 | HCDR2 | HCDR3 | V gene | gene | Divergence | patient | isotype |
| E_3B1 | SNYMS | VLYSGGSTFYADSVKG | DAQVYGMDV (SEQ ID NO: 43) | IGHV3-66 | IGHJ6 | 0.023973 | E | G1 |
| | (SEQ ID | (SEQ ID NO: 42) | | | | | | |
| | NO: 41) | | | | | | | |
| E_3A3 | RNYMS | VIYSGGSTYYADSVKG | DLDTAGGMDV (SEQ ID NO: 46) | IGHV3-66 | IGHJ6 | 0.010239 | — | — |
| | (SEQ ID | (SEQ ID NO: 45) | | | | | | |
| | NO: 44) | | | | | | | |
| E_3H4 | SNYMS | VIYSGGSTYYADSVKG | DLLEQGGMDV (SEQ ID NO: 47) | IGHV3-66 | IGHJ6 | 0.006826 | E | G1 |
| | (SEQ ID | (SEQ ID NO: 45) | | | | | | |
| | NO: 41) | | | | | | | |
| A_2F1 | SNYMS | VIYSGGSTFYADSVKG | DLMEAGGMDV (SEQ ID NO: 49) | IGHV3-53 | IGHJ6 | 0.030822 | — | — |
| | (SEQ ID | (SEQ ID NO: 48) | | | | | | |
| | NO: 41) | | | | | | | |
| A_1H4 | SNYMS | GIYSGGSTYYADSVKG | DLQEAGAFDI (SEQ ID NO: 51) | IGHV3-66 | IGHJ3 | 0.027304 | — | — |
| | (SEQ ID | (SEQ ID NO: 50) | | | | | | |
| | NO: 41) | | | | | | | |
| E_4H2 | SYWMS | NIKQDGSEKYYVDSVKG | HRWLRGEIDY (SEQ ID NO: 54) | IGHV3-7 | IGHJ4 | 0.003401 | E | G1 |
| | (SEQ ID | (SEQ ID NO: 53) | | | | | | |
| | NO: 52) | | | | | | | |
| A_1G5 | DYYMS | VISYDGSNKYYADSVKG | SSWLRGAFDY (SEQ ID NO: 57) | IGHV3-30 | IGHJ4 | 0.061017 | — | — |
| | (SEQ ID | (SEQ ID NO: 56) | | | | | | |
| | NO: 55) | | | | | | | |
| E_4G3 | SYWIG | IIYPGDSDTRYSPSFQG | LSSSYYGWFDP (SEQ ID NO: 60) | IGHV5-51 | IGHJ5 | 0.006826 | — | — |
| | (SEQ ID | (SEQ ID NO: 59) | | | | | | |
| | NO: 58) | | | | | | | |
| E_3B11 | SYWIA | IIYPGDSDTRYSPSFQG | YSSSPNGWFDP (SEQ ID NO: 62) | IGHV5-51 | IGHJ5 | 0.010239 | E | G1 |
| | (SEQ ID | (SEQ ID NO: 59) | | | | | | |
| | NO: 61) | | | | | | | |
| A_1C12 | SNAIS | RIIPIFGTANYAQKFQG | DVIESPLYGMDV (SEQ ID NO: | IGHV1-69 | IGHJ6 | 0.027027 | A | G1 |
| | (SEQ ID | (SEQ ID NO: 64) | 65) | | | | | |
| | NO: 63) | | | | | | | |
| E_4B2 | SFAIT | RIIPILGIANYAQKFQG | EFSGGDNTGFDY (SEQ ID NO: | IGHV1-69 | IGHJ4 | 0.023649 | E | G1 |
| | (SEQ ID | (SEQ ID NO: 67) | 68) | | | | | |
| | NO: 66) | | | | | | | |
| E_4D10 | SHYMH | IINPSGGSTSYAQKFQG | DGYFVPARSAFDI (SEQ ID NO: | IGHV1-46 | IGHJ3 | 0.013652 | E | M |
| | (SEQ ID | (SEQ ID NO: 70) | 71) | | | | | |
| | NO: 69) | | | | | | | |
| A_2A1 | DYAMH | GISWNSGTIGYADSVKG | DITMVREAYGMDV (SEQ ID NO: | IGHV3-9 | IGHJ6 | 0.033557 | — | — |
| | (SEQ ID | (SEQ ID NO: 73) | 74) | | | | | |
| | NO: 72) | | | | | | | |
| A_1H11 | DYAMH | GTSWNSGTIGYADSVKG | DKGQIRESYGMDV (SEQ ID NO: | IGHV3-9 | IGHJ6 | 0.071186 | — | — |
| | (SEQ ID | (SEQ ID NO: 75) | 76) | | | | | |
| | NO: 72) | | | | | | | |
| A_1B10 | DYAMH | GTDWNSGTIGYADSVKG | DLGGVVERYGMDV (SEQ ID | IGHV3-9 | IGHJ6 | 0.031802 | — | — |
| | (SEQ ID | (SEQ ID NO: 77) | NO: 78) | | | | | |
| | NO: 72) | | | | | | | |
| A_1H10 | SYYIH | IINPDAGSTTYAQKFQG | DLYGLPGRAAFDI (SEQ ID NO: | IGHV1-46 | IGHJ3 | 0.037288 | — | — |
| | (SEQ ID | (SEQ ID NO: 80) | 81) | | | | | |
| | NO: 79) | | | | | | | |
| E_3A12 | SNYMS | VIYSGGSTYYADSVKG | GDGSGDYYYGMDV (SEQ ID | IGHV3-53 | IGHJ6 | 0.006849 | E | A2 |
| | (SEQ ID | (SEQ ID NO: 45) | NO: 82) | | | | | |
| | NO: 41) | | | | | | | |
| A_1B1 | NYWIG | ITYPGDSDTRYSPSFQG | HLDWNAPRGPFDI (SEQ ID NO: | IGHV5-51 | IGHJ3 | 0.013699 | — | — |
| | (SEQ ID | (SEQ ID NO: 59) | 84) | | | | | |
| | NO: 83) | | | | | | | |
| A_1C11 | DYAMH | GISWNSGTIGYADSVKG | DIFRTEWLQYGMDV (SEQ ID | IGHV3-9 | IGHJ6 | 0.027119 | — | — |
| | (SEQ ID | (SEQ ID NO: 73) | NO: 85) | | | | | |
| | NO: 72) | | | | | | | |
| E_3F11 | DYAMH | GSSWNSGTIGYADSVKG | DMGRGNDNNLAFDI (SEQ ID | IGHV3-9 | IGHJ3 | 0.037543 | E | G1 |
| | (SEQ ID | (SEQ ID NO: 86) | NO: 87) | | | | | |
| | NO: 72) | | | | | | | |
| E_3G9 | SYYMH | IINPSGGSTSYAQKFQG | EGVWDSSGYSSFDY (SEQ ID | IGHV1-46 | IGHJ4 | 0.013514 | E | A1 |
| | (SEQ ID | (SEQ ID NO: 70) | NO: 89) | | | | | |
| | NO: 88) | | | | | | | |
| E_4C8 | DYAMH | GVTWNSGSIGYADSVKG | DISPMLRGDNYGMDV (SEQ ID | IGHV3-9 | IGHJ6 | 0.016949 | E | G1 |
| | (SEQ ID | (SEQ ID NO: 90) | NO: 91) | | | | | |
| | NO: 72) | | | | | | | |
| E_4A8 | DYAMH | SVTWNSGNIGYADSVKG | DISSMLRGDNYCMDV (SEQ ID | IGHV3-9 | IGHJ6 | 0.047619 | — | — |
| | (SEQ ID | (SEQ ID NO: 92) | NO: 93) | | | | | |
| | NO: 72) | | | | | | | |
| E_3F1 | SYAIS | RIIPILGIANYAQKFQG | DRGYSDYGSNPFFDY (SEQ ID | IGHV1-69 | IGHJ4 | 0.047458 | — | — |
| | (SEQ ID | (SEQ ID NO: 67) | NO: 95) | | | | | |
| | NO: 94) | | | | | | | |
| E_4H4 | SYAIS | RIIPILGIANYAQKFQG | GIGYSGSGSNDYFDS (SEQ ID | IGHV1-69 | IGHJ4 | 0.03367 | — | — |
| | (SEQ ID | X | NO: 97) | | | | | |
| | NO: 94) | (SEQ ID NO: 96) | | | | | | |
| A_1F1 | DYAMH | GISWNSGIIGYADSVKG | DIRGYSGYDDPGAFDI (SEQ ID | IGHV3-9 | IGHJ3 | 0.010067 | — | — |
| | (SEQ ID | (SEQ ID NO: 98) | NO: 99) | | | | | |
| | NO: 72) | | | | | | | |
| E_4B4 | DYAMH | GSSWNSGSIGYADSVKG | GKSPLDYDQTMGAFDI (SEQ ID | IGHV3-9 | IGHJ3 | 0.027119 | E | A1 |
| | (SEQ ID | (SEQ ID NO: 100) | NO: 101) | | | | | |
| | NO: 72) | | | | | | | |
| A_1A11 | DYAMS | FIRSKAYGGTTEYAASV | DEDSGTLLPGFYYYDMDV (SEQ | IGHV3-49 | IGHJ6 | 0.003322 | A | G1 |
| | (SEQ ID | KG | ID NO: 104) | | | | | |
| | NO: 102) | (SEQ ID NO: 103) | | | | | | |
| E_4D12 | TYWIN | RIDPSDSYTNYSPSFQG | GDYYDNSDYSGLSEYFQH (SEQ | IGHV5- | IGHJ1 | 0.013605 | E | G1 |
| | (SEQ ID | (SEQ ID NO: 106) | ID NO: 107) | 10-1 | | | | |
| | NO: 105) | | | | | | | |
| E_3H31 | RYAMH | WINAGNGKTKYSQKFQG | ALYYYDSSGSTQSDDAFDI (SEQ | IGHV1-3 | IGHJ3 | 0.016949 | E | G1 |
| | (SEQ ID | (SEQ ID NO: 109) | ID NO: 110) | | | | | |
| | NO: 108) | | | | | | | |
| E_4F91 | SNYMS | VIYSGGSTYYADSVKG | DGQRMAAAGTEDYYYGMDV | IGHV3-66 | IGHJ6 | 0.003413 | E | G1\|A1\| |
| | (SEQ ID | (SEQ ID NO: 45) | (SEQ ID NO: 111) | | | | | A2 |
| | NO: 41) | | | | | | | |
| A_1H12 | DYAMH | GVTWNSGTIGYADSVKG | DIMGDGSPSLHYYYYGMDV | IGHV3-9 | IGHJ6 | 0.033557 | — | — |
| | (SEQ ID | (SEQ ID NO: 112) | (SEQ ID NO: 113) | | | | | |
| | NO: 72) | | | | | | | |
| E_4F9 | SNYMS | VIYIGGSTYYSYSVKG | DRQRMAAAGTEDYYYGMDV | IGHV3-66 | IGHJ6 | 0.044369 | — | — |
| | (SEQ ID | (SEQ ID NO: 114) | (SEQ ID NO: 115) | | | | | |
| | NO: 41) | | | | | | | |
| A_2G3 | DYGMT | GINWNGGTTGYADSVKG | IYCGDDCYSLVIWGDAFDI (SEQ | IGHV3-20 | IGHJ3 | 0.023891 | — | — |
| | (SEQ ID | (SEQ ID NO: 117) | ID NO: 118) | | | | | |
| | NO: 116) | | | | | | | |
| A_1A1 | DYAMH | GISWNSGTIGYADSVKG | DENRGYSSRWYDPEYYGMDV | IGHV3-9 | IGHJ6 | 0.006826 | A | G1 |
| | (SEQ ID | (SEQ ID NO: 73) | (SEQ ID NO: 119) | | | | | |
| | NO: 72) | | | | | | | |
| A_2H4 | VYGMH | VISYDGSNKYYADSVKG | GGPRPVVKAYGELDYYGMDV | IGHV3-30 | IGHJ6 | 0.030928 | — | — |
| | (SEQ ID | (SEQ ID NO: 56) | (SEQ ID NO: 121) | | | | | |
| | NO: 120) | | | | | | | |
| A_1G9 | DYAMH | GTSWNSGTIGYADSVRG | YGTEGLYDFRSGYGHYGMDV | IGHV3-9 | IGHJ6 | 0.03413 | — | — |
| | (SEQ ID | (SEQ ID NO: 122) | (SEQ ID NO: 123) | | | | | |
| | NO: 72) | | | | | | | |
| A_1H2 | RYAIS | GIIPIFGTANYAQKFQG | ERTYCSSTSCYAGYYYYGMDV | IGHV1-69 | IGHJ6 | 0.016892 | A | G1\|A1 |
| | (SEQ ID | (SEQ ID NO: 125) | (SEQ ID NO: 126) | | | | | |
| | NO: 124) | | | | | | | |

TABLE 4
Class-switched IGH clonotypes homologous to E-3B1

| | | | | | | | | Substi- |
|---|---|---|---|---|---|---|---|---|
| | | | | | | | | tution |
| Patient | HCDR1 | HCDR2 | HCDR3 | V gene | J gene | Divergence | Isotype | in HCDR3 |
| A | SNYMS | VIYSGGSTYYADSVKG | DLAVYGMDV | IGHV3-53 | IGHJ6 | 0.004386 | G1 | 0.222222 |
| | (SEQ ID | (SEQ ID NO: 45) | (SEQ ID NO: 36) | | | | | |
| | NO: 41) | | | | | | | |
| A | SNYMS | VIYSGGSTYYADSVKG | DLDYYGMDV | IGHV3-53 | IGHJ6 | 0 | G1 | 0.333333 |
| | (SEQ ID | (SEQ ID NO: 45) | (SEQ ID NO: 28) | | | | | |
| | NO: 41) | | | | | | | |
| A | SNYMS | VIYSGGSTFYADSVKG | DLGDYGMDV | IGHV3-53 | IGHJ6 | 0.008772 | G1 | 0.333333 |
| | (SEQ ID | (SEQ ID NO: 48) | (SEQ ID NO: 34) | | | | | |
| | NO: 41) | | | | | | | |
| A | SNYMS | VIYSGGSTYYADSVKG | DLQVYGMDV | IGHV3-53 | IGHJ6 | 0 | G1 | 0.111111 |
| | (SEQ ID | (SEQ ID NO: 45) | (SEQ ID NO: | | | | | |
| | NO: 41) | | 127) | | | | | |
| A | SNYMS | DIYSGGSTDYADSVKG | DLSYYGMDV | IGHV3-53 | IGHJ6 | 0.008772 | G1 | 0.333333 |
| | (SEQ ID | (SEQ ID NO: 128) | (SEQ ID NO: 33) | | | | | |
| | NO: 41) | | | | | | | |
| A | SNYMS | VIYSGGSTYYADSVKG | DLSYYGMDV | IGHV3-53 | IGHJ6 | 0 | G1 | 0.333333 |
| | (SEQ ID | (SEQ ID NO: 45) | (SEQ ID NO: 33) | | | | | |
| | NO: 41) | | | | | | | |
| A | SNYMN | VIYSGGSTFYADSVKG | DLSYYGMDV | IGHV3-53 | IGHJ6 | 0.030702 | G1 | 0.333333 |
| | (SEQ ID | (SEQ ID NO: 48) | (SEQ ID NO: 33) | | | | | |
| | NO: 129) | | | | | | | |
| A | SNYMS | VIYSGGSTYYADSVKG | DLVAYGMDV | IGHV3-53 | IGHJ6 | 0 | G1 | 0.333333 |
| | (SEQ ID | (SEQ ID NO: 45) | (SEQ ID NO: 29) | | | | | |
| | NO: 41) | | | | | | | |
| A | SNYMS | VIYAGGTTDYADSVKG | DLVAYGMDV | IGHV3-53 | IGHJ6 | 0.039474 | G1 | 0.333333 |
| | (SEQ ID | (SEQ ID NO: 130) | (SEQ ID NO: 29) | | | | | |
| | NO: 41) | | | | | | | |
| A | SNYMS | VIYSGGSTYYADSVKG | DLVDYGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 131) | | | | | |
| A | SNYMS | VIYSGGSTYYADSVKG | DLVIYGMDV | IGHV3-53 | IGHJ6 | 0 | G1 | 0.333333 |
| | (SEQ ID | (SEQ ID NO: 45) | (SEQ ID NO: 39) | | | | | |
| | NO: 41) | | | | | | | |
| A | SNYMS | VIYSGGSTYYADSVKG | DLVIYGMDV | IGHV3-53 | IGHJ6 | 0.008772 | G1 | 0.333333 |
| | (SEQ ID | (SEQ ID NO: 45) | (SEQ ID NO: 39) | | | | | |
| | NO: 41) | | | | | | | |
| A | SNYMS | VIYSGGSTYYADSVKG | DLVIYGMDV | IGHV3-53 | IGHJ6 | 0 | G1 | 0.333333 |
| | (SEQ ID | (SEQ ID NO: 45) | (SEQ ID NO: 39) | | | | | |
| | NO: 41) | | | | | | | |
| A | SNYMS | VIYSGGSTYYADSVKG | DLVIYGMDV | IGHV3-53 | IGHJ6 | 0.004386 | G1 | 0.333333 |
| | (SEQ ID | (SEQ ID NO: 45) | (SEQ ID NO: 39) | | | | | |
| | NO: 41) | | | | | | | |
| A | SNYMS | VIYSGGSTYYADSVKG | DLVVMGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 132) | | | | | |
| A | SNYMS | VIYSGGSTYYADSVKG | DLYYYGMDV | IGHV3-53 | IGHJ6 | 0 | G1 | 0.333333 |
| | (SEQ ID | (SEQ ID NO: 45) | (SEQ ID NO: 27) | | | | | |
| | NO: 41) | | | | | | | |
| A | SNYMS | VIYSGGSTYYADSVKG | DLYYYGMDV | IGHV3-53 | IGHJ6 | 0 | G1 | 0.333333 |
| | (SEQ ID | (SEQ ID NO: 45) | (SEQ ID NO: 27) | | | | | |
| | NO: 41) | | | | | | | |
| A | SNYMS | VIYSGGSTYYADSVKG | DLYYYGMDV | IGHV3-53 | IGHJ6 | 0.004386 | G1 | 0.333333 |
| | (SEQ ID | (SEQ ID NO: 45) | (SEQ ID NO: 27) | | | | | |
| | NO: 41) | | | | | | | |
| A | SNYMT | IIYSGGSTYYADSVKG | DLYYYGMDV | IGHV3-53 | IGHJ6 | 0.017544 | G1 | 0.333333 |
| | (SEQ ID | (SEQ ID NO: 134) | (SEQ ID NO: 27) | | | | | |
| | NO: 133) | | | | | | | |
| A | SNYMS | VIYSGGSTYYADSVKG | DLYYYGMDV | IGHV3-53 | IGHJ6 | 0 | G1 | 0.333333 |
| | (SEQ ID | (SEQ ID NO: 45) | (SEQ ID NO: 27) | | | | | |
| | NO: 41) | | | | | | | |
| A | SNYMS | VIYSGGSTFYADSVKG | DLYYYGMDV | IGHV3-53 | IGHJ6 | 0.004386 | G1 | 0.333333 |
| | (SEQ ID | (SEQ ID NO: 48) | (SEQ ID NO: 27) | | | | | |
| | NO: 41) | | | | | | | |
| A | SNYMS | IIYSGGSTFYADSVKG | DLYYYGMDV | IGHV3-53 | IGHJ6 | 0.013158 | G1 | 0.333333 |
| | (SEQ ID | (SEQ ID NO: 135) | (SEQ ID NO: 27) | | | | | |
| | NO: 41) | | | | | | | |
| A | SNYMS | VIYSGGSTFYADSVKG | DLYYYGMDV | IGHV3-53 | IGHJ6 | 0.004386 | G1 | 0.333333 |
| | (SEQ ID | (SEQ ID NO: 48) | (SEQ ID NO: 27) | | | | | |
| | NO: 41) | | | | | | | |
| A | SNYMS | VIYSGGSTYYADSVKG | DRDYYGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 136) | | | | | |
| B | SNYMS | VIYSGGSTYYADSVKG | DLVAYGMDV | IGHV3-53 | IGHJ6 | 0.008772 | G1 | 0.333333 |
| | (SEQ ID | (SEQ ID NO: 45) | (SEQ ID NO: 29) | | | | | |
| | NO: 41) | | | | | | | |
| B | SNYMS | VIYSGGSTYYADSVKG | DLVVYGMDV | IGHV3-53 | IGHJ6 | 0 | G1 | 0.222222 |
| | (SEQ ID | (SEQ ID NO: 45) | (SEQ ID NO: 32) | | | | | |
| | NO: 41) | | | | | | | |
| B | SNYMS | VIYSGGSTDYADSVKG | DLVVYGMDV | IGHV3-53 | IGHJ6 | 0.004386 | G1 | 0.222222 |
| | (SEQ ID | (SEQ ID NO: 137) | (SEQ ID NO: 32) | | | | | |
| | NO: 41) | | | | | | | |
| B | SNYMS | VIYSGGSTYYADSVKG | DLVVYGMDV | IGHV3-53 | IGHJ6 | 0 | G1 | 0.222222 |
| | (SEQ ID | (SEQ ID NO: 45) | (SEQ ID NO: 32) | | | | | |
| | NO: 41) | | | | | | | |
| B | SNYMS | VIYSGGSTYYADPVKG | DLVVYGMDV | IGHV3-53 | IGHJ6 | 0.004386 | G1 | 0.222222 |
| | (SEQ ID | (SEQ ID NO: 138) | (SEQ ID NO: 32) | | | | | |
| | NO: 41) | | | | | | | |
| B | SNYMS | VIYSGGSTYYADSVKG | DLYYYGMDV | IGHV3-53 | IGHJ6 | 0 | G1 | 0.333333 |
| | (SEQ ID | (SEQ ID NO: 45) | (SEQ ID NO: 27) | | | | | |
| | NO: 41) | | | | | | | |
| D | SNYMN | VIYSGGSTYYTDSVKG | DLHYYGMDV | IGHV3-53 | IGHJ6 | 0.013158 | G1 | 0.333333 |
| | (SEQ ID | (SEQ ID NO: 139) | (SEQ ID NO: | | | | | |
| | NO: 129) | | 140) | | | | | |
| D | SNYMT | VIYSGGSTYYADSVKG | DLVYYGMDV | IGHV3-53 | IGHJ6 | 0.004386 | G1 | 0.333333 |
| | (SEQ ID | (SEQ ID NO: 45) | (SEQ ID NO: 31) | | | | | |
| | NO: 133) | | | | | | | |
| E | SNYMS | VIYSGGSTYYADSVKG | DLAVYGMDV | IGHV3-66 | IGHJ6 | 0.017543 | G1 | 0.222222 |
| | (SEQ ID | (SEQ ID NO: 45) | (SEQ ID NO: 36) | | | | | |
| | NO: 41) | | | | | | | |
| E | SNYMS | VIYSGGSTYYADSVKG | DLAYYGMDV | IGHV3-66 | IGHJ6 | 0.008772 | A1 | 0.333333 |
| | (SEQ ID | (SEQ ID NO: 45) | (SEQ ID NO: | | | | | |
| | NO: 41) | | 141) | | | | | |
| E | SNYMS | VIYSGGTTYYADSVKG | DLDYYGMDV | IGHV3-53 | IGHJ6 | 0.004386 | A1 | 0.333333 |
| | (SEQ ID | (SEQ ID NO: 142) | (SEQ ID NO: 28) | | | | | |
| | NO: 41) | | | | | | | |
| E | SNYMS | VIYSGGSIFYADSVKG | DLGDYGMDV | IGHV3-53 | IGHJ6 | 0.030702 | A1 | 0.333333 |
| | (SEQ ID | (SEQ ID NO: 143) | (SEQ ID NO: 34) | | | | | |
| | NO: 41) | | | | | | | |
| E | SNYMC | VIYSGGSTYYADSVKG | DLGDYGMDV | IGHV3-53 | IGHJ6 | 0.004386 | G1 | 0.333333 |
| | (SEQ ID | (SEQ ID NO: 45) | (SEQ ID NO: 34) | | | | | |
| | NO: 144) | | | | | | | |
| E | SNYMS | VIYSGGSTYYADSVKG | DLGPYGMDV | IGHV3-53 | IGHJ6 | 0.008772 | G1 | 0.333333 |
| | (SEQ ID | (SEQ ID NO: 45) | (SEQ ID NO: 38) | | | | | |
| | NO: 41) | | | | | | | |
| E | SNYMS | VIYSGGSTYYADSVKG | DLGSYGMDV | IGHV3-53 | IGHJ6 | 0.008772 | A2 | 0.333333 |
| | (SEQ ID | (SEQ ID NO: 45) | (SEQ ID NO: | | | | | |
| | NO: 41) | | 145) | | | | | |
| E | SNYMN | VIYSGGSTYYADSVKG | DLPYYGMDV | IGHV3-66 | IGHJ6 | 0.004386 | G1 | 0.333333 |
| | (SEQ ID | (SEQ ID NO: 45) | (SEQ ID NO: | | | | | |
| | NO: 129) | | 146) | | | | | |
| E | SNYMS | VIYSGGSTYYADSVKG | DLTVYGMDV | IGHV3-53 | IGHJ6 | 0.008772 | A1 | 0.222222 |
| | (SEQ ID | (SEQ ID NO: 45) | (SEQ ID NO: | | | | | |
| | NO: 41) | | 147) | | | | | |
| E | SNYMS | VIYSGGSTYYADSVKG | DLVAYGMDV | IGHV3-53 | IGHJ6 | 0.004386 | G1 | 0.333333 |
| | (SEQ ID | (SEQ ID NO: 45) | (SEQ ID NO: 29) | | | | | |
| | NO: 41) | | | | | | | |
| E | SNYMS | VIYSGGSTYYADSVKG | DLVAYGMDV | IGHV3-53 | IGHJ6 | 0.008772 | G1 | 0.333333 |
| | (SEQ ID | (SEQ ID NO: 45) | (SEQ ID NO: 29) | | | | | |
| | NO: 41) | | | | | | | |
| E | SNYMS | VIYSGGSTYYADSVKG | DLVVLGMDV | IGHV3-53 | IGHJ6 | 0.008772 | A2 | 0.333333 |
| | (SEQ ID | (SEQ ID NO: 45) | (SEQ ID NO: 40) | | | | | |
| | NO: 41) | | | | | | | |
| E | SNYMT | LIYSGGSTYYADSVKG | DLVVWGMDV | IGHV3-53 | IGHJ6 | 0.039474 | G1 | 0.333333 |
| | (SEQ ID | (SEQ ID NO: 148) | (SEQ ID NO: | | | | | |
| | NO: 133) | | 149) | | | | | |
| E | SNYMT | VIYSGGSTYYADSVKG | DLVVYGMDV | IGHV3-53 | IGHJ6 | 0.013158 | A2 | 0.222222 |
| | (SEQ ID | (SEQ ID NO: 45) | (SEQ ID NO: 32) | | | | | |
| | NO: 133) | | | | | | | |
| E | SNYMS | VLYSGGSTYYADSVKG | DLVYYGMDV | IGHV3-66 | IGHJ6 | 0.013158 | A1 | 0.333333 |
| | (SEQ ID | (SEQ ID NO: 150) | (SEQ ID NO: 31) | | | | | |
| | NO: 41) | | | | | | | |
| F | SNYMS | VIYSGGSTYYADSVKG | DLGDYGMDV | IGHV3-53 | IGHJ6 | 0 | G1 | 0.333333 |
| | (SEQ ID | (SEQ ID NO: 45) | (SEQ ID NO: 34) | | | | | |
| | NO: 41) | | | | | | | |
| F | RNYMS | IIYSGGSTFYADSVKG | DLSYYGMDV | IGHV3-53 | IGHJ6 | 0.017544 | G1 | 0.333333 |
| | (SEQ ID | (SEQ ID NO: 135) | (SEQ ID NO: 33) | | | | | |
| | NO: 44) | | | | | | | |
| F | SNYMS | VIYSGGSTYYADSVKG | DLVYYGMDV | IGHV3-53 | IGHJ6 | 0 | G1 | 0.333333 |
| | (SEQ ID | (SEQ ID NO: 45) | (SEQ ID NO: 31) | | | | | |
| | NO: 41) | | | | | | | |
| F | SNYMS | VIYSGGSTYYADSVKG | DLVYYGMDV | IGHV3-53 | IGHJ6 | 0.004386 | G1 | 0.333333 |
| | (SEQ ID | (SEQ ID NO: 45) | (SEQ ID NO: 31) | | | | | |
| | NO: 41) | | | | | | | |
| G | SNYMN | IIYSGGTTYYADSVKG | DLYYYGMDV | IGHV3-66 | IGHJ6 | 0.013274 | A1 | 0.333333 |
| | (SEQ ID | (SEQ ID NO: 151) | (SEQ ID NO: 27) | | | | | |
| | NO: 129) | | | | | | | |
| G | SNYMS | VIYSGGSTYYADSVKG | DLYYYGMDV | IGHV3-53 | IGHJ6 | 0.004386 | G1 | 0.333333 |
| | (SEQ ID | (SEQ ID NO: 45) | (SEQ ID NO: 27) | | | | | |
| | NO: 41) | | | | | | | |
| G | SNYMS | VIYSGGSTYYADSVKG | DLYYYGMDV | IGHV3-53 | IGHJ6 | 0 | A1 | 0.333333 |
| | (SEQ ID | (SEQ ID NO: 45) | (SEQ ID NO: 27) | | | | | |
| | NO: 41) | | | | | | | |
| G | SNYMN | VIYSGGSTYYADSVKG | DVVVWGMDV | IGHV3-53 | IGHJ6 | 0.013158 | G1 | 0.333333 |
| | (SEQ ID | (SEQ ID NO: 45) | (SEQ ID NO: | | | | | |
| | NO: 129) | | 152) | | | | | |
| H | SNYMS | VIYSGGSTYYADSVKG | DLGDYGMDV | IGHV3-53 | IGHJ6 | 0.004386 | G1 | 0.333333 |
| | (SEQ ID | (SEQ ID NO: 45) | (SEQ ID NO: 34) | | | | | |
| | NO: 41) | | | | | | | |
| H | SNYMS | IIYSGGSTYYADSVKG | DLIMYGMDV | IGHV3-53 | IGHJ6 | 0.008772 | G1 | 0.333333 |
| | (SEQ ID | (SEQ ID NO: 134) | (SEQ ID NO: | | | | | |
| | NO: 41) | | 153) | | | | | |
| H | SNYMS | VIYSGGTTYYADSVKG | DLQDYGMDV | IGHV3-53 | IGHJ6 | 0.004386 | G1 | 0.222222 |
| | (SEQ ID | (SEQ ID NO: 142) | (SEQ ID NO: | | | | | |
| | NO: 41) | | 154) | | | | | |
| H | RNYMS | VIYSGGSTYYADSVKG | DLVVYGMDV | IGHV3-53 | IGHJ6 | 0.008772 | G1 | 0.222222 |
| | (SEQ ID | (SEQ ID NO: 45) | (SEQ ID NO: 32) | | | | | |
| | NO: 44) | | | | | | | |
| I | SNYMS | VIYSGGSTYYADSVKG | DAVSYGMDV | IGHV3-53 | IGHJ6 | 0.004386 | G1 | 0.222222 |
| | (SEQ ID | (SEQ ID NO: 45) | (SEQ ID NO: 35) | | | | | |
| | NO: 41) | | | | | | | |
| I | SNYMS | VIYSGGSTYYADSVKG | DAVSYGMDV | IGHV3-53 | IGHJ6 | 0.008772 | G1 | 0.222222 |
| | (SEQ ID | (SEQ ID NO: 45) | (SEQ ID NO: 35) | | | | | |
| | NO: 41) | | | | | | | |
| I | SNYMS | VIYSGGSTYYADSVKG | DLDYYGMDV | IGHV3-66 | IGHJ6 | 0.004386 | G1 | 0.333333 |
| | (SEQ ID | (SEQ ID NO: 45) | (SEQ ID NO: 28) | | | | | |
| | NO: 41) | | | | | | | |
| I | SNYMS | VIYSGGSTYYADSVKG | DLDYYGMDV | IGHV3-66 | IGHJ6 | 0 | G1 | 0.333333 |
| | (SEQ ID | (SEQ ID NO: 45) | (SEQ ID NO: 28) | | | | | |
| | NO: 41) | | | | | | | |
| I | SNYMT | LIYSGGSTYYADSVKG | DLGPYGMDV | IGHV3-53 | IGHJ6 | 0.008772 | G3 | 0.333333 |
| | (SEQ ID | (SEQ ID NO: 148) | (SEQ ID NO: 38) | | | | | |
| | NO: 133) | | | | | | | |
| I | SNYMS | VIYSGGSTFYADSVKG | DLGPYGMDV | IGHV3-66 | IGHJ6 | 0.013158 | G1 | 0.333333 |
| | (SEQ ID | (SEQ ID NO: 48) | (SEQ ID NO: 38) | | | | | |
| | NO: 41) | | | | | | | |
| I | SNYMS | VIYSGGSTFYADSVKG | DLGPYGMDV | IGHV3-53 | IGHJ6 | 0.008772 | A1 | 0.333333 |
| | (SEQ ID | (SEQ ID NO: 48) | (SEQ ID NO: 38) | | | | | |
| | NO: 41) | | | | | | | |
| I | SNYMS | LIYSGGSTYYADSVKG | DLVIYGMDV | IGHV3-66 | IGHJ6 | 0.004386 | G1 | 0.333333 |
| | (SEQ ID | (SEQ ID NO: 148) | (SEQ ID NO: 39) | | | | | |
| | NO: 41) | | | | | | | |
| I | RNYMN | VIYSGGSTYYADSVKG | DLVIYGMDV | IGHV3-66 | IGHJ6 | 0.013158 | G1 | 0.333333 |
| | (SEQ ID | (SEQ ID NO: 45) | (SEQ ID NO: 39) | | | | | |
| | NO: 155) | | | | | | | |
| I | SNYMS | VIYSGGSTYYADSVKG | DLVIYGMDV | IGHV3-66 | IGHJ6 | 0.004386 | G1 | 0.333333 |
| | (SEQ ID | (SEQ ID NO: 45) | (SEQ ID NO: 39) | | | | | |
| | NO: 41) | | | | | | | |
| I | SNYMS | VIYSGGSTYYADSVKG | DLVIYGMDV | IGHV3-66 | IGHJ6 | 0.004386 | G1 | 0.333333 |
| | (SEQ ID | (SEQ ID NO: 45) | (SEQ ID NO: 39) | | | | | |
| | NO: 41) | | | | | | | |
| I | SNYMS | VIYSGGSTYYADSVKG | DLVVLGMDV | IGHV3-53 | IGHJ6 | 0 | G1 | 0.333333 |
| | (SEQ ID | (SEQ ID NO: 45) | (SEQ ID NO: 40) | | | | | |
| | NO: 41) | | | | | | | |
| I | SNYMT | VIYSGGSTYYADSVKG | DRVVYGMDV | IGHV3-66 | IGHJ6 | 0.008772 | G1 | 0.222222 |
| | (SEQ ID | (SEQ ID NO: 45) | (SEQ ID NO: | | | | | |
| | NO: 133) | | 156) | | | | | |
| I | SNYMS | IIYSGGSTYYADSVKG | DSPYYGMDV | IGHV3-53 | IGHJ6 | 0.013158 | G2 | 0.333333 |
| | (SEQ ID | (SEQ ID NO: 134) | (SEQ ID NO: | | | | | |
| | NO: 41) | | 157) | | | | | |
| J | NNYMS | IIYNDGSTYYADSVKG | DAVLTGMDV | IGHV3-53 | IGHJ6 | 0.065789 | A1 | 0.333333 |
| | (SEQ ID | (SEQ ID NO: 159) | (SEQ ID NO: | | | | | |
| | NO: 158) | | 160) | | | | | |
| J | TNYIS | IIYSGGSTYYADSVKG | DAVLTGMDV | IGHV3-53 | IGHJ6 | 0.048246 | A1 | 0.333333 |
| | (SEQ ID | (SEQ ID NO: 134) | (SEQ ID NO: | | | | | |
| | NO: 161) | | 160) | | | | | |
| J | GNYMC | VIFADGRAYYADSVRG | DMADYGMDV | IGHV3-66 | IGHJ6 | 0.105263 | G2 | 0.333333 |
| | (SEQ ID | (SEQ ID NO: 163) | (SEQ ID NO: | | | | | |
| | NO: 162) | | 164) | | | | | |
| K | SNYMS | VIYSGGSTFYADSVKG | DAASYGMDV | IGHV3-53 | IGHJ6 | 0.004386 | G3 | 0.222222 |
| | (SEQ ID | (SEQ ID NO: 48) | (SEQ ID NO: | | | | | |
| | NO: 41) | | 165) | | | | | |
| K | SNYMS | VIYSGGSTFYADSVKG | DAASYGMDV | IGHV3-53 | IGHJ6 | 0.008772 | G1 | 0.222222 |
| | (SEQ ID | (SEQ ID NO: 48) | (SEQ ID NO: | | | | | |
| | NO: 41) | | 165) | | | | | |
| K | SNYMS | VIYSGGSTFYADSVKG | DAASYGMDV | IGHV3-53 | IGHJ6 | 0.004386 | G1 | 0.222222 |
| | (SEQ ID | (SEQ ID NO: 48) | (SEQ ID NO: | | | | | |
| | NO: 41) | | 165) | | | | | |
| K | SNYMS | VIYSGGSTFYVDSVKG | DAASYGMDV | IGHV3-53 | IGHJ6 | 0.008772 | G1 | 0.222222 |
| | (SEQ ID | (SEQ ID NO: 166) | (SEQ ID NO: | | | | | |
| | NO: 41) | | 165) | | | | | |
| K | RNYMS | VIYSGGSTYYADSVKG | DAASYGMDV | IGHV3-53 | IGHJ6 | 0.008772 | G1 | 0.222222 |
| | (SEQ ID | (SEQ ID NO: 45) | (SEQ ID NO: | | | | | |
| | NO: 44) | | 165) | | | | | |
| K | RNYMS | VIYSGGSTYYADSVKG | DAASYGMDV | IGHV3-53 | IGHJ6 | 0.008772 | G1 | 0.222222 |
| | (SEQ ID | (SEQ ID NO: 45) | (SEQ ID NO: | | | | | |
| | NO: 44) | | 165) | | | | | |
| K | SNYMS | VIYSGGSTYYADSVKG | DAASYGMDV | IGHV3-53 | IGHJ6 | 0.004386 | G1 | 0.222222 |
| | (SEQ ID | (SEQ ID NO: 45) | (SEQ ID NO: | | | | | |
| | NO: 41) | | 165) | | | | | |
| K | SNYMS | VIYSGGSTYYADSVKG | DAASYGMDV | IGHV3-53 | IGHJ6 | 0.008772 | G1 | 0.222222 |
| | (SEQ ID | (SEQ ID NO: 45) | (SEQ ID NO: | | | | | |
| | NO: 41) | | 165) | | | | | |
| K | SNYMS | VIYSGGSTYYADSVKG | DAASYGMDV | IGHV3-53 | IGHJ6 | 0.008772 | G1 | 0.222222 |
| | (SEQ ID | (SEQ ID NO: 45) | (SEQ ID NO: | | | | | |
| | NO: 41) | | 165) | | | | | |
| K | SNYMR | VIYSGGSTYYADSVKG | DAQSYGMDD | IGHV3-66 | IGHJ6 | 0.004386 | G1 | 0.222222 |
| | (SEQ ID | (SEQ ID NO: 45) | (SEQ ID NO: | | | | | |
| | NO: 167) | | 168) | | | | | |
| K | SNYMR | LIYSGGSTYYADSVKG | DAQSYGMDV | IGHV3-66 | IGHJ6 | 0.008772 | G1 | 0.111111 |
| | (SEQ ID | (SEQ ID NO: 148) | (SEQ ID NO: | | | | | |
| | NO: 167) | | 169) | | | | | |
| K | SNYMR | VIYSGGSTYYADSAKG | DAQSYGMDV | IGHV3-66 | IGHJ6 | 0.008772 | G1 | 0.111111 |
| | (SEQ ID | (SEQ ID NO: 170) | (SEQ ID NO: | | | | | |
| | NO: 167) | | 169) | | | | | |
| K | RNYMS | VIYSGGSTYYADSVKG | DAQSYGMDV | IGHV3-66 | IGHJ6 | 0.008772 | G1 | 0.111111 |
| | (SEQ ID | (SEQ ID NO: 45) | (SEQ ID NO: | | | | | |
| | NO: 44) | | 169) | | | | | |
| K | SNYMI | VIYSGGSTYYADSVKG | DAQSYGMDV | IGHV3-66 | IGHJ6 | 0.013158 | G1 | 0.111111 |
| | (SEQ ID | (SEQ ID NO: 45) | (SEQ ID NO: | | | | | |
| | NO: 171) | | 169) | | | | | |
| K | SNYMN | VIYSGGSTYYADSVKG | DAQSYGMDV | IGHV3-66 | IGHJ6 | 0.008772 | G1 | 0.111111 |
| | (SEQ ID | (SEQ ID NO: 45) | (SEQ ID NO: | | | | | |
| | NO: 129) | | 169) | | | | | |
| K | SNYMN | VIYSGGSTYYADSVKG | DAQSYGMDV | IGHV3-66 | IGHJ6 | 0.008772 | G1 | 0.111111 |
| | (SEQ ID | (SEQ ID NO: 45) | (SEQ ID NO: | | | | | |
| | NO: 129) | | 169) | | | | | |
| K | SNYMR | VIYSGGSTYYADSVKG | DAQSYGMDV | IGHV3-66 | IGHJ6 | 0.004386 | G1 | 0.111111 |
| | (SEQ ID | (SEQ ID NO: 45) | (SEQ ID NO: | | | | | |
| | NO: 167) | | 169) | | | | | |
| K | SNYMR | VIYSGGSTYYADSVKG | DAQSYGMDV | IGHV3-66 | IGHJ6 | 0.004386 | G1 | 0.111111 |
| | (SEQ ID | (SEQ ID NO: 45) | (SEQ ID NO: | | | | | |
| | NO: 167) | | 169) | | | | | |
| K | SNYMR | VIYSGGSTYYADSVKG | DAQSYGMDV | IGHV3-66 | IGHJ6 | 0.008772 | G1 | 0.111111 |
| | (SEQ ID | (SEQ ID NO: 45) | (SEQ ID NO: | | | | | |
| | NO: 167) | | 169) | | | | | |
| K | SNYMR | VIYSGGSTYYADSVKG | DAQSYGMDV | IGHV3-66 | IGHJ6 | 0.008772 | G1 | 0.111111 |
| | (SEQ ID | (SEQ ID NO: 45) | (SEQ ID NO: | | | | | |
| | NO: 167) | | 169) | | | | | |
| K | SNYMR | VIYSGGSTYYADSVRG | DAQSYGMDV | IGHV3-66 | IGHJ6 | 0.008772 | G1 | 0.111111 |
| | (SEQ ID | (SEQ ID NO: 172) | (SEQ ID NO: | | | | | |
| | NO: 167) | | 169) | | | | | |
| K | SNYMR | VIYSGGSTYYADSVRG | DAQSYGMDV | IGHV3-66 | IGHJ6 | 0.013158 | G1 | 0.111111 |
| | (SEQ ID | (SEQ ID NO: 172) | (SEQ ID NO: | | | | | |
| | NO: 167) | | 169) | | | | | |
| K | SNYMS | VIYSGGSTYYADSVKG | DIVIYGMDV | IGHV3-66 | IGHJ6 | 0.008772 | G1 | 0.333333 |
| | (SEQ ID | (SEQ ID NO: 45) | (SEQ ID NO: | | | | | |
| | NO: 41) | | 173) | | | | | |
| K | SNYMS | VIYIGGSTYYADSVKG | DLDYYGMDV | IGHV3-53 | IGHJ6 | 0.017544 | G1 | 0.333333 |
| | (SEQ ID | (SEQ ID NO: 174) | (SEQ ID NO: 28) | | | | | |
| | NO: 41) | | | | | | | |
| K | SNYMS | VIYSGGSTFYADSVKG | DLDYYGMDV | IGHV3-53 | IGHJ6 | 0.004386 | G1 | 0.333333 |
| | (SEQ ID | (SEQ ID NO: 48) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.004386 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.008772 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.013158 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.004386 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.004386 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.00885 | G1 | 0.333333 |
| | (SEQ ID | (SEQ ID NO: 45) | (SEQ ID NO: 28) | | | | | |
| | NO: 41) | | | | | | | |
| K | SNYMS | VIYSGGSTYYADSVKG | DLDYYGMDV | IGHV3-53 | IGHJ6 | 0.004386 | G1 | 0.333333 |
| | (SEQ ID | (SEQ ID NO: 45) | (SEQ ID NO: 28) | | | | | |
| | NO: 41) | | | | | | | |
| K | SNYMT | VIYSGGSTYYADSVKG | DLDYYGMDV | IGHV3-53 | IGHJ6 | 0.004386 | G1 | 0.333333 |
| | (SEQ ID | (SEQ ID NO: 45) | (SEQ ID NO: 28) | | | | | |
| | NO: 133) | | | | | | | |
| K | SNYMC | VIYSGGSTYYADSVKG | DLQYRGMDV | IGHV3-53 | IGHJ6 | 0.008772 | G1 | 0.333333 |
| | (SEQ ID | (SEQ ID NO: 45) | (SEQ ID NO: | | | | | |
| | NO: 144) | | 175) | | | | | |
| K | SNYMS | VIYSGGSTYYADSVKG | DLQYRGMDV | IGHV3-53 | IGHJ6 | 0.004386 | G1 | 0.333333 |
| | (SEQ ID | (SEQ ID NO: 45) | (SEQ ID NO: | | | | | |
| | NO: 41) | | 175) | | | | | |
| K | SNYMS | IIYSGGSAFYTDSVKG | DLVVGGMDV | IGHV3-53 | IGHJ6 | 0.02193 | G1 | 0.333333 |
| | (SEQ ID | (SEQ ID NO: 176) | (SEQ ID NO: | | | | | |
| | NO: 41) | | 177) | | | | | |
| K | SNYMS | VIYSGGSTFYADSVKG | DLYYYGMDV | IGHV3-53 | IGHJ6 | 0.008772 | G1 | 0.333333 |
| | (SEQ ID | (SEQ ID NO: 48) | (SEQ ID NO: 27) | | | | | |
| | NO: 41) | | | | | | | |
| K | SNYMS | VIYSGGSTYYADSVKG | DNPMYGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 178) | | | | | |
| K | SHYMS | LIYGGHDTNYADSVKG | DRPLYGMDV | IGHV3-53 | IGHJ6 | 0.061404 | G1 | 0.333333 |
| | (SEQ ID | (SEQ ID NO: 180) | (SEQ ID NO: | | | | | |
| | NO: 179) | | 181) | | | | | |
| K | SNYMS | VIYSGGSTFYADSVKG | DRVVRGMDV | IGHV3-66 | IGHJ6 | 0.004386 | G1 | 0.333333 |
| | (SEQ ID | (SEQ ID NO: 48) | (SEQ ID NO: | | | | | |
| | NO: 41) | | 182) | | | | | |
| K | SNYMS | VIYSGGSTYYADSVKG | DRVVRGMDV | IGHV3-66 | IGHJ6 | 0.004386 | G2 | 0.333333 |
| | (SEQ ID | (SEQ ID NO: 45) | (SEQ ID NO: | | | | | |
| | NO: 41) | | 182) | | | | | |
| M | SNYMS | VIYSGGSTYYEDSVKG | DLDYYGMDV | IGHV3-53 | IGHJ6 | 0.017544 | G1 | 0.333333 |
| | (SEQ ID | (SEQ ID NO: 183) | (SEQ ID NO: 28) | | | | | |
| | NO: 41) | | | | | | | |
| M | RNYMS | VIYSGGSTYYADSVKG | DLSAYGMDV | IGHV3-53 | IGHJ6 | 0.013158 | G1 | 0.333333 |
| | (SEQ ID | (SEQ ID NO: 45) | (SEQ ID NO: | | | | | |
| | NO: 44) | | 184) | | | | | |
| M | SNYMS | VIYSGGSTYYADSVKG | DLSAYGMDV | IGHV3-53 | IGHJ6 | 0.004386 | G1 | 0.333333 |
| | (SEQ ID | (SEQ ID NO: 45) | (SEQ ID NO: | | | | | |
| | NO: 41) | | 184) | | | | | |
| M | SNYMS | VIYSGGSTYYADSVKG | DLSAYGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 184) | | | | | |
| O | SNYMS | VIYSGGSTYYADSVKG | DLIVYGMDV | IGHV3-66 | IGHJ6 | 0.013158 | G1 | 0.222222 |
| | (SEQ ID | (SEQ ID NO: 45) | (SEQ ID NO: | | | | | |
| | NO: 41) | | 185) | | | | | |
| O | SNYMS | VIYSGGSTYYADSVKG | DLMVRGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 186) | | | | | |
| O | SNYMS | VIYSGGSTYYADSVKG | DLSYYGMDV | IGHV3-53 | IGHJ6 | 0 | G1 | 0.333333 |
| | (SEQ ID | (SEQ ID NO: 45) | (SEQ ID NO: 33) | | | | | |
| | NO: 41) | | | | | | | |
| P | SNYMS | VIYSGGSTYYADSVKG | DAVSYGMDV | IGHV3-53 | IGHJ6 | 0 | G1 | 0.222222 |
| | (SEQ ID | (SEQ ID NO: 45) | (SEQ ID NO: 35) | | | | | |
| | NO: 41) | | | | | | | |
| P | SNYMS | VIYSGGSTYYADSVKG | DTDKYGMDV | IGHV3-66 | IGHJ6 | 0 | G3 | 0.333333 |
| | (SEQ ID | (SEQ ID NO: 45) | (SEQ ID NO: | | | | | |
| | NO: 41) | | 187) | | | | | |
| P | SNYMS | VIYSGGSTYYADSVKG | DTDYYGMDV | IGHV3-53 | IGHJ6 | 0 | G1 | 0.333333 |
| | (SEQ ID | (SEQ ID NO: 45) | (SEQ ID NO: | | | | | |
| | NO: 41) | | 188) | | | | | |

TABLE 5
Human mAbs reactive against MERS-CoV RBD

| Clone | V gene | J gene | Divergence |
|---|---|---|---|
| 4 | IGHV3-23 | IGHJ4 | 0.075862 |
| 13 | IGHV3-23 | IGHJ4 | 0.061224 |
| 28 | IGHV3-30 | IGHJ6 | 0.013559 |
| 34 | IGHV3-21 | IGHJ3 | 0.057627 |
| 36 | IGHV3-23 | IGHJ4 | 0.064626 |
| 38 | IGHV3-23 | IGHJ4 | 0.088136 |
| 42 | IGHV3-21 | IGHJ4 | 0.061433 |
| 103 | IGHV3-23 | IGHJ4 | 0.050847 |
| 119 | IGHV3-9 | IGHJ6 | 0.087838 |
| 180 | IGHV6-1 | IGHJ4 | 0.009836 |
| 113 | IGHV3-30 | IGHJ4 | 0.00339 |
| 121 | IGHV1-69 | IGHJ4 | 0.128028 |
| 6 | IGHV4-39 | IGHJ5 | 0.016892 |
| 25 | IGHV4-39 | IGHJ5 | 0.016892 |
| 10-1 | IGHV1-69 | IGHJ5 | 0.006757 |
| 20-1 | IGHV1-69 | IGHJ5 | 0.006757 |
| 38-1 | IGHV1-69 | IGHJ5 | 0.006757 |
| 39 | IGHV1-69 | IGHJ5 | 0.010135 |
| 40 | IGHV1-69 | IGHJ5 | 0.006757 |
| 11 | IGHV4-39 | IGHJ4 | 0.020067 |
| 26 | IGHV4-39 | IGHJ4 | 0.036789 |
| 21 | IGHV1-69 | IGHJ5 | 0.003378 |
| 17 | IGHV1-69 | IGHJ3 | 0.010135 |
| 30 | IGHV1-69 | IGHJ3 | 0.020339 |
| 33 | IGHV1-69 | IGHJ3 | 0.016949 |
| 41 | IGHV1-69 | IGHJ3 | 0.013514 |
| 46 | IGHV4-39 | IGHJ4 | 0.016722 |
| 47 | IGHV4-39 | IGHJ4 | 0.016722 |
| 48 | IGHV4-39 | IGHJ4 | 0.020067 |
| 7 | IGHV3-21 | IGHJ6 | 0.010274 |
| 9 | IGHV1-69 | IGHJ4 | 0.017065 |
| 31 | IGHV1-69 | IGHJ5 | 0.020339 |
| 35 | IGHV3-21 | IGHJ6 | 0.006849 |
| 42-1 | IGHV4-39 | IGHJ5 | 0.020067 |
| 10 | IGHV4-39 | IGHJ5 | 0.016722 |
| 15 | IGHV1-69 | IGHJ4 | 0.003413 |
| 20 | IGHV1-69 | IGHJ4 | 0.020619 |

TABLE 6
Statistics for the pre-processing of the IGH NGS data

| | | UMI-processed read | | | Sampled unique |
|---|---|---|---|---|---|
| | | (functional filtering | Unique consensus | Sampled UMI- | consensus |
| Sample | Raw read | performed) | sequence # | processed read | sequence # |
| A_d11 | 10,213,428 | 1,678,431 | 353,052 | 250,000 | 87,520 |
| A_d17 | 4,718,128 | 404,665 | 100,031 | 250,000 | 69,541 |
| A_d45 | 2,446,168 | 215,355 | 99,530 | 215,355 | 99,530 |
| B_d10 | 3,918,963 | 148,132 | 45,698 | 148,132 | 45,698 |
| B_d19 | 3,970,211 | 460,397 | 298,830 | 250,000 | 171,877 |
| C_d6 | 4,206,074 | 538,240 | 310,825 | 250,000 | 157,012 |
| C_d15 | 4,369,267 | 466,795 | 210,434 | 250,000 | 120,680 |
| D_d6 | 4,308,679 | 457,369 | 160,539 | 250,000 | 103,612 |
| D_d28 | 3,593,294 | 142,798 | 84,579 | 142,798 | 84,579 |
| E_d23 | 4,937,896 | 782,329 | 262,323 | 250,000 | 104,363 |
| E_d44 | 3,274,130 | 543,191 | 253,671 | 250,000 | 137,775 |
| E_d99 | 3,900,483 | 276,160 | 58,633 | 250,000 | 54,760 |
| F_d14 | 2,454,273 | 179,398 | 98,942 | 179,398 | 98,942 |
| F_d36 | 2,060,695 | 187,156 | 142,352 | 187,156 | 142,352 |
| G_d9 | 4,698,663 | 626,689 | 223,449 | 250,000 | 104,310 |
| G_d22 | 3,577,375 | 529,997 | 296,335 | 250,000 | 155,254 |
| H_d9 | 4,185,556 | 395,267 | 133,213 | 250,000 | 90,817 |
| I_d13 | 5,441,386 | 299,425 | 63,173 | 250,000 | 56,503 |
| J_d41 | 4,714,078 | 658,438 | 307,010 | 250,000 | 135,413 |
| K_d21 | 3,195,622 | 164,637 | 37,241 | 164,637 | 37,241 |
| L_d25 | 3,489,840 | 550,048 | 235,679 | 250,000 | 119,050 |
| M_d22 | 3,348,344 | 152,722 | 31,635 | 152,722 | 31,635 |
| N_d34 | 3,185,492 | 511,609 | 261,368 | 250,000 | 141,939 |
| O_d15 | 2,884,991 | 171,460 | 32,534 | 171,460 | 32,534 |
| P_d16 | 2,453,761 | 135,942 | 69,392 | 135,942 | 69,392 |
| Q_d35 | 2,497,207 | 189,122 | 71,993 | 189,122 | 71,993 |

TABLE 7
Statistics for the pre-processing of the IGκ and IGλ NGS data

| | Kappa chain (IGκ) repertoire | | | Lambda chain (IGλ) repertoire | | |
|---|---|---|---|---|---|---|
| | | UMI-processed | | | UMI-processed | |
| | | read | | | read | |
| | | (functional | Unique | | (functional | Unique |
| | | filtering | consensus | | filtering | consensus |
| Sample | Raw read | performed) | sequence # | Raw read | performed) | sequence # |
| A_d11 | 1,147,464 | 8,354 | 2,662 | 2,085,248 | 36,557 | 6,826 |
| A_d17 | 1,916,919 | 19,954 | 5,487 | 1,489,720 | 13,881 | 4,966 |
| A_d45 | 1,315,147 | 46,260 | 19,151 | 1,496,933 | 72,241 | 22,959 |
| B_d10 | 1,298,486 | 18,187 | 6,439 | 961,491 | 12,980 | 1,923 |
| B_d19 | 1,223,146 | 25,509 | 11,159 | 3,590,964 | 357,920 | 56,717 |
| C_d6 | 1,553,360 | 126,170 | 33,153 | 814,108 | 58,115 | 15,939 |
| C_d15 | 1,508,906 | 158,103 | 29,785 | 925,777 | 37,942 | 6,198 |
| D_d6 | 1,628,458 | 44,235 | 19,369 | 1,234,487 | 50,927 | 16,988 |
| D_d28 | 1,189,263 | 81,625 | 25,463 | 1,022,841 | 92,332 | 21,447 |
| E_d23 | 2,916,519 | 58,366 | 12,820 | 1,536,592 | 35,106 | 9,761 |
| E_d44 | 1,634,121 | 64,292 | 19,155 | 1,543,971 | 139,647 | 26,723 |
| E_d99 | 1,224,919 | 30,077 | 13,879 | 1,624,470 | 74,612 | 21,789 |
| F_d14 | 1,439,098 | 8,848 | 2,555 | 1,035,486 | 7,955 | 3,644 |
| F_d36 | 1,340,700 | 62,808 | 21,018 | 889,350 | 48,016 | 13,574 |
| G_d9 | 2,265,376 | 16,048 | 4,147 | 1,310,891 | 8,694 | 4,571 |
| G_d22 | 1,591,445 | 44,963 | 13,327 | 1,028,691 | 16,260 | 5,889 |

TABLE 8
The RBD-binding prediction clones

| | | | | | | | Mapped | Mapped |
|---|---|---|---|---|---|---|---|---|
| Clone | HCDR1 | HCDR2 | HCDR3 | V gene | J gene | Divergence | patient | isotype |
| P-003 | GFYIH (SEQ ID | RINPDSGATDYAQKFQG (SEQ ID NO: 190) | GDLRD (SEQ ID NO: 191) | IGHV1-2 | IGHJ4 | 0.02863 | E | A2 |
| | NO: 189) | | | | | | | |
| P-004 | GYYMH (SEQ ID | RINPNSGGTNYAQKFQG (SEQ ID NO: 193) | GHMDV (SEQ ID NO: 194) | IGHV1-2 | IGHJ6 | 0.002212 | A | G1 |
| | NO: 192) | | | | | | | |
| P-006 | GYYMH (SEQ ID | RINPNSGGTNYAQKFQG (SEQ ID NO: 193) | RNMDV (SEQ ID NO: 195) | IGHV1-2 | IGHJ6 | 0.002963 | A | G1 |
| | NO: 192) | | | | | | | |
| P-009 | SNYMS (SEQ ID | VIYSGGSTYYADSVKG (SEQ ID NO: 45) | DAFGMDV (SEQ ID NO: 196) | IGHV3-53 | IGHJ6 | 0.005679 | E | G1 |
| | NO: 41) | | | | | | | |
| P-014 | SYSMN (SEQ ID | YIYRRDSSIFYADSVKG (SEQ ID NO: 198) | EDWQSLDY (SEQ ID NO: 199) | IGHV3-48 | IGHJ4 | 0.140969 | A | A1 |
| | NO: 197) | | | | | | | |
| P-021 | SYWIG (SEQ ID | IIYPGDSDTRYSPSFQG (SEQ ID NO: 59) | WDSRAFDI (SEQ ID NO: 200) | IGHV5-51 | IGHJ3 | 0 | A | G1 |
| | NO: 58) | | | | | | | |
| P-022 | TYWIG (SEQ ID | IIYPGDSDTRYSPSFQG (SEQ ID NO: 59) | YNSGWLDF (SEQ ID NO: 202) | IGHV5-51 | IGHJ4 | 0.013216 | E | G1 |
| | NO: 201) | | | | | | | |
| P-023 | SYGMH (SEQ ID | VIWFDERNRYYSDSVKG (SEQ ID NO: | ANNYFPFDY (SEQ ID NO: 205) | IGHV3-33 | IGHJ4 | 0.034783 | A | A1 |
| | NO: 203) | 204) | | | | | | |
| P-026 | SNYMS (SEQ ID | VIYSGGSTYYADSVKG (SEQ ID NO: 45) | DAQRYGMDV (SEQ ID NO: 206) | IGHV3-53 | IGHJ6 | 0.008772 | E | A1 |
| | NO: 41) | | | | | | | |
| P-027 | SNYMS (SEQ ID | VLYSGGSTFYADSVKG (SEQ ID NO: 42) | DAQVYGMDV (SEQ ID NO: 43) | IGHV3-66 | IGHJ6 | 0.013158 | E | G1 |
| | NO: 41) | | | | | | | |
| P-031 | SNYMS (SEQ ID | VIYSGGSTYYADSVKG (SEQ ID NO: 45) | DLAVYGMDV (SEQ ID NO: 36) | IGHV3-53 | IGHJ6 | 0.010965 | A\|E | G1 |
| | NO: 41) | | | | | | | |
| P-032 | SNYMS (SEQ ID | VIYSGGSTYYADSVKG (SEQ ID NO: 45) | DLAVYGMDV (SEQ ID NO: 36) | IGHV3-53 | IGHJ6 | 0.010965 | A\|E | G1 |
| | NO: 41) | | | | | | | |
| P-035 | SNYMT (SEQ ID | VIYSGGSTFYADSVKG (SEQ ID NO: 48) | DLGPGGMDV (SEQ ID NO: 207) | IGHV3-53 | IGHJ6 | 0.02193 | E | G1 |
| | NO: 133) | | | | | | | |
| P-036 | SNYMS (SEQ ID | VIYSGGSTYYADSVKG (SEQ ID NO: 45) | DLGPYGMDV (SEQ ID NO: 38) | IGHV3-53 | IGHJ6 | 0.008772 | E | G1 |
| | NO: 41) | | | | | | | |
| P-042 | SNYMN (SEQ ID | VIYSGGSTYYADSVKG (SEQ ID NO: 45) | DLPYYGMDV (SEQ ID NO: 146) | IGHV3-66 | IGHJ6 | 0.004386 | E | G1 |
| | NO: 129) | | | | | | | |
| P-046 | RNYMS (SEQ ID | VIYSGGSTYYADTVKG (SEQ ID NO: 208) | DLSAYGMDV (SEQ ID NO: 184) | IGHV3-66 | IGHJ6 | 0.013158 | E | G1 |
| | NO: 44) | | | | | | | |
| P-047 | SNYMN (SEQ ID | VIYSGGSTFYADSVKG (SEQ ID NO: 48) | DLSELGVDY (SEQ ID NO: 209) | IGHV3-66 | IGHJ4 | 0.008772 | E | G2 |
| | NO: 129) | | | | | | | |
| P-048 | SNYMN (SEQ ID | VIYSGGSTFYADSVKG (SEQ ID NO: 48) | DLSYYGMDV (SEQ ID NO: 33) | IGHV3-53 | IGHJ6 | 0.030702 | A | G1 |
| | NO: 129) | | | | | | | |
| P-049 | SNYMS (SEQ ID | IIYSGGSTFYADSVKG (SEQ ID NO: 135) | DLTIFGMDV (SEQ ID NO: 210) | IGHV3-53 | IGHJ6 | 0.017544 | A | G1 |
| | NO: 41) | | | | | | | |
| P-050 | SNYMS (SEQ ID | VIYSGGSTYYADSVKG (SEQ ID NO: 45) | DLTVYGMDV (SEQ ID NO: 147) | IGHV3-53 | IGHJ6 | 0.008772 | E | A1 |
| | NO: 41) | | | | | | | |
| P-053 | SNYMS (SEQ ID | VIYAGGTTDYADSVKG (SEQ ID NO: 130) | DLVAYGMDV (SEQ ID NO: 29) | IGHV3-53 | IGHJ6 | 0.039474 | A | G1 |
| | NO: 41) | | | | | | | |
| P-055 | DYYMS (SEQ ID | YISSISSYTNYADSVKG (SEQ ID NO: 211) | DLVGGAFDI (SEQ ID NO: 212) | IGHV3-11 | IGHJ3 | 0.004329 | E | G1 |
| | NO: 55) | | | | | | | |
| P-056 | SNYMS (SEQ ID | VIYSGGSTFYADSVKG (SEQ ID NO: 48) | DLVVLGMDV (SEQ ID NO: 40) | IGHV3-66 | IGHJ6 | 0.008772 | E | A2 |
| | NO: 41) | | | | | | | |
| P-060 | SNYMT (SEQ ID | VIYSGGSTYYADSVKG (SEQ ID NO: 45) | DLVVRGVDI (SEQ ID NO: 213) | IGHV3-53 | IGHJ3 | 0.013158 | A | G1 |
| | NO: 133) | | | | | | | |
| P-061 | SNYMS (SEQ ID | LIYSGGSTYYADSVKG (SEQ ID NO: 148) | DLVVSGMDV (SEQ ID NO: 214) | IGHV3-66 | IGHJ6 | 0.017544 | E | A1 |
| | NO: 41) | | | | | | | |
| P-062 | SNYMT (SEQ ID | LIYSGGSTYYADSVKG (SEQ ID NO: 148) | DLVVWGMDV (SEQ ID NO: 149) | IGHV3-53 | IGHJ6 | 0.039474 | E | G1 |
| | NO: 133) | | | | | | | |
| P-063 | SNYMT (SEQ ID | VIYSGGSTYYADSVKG (SEQ ID NO: 45) | DLVVYGMDV (SEQ ID NO: 32) | IGHV3-53 | IGHJ6 | 0.013158 | E | A2 |
| | NO: 133) | | | | | | | |
| P-065 | SNYMS (SEQ ID | VLYSGGSTYYADSVKG (SEQ ID NO: 150) | DLVYYGMDV (SEQ ID NO: 31) | IGHV3-66 | IGHJ6 | 0.013158 | E | A1 |
| | NO: 41) | | | | | | | |
| P-068 | SNYMS (SEQ ID | VIYSGGSTFYADSVKG (SEQ ID NO: 48) | DLYYYGMDV (SEQ ID NO: 27) | IGHV3-53 | IGHJ6 | 0.004386 | A | G1 |
| | NO: 41) | | | | | | | |
| P-069 | SNYMS (SEQ ID | VIYSGGSTYYADSVKG (SEQ ID NO: 45) | DLYYYGMDV (SEQ ID NO: 27) | IGHV3-53 | IGHJ6 | 0.000731 | A | M\|G1 |
| | NO: 41) | | | | | | | |
| P-070 | SNYMS (SEQ ID | IIYSGGSTFYADSVKG (SEQ ID NO: 135) | DLYYYGMDV (SEQ ID NO: 27) | IGHV3-53 | IGHJ6 | 0.013158 | A | G1 |
| | NO: 41) | | | | | | | |
| P-073 | RNYMS (SEQ ID | VIYSGGSTYYADSVKG (SEQ ID NO: 45) | DVPIYGMDV (SEQ ID NO: 215) | IGHV3-53 | IGHJ6 | 0.013158 | A | G1 |
| | NO: 44) | | | | | | | |
| P-074 | SNYMS (SEQ ID | VIYSGGSTDYADSVKG (SEQ ID NO: 137) | DVVVYGMDV (SEQ ID NO: 216) | IGHV3-53 | IGHJ6 | 0.013158 | E | A1 |
| | NO: 41) | | | | | | | |
| P-075 | SNYMS (SEQ ID | VIYSGGSTFYSDSVKG (SEQ ID NO: 217) | DWGEYYFDY (SEQ ID NO: 218) | IGHV3-66 | IGHJ4 | 0.008772 | E | A1 |
| | NO: 41) | | | | | | | |
| P-077 | SNYMS (SEQ ID | VIYSGGSTFYADSVKG (SEQ ID NO: 48) | ELGVYGMDV (SEQ ID NO: 219) | IGHV3-53 | IGHJ6 | 0.013158 | A | G1 |
| | NO: 41) | | | | | | | |
| P-078 | SNYMS (SEQ ID | VIYSGGSTFYADSVKG (SEQ ID NO: 48) | ELYYYGMDV (SEQ ID NO: 220) | IGHV3-53 | IGHJ6 | 0.004386 | A | G1 |
| | NO: 41) | | | | | | | |
| P-082 | SNYMS (SEQ ID | IIYSGGSTFYADSVKG (SEQ ID NO: 135) | GYGDYYFDY (SEQ ID NO: 221) | IGHV3-66 | IGHJ4 | 0.013216 | E | A1 |
| | NO: 41) | | | | | | | |
| P-085 | SYWIG (SEQ ID | IIYPGDSDTRYSPSFQG (SEQ ID NO: 59) | QDSGWAFDY (SEQ ID NO: 222) | IGHV5-51 | IGHJ4 | 0.001087 | A | G3\|G1 |
| | NO: 58) | | | | | | | |
| P-087 | SNYMS (SEQ ID | LIYSGGSTFYADSVKG (SEQ ID NO: 223) | SLEYYGMDV (SEQ ID NO: 224) | IGHV3-53 | IGHJ6 | 0.00885 | E | G1 |
| | NO: 41) | | | | | | | |
| P-089 | SNWIG (SEQ ID | IIYPGDSDTRYSPSFQG (SEQ ID NO: 59) | VGDGYPFDY (SEQ ID NO: 226) | IGHV5-51 | IGHJ4 | 0.008772 | E | A1 |
| | NO: 225) | | | | | | | |
| P-090 | SSNWWS (SEQ ID | EIYHSGSTNYNPSLKS (SEQ ID NO: 228) | VPQADAFDI (SEQ ID NO: 229) | IGHV4-4 | IGHJ3 | 0 | A | G1 |
| | NO: 227) | | | | | | | |
| P-094 | SYWIG (SEQ ID | IIYPGDSDTRYSPSFQG (SEQ ID NO: 59) | APATYASFDY (SEQ ID NO: 230) | IGHV5-51 | IGHJ4 | 0.013274 | E | G1 |
| | NO: 58) | | | | | | | |
| P-095 | SGDYYWS (SEQ | YIYYSGSTYYNPSLKS (SEQ ID NO: 232) | AQWLRGHHDY (SEQ ID NO: 233) | IGHV4-30-4 | IGHJ4 | 0.001431 | A | G3\|G1 |
| | ID NO: 231) | | | | | | | |
| P-100 | SNYMS (SEQ ID | VIYSGGSTYYADSVKG (SEQ ID NO: 45) | DLDIVGAFDI (SEQ ID NO: 234) | IGHV3-66 | IGHJ3 | 0.002193 | E | G1 |
| | NO: 41) | | | | | | | |
| P-104 | SNYMS (SEQ ID | VIYSGGSTYYADSVKG (SEQ ID NO: 45) | DLDTAGGMDV (SEQ ID NO: 46) | IGHV3-66 | IGHJ6 | 0.02193 | E | A1 |
| | NO: 41) | | | | | | | |
| P-111 | SNYMN (SEQ ID | VIYSGGTTYYADSVKG (SEQ ID NO: 142) | DLEILGGMDV (SEQ ID NO: 235) | IGHV3-53 | IGHJ6 | 0.026316 | A | G1 |
| | NO: 129) | | | | | | | |
| P-117 | SNYMS (SEQ ID | VIYSGGSTYYADSVKG (SEQ ID NO: 45) | DLLEQGGMDV (SEQ ID NO: 47) | IGHV3-66 | IGHJ6 | 0.006579 | E | G1 |
| | NO: 41) | | | | | | | |
| P-130 | SNYMS (SEQ ID | VIYSGGSTFYADSVKG (SEQ ID NO: 48) | DLMAAGGMDV (SEQ ID NO: 236) | IGHV3-53 | IGHJ6 | 0.015351 | A | G1 |
| | NO: 41) | | | | | | | |
| P-136 | SNYMS (SEQ ID | LIYSGGSTFYADSVKG (SEQ ID NO: 223) | DLMAAGGMDV (SEQ ID NO: 236) | IGHV3-53 | IGHJ6 | 0.026316 | A | G1 |
| | NO: 41) | | | | | | | |
| P-137 | SNYMS (SEQ ID | VIYSGGSTFYADSVKG (SEQ ID NO: 48) | DLMAAGGMDV (SEQ ID NO: 236) | IGHV3-53 | IGHJ6 | 0.015351 | A | G1 |
| | NO: 41) | | | | | | | |
| P-138 | SNYMS (SEQ ID | VIYSGGSRYYADSVKG (SEQ ID NO: 237) | DLMAAGGMDV (SEQ ID NO: 236) | IGHV3-53 | IGHJ6 | 0.026316 | A | G1 |
| | NO: 41) | | | | | | | |
| P-139 | SNYMS (SEQ ID | VIYSGGTTYYADSVKG (SEQ ID NO: 142) | DLMAAGGMDV (SEQ ID NO: 236) | IGHV3-53 | IGHJ6 | 0.02193 | A | G1 |
| | NO: 41) | | | | | | | |
| P-146 | RNYMS (SEQ ID | VIYSGGSTYYADFVKG (SEQ ID NO: 238) | DLMAAGGMDV (SEQ ID NO: 236) | IGHV3-53 | IGHJ6 | 0.04386 | A | G1 |
| | NO: 44) | | | | | | | |
| P-168 | RNYMS (SEQ ID | VIYSGGSTFYADSVKG (SEQ ID NO: 48) | DLQEAGAFDI (SEQ ID NO: 51) | IGHV3-53 | IGHJ3 | 0.017544 | A | A1 |
| | NO: 44) | | | | | | | |
| P-182 | GYYMH (SEQ ID | WINPNSGGTNYAQKFQG (SEQ ID NO: | DLSNVVFFDS (SEQ ID NO: 240) | IGHV1-2 | IGHJ4 | 0.004405 | A | G1 |
| | NO: 192) | 239) | | | | | | |
| P-186 | SYWMS (SEQ ID | NIKQDGSEKYYVDSVKG (SEQ ID NO: 53) | DRWLRGDMDV (SEQ ID NO: 241) | IGHV3-7 | IGHJ6 | 0 | A | G1 |
| | NO: 52) | | | | | | | |
| P-194 | NAWMS (SEQ ID | RIKTKTDGGTTDYAAPVKG (SEQ ID NO: | EWGYYDSLDY (SEQ ID NO: 244) | IGHV3-15 | IGHJ4 | 0.012658 | G1 | |
| | NO: 242) | 243) | | | | | | |
| P-196 | DYYMS (SEQ ID | YISSSGSTIYYADSVKG (SEQ ID NO: 245) | GEWLRGGFDP (SEQ ID NO: 246) | IGHV3-11 | IGHJ5 | 0 | A | M\|G3\|G1 |
| | NO: 55) | | | | | | | |
| P-199 | SYYMH (SEQ ID | IIDPSGGSTSYAQKFQG (SEQ ID NO: 247) | HDISPYYFDY (SEQ ID NO: 248) | IGHV1-46 | IGHJ4 | 0.004484 | A | G1 |
| | NO: 88) | | | | | | | |
| P-201 | SYWIG (SEQ ID | IIYPGDSDTRYSPSFQG (SEQ ID NO: 59) | HENLYYGMDV (SEQ ID NO: 249) | IGHV5-51 | IGHJ6 | 0 | A | M\|G1 |
| | NO: 58) | | | | | | | |
| P-207 | SYWMS (SEQ ID | NIKQDGSEKYYVDSVKG (SEQ ID NO: 53) | HRWLRGEIDY (SEQ ID NO: 54) | IGHV3-7 | IGHJ4 | 0 | E | G1 |
| | NO: 52) | | | | | | | |
| P-224 | SSSYYWG (SEQ | TFYYSRSTYYNPSLKS (SEQ ID NO: 251) | LEWLRGHFDY (SEQ ID NO: 252) | IGHV4-39 | IGHJ4 | 0.012987 | E | A1 |
| | ID NO: 250) | | | | | | | |
| P-230 | SYWIG (SEQ ID | IIYPGDSDTRYSPSFQG (SEQ ID NO: 59) | MWSGVTAFDI (SEQ ID NO: 253) | IGHV5-51 | IGHJ3 | 0 | E | M |
| | NO: 58) | | | | | | | |
| P-231 | SSSYYWG (SEQ | SIYYSGSTYYNPSLKS (SEQ ID NO: 254) | NEWLRGPFDY (SEQ ID NO: 255) | IGHV4-39 | IGHJ4 | 0.017316 | A | G1 |
| | ID NO: 250) | | | | | | | |
| P-233 | SYDIN (SEQ ID | WMNPNSGNTGYAQKFQG (SEQ ID NO: | NPGGSGQFDP (SEQ ID NO: 258) | IGHV1-8 | IGHJ5 | 0.03125 | A | M |
| | NO: 256) | 257) | | | | | | |
| P-234 | RNYMS (SEQ ID | VIYSGGSTFYADSVKG (SEQ ID NO: 48) | PVMSRDGMDV (SEQ ID NO: 259) | IGHV3-66 | IGHJ6 | 0.011852 | E | G1 |
| | NO: 44) | | | | | | | |
| P-235 | SNYMS (SEQ ID | VIYPGGTTYYADSVKG (SEQ ID NO: 260) | QLPFGDYFDY (SEQ ID NO: 261) | IGHV3-53 | IGHJ4 | 0.030973 | A | G1 |
| | NO: 41) | | | | | | | |
| P-242 | SNFMS (SEQ ID | VIYSGGSTYYADSVKG (SEQ ID NO: 45) | QRWRQGWFDP (SEQ ID NO: 263) | IGHV3-53 | IGHJ5 | 0.004425 | A | G1 |
| | NO: 262) | | | | | | | |
| P-243 | SSSYYWG (SEQ | SIYYSGSTYYNPSLKS (SEQ ID NO: 254) | REWLRGHVDV (SEQ ID NO: 264) | IGHV4-39 | IGHJ6 | 0 | E | G1 |
| | ID NO: 250) | | | | | | | |
| P-246 | SSSYYWG (SEQ | SIYYSGSTYYNPSLKS (SEQ ID NO: 254) | RKWLRGAFDI (SEQ ID NO: 265) | IGHV4-39 | IGHJ3 | 0 | E | G1 |
| | ID NO: 250) | | | | | | | |
| P-251 | YYWIG (SEQ ID | IIYPGDSDTRYSPSFQG (SEQ ID NO: 59) | RSTTVGWLDY (SEQ ID NO: 267) | IGHV5-51 | IGHJ4 | 0.008734 | E | G1 |
| | NO: 266) | | | | | | | |
| P-252 | SYWMS (SEQ ID | NIKQDGSEKYYVDSVKG (SEQ ID NO: 53) | RVYYYGWLDV (SEQ ID NO: 268) | IGHV3-7 | IGHJ6 | 0.001456 | A | G3\|G1 |
| | NO: 52) | | | | | | | |
| P-261 | SYGIH (SEQ ID | LISYDGSDKYYADPVKG (SEQ ID NO: 270) | SSWLRGAFDY (SEQ ID NO: 57) | IGHV3-30 | IGHJ4 | 0.038961 | A | G1 |
| | NO: 269) | | | | | | | |
| P-268 | SYYMH (SEQ ID | IINPSGGSTSYAQKFQG (SEQ ID NO: 70) | SSWYKLGFDP (SEQ ID NO: 271) | IGHV1-46 | IGHJ5 | 0 | E | G1 |
| | NO: 88) | | | | | | | |
| P-269 | SSSYYWG (SEQ | SIYYSGSTYYNPSLKS (SEQ ID NO: 254) | TPWLRGAFDY (SEQ ID NO: 272) | IGHV4-39 | IGHJ4 | 0.001082 | E | G3\|G1\|A1 |
| | ID NO: 250) | | | | | | | |
| P-275 | SYEMN (SEQ ID | YISSSGSTIYYADSVKG (SEQ ID NO: 245) | TQWLRGAFDI (SEQ ID NO: 274) | IGHV3-48 | IGHJ3 | 0 | A | G1 |
| | NO: 273) | | | | | | | |
| P-315 | VNYMT (SEQ ID | LIYSGGSTYYADSVKG (SEQ ID NO: 148) | VLPYGDYADF (SEQ ID NO: 276) | IGHV3-53 | IGHJ4 | 0.022026 | E | A1 |
| | NO: 275) | | | | | | | |
| P-317 | SNYMS (SEQ ID | LIYSGGSTYYADSVKG (SEQ ID NO: 148) | VLPYGDYVDY (SEQ ID NO: 277) | IGHV3-53 | IGHJ4 | 0.008811 | A | G1 |
| | NO: 41) | | | | | | | |
| P-319 | SNWIA (SEQ ID | IIYPGDSITITYSPSFQG (SEQ ID NO: 279) | ALGHIGSGYDY (SEQ ID NO: 280) | IGHV5-51 | IGHJ4 | 0.04386 | E | G1 |
| | NO: 278) | | | | | | | |
| P-320 | SHWIG (SEQ ID | IIYPGDSDTRYSPSFQG (SEQ ID NO: 59) | APSGYYNWFDP (SEQ ID NO: 282) | IGHV5-51 | IGHJ5 | 0.008772 | A | G1 |
| | NO: 281) | | | | | | | |
| P-321 | SYGMH (SEQ ID | IISYDGSNKYYADSVKG (SEQ ID NO: 283) | AQSWTHWYFDL (SEQ ID NO: 284) | IGHV3-30 | IGHJ2 | 0.004348 | E | G1 |
| | NO: 203) | | | | | | | |
| P-322 | HYAIS (SEQ ID | RIIPMLDISNYAQKFKG (SEQ ID NO: 286) | DHTILPKGMDV (SEQ ID NO: 287) | IGHV1-69 | IGHJ6 | 0.044053 | E | G2 |
| | NO: 285) | | | | | | | |
| P-324 | DYAMS (SEQ ID | FIRSKAYGGTTEYAASVKG (SEQ ID NO: | DLRGSSGWYDI (SEQ ID NO: 288) | IGHV3-49 | IGHJ3 | 0.004219 | E | A2 |
| | NO: 102) | 103) | | | | | | |
| P-326 | SYAMH (SEQ ID | VISSDGGNKYYADSVKG (SEQ ID NO: | DTLLLVDAFDI (SEQ ID NO: 291) | IGHV3-30-3 | IGHJ3 | 0.008658 | A | G1 |
| | NO: 289) | 290) | | | | | | |
| P-327 | DYQMS (SEQ ID | YISSSSSYTNYADSVKG (SEQ ID NO: 293) | DWGYSSPRFDY (SEQ ID NO: 294) | IGHV3-11 | IGHJ4 | 0.008658 | E | G1 |
| | NO: 292) | | | | | | | |
| P-331 | SYWIG (SEQ ID | IITRYPGDSDYSPSFQG (SEQ ID NO: 59) | HGNWANSDLDY (SEQ ID NO: 295) | IGHV5-51 | IGHJ4 | 0.015217 | A | G1 |
| | NO: 58) | | | | | | | |
| P-333 | SYWIA (SEQ ID | IIYPGDSDTRYSPSFQG (SEQ ID NO: 59) | LPSSWYNWFDP (SEQ ID NO: 296) | IGHV5-51 | IGHJ5 | 0.0131 | E | G1 |
| | NO: 61) | | | | | | | |
| P-335 | SDWIG (SEQ ID | IIYPGDSDTRYSPSFQG (SEQ ID NO: 59) | MLCGGDCPFDY (SEQ ID NO: 298) | IGHV5-51 | IGHJ4 | 0.00885 | E | A1 |
| | NO: 297) | | | | | | | |
| P-338 | SYWIG (SEQ ID | IIYPGDSDTRYSPSFQG (SEQ ID NO: 59) | SIVTTNAGFDF (SEQ ID NO: 299) | IGHV5-51 | IGHJ4 | 0.008811 | E | G1 |
| | NO: 58) | | | | | | | |
| P-340 | SYWIG (SEQ ID | IIYPGDSDTRYSPSFQG (SEQ ID NO: 59) | SSSGPHDAFDI (SEQ ID NO: 300) | IGHV5-51 | IGHJ3 | 0 | E | M |
| | NO: 58) | | | | | | | |
| P-341 | SNYMS (SEQ ID | VIYSGGSTFYADSVKG (SEQ ID NO: 48) | VLPLYGDYLDY (SEQ ID NO: 301) | IGHV3-53 | IGHJ4 | 0.004405 | E | A1 |
| | NO: 41) | | | | | | | |
| P-342 | SYGIT (SEQ ID | WISAYNGNTKYAQKLQG (SEQ ID NO: | VMGIAVAGTVV (SEQ ID NO: 304) | IGHV1-18 | IGHJ6 | 0.015487 | A | G1 |
| | NO: 302) | 303) | | | | | | |
| P-345 | SYAMH (SEQ ID | AISSNGGSTYYANSVKG (SEQ ID NO: 305) | VPDDLIWYFDL (SEQ ID NO: 306) | IGHV3-64 | IGHJ2 | 0.001449 | E | G3\|G1 |
| | NO: 289) | | | | | | | |
| P-346 | SYAMH (SEQ ID | AISSNGGSTYYANSVKG (SEQ ID NO: 305) | VPDDLNWYFDL (SEQ ID NO: 307) | IGHV3-64 | IGHJ2 | 0.002899 | E | G1 |
| | NO: 289) | | | | | | | |
| P-348 | SYGIS (SEQ ID | WISAYNGNTNYAQKLQG (SEQ ID NO: | VVELGIGWFDP (SEQ ID NO: 310) | IGHV1-18 | IGHJ5 | 0 | A | G1 |
| | NO: 308) | 309) | | | | | | |
| P-349 | STSFHWG (SEQ | TISYSGRAYHNPSLKS (SEQ ID NO: 312) | WNSHYYYGMHV (SEQ ID NO: 313) | IGHV4-39 | IGHJ6 | 0.081897 | A | G2 |
| | ID NO: 311) | | | | | | | |
| P-353 | SYWIA (SEQ ID | IIYPGDSDTRYSPSFQG (SEQ ID NO: 59) | YSSSPNGWFDP (SEQ ID NO: 62) | IGHV5-5I | IGHJ5 | 0.006579 | E | G1 |
| | NO: 61) | | | | | | | |
| P-357 | NYAMS (SEQ ID | AISGSGGSTYYADSVKG (SEQ ID NO: 315) | AIAAAGYWVFDY (SEQ ID NO: 316) | IGHV3-23 | IGHJ4 | 0.004386 | E | G1 |
| | NO: 314) | | | | | | | |
| P-358 | KCVMS (SEQ ID | SISDGGDNINDADSVKG (SEQ ID NO: 318) | AKSGSDRHVFEI (SEQ ID NO: 319) | IGHV3-23 | IGHJ3 | 0 104348 | A | G2 |
| | NO: 317) | | | | | | | |
| P-362 | SVDYYWS (SEQ | YIYYSGSTYYNPSLKS (SEQ ID NO: 232) | DLRWGRGGGMDV (SEQ ID NO: 321) | IGHV4-30-4 | IGHJ6 | 0.029915 | A | G1 |
| | ID NO: 320) | | | | | | | |
| P-363 | DYAMH (SEQ ID | GISWNSGNIGYADSVKG (SEQ ID NO: 322) | DSLGELLSGMDV (SEQ ID NO: 323) | IGHV3-9 | IGHJ6 | 0.004292 | E | G1 |
| | NO: 72) | | | | | | | |
| P-365 | DYAMH (SEQ ID | GISWNSGSIGYADSVKG (SEQ ID NO: 324) | DSSAGHGDYFDY (SEQ ID NO: 325) | IGHV3-9 | IGHJ4 | 0.004329 | A | G1 |
| | NO: 72) | | | | | | | |
| P-366 | DYAMH (SEQ ID | GISWNSGGIAYADSVKG (SEQ ID NO: 326) | DSSAGHGDYFDY (SEQ ID NO: 325) | IGHV3-9 | IGHJ4 | 0.008658 | A | G1 |
| | NO: 72) | | | | | | | |
| P-369 | SNAIS (SEQ ID | RIIPIFGTANYAQKFQG (SEQ ID NO: 64) | DVIESPLYGMDV (SEQ ID NO: 65) | IGHV1-69 | IGHJ6 | 0.030837 | A | G1 |
| | NO: 63) | | | | | | | |
| P-382 | SFAIT (SEQ ID | RIIPILGIANYAQKFQG (SEQ ID NO: 67) | EFSGGDNTGFDY (SEQ ID NO: 68) | IGHV1-69 | IGHJ4 | 0.008811 | E | G1 |
| | NO: 66) | | | | | | | |
| P-385 | RNYMS (SEQ ID | VIYSGGTTYYTDSVKG (SEQ ID NO: 327) | GDILTAPPPIDY (SEQ ID NO: 328) | IGHV3-66 | IGHJ4 | 0.017621 | E | A1 |
| | NO: 44) | | | | | | | |
| P-387 | SNIVTWI (SEQ ID | RTYYRSKWYNDYAVSVKS (SEQ ID NO: | GRFGGYFYGMDV (SEQ ID NO: 331) | IGHV6-1 | IGHJ6 | 0.064655 | A | G2 |
| | NO: 329) | 330) | | | | | | |
| P-388 | DYAMH (SEQ ID | GISWNSGSIGYADSVKG (SEQ ID NO: 324) | GRLGELLDAFDI (SEQ ID NO: 332) | IGHV3-9 | IGHJ3 | 0 | A | M\|GI |
| | NO: 72) | | | | | | | |
| P-389 | SYWMH (SEQ ID | RINGDGSDTGYADSLRA (SEQ ID NO: 334) | GVDYGRGAVLQH (SEQ ID NO: 335) | IGHV3-74 | IGHJ1 | 0.073913 | A | A2 |
| | NO: 333) | | | | | | | |
| P-392 | DYWIG (SEQ ID | IIYPGDSDTRYSPSFQG (SEQ ID NO: 59) | HSLADPVHWFDP (SEQ ID NO: 337) | IGHV5-51 | IGHJ5 | 0.017391 | E | A1 |
| | NO: 336) | | | | | | | |
| P-394 | SYWIG (SEQ ID | IIYPGDSDTRYSPSFQG (SEQ ID NO: 59) | LESIAAAGWADY (SEQ ID NO: 338) | IGHV5-51 | IGHJ4 | 0 | E | A1 |
| | NO: 58) | | | | | | | |
| P-395 | SYWIG (SEQ ID | IINPGDSETIYSPSFQG (SEQ ID NO: 339) | LGSGGSHNWFDP (SEQ ID NO: 340) | IGHV5-51 | IGHJ5 | 0.017467 | E | G1 |
| | NO: 58) | | | | | | | |
| P-398 | SGDYYWN (SEQ | YIYYSGSTYYNPSLKS (SEQ ID NO: 232) | SSPLVVTDAFDI (SEQ ID NO: 342) | IGHV4-30-4 | IGHJ3 | 0.006579 | A | G1 |
| | ID NO: 341) | | | | | | | |
| P-400 | SNFMS (SEQ ID | VIYSGGSTYYADSVKG (SEQ ID NO: 45) | VGWGYDSEYFDL (SEQ ID NO: 343) | IGHV3-53 | IGHJ2 | 0.024229 | E | A1\|A2 |
| | NO: 262) | | | | | | | |
| P-404 | SNSAAWN (SEQ | RTYYRFKWYYDYALSLES (SEQ ID NO: | VSAPGPRGWFDP (SEQ ID NO: 346) | IGHV6-1 | IGHJ5 | 0.050633 | E | G1 |
| | ID NO: 344) | 345) | | | | | | |
| P-406 | SNYMS (SEQ ID | LIYSGGSTYYADSVKG (SEQ ID NO: 148) | ALEVNAFGDYFDY (SEQ ID NO: 347) | IGHV3-66 | IGHJ4 | 0.004405 | E | A1 |
| | NO: 41) | | | | | | | |
| P-408 | SYYMH (SEQ ID | IINPGGGSTSYAQKFQG (SEQ ID NO: 348) | DAGYVPTTGGMDV (SEQ ID NO: 349) | IGHV1-46 | IGHJ6 | 0.017621 | E | G1 |
| | NO: 88) | | | | | | | |
| P-409 | TYYWS (SEQ ID | YIYNSGSTNYNPSLKS (SEQ ID NO: 351) | DANLSGSFDALDI (SEQ ID NO: 32) | IGHV4-59 | IGHJ3 | 0.061404 | E | G1 |
| | NO: 350) | | | | | | | |
| P-410 | DYAMH (SEQ ID | GISWNSGTIGYADSVKG (SEQ ID NO: 73) | DGGAVAETYGMDV (SEQ ID NO: 353) | IGHV3-9 | IGHJ6 | 0.008621 | E | G1 |
| | NO: 72) | | | | | | | |
| P-411 | SHYMH (SEQ ID | IINPSGGSTSYAQKFQG (SEQ ID NO: 70) | DGYFVPARSAFDI (SEQ ID NO: 71) | IGHV1-46 | IGHJ3 | 0.008811 | E | M |
| | NO: 69) | | | | | | | |
| P-435 | SYYMH (SEQ ID | IINPDAGSTTYAQKFQG (SEQ ID NO: 80) | DLYGLPGRAAFDI (SEQ ID NO: 81) | IGHV1-46 | IGHJ3 | 0.022026 | A | G1 |
| | NO: 88) | | | | | | | |
| P-440 | NHYMH (SEQ ID | IINPSGGSTSYAQKFQG (SEQ ID NO: 70) | DRWFIPQSGYFDL (SEQ ID NO: 355) | IGHV1-46 | IGHJ2 | 0.011013 | A | G1 |
| | NO: 354) | | | | | | | |
| P-441 | SYYMH (SEQ ID | IINPSGGSTSYAQKFQG (SEQ ID NO: 70) | DSYYLPAMGPFDY (SEQ ID NO: 36) | IGHV1-46 | IGHJ4 | 0 | A | G1 |
| | NO: 88) | | | | | | | |
| P-447 | SYYMH (SEQ ID | IINPSGGSTSYAQKFQG (SEQ ID NO: 70) | GAWGVPAASPSDP (SEQ ID NO: 357) | IGHV1-46 | IGHJ5 | 0 | E | G1 |
| | NO: 88) | | | | | | | |
| P-448 | SNYMS (SEQ ID | VIYSGGSTYYADSVKG (SEQ ID NO: 45) | GDGSGDYYYGMDV (SEQ ID NO: 82) | IGHV3-53 | IGHJ6 | 0 | E | A2 |
| | NO: 41) | | | | | | | |
| P-449 | SNYMS (SEQ ID | VIYSGGSTFYADSVKG (SEQ ID NO: 48) | GDGSGDYYYGMDV (SEQ ID NO: 82) | IGHV3-53 | IGHJ6 | 0.008811 | E | A2 |
| | NO: 41) | | | | | | | |
| P-453 | SYYMH (SEQ ID | IINPSGGSTSYAQKFQA (SEQ ID NO: 358) | GGVVPAASSAFDI (SEQ ID NO: 359) | IGHV1-46 | IGHJ3 | 0.017699 | E | G1 |
| | NO: 88) | | | | | | | |
| P-454 | SYAMH (SEQ ID | VISYDGSNKYYADSVKG (SEQ ID NO: 56) | GKWYSSPLEYFDY (SEQ ID NO: 360) | IGHV3-30-3 | IGHJ4 | 0.008621 | A | G1 |
| | NO: 289) | | | | | | | |
| P-455 | DYAMH (SEQ ID | AISWNSGSIDYADSVKG (SEQ ID NO: 361) | GLLAEFVVPTLDY (SEQ ID NO: 362) | IGHV3-9 | IGHJ4 | 0.008696 | E | A1 |
| | NO: 72) | | | | | | | |
| P-456 | SYWIS (SEQ ID | RIDPSDSYTNYSPSFQG (SEQ ID NO: 106) | GQQWLSNNWYFDL (SEQ ID NO: 364) | IGHV5-10-1 | IGHJ2 | 0.001096 | E | M\|G3\|G1 |
| | NO: 363) | | | | | | | |
| P-458 | SYWIG (SEQ ID | IIYPGDSDTRYSPSFQG (SEQ ID NO: 59) | HLDWNAPRGAFDI (SEQ ID NO: 36) | IGHV5-51 | IGHJ3 | 0 | A | G1 |
| | NO: 58) | | | | | | | |
| P-461 | SYWIG (SEQ ID | IIYPGDSDTRYSPSFQG (SEQ ID NO: 59) | HLDWNAPRGPFDI (SEQ ID NO: 84) | IGHV5-51 | IGHJ3 | 0 | A | G1 |
| | NO: 58) | | | | | | | |
| P-468 | SSNWWS (SEQ ID | EIYHSGSTNYNPSLKS (SEQ ID NO: 228) | LGHGDPGLRYFDL (SEQ ID NO: 366) | IGHV4-4 | IGHJ2 | 0 | E | G1 |
| | NO: 227) | | | | | | | |
| P-472 | SSNWWS (SEQ ID | EIFHSGSASYNPSLKS (SEQ ID NO: 367) | LGHGDPGLRYFDL (SEQ ID NO: 366) | IGHV4-4 | IGHJ2 | 0.022026 | E | A1 |
| | NO: 227) | | | | | | | |
| P-475 | NAWMS (SEQ ID | RIKSKTDGGTTDYAAPVKG (SEQ ID NO: | NDVIQYYHYGMDV (SEQ ID NO: 369) | IGHV3-15 | IGHJ6 | 0.004348 | A | G1 |
| | NO: 242) | 368) | | | | | | |
| P-476 | NAWMS (SEQ ID | RIKSKTDGGTTDYAAPVKG (SEQ ID NO: | NDVLQYYYYGMDV (SEQ ID NO: 370) | IGHV3-15 | IGHJ6 | 0 | A | G1 |
| | NO: 242) | 368) | | | | | | |
| P-477 | DFAMS (SEQ ID | FIRGTAYGGTTEYAASVKG (SEQ ID NO: | NHMTTVTWLGADI (SEQ ID NO: 373) | IGHV3-49 | IGHJ3 | 0.013043 | E | G1 |
| | NO: 371) | 372) | | | | | | |
| P-481 | GYYMH (SEQ ID | RINPNSGGTNYAQKFQG (SEQ ID NO: 193) | PGSISLVRGVRDV (SEQ ID NO: 374) | IGHV1-2 | IGHJ6 | 0 | E | G3 |
| | NO: 192) | | | | | | | |
| P-483 | NAWMS (SEQ ID | RIKSKTDGGTTDYAAPVKG (SEQ ID NO: | SDILQYYYYGMDV (SEQ ID NO: 375) | IGHV3-15 | IGHJ6 | 0.002128 | E | M |
| | NO: 242) | 368) | | | | | | |
| P-485 | NYGMH (SEQ ID | GVSYDGSDKYYADSVKG (SEQ ID NO: | TVATHYYYYGMDV (SEQ ID NO: 378) | IGHV3-30 | IGHJ6 | 0.030303 | E | G3 |
| | NO: 376) | 377) | | | | | | |
| P-487 | SYAIS (SEQ ID | RIIPILGIANYAQKFQG (SEQ ID NO: 67) | AALYGDYEEGYFDY (SEQ ID NO: 379) | IGHV1-69 | IGHJ4 | 0 | E | G1 |
| | NO: 94) | | | | | | | |
| P-488 | SYGMH (SEQ ID | VISYDGSNKYYADSVKG (SEQ ID NO: 56) | AGYSYGYPEIYFDY (SEQ ID NO: 380) | IGHV3-30 | IGHJ4 | 0.00622 | E | G1 |
| | NO: 203) | | | | | | | |
| P-489 | DYAMH (SEQ ID | GISWNSGTIGYADSVKG (SEQ ID NO: 73) | ALQPMDGGEYYFDY (SEQ ID NO: 381) | IGHV3-9 | IGHJ4 | 0.004348 | E | A1 |
| | NO: 72) | | | | | | | |
| P-491 | DYAMY (SEQ ID | GSSWNSGTIGYADSVKG (SEQ ID NO: 86) | DAGVTEYYYYGMDV (SEQ ID NO: 383) | IGHV3-9 | IGHJ6 | 0.034483 | A | G1 |
| | NO: 382) | | | | | | | |
| P-499 | DYAMH (SEQ ID | GISWNSGTIGYADSVKG (SEQ ID NO: 73) | DIGFGELLSYGMDV (SEQ ID NO: 384) | IGHV3-9 | IGHJ6 | 0.004292 | A | M |
| | NO: 72) | | | | | | | |
| P-500 | DYAMH (SEQ ID | GISWNSGTIGYADSVKG (SEQ ID NO: 73) | DIRKGDGFEFYFDY (SEQ ID NO: 385) | IGHV3-9 | IGHJ4 | 0.008584 | E | A2 |
| | NO: 72) | | | | | | | |
| P-506 | DYAMH (SEQ ID | GSSWNSGTIGYADSVKG (SEQ ID NO: 86) | DMGRGNDNNLAFDI (SEQ ID NO: 87) | IGHV3-9 | IGHJ3 | 0.038961 | E | G1 |
| | NO: 72) | | | | | | | |
| P-507 | SYAMS (SEQ ID | AISGSGGSTYYADSVKG (SEQ ID NO: 315) | DPMVRGPSFDYFDY (SEQ ID NO: 387) | IGHV3-23 | IGHJ4 | 0 | A | G3\|G1\|A1 |
| | NO: 386) | | | | | | | |
| P-511 | RYGMH (SEQ ID | VISYDGSNKYYVDSVKG (SEQ ID NO: | DVPLGIAATYLFDY (SEQ ID NO: 390) | IGHV3-33 | IGHJ4 | 0.017316 | E | G1 |
| | NO: 388) | 389) | | | | | | |
| P-512 | SNYMS (SEQ ID | VIYSGGSTFYADSVKG (SEQ ID NO: 48) | EAGMGAAAGTAFDY (SEQ ID NO: 391) | IGHV3-53 | IGHJ4 | 0.004386 | E | G1 |
| | NO: 41) | | | | | | | |
| P-513 | SYYMH (SEQ ID | IINPSGGSTSYAQKFQG (SEQ ID NO: 70) | EGVWDSSGYSSFDY (SEQ ID NO: 89) | IGHV1-46 | IGHJ4 | 0.013216 | E | A1 |
| | NO: 88) | | | | | | | |
| P-524 | DYAMH (SEQ ID | GISWNSGSIVYADSVKG (SEQ ID NO: 392) | GHTAMHYYYYGMDV (SEQ ID NO: 393) | IGHV3-9 | IGHJ6 | 0.008696 | E | G1 |
| | NO: 72) | | | | | | | |
| P-526 | SYWIG (SEQ ID | IIYPGDSDTRYSPSFQG (SEQ ID NO: 59) | HEGACSGGSCGIDY (SEQ ID NO: 394) | IGHV5-51 | IGHJ4 | 0 | A | G1 |
| | NO: 58) | | | | | | | |
| P-529 | NYGMH (SEQ ID | VISYDGSNKYYADSVKG (SEQ ID NO: 56) | NIYSYAYPQYYFDY (SEQ ID NO: 395) | IGHV3-30 | IGHJ4 | 0.021645 | A | G1 |
| | NO: 376) | | | | | | | |
| P-533 | NYGMH (SEQ ID | GVSYDGSDKYYADSVKG (SEQ ID NO: | TVATHYYYYYGMDV (SEQ ID NO: 396) | IGHV3-30 | IGHJ6 | 0.030303 | E | G3 |
| | NO: 376) | 377) | | | | | | |
| P-547 | SYWIG (SEQ ID | IIYPGDSDTRYSPSFQG (SEQ ID NO: 59) | AGDSSGWAPLDAFDI (SEQ ID NO: 397) | IGHV5-51 | IGHJ3 | 0.013274 | A | G1 |
| | NO: 58) | | | | | | | |
| P-548 | SYGMH (SEQ ID | VISYDGSNKYYADSVKG (SEQ ID NO: 56) | APIGYCTNGVCYFDY (SEQ ID NO: 398) | IGHV3-30 | IGHJ4 | 0 | A | WI |
| | NO: 203) | | | | | | | |
| P-550 | SYAIS (SEQ ID | RIIPILGIANFIANYAQKFQG (SEQ ID NO: | DDYSNYDYYYYGMDV (SEQ ID NO: 400) | IGHV1-69 | IGHJ6 | 0.050209 | E | A1 |
| | NO: 94) | 399) | | | | | | |
| P-561 | DYAMH (SEQ ID | GVTWNSGSIGYADSVKG (SEQ ID NO: 90) | DISPMLRGDNYGMDV (SEQ ID NO: 91) | IGHV3-9 | IGHJ6 | 0.017167 | E | G1 |
| | NO: 72) | | | | | | | |
| P-591 | SNYMS (SEQ ID | VIYSGGSTYYADSVKG (SEQ ID NO: 45) | DLRDSSGYSFGAFDI (SEQ ID NO: 401) | IGHV3-53 | IGHJ3 | 0 | E | A1 |
| | NO: 41) | | | | | | | |
| P-592 | SYGMH (SEQ ID | FISYDGSNKYYADSVKG (SEQ ID NO: 402) | DMAVAGYYYYYGMDV (SEQ ID NO: 403) | IGHV3-33 | IGHJ6 | 0.00868 | E | G1 |
| | NO: 203) | | | | | | | |
| P-610 | SYYMH (SEQ ID | IINPSGGSRSYAQKFQG (SEQ ID NO: 404) | DYDYVWGSYPNAFDI (SEQ ID NO: 405) | IGHV1-46 | IGHJ3 | 0.008811 | A | G1 |
| | NO: 88) | | | | | | | |
| P-611 | SYYMH (SEQ ID | IINPSGGSTSYAQKFQG (SEQ ID NO: 70) | DYDYVWGSYPNAFDI (SEQ ID NO: 405) | IGHV1-46 | IGHJ3 | 0 | A | G1 |
| | NO: 88) | | | | | | | |
| P-614 | SYAIS (SEQ ID | GIIPMFGTANYAQKFQG (SEQ ID NO: 406) | ERSVTKNLYYYGMDV (SEQ ID NO: 407) | IGHV1-69 | IGHJ6 | 0.004405 | A | G1 |
| | NO: 94) | | | | | | | |
| P-616 | SYAIS (SEQ ID | GIIPIFGTANYAQKFQG (SEQ ID NO: 125) | FPTYHDILTGYEVDY (SEQ ID NO: 408) | IGHV1-69 | IGHJ4 | 0 | E | G1 |
| | NO: 94) | | | | | | | |
| P-621 | SYAIS (SEQ ID | RIIPILGIANYAQKFQG (SEQ ID NO: 67) | GIGYSGSGSNDYFDS (SEQ ID NO: 97) | IGHV1-69 | IGHJ4 | 0.002212 | E | G1 |
| | NO: 94) | | | | | | | |
| P-629 | NYAIS (SEQ ID | RIIPILGIANYAQKFQG (SEQ ID NO: 67) | GIGYSGSGSNDYFDY (SEQ ID NO: 410) | IGHV1-69 | IGHJ4 | 0.004425 | E | G3 |
| | NO: 409) | | | | | | | |
| P-631 | SYGMH (SEQ ID | VISYDGSNEYYADSVKG (SEQ ID NO: 411) | GPWYSSGWYYQGFED (SEQ ID NO: 412) | IGHV3-33 | IGHJ4 | 0.004348 | E | G3 |
| | NO: 203) | | | | | | | |
| P-634 | SYAIS (SEQ ID | RIIPMFGIANYAQKFQG (SEQ ID NO: 413) | HKYEYYDSSGYPFDY (SEQ ID NO: 414) | IGHV1-69 | IGHJ4 | 0.011111 | E | G1 |
| | NO: 94) | | | | | | | |
| P-637 | SYWIG (SEQ ID | IIYPGDSDTRYSPSFQG (SEQ ID NO: 59) | LHRPYGDLQYNWFDP (SEQ ID NO: 415) | IGHV5-51 | IGHJ5 | 0.0131 | E | G1 |
| | NO: 58) | | | | | | | |
| P-640 | SYWIG (SEQ ID | IIYPGDSDTRYSPSFQG (SEQ ID NO: 59) | PPNSSGANFRNAFDI (SEQ ID NO: 416) | IGHV5-51 | IGHJ3 | 0 | A | G1 |
| | NO: 58) | | | | | | | |
| P-641 | GYYMH (SEQ ID | WINPNSGGTNYAQKFQG (SEQ ID NO: | PPPTVPHYYYYGMDV (SEQ ID NO: 417) | IGHV1-2 | IGHJ6 | 0 | A | G1\|G2 |
| | NO: 192) | 239) | | | | | | |
| P-649 | NAWMS (SEQ ID | RIKSKTDGGTTDYAAPVKG (SEQ ID NO: | AGRTKRNYYYYYGMDV (SEQ ID NO: 418) | IGHV3-15 | IGHJ6 | 0 | E | G1 |
| | NO: 242) | 368) | | | | | | |
| P-651 | SYAIS (SEQ ID | GIIPIFGTANYAQKFQG (SEQ ID NO: 125) | DHRILSAGYYYYGMDV (SEQ ID NO: 419) | IGHV1-69 | IGHJ6 | 0 | E | A2 |
| | NO: 94) | | | | | | | |
| P-653 | DYAMH (SEQ ID | GITWNSGSIGYADSVKG (SEQ ID NO: 420) | DIGPYDFWSRSYGMDV (SEQ ID NO: 421) | IGHV3-9 | IGHJ6 | 0.00431 | A | G1 |
| | NO: 72) | | | | | | | |
| P-659 | SYATH (SEQ ID | VISSDGSKKYYADSVKG (SEQ ID NO: 423) | DLVPWLVVKFHYGVDV (SEQ ID NO: 424) | IGHV3-30 | IGHJ6 | 0.069869 | E | G2\|A2 |
| | NO: 422) | | | | | | | |
| P-662 | DYAMH (SEQ ID | GISWNSGSIGYADSVKG (SEQ ID NO: 324) | DRAVREGYNYYYGMDV (SEQ ID NO: 425) | IGHV3-9 | IGHJ6 | 0 | A | G1 |
| | NO: 72) | | | | | | | |
| P-663 | TYAMS (SEQ ID | AISGSGGNTYYADSVKG (SEQ ID NO: 427) | DRWRESSGWYPDAFDI (SEQ ID NO: 428) | IGHV3-23 | IGHJ3 | 0.017316 | E | G1 |
| | NO: 426) | | | | | | | |
| P-666 | SYWMS (SEQ ID | NIKQDGSEKYYVDSVKG (SEQ ID NO: 53) | DVRYDSSGYYDIFRDY (SEQ ID NO: 429) | IGHV3-7 | IGHJ4 | 0.002597 | A | G1 |
| | NO: 52) | | | | | | | |
| P-667 | NHAMY (SEQ ID | VISYDGSKEYYADSVKG (SEQ ID NO: 431) | EEGGSYFTHYYYGMDV (SEQ ID NO: 432) | IGHV3-30-3 | IGHJ6 | 0.034632 | A | G1 |
| | NO: 430) | | | | | | | |
| P-668 | SYAIS (SEQ ID | GIIPIFGTANYAQKFQG (SEQ ID NO: 125) | GGATYCSGGSCYSFDH (SEQ ID NO: 433) | IGHV1-69 | IGHJ4 | 0.00885 | E | G1 |
| | NO: 94) | | | | | | | |
| P-669 | SYAIS (SEQ ID | GIIPIFGTANYAQKFQG (SEQ ID NO: 125) | GGATYCSGGSCYSFDY (SEQ ID NO: 434) | IGHV1-69 | IGHJ4 | 0.004425 | E | G1 |
| | NO: 94) | | | | | | | |
| P-670 | DYAMH (SEQ ID | GSSWNSGSIGYADSVKG (SEQ ID NO: 100) | GKSPLDYDQTMGAFDI (SEQ ID NO: 101) | IGHV3-9 | IGHJ3 | 0.013043 | E | A1 |
| | NO: 72) | | | | | | | |
| P-678 | DYAMH (SEQ ID | GSSWNSGSIGYADSVKG (SEQ ID NO: 100) | GKSPLDYDQTMGAFDI (SEQ ID NO: 101) | IGHV3-9 | IGHJ3 | 0.013043 | E | A1 |
| | NO: 72) | | | | | | | |
| P-679 | DYAMH (SEQ ID | GISWNSGFMGYADSVKG (SEQ ID NO: | GLYQVRYKYYYYALDV (SEQ ID NO: 436) | IGHV3-9 | IGHJ6 | 0 106667 | A | A1 |
| | NO: 72) | 435) | | | | | | |
| P-680 | SYWIG (SEQ ID | IIYPGDSDTRYSPSFQG (SEQ ID NO: 59) | HNTIFGVLGSDYGMDV (SEQ ID NO: 437) | IGHV5-51 | IGHJ6 | 0 | E | A1 |
| | NO: 58) | | | | | | | |
| P-681 | SHWIS (SEQ ID | RIDPSDSYTNYSPSFQG (SEQ ID NO: 106) | HTLLGELSSPTNWFDP (SEQ ID NO: 439) | IGHV5-10-1 | IGHJ5 | 0.017544 | E | G1 |
| | NO: 438) | | | | | | | |
| P-683 | SSSYYWG (SEQ | SIYYSGSTYYNPSLKS (SEQ ID NO: 254) | RVRQWLVRPSWAAFDI (SEQ ID NO: 440) | IGHV4-39 | IGHJ3 | 0 | E | A1 |
| | ID NO: 250) | | | | | | | |
| P-688 | DYAMS (SEQ ID | FIRSKAYGGTTEYAASVKG (SEQ ID NO: | VDGLSSGSYLLPSIDY (SEQ ID NO: 441) | IGHV3-49 | IGHJ4 | 0.002119 | E | G1 |
| | NO: 102) | 103) | | | | | | |
| P-690 | GYYMH (SEQ ID | WINPNSGGTNYAQKFQG (SEQ ID NO: | VPYYYDSSGHRGGMDV (SEQ ID NO: 442) | IGHV1-2 | IGHJ6 | 0.00177 | A | M1\|G3\|G1 |
| | NO: 192) | 239) | | | | | | |
| P-697 | SYGIS (SEQ ID | WISAYNGNTNYAQKLQG (SEQ ID NO: | DRPDYDYVWGSLVPFDY (SEQ ID NO: 443) | IGHV1-18 | IGHJ4 | 0.013216 | A | G1 |
| | NO: 308) | 309) | | | | | | |
| P-698 | GYYMH (SEQ ID | RINPNSGGTNYAQKFQG (SEQ ID NO: 193) | DYYASGSYSPEDYGMDV (SEQ ID NO: 444) | IGHV1-2 | IGHJ6 | 0 | A | G1 |
| | NO: 192) | | | | | | | |
| P-701 | GYYMH (SEQ ID | RINPNSGGTNYAQKFQG (SEQ ID NO: 193) | DYYASGSYSPEDYGMDV (SEQ ID NO: 444) | IGHV1-2 | IGHJ6 | 0 | A | G1 |
| | NO: 192) | | | | | | | |
| P-702 | DYAMH (SEQ ID | GISWNSGRIGYADSVKG (SEQ ID NO: 445) | EGTGDGYNLLIGGAFDI (SEQ ID NO: 446) | IGHV3-9 | IGHJ3 | 0.017316 | A | G1 |
| | NO: 72) | | | | | | | |
| P-705 | TYGMH (SEQ ID | VISYDGSNKYYADSVKG (SEQ ID NO: 56) | GAFYYYGSGSYHYGMDV (SEQ ID NO: 448) | IGHV3-30 | IGHJ6 | 0.004348 | A | G1 |
| | NO: 447) | | | | | | | |
| P-708 | SYAIS (SEQ ID | GIIPIFGTANYAQKFQG (SEQ ID NO: 125) | PEWDYGDPLGYYYGMDV (SEQ ID NO: 449) | IGHV1-69 | IGHJ6 | 0.002232 | A | G1 |
| | NO: 94) | | | | | | | |
| P-712 | SYSMN (SEQ ID | SISSSSSYIYYADSVKG (SEQ ID NO: 450) | VPAMEDGDYYYYYGMDV (SEQ ID NO: 451) | IGHV3-21 | IGHJ6 | 0 | E | G2 |
| | NO: 197) | | | | | | | |
| P-714 | RYAIS (SEQ ID | RIIPILGIANYAQKFQG (SEQ ID NO: 67) | YDFWSGQNTNYYYVLDV (SEQ ID NO: 452) | IGHV1-69 | IGHJ6 | 0.004505 | E | G1 |
| | NO: 124) | | | | | | | |
| P-716 | DYAMS (SEQ ID | FIRSKAYGGTTEYAASVKG (SEQ ID NO: | DEDSGTLLPGFYYYDMDV (SEQ ID NO: 104) | IGHV3-49 | IGHJ6 | 0 | A | G1 |
| | NO: 102) | 103) | | | | | | |
| P-722 | DYAMS (SEQ ID | FIRSKAYGGTTEYAASVKG (SEQ ID NO: | DEDSGTLLPGFYYYGMDV (SEQ ID NO: 453) | IGHV3-49 | IGHJ6 | 0.004219 | A | M\|G1 |
| | NO: 102) | 103) | | | | | | |
| P-724 | SYYMH (SEQ ID | IINPSGGSTSYSQKFQG (SEQ ID NO: 454) | DGIAAAGTEYYYYYGMDV (SEQ ID NO: 455) | IGHV1-46 | IGHJ6 | 0.008811 | A | G1 |
| | NO: 88) | | | | | | | |
| P-726 | SYYMH (SEQ ID | IINPSGGSTSYAQKFQG (SEQ ID NO: 70) | DGIAAGGTEYYYYYGMDV (SEQ ID NO: 456) | IGHV1-46 | IGHJ6 | 0.004405 | A | G1 |
| | NO: 88) | | | | | | | |
| P-731 | SYGMH (SEQ ID | VISYDGSNKYYADSVKG (SEQ ID NO: 56) | DITFDWLGVWYYYYGMDV (SEQ ID NO: 457) | IGHV3-30 | IGHJ6 | 0 | A | G3 |
| | NO: 203) | | | | | | | |
| P-735 | SYAIS (SEQ ID | GIIPIFGTANYAQKFQG (SEQ ID NO: 125) | EKAVAGPRPSYYYYGMDV (SEQ ID NO: 458) | IGHV1-69 | IGHJ6 | 0 | E | G1 |
| | NO: 94) | | | | | | | |
| P-736 | SGNYYWS (SEQ | YIYYSGSTNYNPSLKS (SEQ ID NO: 460) | ETYYYDSSGYYGSDAFDI (SEQ ID NO: 461) | IGHV4-61 | IGHJ3 | 0.017094 | A | G1 |
| | ID NO: 459) | | | | | | | |
| P-739 | TYWIN (SEQ ID | RIDPSDSYTNYSPSFQG (SEQ ID NO: 106) | GDYYDNSDYSGLSEYFQH (SEQ ID NO: 107) | IGHV5-10-1 | IGHJ1 | 0.015351 | E | G1 |
| | NO: 105) | | | | | | | |
| P-760 | SYWMS (SEQ ID | NIEQDGSEKYYVDSVKG (SEQ ID NO: | IYGYYDRSGYYYGEYFQH (SEQ ID NO: 463) | IGHV3-7 | IGHJ1 | 0.008734 | E | G1 |
| | NO: 52) | 462) | | | | | | |
| P-761 | GYYMH (SEQ ID | WINPNSGGTNYAQKFQG (SEQ ID NO: | LPFPYYYDSSGYYAAFDI (SEQ ID NO: 464) | IGHV1-2 | IGHJ3 | 0 | A | G1 |
| | NO: 192) | 239) | | | | | | |
| P-762 | DYAMS (SEQ ID | FIRGKAYGGTSEYAASVKG (SEQ ID NO: | NIALVVYGMRLDYYGMDV (SEQ ID NO: 466) | IGHV3-49 | IGHJ6 | 0.025532 | A | G1 |
| | NO: 102) | 465) | | | | | | |
| P-765 | SYAIS (SEQ ID | GIIPMFGTANYAQKFQG (SEQ ID NO: 406) | RIVVVPAGPWFYYYGMDV (SEQ ID NO: 467) | IGHV1-69 | IGHJ6 | 0.008969 | A | G1 |
| | NO: 94) | | | | | | | |
| P-771 | RYAMH (SEQ ID | WINAGNGKTKYSQKFQG (SEQ ID NO: | ALYYYDSSGSTQSDDAFDI (SEQ ID NO: 110) | IGHV1-3 | IGHJ3 | 0.00885 | E | G1 |
| | NO: 108) | 109) | | | | | | |
| P-773 | RYAMH (SEQ ID | WINAGNGNTKYSQKFQG (SEQ ID NO: | ALYYYDSSGSTQSDDAFDI (SEQ ID NO: 110) | IGHV1-3 | IGHJ3 | 0.013274 | E | G1 |
| | NO: 108) | 468) | | | | | | |
| P-791 | SNYMS (SEQ ID | VIYSGGSTYYADSVKG (SEQ ID NO: 45) | DGQRMAAAGTEDYYYGMDV (SEQ ID NO: 111) | IGHV3-66 | IGHJ6 | 0.001096 | E | G1\|A1\|A2 |
| | NO: 41) | | | | | | | |
| P-796 | SNYMS (SEQ ID | VIYSGGSTYYADSVKG (SEQ ID NO: 45) | DGQRMAAAGTEDYYYGMDV (SEQ ID NO: 111) | IGHV3-66 | IGHJ6 | 0.001096 | E | G1\|A1\|A2 |
| | NO: 41) | | | | | | | |
| P-810 | DYAMH (SEQ ID | GISWNSGTIGYADSVKG (SEQ ID NO: 73) | DTGMRYSSGWYGDDYGMDV (SEQ ID NO: 469) | IGHV3-9 | IGHJ6 | 0.004329 | A | G1 |
| | NO: 72) | | | | | | | |
| P-819 | SYAIS (SEQ ID | GIIPIFGTANYAQKFQG (SEQ ID NO: 125) | ERRCGDCYEPHYYYYGMDV (SEQ ID NO: 470) | IGHV1-69 | IGHJ6 | 0 | E | A1 |
| | NO: 94) | | | | | | | |
| P-829 | SYGMH (SEQ ID | VISYDGSNKYYADSVKG (SEQ ID NO: 56) | VLADYGDYHVSLGYYGMDV (SEQ ID NO: 471) | IGHV3-30 | IGHJ6 | 0 | A | G1 |
| | NO: 203) | | | | | | | |
| P-830 | SYGIS (SEQ ID | WISAYNGNTNYAQKLQG (SEQ ID NO: | VLYYYDRSGYYSSESDFQH (SEQ ID NO: 472) | IGHV1-18 | IGHJ1 | 0 | A | G1 |
| | NO: 308) | 309) | | | | | | |
| P-833 | DYAMH (SEQ ID | GISWNSGTIGYADSVKG (SEQ ID NO: 73) | AGGPLDGSGSYSQPEYYFDY (SEQ ID NO: 473) | IGHV3-9 | IGHJ4 | 0.004348 | E | A2 |
| | NO: 72) | | | | | | | |
| P-835 | SYGMH (SEQ ID | VISYDGSNKYYADSVKG (SEQ ID NO: 56) | ATQRLYYYASGSFLPDAFDI (SEQ ID NO: 474) | IGHV3-30 | IGHJ3 | 0 | E | G1 |
| | NO: 203) | | | | | | | |
| P-837 | TYGMH (SEQ ID | VISYDGSNKYYADSVKG (SEQ ID NO: 56) | ATQRLYYYGSGSYLPDAFDI (SEQ ID NO: 475) | IGHV3-30 | IGHJ3 | 0.005797 | E | G1 |
| | NO: 447) | | | | | | | |
| P-839 | DYAMH (SEQ ID | GISWNSGTIGYADSVKG (SEQ ID NO: 73) | DENRGYSSRWYDPEYYGMDV (SEQ ID NO: 119) | IGHV3-9 | IGHJ6 | 0.004329 | A | G1 |
| | NO: 72) | | | | | | | |
| P-841 | DYAMH (SEQ ID | GISWNSGTIGYADSVKG (SEQ ID NO: 73) | DENRGYSSSWYDPEYYGMDV (SEQ ID NO: 476) | IGHV3-9 | IGHJ6 | 0.006494 | A | G1 |
| | NO: 72) | | | | | | | |
| P-842 | DYAMH (SEQ ID | GITWNSGSIGYADSVKG (SEQ ID NO: 420) | DENRGYSSSWYDPEYYGMDV (SEQ ID NO: 476) | IGHV3-9 | IGHJ6 | 0.008658 | A | G1 |
| | NO: 72) | | | | | | | |
| P-845 | DYAMH (SEQ ID | GISWNSGTIGYADSVKG (SEQ ID NO: 73) | DIGPEGGYSWRRGVYYGMDV (SEQ ID NO: 477) | IGHV3-9 | IGHJ6 | 0.008584 | A | G1 |
| | NO: 72) | | | | | | | |
| P-846 | DYAMH (SEQ ID | GISWNSGTIGYADSVKG (SEQ ID NO: 73) | DISTYYGSGSYYDEDYGMDV (SEQ ID NO: 478) | IGHV3-9 | IGHJ6 | 0.012876 | E | G1 |
| | NO: 72) | | | | | | | |
| P-847 | DYAMH (SEQ ID | GISWNSGTIGYADSVKG (SEQ ID NO: 73) | DVPTYYYDSSGWAEHYGMDV (SEQ ID NO: 479) | IGHV3-9 | IGHJ6 | 0.00431 | A | G1 |
| | NO: 72) | | | | | | | |
| P-851 | SYSIT (SEQ ID | RIIPILGIANFAQKFQG (SEQ ID NO: 481) | ESGGHYYGSGSYYNSNWFDP (SEQ ID NO: 482) | IGHV1-69 | IGHJ5 | 0.013216 | E | A1 |
| | NO: 480) | | | | | | | |
| P-858 | SYSMN (SEQ ID | SISSSSSYIYYADSVKG (SEQ ID NO: 450) | VGEGPTVAQDDYYYYYDMDV (SEQ ID NO: 483) | IGHV3-21 | IGHJ6 | 0 | E | G1\|A1 |
| | NO: 197) | | | | | | | |
| P-859 | SYGIS (SEQ ID | WISAYNGNTNYAQKLQG (SEQ ID NO: | VSFYYDSSGYYSANGNGMDV (SEQ ID NO: 484) | IGHV1-18 | IGHJ6 | 0 | E | G1 |
| | NO: 308) | 309) | | | | | | |
| P-867 | DYGMS (SEQ ID | GINWNGGNTGYADSVKG (SEQ ID NO: | AAEGKLRYFDWLFFADYGMDV (SEQ ID NO: 487) | IGHV3-20 | IGHJ6 | 0.01087 | E | G1 |
| | NO: 485) | 486) | | | | | | |
| P-868 | SYAMS (SEQ ID | AISGSGGSTYYADSVKG (SEQ ID NO: 315) | ANGYCSSTSCLDYYYYYGMDV (SEQ ID NO: 488) | IGHV3-23 | IGHJ6 | 0 | E | G1 |
| | NO: 386) | | | | | | | |
| P-875 | NAWMS (SEQ ID | RIKSKTDGGTTDYAAPVKG (SEQ ID NO: | DKAGYCSSTSCYARELDAFDI (SEQ ID NO: 489) | IGHV3-15 | IGHJ3 | 0 | E | M\|G1\|A2 |
| | NO: 242) | 368) | | | | | | |
| P-878 | NAWMS (SEQ ID | RIKSKTDGGTTDYAAPVKG (SEQ ID NO: | DKAGYCSSTSCYARELDAFDI (SEQ ID NO: 489) | IGHV3-15 | IGHJ3 | 0 | E | M\|G1\|A2 |
| | NO: 242) | 368) | | | | | | |
| P-890 | RYAIS (SEQ ID | GIIPIFGTANYAQKFQG (SEQ ID NO: 125) | ERTYCSSTSCYAGYYYYGMDV (SEQ ID NO: 126) | IGHV1-69 | IGHJ6 | 0.004405 | A | G1\|A1 |
| | NO: 124) | | | | | | | |
| P-892 | RYAIS (SEQ ID | GIIPIFGTANYAQKFQD (SEQ ID NO: 490) | ERTYCSSTSCYAGYYYYGMDV (SEQ ID NO: 126) | IGHV1-69 | IGHJ6 | 0.017621 | A | G1 |
| | NO: 124) | | | | | | | |
| P-911 | RYAIS (SEQ ID | GIIPIFGTANYAQKFQG (SEQ ID NO: 125) | ERTYCSSTSCYAGYYYYGMDV (SEQ ID NO: 126) | IGHV1-69 | IGHJ6 | 0.004405 | A | G1\|A1 |
| | NO: 124) | | | | | | | |
| P-912 | RYAIS (SEQ ID | GIIPIFGTANYAQKFQD (SEQ ID NO: 490) | ERTYCSSTSCYAGYYYYGMDV (SEQ ID NO: 126) | IGHV1-69 | IGHJ6 | 0.017621 | A | G1 |
| | NO: 124) | | | | | | | |
| P-919 | DYAMH (SEQ ID | GISWNSGTIGYADSVKG (SEQ ID NO: 73) | DIAPHYYDILTGYYEGAWGFDY (SEQ ID NO: 491) | IGHV3-9 | IGHJ4 | 0.012876 | A | G1 |
| | NO: 72) | | | | | | | |
| P-920 | SYGMH (SEQ ID | VISSDGSNKYYADSVKG (SEQ ID NO: 492) | DLGVVPAASRWDDYYYYYGMDV (SEQ ID NO: | IGHV3-30 | IGHJ6 | 0.010823 | E | G1 |
| | NO: 203) | | 493) | | | | | |
| P-922 | SYGIS (SEQ ID | WISAYNGNTNYAQKLQG (SEQ ID NO: | DRENLSIFGVSQRLTRYYGMDV (SEQ ID NO: 494) | IGHV1-18 | IGHJ6 | 0.008811 | E | G1 |
| | NO: 308) | 309) | | | | | | |
| P-924 | SYAIS (SEQ ID | GIIPIFGTANYAQKFKG (SEQ ID NO: 495) | EFDLVVVPAATTQYYYYGMDV (SEQ ID NO: | IGHV1-69 | IGHJ6 | 0.004405 | A | G1 |
| | NO: 94) | | 496) | | | | | |
| P-926 | TSGVGVG (SEQ | LIYWDDDKRYSPSLKS (SEQ ID NO: 498) | SPDRRYYDILTGYSNLYWYFDL (SEQ ID NO: 499) | IGHV2-5 | IGHJ2 | 0 | A | M |
| | ID NO: 497) | | | | | | | |
| P-929 | SYAMS (SEQ ID | AISGSGGSTYYADSVKG (SEQ ID NO: 315) | ALYDSSGYYRPGRDFYYYYAMDV (SEQ ID NO: | IGHV3-23 | IGHJ6 | 0 | A | G1 |
| | NO: 386) | | 500) | | | | | |
| P-930 | DYAMH (SEQ ID | GISWNSGTIGYADSVKG (SEQ ID NO: 73) | DIKKLYYDILTGYYNDADYGMDV (SEQ ID NO: | IGHV3-9 | IGHJ6 | 0.004292 | A | G3 |
| | NO: 72) | | 501) | | | | | |
| P-932 | DYAMH (SEQ ID | GISWNSGVIGYADSVKG (SEQ ID NO: 502) | DIKRFYYDILTGYYNDADYGMDV (SEQ ID NO: | IGHV3-9 | IGHJ6 | 0.008584 | A | G3 |
| | NO: 72) | | 503) | | | | | |
| P-935 | NAWMS (SEQ ID | RIKSKTDGGTTDYAAPVKG (SEQ ID NO: | DVSGGYYGSGGYYKYYYYYGMDV (SEQ ID NO: | IGHV3-15 | IGHJ6 | 0 | A | G3 |
| | NO: 242) | 368) | 504) | | | | | |
| P-937 | DYYIH (SEQ ID | RINPNSGGTNYAQKFQG (SEQ ID NO: 193) | EWYDSSGYYSTWSYYYGMDV (SEQ ID NO: | IGHV1-2 | IGHJ6 | 0.008811 | E | G1 |
| | NO: 505) | | 506) | | | | | |
| P-939 | SYWMS (SEQ ID | NIKQDGSEKYYVDSVKG (SEQ ID NO: 53) | EGGPNYYDSSGYYYDSYYYGMDV (SEQ ID NO: | IGHV3-7 | IGHJ6 | 0 | A | G1 |
| | NO: 52) | | 507) | | | | | |
| P-940 | SYWMS (SEQ ID | NIKQDGSEKYYVDSVKG (SEQ ID NO: 53) | EGGPNYYDSSGYYYDYYYYGMDV (SEQ ID NO: | IGHV3-7 | IGHJ6 | 0.004329 | A | G1 |
| | NO: 52) | | 508) | | | | | |
| P-941 | SYWIG (SEQ ID | IIYPGDSDTRYSPSFQG (SEQ ID NO: 59) | HPPDYYGSGSYYNGGPGMGGMDV (SEQ ID NO: | IGHV5-51 | IGHJ6 | 0.002174 | A | M\|G1 |
| | NO: 58) | | 509) | | | | | |
| P-945 | SYAIS (SEQ ID | GIIPIFGTANYAQKFQG (SEQ ID NO: 125) | VAERVHYDILTGYYPYYYYAMDV (SEQ ID NO: | IGHV1-69 | IGHJ6 | 0.00885 | E | G1 |
| | NO: 94) | | 510) | | | | | |

TABLE 9
Primers used in the study
Primers used for the amplification of antibody gene

| Name | Sequence | Step |
|---|---|---|
| IgM-RT | TGACTGGAGTTCAGACGTGTGCTCTTCCGATCTNNNNTNNNNTNNNNGAAGGAAGTCCTGTGCGAG (SEQ ID NO: | RT |
| | 511) | |
| IgG-RT | TGACTGGAGTTCAGACGTGTGCTCTTCCGATCTNNNNTNNNNTNNNNGGGAAGTAGTCCTTGACCA (SEQ ID NO: | RT |
| | 512) | |
| IgA-RT | TGACTGGAGTTCAGACGTGTGCTCTTCCGATCTNNNNTNNNNTNNNNGGGGAAGAAGCCCTGGAC (SEQ ID NO: | RT |
| | 513) | |
| IgD-RT | TGACTGGAGTTCAGACGTGTGCTCTTCCGATCTNNNNTNNNNTNNIVNTGGGTGGTACCCAGTTATCAA (SEQ ID | RT |
| | NO: 514) | |
| IgE-RT | TGACTGGAGTTCAGACGTGTGCTCTTCCGATCTNNNNTNNNNTNNNNAAGTAGCCCGTGGCCAGG (SEQ ID NO: | RT |
| | 515) | |
| VH1 | ACACTCTTTCCCTACACGACGCTCTTCCGATCTGGCCTCAGTGAAGGTCTCCTGCAAG (SEQ ID NO: 516) | 2nd strand synthesis |
| VH2 | ACACTCTTTCCCTACACGACGCTCTTCCGATCTGTCTGGTCCTACGCTGGTGAACCC (SEQ ID NO: 517) | 2nd strand synthesis |
| VH3 | ACACTCTTTCCCTACACGACGCTCTTCCGATCTCTGGGGGGTCCCTGAGACTCTCCTG (SEQ ID NO: 518) | 2nd strand synthesis |
| VH4 | ACACTCTTTCCCTACACGACGCTCTTCCGATCTCTTCGGAGACCCTGTCCCTCACCTG (SEQ ID NO: 519) | 2nd strand synthesis |
| VH5 | ACACTCTTTCCCTACACGACGCTCTTCCGATCTCGGGGAGTCTCTGAAGATCTCCTGT (SEQ ID NO: 520) | 2nd strand synthesis |
| VH6 | ACACTCTTTCCCTACACGACGCTCTTCCGATCTTCGCAGACCCTCTCACTCACCTGTG (SEQ ID NO: 521) | 2nd strand synthesis |
| LC-RT | CACCAGTGTGGCCTTGTTGGCTTG (SEQ ID NO: 522) | RT |
| KC-RT | GTTTCTCGTAGTCTGCTTTGCTCA (SEQ ID NO: 523) | RT |
| VK1-fwd | ACACTCTTTCCCTACACGACGCTCTTCCGATCTATGAGGGTCCCCGCTCAGCTGCTGG (SEQ ID NO: 524) | First round of PCR |
| VK2-fwd | ACACTCTTTCCCTACACGACGCTCTTCCGATCTATGAGGCTCCCCGCTCAGCTGCTGG (SEQ ID NO: 525) | First round of PCR |
| VK3-fwd | ACACTCTTTCCCTACACGACGCTCTTCCGATCTATGAGGGTCCCTGCTCAGCTGCTGG (SEQ ID NO: 526) | First round of PCR |
| VK4-fwd | ACACTCTTTCCCTACACGACGCTCTTCCGATCTATGAGGCTCCCTGCTCAGCTGCTGG (SEQ ID NO: 527) | First round of PCR |
| VK5-fwd | ACACTCTTTCCCTACACGACGCTCTTCCGATCTCTCTTCCTCCTGCTACTCTGGCTCCCAG (SEQ ID NO: 528) | First round of PCR |
| VK6-fwd | ACACTCTTTCCCTACACGACGCTCTTCCGATCTATTTCTCTGTTGCTCTGGATCTCTG (SEQ ID NO: 529) | First round of PCR |
| VK7-fwd | ACACTCTTTCCCTACACGACGCTCTTCCGATCTAGCTCCTGGGGCTCCTGCTGCTCTG (SEQ ID NO: 530) | First round of PCR |
| VK8-fwd | ACACTCTTTCCCTACACGACGCTCTTCCGATCTATGAGGCTCCCTGCTCAGCTCTTGG (SEQ ID NO: 531) | First round of PCR |
| VK9-fwd | ACACTCTTTCCCTACACGACGCTCTTCCGATCTATGGGGTCCCAGGTTCACCTCCTC (SEQ ID NO: 532) | First round of PCR |
| VK10-fwd | ACACTCTTTCCCTACACGACGCTCTTCCGATCTATGTTGCCATCACAACTCATTGGG (SEQ ID NO: 533) | First round of PCR |
| VK1-rev | TGACTGGAGTTCAGACGTGTGCTCTTCCGATCTNNNNTNNNNTNNNNTTTGATATCCACCTTGGTCCC (SEQ ID | |
| | NO: 534) | First round of PCR |
| VK2-rev | TGACTGGAGTTCAGACGTGTGCTCTTCCGATCTNNNNTNNNNTNNNNTTTAATCTCCAGTCGTGTCCC (SEQ ID | |
| | NO: 535) | First round of PCR |
| VK3-rev | TGACTGGAGTTCAGACGTGTGCTCTTCCGATCTNNNNTNNNNTNNNNTGATCTCCAGCTTGGTCCCC (SEQ ID | First round of PCR |
| | NO: 536) | |
| VL1-fwd | ACACTCTTTCCCTACACGACGCTCTTCCGATCTGGTCCTGGGCCCAGTCTGTGCTG (SEQ ID NO: 537) | First round of PCR |
| VL2-fwd | ACACTCTTTCCCTACACGACGCTCTTCCGATCTGGTCCTGGGCCCAGTCTGCCCTG (SEQ ID NO: 538) | First round of PCR |
| VL3-fwd | ACACTCTTTCCCTACACGACGCTCTTCCGATCTGCTCTGTGACCTCCTATGAGCTG (SEQ ID NO: 539) | First round of PCR |
| VL4-fwd | ACACTCTTTCCCTACACGACGCTCTTCCGATCTGGTCTCTCTCGCAGCCTGTGCTG (SEQ ID NO: 540) | First round of PCR |
| VL5-fwd | ACACTCTTTCCCTACACGACGCTCTTCCGATCTGGTCTCTCTCCCAGCCTGTGCTG (SEQ ID NO: 541) | First round of PCR |
| VL6-fwd | ACACTCTTTCCCTACACGACGCTCTTCCGATCTGGTCTCTCTCGCAGCTTGTGCTG (SEQ ID NO: 542) | First round of PCR |
| VL7-fwd | ACACTCTTTCCCTACACGACGCTCTTCCGATCTGGTCTCTCTCCCAGCTTGTGCTG (SEQ ID NO: 543) | First round of PCR |
| VL8-fwd | ACACTCTTTCCCTACACGACGCTCTTCCGATCTGTTCTTGGGCCAATTTTATGCTG (SEQ ID NO: 544) | First round of PCR |
| VL9-fwd | ACACTCTTTCCCTACACGACGCTCTTCCGATCTGGTCCAATTCCCAGGCTGTGGTG (SEQ ID NO: 545) | First round of PCR |
| VL10-fwd | ACACTCTTTCCCTACACGACGCTCTTCCGATCTGGTCCAATTCTCAGGCTGTGGTG (SEQ ID NO: 546) | First round of PCR |
| VL11-fwd | ACACTCTTTCCCTACACGACGCTCTTCCGATCTGAGTGGATTCTCAGACTGTGGTG (SEQ ID NO: 547) | First round of PCR |
| VL12-fwd | ACACTCTTTCCCTACACGACGCTCTTCCGATCTGGTCCTGGGCCCAGTCTGTCGTG (SEQ ID NO: 548) | First round of PCR |
| VL13-fwd | ACACTCTTTCCCTACACGACGCTCTTCCGATCTAGTGTCAGTGGTCCAGGCAGGGC (SEQ ID NO: 549) | First round of PCR |
| VL14-fwd | ACACTCTTTCCCTACACGACGCTCTTCCGATCTACAGGATCCTGGGCTCAGTCTGC (SEQ ID NO: 550) | First round of PCR |
| VL15-fwd | ACACTCTTTCCCTACACGACGCTCTTCCGATCTCTCCTATGAGCTGACTCAGCCAC (SEQ ID NO: 551) | First round of PCR |
| VL16-fwd | ACACTCTTTCCCTACACGACGCTCTTCCGATCTTCTTCTGAGCTGACTCAGGACCC (SEQ ID NO: 552) | First round of PCR |
| VL17-fwd | ACACTCTTTCCCTACACGACGCTCTTCCGATCTAGGTCTCTGTGCTCTGCCTGTGC (SEQ ID NO: 553) | First round of PCR |
| VL18-fwd | ACACTCTTTCCCTACACGACGCTCTTCCGATCTAGGTTCCCTCTCGCAGCCTGTGC (SEQ ID NO: 554) | First round of PCR |
| VL1-rev | TGACTGGAGTTCAGACGTGTGCTCTTCCGATCTNNNNTNNNNTNNNNAGGACGGTGACCTTGGTCCC (SEQ ID | First round of PCR |
| | NO: 555) | |
| VL2-rev | TGACTGGAGTTCAGACGTGTGCTCTTCCGATCTNNNNTNNNNTNNNNAGGACGGTCAGCTGGGTCCC (SEQ ID | First round of PCR |
| | NO: 556) | |
| VL3-rev | TGACTGGAGTTCAGACGTGTGCTCTTCCGATCTNNNNTNNNNTNNNNAGGACGGTCACCTTGGTGCC (SEQ ID | First round of PCR |
| | NO: 557) | |
| VL4-rev | TGACTGGAGTTCAGACGTGTGCTCTTCCGATCTNNNNTNNNNTNNNNAGGACGGTCAGCTGGGTGCC (SEQ ID | First round of PCR |
| | NO: 558) | |
| Illumida adaptor | AATGATACGGCGACCACCGAGATCTACAC[i5 index]ACACTCTTTCCCTACACGACGCTCTTCCGATC (SEQ ID | Second round of PCR |
| amp-fwd | NOS 559-560, respectively) | |
| Illumida adaptor | CAAGCAGAAGACGGCATACGAGAT[i7 index]GTGACTGGAGTTCAGACGTGTGCTCTTCCG (SEQ ID NOS 561- | Second round of PCR |
| amp-rev | 562, respectively) | |

Primers used for the amplification of VH from the phage library

| Name | Sequence | Step |
|---|---|---|
| VH-fwd | AATGATACGGCGACCACCGAGATCTACAC[8 mer Index sequence] | V_{H} amplification for NGS |
| | ACACTCTTTCCCTACACGACGCTCTTCCGATCT (SEQ ID NOS 563-564, respectively) | |
| VH-rev | CAAGCAGAAGACGGCATACGAGAT[8 mer Index sequence]GTGACTGGAGTTCAGACGTGTGCTCTTCCGATCT | V_{H} amplification for NGS |
| | (SEQ ID NOS 565-566, respectively) | |

Primers used for the construction of human scFv libraries

| Name | Sequence | Step |
|---|---|---|
| VL1-fwd | GGGCCCAGGCGGCCGAGCTCGTGCTGACTCAGCCACCC (SEQ ID NO: 567) | First round of PCR |
| VL2-fwd | GGGCCCAGGCGGCCGAGCTCGTGCTGACGCAGCCGC (SEQ ID NO: 568) | First round of PCR |
| VL3-fwd | GGGCCCAGGCGGCCGAGCTCGTCGTGACGCAGCCGC (SEQ ID NO: 569) | First round of PCR |
| VL4-fwd | GGGCCCAGGCGGCCGAGCTCGTGTTGACGCAGCCGCC (SEQ ID NO: 570) | First round of PCR |
| VL5-fwd | GGGCCCAGGCGGCCGAGCTCGGGCTGACTCAGCCACCC (SEQ ID NO: 571) | First round of PCR |
| VL6-fwd | GGGCCCAGGCGGCCGAGCTCGCCCTGACTCAGCCTCGC (SEQ ID NO: 572) | First round of PCR |
| VL7-fwd | GGGCCCAGGCGGCCGAGCTCGCCCTGACTCAGCCTGCC (SEQ ID NO: 573) | First round of PCR |
| VL8-fwd | GGGCCCAGGCGGCCGAGCTCGCCCTGACTCAGCCTCCC (SEQ ID NO: 574) | First round of PCR |
| VL9-fwd | GGGCCCAGGCGGCCGAGCTCGAGCTGACTCAGCCACCCTC (SEQ ID NO: 575) | First round of PCR |
| VL10-fwd | GGGCCCAGGCGGCCGAGCTCGAGCTGACACAGCCACCCTC (SEQ ID NO: 576) | First round of PCR |
| VLII-fwd | GGGCCCAGGCGGCCGAGCTCGAGCTGACTCAGCCACACTC (SEQ ID NO: 577) | First round of PCR |
| VL12-fwd | GGGCCCAGGCGGCCGAGCTCGAGCTGACTCAGGACCCTGC (SEQ ID NO: 578) | First round of PCR |
| VL13-fwd | GGGCCCAGGCGGCCGAGCTCGAGCTGACACAGCTACCCTCG (SEQ ID NO: 579) | First round of PCR |
| VL14-fwd | GGGCCCAGGCGGCCGAGCTCGAGCTGATGCAGCCACCC (SEQ ID NO: 580) | First round of PCR |
| VL15-fwd | GGGCCCAGGCGGCCGAGCTCGAGCTGACACAGCCATCCTCA (SEQ ID NO: 581) | First round of PCR |
| VL16-fwd | GGGCCCAGGCGGCCGAGCTCGAGCTGACTCAGCCACTCTCA (SEQ ID NO: 582) | First round of PCR |
| VL17-fwd | GGGCCCAGGCGGCCGAGCTCGTGCTGACTCAGCCCCCG (SEQ ID NO: 583) | First round of PCR |
| VL18-fwd | GGGCCCAGGCGGCCGAGCTCGTGCTGACTCAATCATCCTCT (SEQ ID NO: 584) | First round of PCR |
| VL19-fwd | GGGCCCAGGCGGCCGAGCTCGTGCTGACTCAATCGCCCTCT (SEQ ID NO: 585) | First round of PCR |
| VL20-fwd | GGGCCCAGGCGGCCGAGCTCGTGCTGACTCAGCCACCTTC (SEQ ID NO: 586) | First round of PCR |
| VL2I-fwd | GGGCCCAGGCGGCCGAGCTCGTGCTGACTCAGCCAACCTC (SEQ ID NO: 587) | First round of PCR |
| VL22-fwd | GGGCCCAGGCGGCCGAGCTCGTGCTGACTCAGCCGGCT (SEQ ID NO: 588) | First round of PCR |
| VL23-fwd | GGGCCCAGGCGGCCGAGCTCGTGCTGACTCAGCCGTCTTC (SEQ ID NO: 589) | First round of PCR |
| VL24-fwd | GGGCCCAGGCGGCCGAGCTCGTGCTGACTCAGCCATCTTCC (SEQ ID NO: 590) | First round of PCR |
| VL25-fwd | GGGCCCAGGCGGCCGAGCTCATGCTGACTCAGCCCCACTC (SEQ ID NO: 591) | First round of PCR |
| VL26-fwd | GGGCCCAGGCGGCCGAGCTCGTGGTGACTCAGGAGCCCTC (SEQ ID NO: 592) | First round of PCR |
| VL27-fwd | GGGCCCAGGCGGCCGAGCTCGTGGTGACCCAGGAGCCATC (SEQ ID NO: 593) | First round of PCR |
| VL1-rev | GGAAGATCTAGAGGAACCACCGCCTAGGACGGTGACCTTGGTCC (SEQ ID NO: 594) | First round of PCR |
| VL2-rev | GGAAGATCTAGAGGAACCACCGCCTAGGACGGTCAGCTTGGTCC (SEQ ID NO: 595) | First round of PCR |
| VL3-rev | GGAAGATCTAGAGGAACCACCGCCGAGGACGGTCACCTTGGTG (SEQ ID NO: 596) | First round of PCR |
| VL4-rev | GGAAGATCTAGAGGAACCACCGCCGAGGACGGTCAGCTGGGTG (SEQ ID NO: 597) | First round of PCR |
| VL5-rev | GGAAGATCTAGAGGAACCACCGCCGAGGGCGGTCAGCTGGG (SEQ ID NO: 598) | First round of PCR |
| VK1-fwd | GGGCCCAGGCGGCCGAGCTCCAGATGACCCAGTCTCCATCT (SEQ ID NO: 599) | First round of PCR |
| VK2-fwd | GGGCCCAGGCGGCCGAGCTCCAGTTGACCCAGTCTCCATCC (SEQ ID NO: 600) | First round of PCR |
| VK3-fwd | GGGCCCAGGCGGCCGAGCTCCAGATGACCCAGTCTCCATCC (SEQ ID NO: 601) | First round of PCR |
| VK4-fwd | GGGCCCAGGCGGCCGAGCTCCAGATGACCCAGTCTCCTTCC (SEQ ID NO: 602) | First round of PCR |
| VK5-fwd | GGGCCCAGGCGGCCGAGCTCCGGATGACCCAGTCTCCATC (SEQ ID NO: 603) | First round of PCR |
| VK6-fwd | GGGCCCAGGCGGCCGAGCTCCGGATGACCCAGTCTCCATTC (SEQ ID NO: 604) | First round of PCR |
| VK7-fwd | GGGCCCAGGCGGCCGAGCTCTGGATGACCCAGTCTCCATCC (SEQ ID NO: 605) | First round of PCR |
| VK8-fwd | GGGCCCAGGCGGCCGAGCTCGTGATGACCCAGACTCCACTC (SEQ ID NO: 606) | First round of PCR |
| VK9-fwd | GGGCCCAGGCGGCCGAGCTCGTGATGACTCAGTCTCCACTC (SEQ ID NO: 607) | First round of PCR |
| VK10-fwd | GGGCCCAGGCGGCCGAGCTCGTGTTGACACAGTCTCCAGC (SEQ ID NO: 608) | First round of PCR |
| VK11-fwd | GGGCCCAGGCGGCCGAGCTCGTGATGACGCAGTCTCCAGC (SEQ ID NO: 609) | First round of PCR |
| VK12-fwd | GGGCCCAGGCGGCCGAGCTCGTGTTGACGCAGTCTCCAG (SEQ ID NO: 610) | First round of PCR |
| VK13-fwd | GGGCCCAGGCGGCCGAGCTCGTAATGACACAGTCTCCAGCC (SEQ ID NO: 611) | First round of PCR |
| VK14-fwd | GGGCCCAGGCGGCCGAGCTCGTGATGACCCAGTCTCCAGAC (SEQ ID NO: 612) | First round of PCR |
| VK15-fwd | GGGCCCAGGCGGCCGAGCTCACACTCACGCAGTCTCCAG (SEQ ID NO: 613) | First round of PCR |
| VK16-fwd | GGGCCCAGGCGGCCGAGCTCGTGCTGACTCAGTCTCCAGAC (SEQ ID NO: 614) | First round of PCR |
| VK-1-rev | GGAAGATCTAGAGGAACCACCTTTGATTTCCACCTTGGTCCC (SEQ ID NO: 615) | First round of PCR |
| VK-2-rev | GGAAGATCTAGAGGAACCACCTTTGATCTCCAGCTTGGTCCC (SEQ ID NO: 616) | First round of PCR |
| VK-3-rev | GGAAGATCTAGAGGAACCACCTTTGATATCCACTTTGGTCCC (SEQ ID NO: 617) | First round of PCR |
| VK-4-rev | GGAAGATCTAGAGGAACCACCTTTGATCTCCACCTTGGTCCC (SEQ ID NO: 618) | First round of PCR |
| VK-5-rev | GGAAGATCTAGAGGAACCACCTTTAATCTCCAGTCGTGTCCC (SEQ ID NO: 619) | First round of PCR |
| VH1-fwd | GGTGGTTCCTCTAGATCTTCCTCCTCTGGTGGCGGTGGCTCGGGCGGTGGTGGGCAGGTTCAGCTGGTGCAGTC | First round of PCR |
| | (SEQ ID NO: 620) | |
| VH2-fwd | GGTGGTTCCTCTAGATCTTCCTCCTCTGGTGGCGGTGGCTCGGGCGGTGGTGGGCAGGTGCAGCTGGTGCAG | First round of PCR |
| | (SEQ ID NO: 621) | |
| VH3-fwd | GGTGGTTCCTCTAGATCTTCCTCCTCTGGTGGCGGTGGCTCGGGCGGTGGTGGGCAGGTCCAGCTGGTACAGTCT | First round of PCR |
| | (SEQ ID NO: 622) | |
| VH4-fwd | GGTGGTTCCTCTAGATCTTCCTCCTCTGGTGGCGGTGGCTCGGGCGGTGGTGGGCAGGTCCAGCTTGTGCAGTC | First round of PCR |
| | (SEQ ID NO: 623) | |
| VH5-fwd | GGTGGTTCCTCTAGATCTTCCTCCTCTGGTGGCGGTGGCTCGGGCGGTGGTGGGCAGATGCAGCTGGTGCAGTC | First round of PCR |
| | (SEQ ID NO: 624) | |
| VH6-fwd | GGTGGTTCCTCTAGATCTTCCTCCTCTGGTGGCGGTGGCTCGGGCGGTGGTGGGCAAATGCAGCTGGTGCAGTC | First round of PCR |
| | (SEQ ID NO: 625) | |
| VH7-fwd | GGTGGTTCCTCTAGATCTTCCTCCTCTGGTGGCGGTGGCTCGGGCGGTGGTGGGCAGGTCCAGCTGGTGCAATC | First round of PCR |
| | (SEQ ID NO: 626) | |
| VH8-fwd | GGTGGTTCCTCTAGATCTTCCTCCTCTGGTGGCGGTGGCTCGGGCGGTGGTGGGCAGGTCCAGCTGGTGCAG | First round of PCR |
| | (SEQ ID NO: 627) | |
| VH9-fwd | GGTGGTTCCTCTAGATCTTCCTCCTCTGGTGGCGGTGGCTCGGGCGGTGGTGGGGAGGTCCAGCTGGTACAGTC | First round of PCR |
| | (SEQ ID NO: 628) | |
| VH10-fwd | GGTGGTTCCTCTAGATCTTCCTCCTCTGGTGGCGGTGGCTCGGGCGGTGGTGGGCAGGTCACCTTGAAGGAGTCT | First round of PCR |
| | (SEQ ID NO: 629) | |
| VH11-fwd | GGTGGTTCCTCTAGATCTTCCTCCTCTGGTGGCGGTGGCTCGGGCGGTGGTGGGCAGATCACCTTGAAGGAGTCT | First round of PCR |
| | (SEQ ID NO: 630) | |
| VH12-fwd | GGTGGTTCCTCTAGATCTTCCTCCTCTGGTGGCGGTGGCTCGGGCGGTGGTGGGCAGGTCACCTTGAGGGAGTC | First round of PCR |
| | (SEQ ID NO: 631) | |
| VH13-fwd | GGTGGTTCCTCTAGATCTTCCTCCTCTGGTGGCGGTGGCTCGGGCGGTGGTGGGCGGGTCACCTTGAGGGAG | First round of PCR |
| | (SEQ ID NO: 632) | |
| VH14-fwd | GGTGGTTCCTCTAGATCTTCCTCCTCTGGTGGCGGTGGCTCGGGCGGTGGTGGGCAGGTGCAGCTGGTGGAG | First round of PCR |
| | (SEQ ID NO: 633) | |
| VH15-fwd | GGTGGTTCCTCTAGATCTTCCTCCTCTGGTGGCGGTGGCTCGGGCGGTGGTGGGCAGGTGCAGCTGTTGGAGTC | First round of PCR |
| | (SEQ ID NO: 634) | |
| VH16-fwd | GGTGGTTCCTCTAGATCTTCCTCCTCTGGTGGCGGTGGCTCGGGCGGTGGTGGGGAGGTGCAGCTGGTGGAG | First round of PCR |
| | (SEQ ID NO: 635) | |
| VH17-fwd | GGTGGTTCCTCTAGATCTTCCTCCTCTGGTGGCGGTGGCTCGGGCGGTGGTGGGGAGGTGCATCTGGTGGAGTC | First round of PCR |
| | (SEQ ID NO: 636) | |
| VH18-fwd | GGTGGTTCCTCTAGATCTTCCTCCTCTGGTGGCGGTGGCTCGGGCGGTGGTGGGGAGGTGCAACTGGTGGAGTC | First round of PCR |
| | (SEQ ID NO: 637) | |
| VH19-fwd | GGTGGTTCCTCTAGATCTTCCTCCTCTGGTGGCGGTGGCTCGGGCGGTGGTGGGGAGGTGCAGCTGTTGGAGTC | First round of PCR |
| | (SEQ ID NO: 638) | |
| VH20-fwd | GGTGGTTCCTCTAGATCTTCCTCCTCTGGTGGCGGTGGCTCGGGCGGTGGTGGGCAGGTGCAGCTGGTGGAC | First round of PCR |
| | (SEQ ID NO: 639) | |
| VH21-fwd | GGTGGTTCCTCTAGATCTTCCTCCTCTGGTGGCGGTGGCTCGGGCGGTGGTGGGCAGGTACAGCTGGTGGAGTC | First round of PCR |
| | (SEQ ID NO: 640) | |
| VH22-fwd | GGTGGTTCCTCTAGATCTTCCTCCTCTGGTGGCGGTGGCTCGGGCGGTGGTGGGGAAGTGCAGCTGGTGGAGTC | First round of PCR |
| | (SEQ ID NO: 641) | |
| VH23-fwd | GGTGGTTCCTCTAGATCTTCCTCCTCTGGTGGCGGTGGCTCGGGCGGTGGTGGGCAGGTGCAGCTGCAGGAG | First round of PCR |
| | (SEQ ID NO: 642) | |
| VH24-fwd | GGTGGTTCCTCTAGATCTTCCTCCTCTGGTGGCGGTGGCTCGGGCGGTGGTGGGCAGGTGCAGCTACAGGAGTC | First round of PCR |
| | (SEQ ID NO: 643) | |
| VH25-fwd | GGTGGTTCCTCTAGATCTTCCTCCTCTGGTGGCGGTGGCTCGGGCGGTGGTGGGCAGGTACAGCTGCAGGAGTC | First round of PCR |
| | (SEQ ID NO: 644) | |
| VH26-fwd | GGTGGTTCCTCTAGATCTTCCTCCTCTGGTGGCGGTGGCTCGGGCGGTGGTGGGCAGCTGCAGCTGCAGGAG | First round of PCR |
| | (SEQ ID NO: 645) | |
| VH27-fwd | GGTGGTTCCTCTAGATCTTCCTCCTCTGGTGGCGGTGGCTCGGGCGGTGGTGGGCAGGTGCAGCTACAGCAGTG | First round of PCR |
| | (SEQ ID NO: 646) | |
| VH28-fwd | GGTGGTTCCTCTAGATCTTCCTCCTCTGGTGGCGGTGGCTCGGGCGGTGGTGGGCAGGTGCAGCTACAACAGTGG | First round of PCR |
| | (SEQ ID NO: 647) | |
| VH29-fwd | GGTGGTTCCTCTAGATCTTCCTCCTCTGGTGGCGGTGGCTCGGGCGGTGGTGGGCGGCTGCAGCTGCAGG (SEQ | First round of PCR |
| | ID NO: 648) | |
| VH30-fwd | GGTGGTTCCTCTAGATCTTCCTCCTCTGGTGGCGGTGGCTCGGGCGGTGGTGGGGAAGTGCAGCTGGTGCAGTC | First round of PCR |
| | (SEQ ID NO: 649) | |
| VH31-fwd | GGTGGTTCCTCTAGATCTTCCTCCTCTGGTGGCGGTGGCTCGGGCGGTGGTGGGGAGGTGCAGCTGGTGCAG | First round of PCR |
| | (SEQ ID NO: 650) | |
| VH32-fwd | GGTGGTTCCTCTAGATCTTCCTCCTCTGGTGGCGGTGGCTCGGGCGGTGGTGGGCAGGTACAGCTGCAGCAGTC | First round of PCR |
| | (SEQ ID NO: 651) | |
| VH33-fwd | GGTGGTTCCTCTAGATCTTCCTCCTCTGGTGGCGGTGGCTCGGGCGGTGGTGGGCAGGTGCAGCTGGTGCAATC | First round of PCR |
| | (SEQ ID NO: 652) | |
| VH1-rev | CCTGGCCGGCCTGGCCACTAGTTGAGGAGACGGTGACCAGG (SEQ ID NO: 653) | First round of PCR |
| VH2-rev | CCTGGCCGGCCTGGCCACTAGTTGAGGAGACAGTGACCAGGG (SEQ ID NO: 654) | First round of PCR |
| VH3-rev | CCTGGCCGGCCTGGCCACTAGTTGAAGAGACGGTGACCATTGT (SEQ ID NO: 655) | First round of PCR |
| VH4-rev | CCTGGCCGGCCTGGCCACTAGTTGAGGAGACGGTGACCGTG (SEQ ID NO: 656) | First round of PCR |
| AMP-VH-fwd | GGTGGTTCCTCTAGATCTTCCTCC (SEQ ID NO: 657) | Second round of PCR |
| AMP-VH-rev | CCTGGCCGGCCTGGCCAC (SEQ ID NO: 658) | Second round of PCR |
| AMP-K/L-fwd | GGGCCCAGGCGGCCGAG (SEQ ID NO: 659) | Second round of PCR |
| AMP-K/L-rev | GGAAGATCTAGAGGAACCACC (SEQ ID NO: 660) | Second round of PCR |
| EXT-fwd | GAGGAGGAGGAGGAGGAGGCGGGGCCCAGGCGGCCGAGCTC (SEQ ID NO: 661) | Overlap extension |
| EXT-rev | GAGGAGGAGGAGGAGGAGCCTGGCCGGCCTGGCCACTAGT (SEQ ID NO: 662) | Overlap extension |

Primers used for the generation of RBD mutants

| Name | Sequence | Step |
|---|---|---|
| RBD-fwd | GGCCCAGGCGGCCTTTACA (SEQ ID NO: 663) | First and second round of |
| | | PCR |
| RBD-rev | GGCCGGCCTGGCCAAAGTTG (SEQ ID NO: 664) | First and second round of |
| | | PCR |
| N354D-fwd | GTGTCTACGCATGGGACAGAAAGAGAATCAGT (SEQ ID NO: 665) | First round of PCR |
| N354D-rev | ACTGATTCTCTTTCTGTCCCATGCGTAGACAC (SEQ ID NO: 666) | First round of PCR |
| D364Y-fwd | GTAACTGTGTAGCGTACTATAGTGTCCTTTAT (SEQ ID NO: 667) | First round of PCR |
| D364Y-rev | ATAAAGGACACTATAGTACGCTACACAGTTAC (SEQ ID NO: 668) | First round of PCR |
| V367F-fwd | TGTGTAGCGGATTATAGTTTCCTTTATAATTCAGC (SEQ ID NO: 669) | First round of PCR |
| V367F-rev | GCTGAATTATAAAGGAAACTATAATCCGCTACACA (SEQ ID NO: 670) | First round of PCR |
| F342L-fwd | TTCGGGGAAGTGCTGAACGCTACCCG (SEQ ID NO: 671) | First round of PCR |
| F342L-rev | CGGGTAGCGTTCAGCACTTCCCCGAA (SEQ ID NO: 672) | First round of PCR |
| R408I-fwd | GAGATGAGGTGATTCAAATCGCGC (SEQ ID NO: 673) | First round of PCR |
| R408I-rev | GCGCGATTTGAATCACCTCATCTC (SEQ ID NO: 674) | First round of PCR |
| W436R-fwd | GGATGTGTTATCGCTAGAAACTCTAACAAC (SEQ ID NO: 675) | First round of PCR |
| W436R-rev | GTTGTTAGAGTTTCTAGCGATAACACATCC (SEQ ID NO: 676) | First round of PCR |
| V341I-fwd | CATTCGGGGAAATCTTTAACGCTACC (SEQ ID NO: 677) | First round of PCR |
| V341I-rev | GGTAGCGTTAAAGATTTCCCCGAATG (SEQ ID NO: 678) | First round of PCR |
| A435S-fwd | GGGATGTGTTATCAGCTGGAACTCTAAC (SEQ ID NO: 679) | First round of PCR |
| A435S-rev | GTTAGAGTTCCAGCTGATAACACATCCC (SEQ ID NO: 680) | First round of PCR |
| G476S-fwd | ATTTATCAGGCTAGCAGCACACCTTG (SEQ ID NO: 681) | First round of PCR |
| G476S-rev | CAAGGTGTGCTGCTAGCCTGATAAAT (SEQ ID NO: 682) | First round of PCR |
| V483A-fwd | CACCTTGCAATGGTGCCGAAGGATTCAA (SEQ ID NO: 683) | First round of PCR |
| V483A-rev | TTGAATCCTTCGGCACCATTGCAAGGTG (SEQ ID NO: 684) | First round of PCR |

TABLE 10
Sequences of light (VL) and heavy (VH) chain variable regions of the
antibodies described herein.

>1H12
VL
ELGLTQPPSASGTPGQRVTISCSGSSSNIGSNTVNWYQQLPGTAPKLLIYSNNQRPSG
VPDRFSGSKSGTSASLAISGLQSEDEADYYCAAWDDSLNGWVFGGGTKLTVL (SEQ
ID NO: 1)
VH
EVQLVESGGGLIQPGRSLRLSCAASGFTFDDYAMHWVRQAPGKGLEWVSGVTWNS
GTIGYADSVKGRFFISRDNAKNSLYLQMNSLRPEDTALYYCVKDIMGDGSPSLHYYY
YGMDVWGQGTTVTVSS (SEQ ID NO: 2)
>A-1A1
VL:
ELELTQPPSVSVSPGQTARITCSADALPKQYAYWYQQKPGQAPVLVIYKDSERPSGIP
ERFSGSNSGNTATLTISRVEAGDEADYYCQVWDSSSDHVVFGGGTKLTVL (SEQ ID
NO: 3)
VH:
QVQLVESGGGLVQPGRSLRLSCAASGFTFDDYAMHWVRQAPGKGLEWVSGISWNS
GTIGYADSVKGRFTISRDNAKNSLYLQMNSLRAEDTALYYCAKDENRGYSSRWYDP
EYYGMDVWGQGTTVTVSS (SEQ ID NO: 4)
>A-1H4
VL:
ELQLTQSPSFLSASVGDRVTITCRASQGISSYLAWYQQKPGKAPKLLIYAASTLQSGV
PSRFSGSGSGTEFTLTISSLQPEDFATYYCQQLNSYPRLTFGGGTKVEIK (SEQ ID
NO: 5)
VH:
EVQLVESGGGLVQPGRSLRLSCAASGFTVSSNYMSWVRQAPGKGLEWVSGIYSGGS
TYYADSVKGRFTISRDNSKNTLYLQMNSLRAEDTAVYYCARDLQEAGAFDIWGQGT
MVTVSS (SEQ ID NO: 6)
>3A3
VL:
ELELMQPPSVSGAPGQRVTISCTGSSSNIGAGYDVHWYQQLPGTAPKLLIYGNSNRPS
GVPDRFSGSKSGTSASLAITGLQAEDEADYYCQSYDSSLSGSVFGGGTKLTVL (SEQ
ID NO: 7)
VH:
EVQLVESGGGLVQPGGSLRLSCAASGLTVSRNYMSWVRQAPGKGLEWVSVIYSGGS
TYYADSVKGRFTISRDNSKNTLYLQMNSLRAEDTAVYYCARDLDTAGGMDVWGQ
GTTVTVSS (SEQ ID NO: 8)
>E-3G9
VL:
ELRMTQSPSSLSASVGDRVTITCRASQSISNYLNWYQQKPGKAPKLLIYAASSLQSGV
PSRFSGSGSGTDFTLTISSLQPEDFATYYCQQSYSTPPTFGQGTKLEIK (SEQ ID NO: 9)
VH:
QMQLVQSGAEVKKPGASVKVSCKASGHTITSYYMHWVRQAPGQGLEWMGIINPSG
GSTSYAQKFQGRVTMTRDTSTSTVYMELSSLRSEDTAVYYCAREGVWDSSGYSSFD
YWGQGTLVTVSS (SEQ ID NO: 10)
>E-3A12
VL:
ELVLTQPPSVSGAPGQRVTISCTGSSSNIGAGYDVHWYQQLPGTAPKLLIYGNSNRPS
GVPDRFSGSKSGTSASLAITGLQAEDEADYYCQSYDSSLSGSVFGGGTKLTVL (SEQ
ID NO: 11)
VH:
EVHLVESGGGLIQPGGSLRLSCAASGVTVSSNYMSWVRQAPGKGLEWVSVIYSGGS
TYYADSVKGRFTISRDNSKNTLYLQMNSLRAEDTAVYYCARGDGSGDYYYGMDV
WGQGTTVTVSS (SEQ ID NO: 12)
>4C10
VL:
ELVVTQPPSVSAAPGQKVTISCSGSSSNIGNNYVSWYQQLPRTAPKLLIYDNNNRPSG
IPDRFSGSKSGTSATLGITGLQTGDEAEYYCGTWDSSLSAVVFGGGTKLTVL (SEQ ID
NO: 13)
VH:
QVQLLESGGGLVQPGGSLRLSCAASGFTFDDYAMHWVRQAPGKGLEWVSGVTWN
SGSIGYADSVKGRFIISRDNAKNSLYLQMNSLRAEDTALYYCAKDISPMLRGDNYGM
DVWGQGTTVTVSS (SEQ ID NO: 14)
>E-4D12
VL:
ELGLTQPPSASGTPGQRVTISCSGSRSNIGSNVVNWYQQLPGTAPKLLIYSNDYRPSG
VPDRFSGSKSGTSASLDISGLQSEDEADYYCAAWDDSLNGSVFGGGTKLTVL (SEQ
ID NO: 15)
VH:
QVQLVQSGAEVKKPGESLRISCKGSGYSFTTYWINWVRQMPGKGLEWMGRIDPSDS
YTNYSPSFQGHVTISADKSISTAYLQWSGLKASDTAMYYCARGDYYDNSDYSGLSE
YFQHWGQGTMVTISS (SEQ ID NO: 16)
>A-2F1
VL:
ELALTQPPSASGSPGQSVTISCTASSSDIGASNHVAWYQQNPGKAPKLMIYGVSGRPS
GVPDRFSGSKSGNTASLTVSGLQAEDEADYYCSSYAGSNILIFGGGTKLTVL (SEQ ID
NO: 17)
VH:
QVQLVQSGGGLIQPGGSLRLSCAASGVTVSSNYMSWVRQAPGKGLEWVSVIYSGGS
TFYADSVKGRFTISRDNSKNTLYLQMNSLRAEDTAVYYCARDLMEAGGMDVWGQ
GSTVTVSS (SEQ ID NO: 18)
>A-2H4
VL:
ELALTQPASVSGSPGQSITISCTGTSSDIGGYEYVSWYQQHPGKAPKLIIYDVRDRPSG
ISNRFSGSKSGNTASLIISSLQAGDEADYYCFSYTSSGTYVFGSATKVTVL (SEQ ID
NO: 19)
VH:
QMQLVESGGGLVQPGRSLRLSCAASGFTFSVYGMHWVRQAPGKGLEWVAVISYDG
SNKYYADSVKGRFSISRDNSKNTLYLQMNSLRGEDTAVYYCAKGGPRPVVKAYGEL
DYYGMDVWGQGTTVTVSS (SEQ ID NO: 20)
>2G3
VL:
ELVLTQPPSASGTPGQRVTISCSGSSSNIGSNTVNWYQQVPGTAPKLLIYSNNQRPSG
VPDRFSGSKSGTSASLAISGLQSEDEADYYCAAWDDSLNIYVFGSGTKVTVL (SEQ
ID NO: 21)
VH:
QVQLVESGGGVVRPGGSLRLSCAASGFTFDDYGMTWVRQVPGKGLEWVSGINWNG
GTTGYADSVKGRFTISRDNAKKSLYLQMNSLRAEDTALYHCARIYCGDDCYSLVIW
GDAFDIWGQGTMVTVSS (SEQ ID NO: 22)
>E-3B1
VL:
EIELTQPPSVSVAPGKTARITCGGNSIGSKSVHWYQQKPGQSPVLVIYQDSKRPSGIPE
RFSGSNSGNTATLTISGTQAMDEADYYCQAWDSSTVVFGGGTKLTVL (SEQ ID
NO: 23)
VH:
QVQLVESGGGLVKPGGSLRLSCAASGFTVSSNYMSWVRQAPGKGLEWVSVLYSGG
STFYADSVKGRFTISRDNSKNTLYLQMNSLRAEDTAVYYCTRDAQVYGMDVWGQG
TTVTVSS (SEQ ID NO: 24)
>E-3H31
VL:
ELVVTQPPSVSGAPGQRVTISCTGSSSNIGAGYDVHWYQQLPGTAPKLLIYGNSNRPS
GVPDRFSGSKSGTSASLAITGLQAEDEADYYCQSYDSSLSGSVFGGGTKLTVL (SEQ
ID NO: 25)
VH:
QMQLVQSGAEVKKPGASVKVSCKASGYTFTRYAMHWVRQAPGQRLEWMGWINA
GNGKTKYSQKFQGRVTITRDTSASTAYMELSSLRSEDTAVYYCARALYYYDSSGST
QSDDAFDIWGQGTMVTVSS (SEQ ID NO: 26)

What is claimed is:

1. An isolated neutralizing antibody that binds SARS-CoV-2.

2. The antibody of claim 1, wherein the antibody is an IgG, IgA, IgA or IgM.

3. The antibody of claim 1, wherein the antibody is an IgG₁, IgA₁, or IgA₂.

4. The antibody of claim 1, wherein the antibody binds to the S1, S2, RBD and/or N proteins of SARS-CoV-2.

5. The antibody of claim 1, wherein the antibody comprises an amino acid sequence having at least 60%, 65%, 70%, 75%, 80%, 85%, 90%, or 95% sequence identity to one or more sequences shown in FIG. 1B-1D, Table 1, Table 3, Table 4, or Table 8, or Table 10, or a functional variant thereof.

6. The antibody of claim 1, wherein the antibody comprises an amino acid sequence having at least 60%, 65%, 70%, 75%, 80%, 85%, 90%, or 95% sequence identity to one or more light chain variable region amino acid sequences and/or one or more heavy chain variable region amino acid sequences shown in Table 10.

7. The antibody of claim 1, wherein the antibody comprises a light chain variable region (VL) having an amino acid sequence selected from SEQ ID NOs: 1, 3, 5, 7, 9, 11, 13, 15, 17, 19, 21, 23, or 25, or an amino acid sequence having at least 60%, 65%, 70%, 75%, 80%, 85%, 90%, or 95% sequence identity to SEQ ID NOs: 1, 3, 5, 7, 9, 11, 13, 15, 17, 19, 21, 23, or 25.

8. The antibody of claim 1, wherein the antibody comprises a heavy chain variable region (VH) having an amino acid sequence selected from SEQ ID NOs: 2, 4, 6, 8, 10, 12, 14, 16, 18, 20, 22, 24, or 26, or an amino acid sequence having at least 60%, 65%, 70%, 75%, 80%, 85%, 90%, or 95% sequence identity to SEQ ID NOs: 2, 4, 6, 8, 10, 12, 14, 16, 18, 20, 22, 24, or 26.

9. The antibody of claim 1, wherein the antibody comprises:
i) a VL amino acid sequence of SEQ ID NO:1 and a VH amino acid sequence of SEQ ID NO:2;
ii) a VL amino acid sequence of SEQ ID NO:3 and a VH amino acid sequence of SEQ ID NO:4;
iii) a VL amino acid sequence of SEQ ID NO:5 and a VH amino acid sequence of SEQ ID NO:6;
iv) a VL amino acid sequence of SEQ ID NO:7 and a VH amino acid sequence of SEQ ID NO:8;
v) a VL amino acid sequence of SEQ ID NO:9 and a VH amino acid sequence of SEQ ID NO:10;
vi) a VL amino acid sequence of SEQ ID NO:11 and a VH amino acid sequence of SEQ ID NO:12;
vii) a VL amino acid sequence of SEQ ID NO:13 and a VH amino acid sequence of SEQ ID NO:14;
viii) a VL amino acid sequence of SEQ ID NO:15 and a VH amino acid sequence of SEQ ID NO:16;
ix) a VL amino acid sequence of SEQ ID NO:17 and a VH amino acid sequence of SEQ ID NO:18;
x) a VL amino acid sequence of SEQ ID NO:19 and a VH amino acid sequence of SEQ ID NO:20;
xi) a VL amino acid sequence of SEQ ID NO:21 and a VH amino acid sequence of SEQ ID NO:22;
xii) a VL amino acid sequence of SEQ ID NO:23 and a VH amino acid sequence of SEQ ID NO:24; or
xiii) a VL amino acid sequence of SEQ ID NO:25 and a VH amino acid sequence of SEQ ID NO:26;
or amino acid sequence having at least 60%, 65%, 70%, 75%, 80%, 85%, 90%, or 95% sequence identity thereto.

10. The antibody of claim 1, wherein the antibody comprises a VH comprising a V gene and/or a J gene in FIG. 1B, FIG. 1F, Table 1, Table 3, Table 4, Table 5, or Table 8, or a functional variant thereof, and a VL comprising a V gene and/or a J gene in FIG. 1D.

11. The antibody of claim 1, wherein the antibody comprises a HCDR3 amino acid sequence in FIG. 1B (SEQ ID NOS 685, 51, 113, 49, 118, 121, 82, 43, 89, 110, 107), or a functional variant thereof.

12. The antibody of claim 1, wherein the antibody comprises a HCDR3 amino acid sequence in Table 1, or a functional variant thereof.

13. The antibody of claim 1, wherein the antibody comprises a heavy chain variable region amino acid sequence having at least 80%, 85%, 90%, or 95% sequence identity to one or more sequences shown in FIG. 1C (SEQ ID NOS 686-700).

14. The antibody of claim 1, wherein the antibody comprises a light chain CDR3 sequence shown in FIG. 1D (SEQ ID NOS 701-708), or a functional variant thereof.

15. The antibody of claim 1, wherein the antibody comprises a HCDR1, HCDR2 or HCDR3 sequence shown in Table 3, or a functional variant thereof.

16. The antibody of claim 1, wherein the antibody comprises a HCDR1, HCDR2 or HCDR3 sequence shown in Table 4, or a functional variant thereof.

17. The antibody of claim 1, wherein the antibody comprises a HCDR1, HCDR2 or HCDR3 sequence shown in Table 8, or a functional variant thereof.

18. The antibody of claim 1, wherein the antibody inhibits binding of SARS-CoV-2 S glycoprotein to ACE2.

19. The antibody of claim 1, wherein the antibody binds to a mutant RBD comprising one or more of the amino acid substitutions V341I, F342L, N354D, D364Y; V367F; A435S; W436R; G476S; V483A; G476S and V483A; N501Y; N439K; K417V; K417V and N439K; K417N; E484K; K417N, E484K, and N501Y; K417T; K417T, E484K, and N501Y; L452R; S477N; E484K; E484Q; or E484Q and L452R, or combinations thereof.

20. The antibody of claim 1, wherein the clonotype is IGHV3-53/IGHV3-66 and IGHJ6.

21. The antibody of claim 1, wherein the antibody is a naïve stereotypic IGHV3-53/IGHV3-66 and IGHJ6 clone.

22. The antibody of claim 1, wherein the antibody is an scFv, Fab, or other antigen binding fragment or format thereof.

23. A pharmaceutical composition comprising the antibody of claim 1.

24. A nucleic acid encoding a heavy chain variable region and/or a light chain variable region of the antibody of claim 1.

25. A vector comprising the nucleic acid of claim 24.

26. A host cell comprising the vector of claim 25.

27. A method for producing an antibody, comprising culturing the host cell of claim 26 under conditions in which the nucleic acids encoding the heavy and light chain variable regions are expressed.

28. An in vitro method for detecting binding of an antibody to SARS-CoV-2 antigens, the method comprising:
i) contacting a cell infected with SARS-CoV-2 with the antibody of claim 1, and detecting binding of the antibody to the cell; or
ii) contacting a recombinant SARS-CoV-2 antigen with the antibody of claim 1, and detecting binding of the antibody to the antigen.

29. A method of inducing an immune response in a subject, the method comprising administering the antibody of claim 1 to a subject.


SEQUENCE LISTING (708 sequences)

SEQ ID NO: 1 - protein, 110 aa, Artificial Sequence
  Description of Artificial Sequence Synthetic polypeptide
ELGLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGWVFGGGTKLTVL

SEQ ID NO: 2 - protein, 128 aa, Artificial Sequence
  Description of Artificial Sequence Synthetic polypeptide
EVQLVESGGGLIQPGRSLRLSCAASGFTFDDYAMHWVRQAPGKGLEWVSGVTWNSGTIGYADSVKGRFFISRDNAKNSLYLQMNSLRPEDTALYYCVKDIMGDGSPSLHYYYYGMDVWGQGTTVTVSS

SEQ ID NO: 3 - protein, 108 aa, Artificial Sequence
  Description of Artificial Sequence Synthetic polypeptide
ELELTQPPSVSVSPGQTARITCSADALPKQYAYWYQQKPGQAPVLVIYKDSERPSGIPERFSGSNSGNTATLTISRVEAGDEADYYCQVWDSSSDHVVFGGGTKLTVL

SEQ ID NO: 4 - protein, 129 aa, Artificial Sequence
  Description of Artificial Sequence Synthetic polypeptide
QVQLVESGGGLVQPGRSLRLSCAASGFTFDDYAMHWVRQAPGKGLEWVSGISWNSGTIGYADSVKGRFTISRDNAKNSLYLQMNSLRAEDTALYYCAKDENRGYSSRWYDPEYYGMDVWGQGTTVTVSS

SEQ ID NO: 5 - protein, 108 aa, Artificial Sequence
  Description of Artificial Sequence Synthetic polypeptide
ELQLTQSPSFLSASVGDRVTITCRASQGISSYLAWYQQKPGKAPKLLIYAASTLQSGVPSRFSGSGSGTEFTLTISSLQPEDFATYYCQQLNSYPRLTFGGGTKVEIK

SEQ ID NO: 6 - protein, 118 aa, Artificial Sequence
  Description of Artificial Sequence Synthetic polypeptide
EVQLVESGGGLVQPGRSLRLSCAASGFTVSSNYMSWVRQAPGKGLEWVSGIYSGGSTYYADSVKGRFTISRDNSKNTLYLQMNSLRAEDTAVYYCARDLQEAGAFDIWGQGTMVTVSS

SEQ ID NO: 7 - protein, 111 aa, Artificial Sequence
  Description of Artificial Sequence Synthetic polypeptide
ELELMQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 8 - protein, 118 aa, Artificial Sequence
  Description of Artificial Sequence Synthetic polypeptide
EVQLVESGGGLVQPGGSLRLSCAASGLTVSRNYMSWVRQAPGKGLEWVSVIYSGGSTYYADSVKGRFTISRDNSKNTLYLQMNSLRAEDTAVYYCARDLDTAGGMDVWGQGTTVTVSS

SEQ ID NO: 9 - protein, 107 aa, Artificial Sequence
  Description of Artificial Sequence Synthetic polypeptide
ELRMTQSPSSLSASVGDRVTITCRASQSISNYLNWYQQKPGKAPKLLIYAASSLQSGVPSRFSGSGSGTDFTLTISSLQPEDFATYYCQQSYSTPPTFGQGTKLEIK

SEQ ID NO: 10 - protein, 123 aa, Artificial Sequence
  Description of Artificial Sequence Synthetic polypeptide
QMQLVQSGAEVKKPGASVKVSCKASGHTITSYYMHWVRQAPGQGLEWMGIINPSGGSTSYAQKFQGRVTMTRDTSTSTVYMELSSLRSEDTAVYYCAREGVWDSSGYSSFDYWGQGTLVTVSS

SEQ ID NO: 11 - protein, 111 aa, Artificial Sequence
  Description of Artificial Sequence Synthetic polypeptide
ELVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 12 - protein, 121 aa, Artificial Sequence
  Description of Artificial Sequence Synthetic polypeptide
EVHLVESGGGLIQPGGSLRLSCAASGVTVSSNYMSWVRQAPGKGLEWVSVIYSGGSTYYADSVKGRFTISRDNSKNTLYLQMNSLRAEDTAVYYCARGDGSGDYYYGMDVWGQGTTVTVSS

SEQ ID NO: 13 - protein, 110 aa, Artificial Sequence
  Description of Artificial Sequence Synthetic polypeptide
ELVVTQPPSVSAAPGQKVTISCSGSSSNIGNNYVSWYQQLPRTAPKLLIYDNNNRPSGIPDRFSGSKSGTSATLGITGLQTGDEAEYYCGTWDSSLSAVVFGGGTKLTVL

SEQ ID NO: 14 - protein, 124 aa, Artificial Sequence
  Description of Artificial Sequence Synthetic polypeptide
QVQLLESGGGLVQPGGSLRLSCAASGFTFDDYAMHWVRQAPGKGLEWVSGVTWNSGSIGYADSVKGRFIISRDNAKNSLYLQMNSLRAEDTALYYCAKDISPMLRGDNYGMDVWGQGTTVTVSS

SEQ ID NO: 15 - protein, 110 aa, Artificial Sequence
  Description of Artificial Sequence Synthetic polypeptide
ELGLTQPPSASGTPGQRVTISCSGSRSNIGSNVVNWYQQLPGTAPKLLIYSNDYRPSGVPDRFSGSKSGTSASLDISGLQSEDEADYYCAAWDDSLNGSVFGGGTKLTVL

SEQ ID NO: 16 - protein, 127 aa, Artificial Sequence
  Description of Artificial Sequence Synthetic polypeptide
QVQLVQSGAEVKKPGESLRISCKGSGYSFTTYWINWVRQMPGKGLEWMGRIDPSDSYTNYSPSFQGHVTISADKSISTAYLQWSGLKASDTAMYYCARGDYYDNSDYSGLSEYFQHWGQGTMVTISS

SEQ ID NO: 17 - protein, 110 aa, Artificial Sequence
  Description of Artificial Sequence Synthetic polypeptide
ELALTQPPSASGSPGQSVTISCTASSSDIGASNHVAWYQQNPGKAPKLMIYGVSGRPSGVPDRFSGSKSGNTASLTVSGLQAEDEADYYCSSYAGSNILIFGGGTKLTVL

SEQ ID NO: 18 - protein, 118 aa, Artificial Sequence
  Description of Artificial Sequence Synthetic polypeptide
QVQLVQSGGGLIQPGGSLRLSCAASGVTVSSNYMSWVRQAPGKGLEWVSVIYSGGSTFYADSVKGRFTISRDNSKNTLYLQMNSLRAEDTAVYYCARDLMEAGGMDVWGQGSTVTVSS

SEQ ID NO: 19 - protein, 110 aa, Artificial Sequence
  Description of Artificial Sequence Synthetic polypeptide
ELALTQPASVSGSPGQSITISCTGTSSDIGGYEYVSWYQQHPGKAPKLIIYDVRDRPSGISNRFSGSKSGNTASLIISSLQAGDEADYYCFSYTSSGTYVFGSATKVTVL

SEQ ID NO: 20 - protein, 129 aa, Artificial Sequence
  Description of Artificial Sequence Synthetic polypeptide
QMQLVESGGGLVQPGRSLRLSCAASGFTFSVYGMHWVRQAPGKGLEWVAVISYDGSNKYYADSVKGRFSISRDNSKNTLYLQMNSLRGEDTAVYYCAKGGPRPVVKAYGELDYYGMDVWGQGTTVTVSS

SEQ ID NO: 21 - protein, 110 aa, Artificial Sequence
  Description of Artificial Sequence Synthetic polypeptide
ELVLTQPPSASGTPGQRVTISCSGSSSNIGSNTVNWYQQVPGTAPKLLIYSNNQRPSGVPDRFSGSKSGTSASLAISGLQSEDEADYYCAAWDDSLNIYVFGSGTKVTVL

SEQ ID NO: 22 - protein, 128 aa, Artificial Sequence
  Description of Artificial Sequence Synthetic polypeptide
QVQLVESGGGVVRPGGSLRLSCAASGFTFDDYGMTWVRQVPGKGLEWVSGINWNGGTTGYADSVKGRFTISRDNAKKSLYLQMNSLRAEDTALYHCARIYCGDDCYSLVIWGDAFDIWGQGTMVTVSS

SEQ ID NO: 23 - protein, 106 aa, Artificial Sequence
  Description of Artificial Sequence Synthetic polypeptide
ELELTQPPSVSVAPGKTARITCGGNSIGSKSVHWYQQKPGQSPVLVIYQDSKRPSGIPERFSGSNSGNTATLTISGTQAMDEADYYCQAWDSSTVVFGGGTKLTVL

SEQ ID NO: 24 - protein, 117 aa, Artificial Sequence
  Description of Artificial Sequence Synthetic polypeptide
QVQLVESGGGLVKPGGSLRLSCAASGFTVSSNYMSWVRQAPGKGLEWVSVLYSGGSTFYADSVKGRFTISRDNSKNTLYLQMNSLRAEDTAVYYCTRDAQVYGMDVWGQGTTVTVSS

SEQ ID NO: 25 - protein, 111 aa, Artificial Sequence
  Description of Artificial Sequence Synthetic polypeptide
ELVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSVFGGGTKLTVL

SEQ ID NO: 26 - protein, 128 aa, Artificial Sequence
  Description of Artificial Sequence Synthetic polypeptide
QMQLVQSGAEVKKPGASVKVSCKASGYTFTRYAMHWVRQAPGQRLEWMGWINAGNGKTKYSQKFQGRVTITRDTSASTAYMELSSLRSEDTAVYYCARALYYYDSSGSTQSDDAFDIWGQGTMVTVSS

SEQ ID NO: 27 - protein, 9 aa, Artificial Sequence
  Description of Artificial Sequence Synthetic peptide
DLYYYGMDV

SEQ ID NO: 28 - protein, 9 aa, Artificial Sequence
  Description of Artificial Sequence Synthetic peptide
DLDYYGMDV

SEQ ID NO: 29 - protein, 9 aa, Artificial Sequence
  Description of Artificial Sequence Synthetic peptide
DLVAYGMDV

SEQ ID NO: 30 - protein, 9 aa, Artificial Sequence
  Description of Artificial Sequence Synthetic peptide
DLVYYGDMV

SEQ ID NO: 31 - protein, 9 aa, Artificial Sequence
  Description of Artificial Sequence Synthetic peptide
DLVYYGMDV

SEQ ID NO: 32 - protein, 9 aa, Artificial Sequence
  Description of Artificial Sequence Synthetic peptide
DLVVYGMDV

SEQ ID NO: 33 - protein, 9 aa, Artificial Sequence
  Description of Artificial Sequence Synthetic peptide
DLSYYGMDV

SEQ ID NO: 34 - protein, 9 aa, Artificial Sequence
  Description of Artificial Sequence Synthetic peptide
DLGDYGMDV

SEQ ID NO: 35 - protein, 9 aa, Artificial Sequence
  Description of Artificial Sequence Synthetic peptide
DAVSYGMDV

SEQ ID NO: 36 - protein, 9 aa, Artificial Sequence
  Description of Artificial Sequence Synthetic peptide
DLAVYGMDV

SEQ ID NO: 37 - protein, 9 aa, Artificial Sequence
  Description of Artificial Sequence Synthetic peptide
DLGDYGDMV

SEQ ID NO: 38 - protein, 9 aa, Artificial Sequence
  Description of Artificial Sequence Synthetic peptide
DLGPYGMDV

SEQ ID NO: 39 - protein, 9 aa, Artificial Sequence
  Description of Artificial Sequence Synthetic peptide
DLVIYGMDV

SEQ ID NO: 40 - protein, 9 aa, Artificial Sequence
  Description of Artificial Sequence Synthetic peptide
DLVVLGMDV

SEQ ID NO: 41 - protein, 5 aa, Artificial Sequence
  Description of Artificial Sequence Synthetic peptide
SNYMS

SEQ ID NO: 42 - protein, 16 aa, Artificial Sequence
  Description of Artificial Sequence Synthetic peptide
VLYSGGSTFYADSVKG

SEQ ID NO: 43 - protein, 9 aa, Artificial Sequence
  Description of Artificial Sequence Synthetic peptide
DAQVYGMDV

SEQ ID NO: 44 - protein, 5 aa, Artificial Sequence
  Description of Artificial Sequence Synthetic peptide
RNYMS

SEQ ID NO: 45 - protein, 16 aa, Artificial Sequence
  Description of Artificial Sequence Synthetic peptide
VIYSGGSTYYADSVKG

SEQ ID NO: 46 - protein, 10 aa, Artificial Sequence
  Description of Artificial Sequence Synthetic peptide
DLDTAGGMDV

SEQ ID NO: 47 - protein, 10 aa, Artificial Sequence
  Description of Artificial Sequence Synthetic peptide
DLLEQGGMDV

SEQ ID NO: 48 - protein, 16 aa, Artificial Sequence
  Description of Artificial Sequence Synthetic peptide
VIYSGGSTFYADSVKG

SEQ ID NO: 49 - protein, 10 aa, Artificial Sequence
  Description of Artificial Sequence Synthetic peptide
DLMEAGGMDV

SEQ ID NO: 50 - protein, 16 aa, Artificial Sequence
  Description of Artificial Sequence Synthetic peptide
GIYSGGSTYYADSVKG

SEQ ID NO: 51 - protein, 10 aa, Artificial Sequence
  Description of Artificial Sequence Synthetic peptide
DLQEAGAFDI

SEQ ID NO: 52 - protein, 5 aa, Artificial Sequence
  Description of Artificial Sequence Synthetic peptide
SYWMS

SEQ ID NO: 53 - protein, 17 aa, Artificial Sequence
  Description of Artificial Sequence Synthetic peptide
NIKQDGSEKYYVDSVKG

SEQ ID NO: 54 - protein, 10 aa, Artificial Sequence
  Description of Artificial Sequence Synthetic peptide
HRWLRGEIDY

SEQ ID NO: 55 - protein, 5 aa, Artificial Sequence
  Description of Artificial Sequence Synthetic peptide
DYYMS

SEQ ID NO: 56 - protein, 17 aa, Artificial Sequence
  Description of Artificial Sequence Synthetic peptide
VISYDGSNKYYADSVKG

SEQ ID NO: 57 - protein, 10 aa, Artificial Sequence
  Description of Artificial Sequence Synthetic peptide
SSWLRGAFDY

SEQ ID NO: 58 - protein, 5 aa, Artificial Sequence
  Description of Artificial Sequence Synthetic peptide
SYWIG

SEQ ID NO: 59 - protein, 17 aa, Artificial Sequence
  Description of Artificial Sequence Synthetic peptide
IIYPGDSDTRYSPSFQG

SEQ ID NO: 60 - protein, 11 aa, Artificial Sequence
  Description of Artificial Sequence Synthetic peptide
LSSSYYGWFDP

SEQ ID NO: 61 - protein, 5 aa, Artificial Sequence
  Description of Artificial Sequence Synthetic peptide
SYWIA

SEQ ID NO: 62 - protein, 11 aa, Artificial Sequence
  Description of Artificial Sequence Synthetic peptide
YSSSPNGWFDP

SEQ ID NO: 63 - protein, 5 aa, Artificial Sequence
  Description of Artificial Sequence Synthetic peptide
SNAIS

SEQ ID NO: 64 - protein, 17 aa, Artificial Sequence
  Description of Artificial Sequence Synthetic peptide
RIIPIFGTANYAQKFQG

SEQ ID NO: 65 - protein, 12 aa, Artificial Sequence
  Description of Artificial Sequence Synthetic peptide
DVIESPLYGMDV

SEQ ID NO: 66 - protein, 5 aa, Artificial Sequence
  Description of Artificial Sequence Synthetic peptide
SFAIT

SEQ ID NO: 67 - protein, 17 aa, Artificial Sequence
  Description of Artificial Sequence Synthetic peptide
RIIPILGIANYAQKFQG

SEQ ID NO: 68 - protein, 12 aa, Artificial Sequence
  Description of Artificial Sequence Synthetic peptide
EFSGGDNTGFDY

SEQ ID NO: 69 - protein, 5 aa, Artificial Sequence
  Description of Artificial Sequence Synthetic peptide
SHYMH

SEQ ID NO: 70 - protein, 17 aa, Artificial Sequence
  Description of Artificial Sequence Synthetic peptide
IINPSGGSTSYAQKFQG

SEQ ID NO: 71 - protein, 13 aa, Artificial Sequence
  Description of Artificial Sequence Synthetic peptide
DGYFVPARSAFDI

SEQ ID NO: 72 - protein, 5 aa, Artificial Sequence
  Description of Artificial Sequence Synthetic peptide
DYAMH

SEQ ID NO: 73 - protein, 17 aa, Artificial Sequence
  Description of Artificial Sequence Synthetic peptide
GISWNSGTIGYADSVKG

SEQ ID NO: 74 - protein, 13 aa, Artificial Sequence
  Description of Artificial Sequence Synthetic peptide
DITMVREAYGMDV

SEQ ID NO: 75 - protein, 17 aa, Artificial Sequence
  Description of Artificial Sequence Synthetic peptide
GTSWNSGTIGYADSVKG

SEQ ID NO: 76 - protein, 13 aa, Artificial Sequence
  Description of Artificial Sequence Synthetic peptide
DKGQIRESYGMDV

SEQ ID NO: 77 - protein, 17 aa, Artificial Sequence
  Description of Artificial Sequence Synthetic peptide
GTDWNSGTIGYADSVKG

SEQ ID NO: 78 - protein, 13 aa, Artificial Sequence
  Description of Artificial Sequence Synthetic peptide
DLGGVVERYGMDV

SEQ ID NO: 79 - protein, 5 aa, Artificial Sequence
  Description of Artificial Sequence Synthetic peptide
SYYIH

SEQ ID NO: 80 - protein, 17 aa, Artificial Sequence
  Description of Artificial Sequence Synthetic peptide
IINPDAGSTTYAQKFQG

SEQ ID NO: 81 - protein, 13 aa, Artificial Sequence
  Description of Artificial Sequence Synthetic peptide
DLYGLPGRAAFDI

SEQ ID NO: 82 - protein, 13 aa, Artificial Sequence
  Description of Artificial Sequence Synthetic peptide
GDGSGDYYYGMDV

SEQ ID NO: 83 - protein, 5 aa, Artificial Sequence
  Description of Artificial Sequence Synthetic peptide
NYWIG

SEQ ID NO: 84 - protein, 13 aa, Artificial Sequence
  Description of Artificial Sequence Synthetic peptide
HLDWNAPRGPFDI

SEQ ID NO: 85 - protein, 14 aa, Artificial Sequence
  Description of Artificial Sequence Synthetic peptide
DIFRTEWLQYGMDV

SEQ ID NO: 86 - protein, 17 aa, Artificial Sequence
  Description of Artificial Sequence Synthetic peptide
GSSWNSGTIGYADSVKG

SEQ ID NO: 87 - protein, 14 aa, Artificial Sequence
  Description of Artificial Sequence Synthetic peptide
DMGRGNDNNLAFDI

SEQ ID NO: 88 - protein, 5 aa, Artificial Sequence
  Description of Artificial Sequence Synthetic peptide
SYYMH

SEQ ID NO: 89 - protein, 14 aa, Artificial Sequence
  Description of Artificial Sequence Synthetic peptide
EGVWDSSGYSSFDY

SEQ ID NO: 90 - protein, 17 aa, Artificial Sequence
  Description of Artificial Sequence Synthetic peptide
GVTWNSGSIGYADSVKG

SEQ ID NO: 91 - protein, 15 aa, Artificial Sequence
  Description of Artificial Sequence Synthetic peptide
DISPMLRGDNYGMDV

SEQ ID NO: 92 - protein, 17 aa, Artificial Sequence
  Description of Artificial Sequence Synthetic peptide
SVTWNSGNIGYADSVKG

SEQ ID NO: 93 - protein, 15 aa, Artificial Sequence
  Description of Artificial Sequence Synthetic peptide
DISSMLRGDNYCMDV

SEQ ID NO: 94 - protein, 5 aa, Artificial Sequence
  Description of Artificial Sequence Synthetic peptide
SYAIS

SEQ ID NO: 95 - protein, 15 aa, Artificial Sequence
  Description of Artificial Sequence Synthetic peptide
DRGYSDYGSNPFFDY

SEQ ID NO: 96 - protein, 18 aa, Artificial Sequence
  Description of Artificial Sequence Synthetic peptide
  MOD_RES (18)..(18): Any amino acid
RIIPILGIANYAQKFQGX

SEQ ID NO: 97 - protein, 15 aa, Artificial Sequence
  Description of Artificial Sequence Synthetic peptide
GIGYSGSGSNDYFDS

SEQ ID NO: 98 - protein, 17 aa, Artificial Sequence
  Description of Artificial Sequence Synthetic peptide
GISWNSGIIGYADSVKG

SEQ ID NO: 99 - protein, 16 aa, Artificial Sequence
  Description of Artificial Sequence Synthetic peptide
DIRGYSGYDDPGAFDI

SEQ ID NO: 100 - protein, 17 aa, Artificial Sequence
  Description of Artificial Sequence Synthetic peptide
GSSWNSGSIGYADSVKG

SEQ ID NO: 101 - protein, 16 aa, Artificial Sequence
  Description of Artificial Sequence Synthetic peptide
GKSPLDYDQTMGAFDI

SEQ ID NO: 102 - protein, 5 aa, Artificial Sequence
  Description of Artificial Sequence Synthetic peptide
DYAMS

SEQ ID NO: 103 - protein, 19 aa, Artificial Sequence
  Description of Artificial Sequence Synthetic peptide
FIRSKAYGGTTEYAASVKG

SEQ ID NO: 104 - protein, 18 aa, Artificial Sequence
  Description of Artificial Sequence Synthetic peptide
DEDSGTLLPGFYYYDMDV

SEQ ID NO: 105 - protein, 5 aa, Artificial Sequence
  Description of Artificial Sequence Synthetic peptide
TYWIN

SEQ ID NO: 106 - protein, 17 aa, Artificial Sequence
  Description of Artificial Sequence Synthetic peptide
RIDPSDSYTNYSPSFQG

SEQ ID NO: 107 - protein, 18 aa, Artificial Sequence
  Description of Artificial Sequence Synthetic peptide
GDYYDNSDYSGLSEYFQH

SEQ ID NO: 108 - protein, 5 aa, Artificial Sequence
  Description of Artificial Sequence Synthetic peptide
RYAMH

SEQ ID NO: 109 - protein, 17 aa, Artificial Sequence
  Description of Artificial Sequence Synthetic peptide
WINAGNGKTKYSQKFQG

SEQ ID NO: 110 - protein, 19 aa, Artificial Sequence
  Description of Artificial Sequence Synthetic peptide
ALYYYDSSGSTQSDDAFDI

SEQ ID NO: 111 - protein, 19 aa, Artificial Sequence
  Description of Artificial Sequence Synthetic peptide
DGQRMAAAGTEDYYYGMDV

SEQ ID NO: 112 - protein, 17 aa, Artificial Sequence
  Description of Artificial Sequence Synthetic peptide
GVTWNSGTIGYADSVKG

SEQ ID NO: 113 - protein, 19 aa, Artificial Sequence
  Description of Artificial Sequence Synthetic peptide
DIMGDGSPSLHYYYYGMDV

SEQ ID NO: 114 - protein, 16 aa, Artificial Sequence
  Description of Artificial Sequence Synthetic peptide
VIYIGGSTYYSYSVKG

SEQ ID NO: 115 - protein, 19 aa, Artificial Sequence
  Description of Artificial Sequence Synthetic peptide
DRQRMAAAGTEDYYYGMDV

SEQ ID NO: 116 - protein, 5 aa, Artificial Sequence
  Description of Artificial Sequence Synthetic peptide
DYGMT

SEQ ID NO: 117 - protein, 17 aa, Artificial Sequence
  Description of Artificial Sequence Synthetic peptide
GINWNGGTTGYADSVKG

SEQ ID NO: 118 - protein, 19 aa, Artificial Sequence
  Description of Artificial Sequence Synthetic peptide
IYCGDDCYSLVIWGDAFDI

SEQ ID NO: 119 - protein, 20 aa, Artificial Sequence
  Description of Artificial Sequence Synthetic peptide
DENRGYSSRWYDPEYYGMDV

SEQ ID NO: 120 - protein, 5 aa, Artificial Sequence
  Description of Artificial Sequence Synthetic peptide
VYGMH

SEQ ID NO: 121 - protein, 20 aa, Artificial Sequence
  Description of Artificial Sequence Synthetic peptide
GGPRPVVKAYGELDYYGMDV

SEQ ID NO: 122 - protein, 17 aa, Artificial Sequence
  Description of Artificial Sequence Synthetic peptide
GTSWNSGTIGYADSVRG

SEQ ID NO: 123 - protein, 20 aa, Artificial Sequence
  Description of Artificial Sequence Synthetic peptide
YGTEGLYDFRSGYGHYGMDV

SEQ ID NO: 124 - protein, 5 aa, Artificial Sequence
  Description of Artificial Sequence Synthetic peptide
RYAIS

SEQ ID NO: 125 - protein, 17 aa, Artificial Sequence
  Description of Artificial Sequence Synthetic peptide
GIIPIFGTANYAQKFQG

SEQ ID NO: 126 - protein, 21 aa, Artificial Sequence
  Description of Artificial Sequence Synthetic peptide
ERTYCSSTSCYAGYYYYGMDV

SEQ ID NO: 127 - protein, 9 aa, Artificial Sequence
  Description of Artificial Sequence Synthetic peptide
DLQVYGMDV

SEQ ID NO: 128 - protein, 16 aa, Artificial Sequence
  Description of Artificial Sequence Synthetic peptide
DIYSGGSTDYADSVKG

SEQ ID NO: 129 - protein, 5 aa, Artificial Sequence
  Description of Artificial Sequence Synthetic peptide
SNYMN

SEQ ID NO: 130 - protein, 16 aa, Artificial Sequence
  Description of Artificial Sequence Synthetic peptide
VIYAGGTTDYADSVKG

SEQ ID NO: 131 - protein, 9 aa, Artificial Sequence
  Description of Artificial Sequence Synthetic peptide
DLVDYGMDV

SEQ ID NO: 132 - protein, 9 aa, Artificial Sequence
  Description of Artificial Sequence Synthetic peptide
DLVVMGMDV

SEQ ID NO: 133 - protein, 5 aa, Artificial Sequence
  Description of Artificial Sequence Synthetic peptide
SNYMT

SEQ ID NO: 134 - protein, 16 aa, Artificial Sequence
  Description of Artificial Sequence Synthetic peptide
IIYSGGSTYYADSVKG

SEQ ID NO: 135 - protein, 16 aa, Artificial Sequence
  Description of Artificial Sequence Synthetic peptide
IIYSGGSTFYADSVKG

SEQ ID NO: 136 - protein, 9 aa, Artificial Sequence
  Description of Artificial Sequence Synthetic peptide
DRDYYGMDV

SEQ ID NO: 137 - protein, 16 aa, Artificial Sequence
  Description of Artificial Sequence Synthetic peptide
VIYSGGSTDYADSVKG

SEQ ID NO: 138 - protein, 16 aa, Artificial Sequence
  Description of Artificial Sequence Synthetic peptide
VIYSGGSTYYADPVKG

SEQ ID NO: 139 - protein, 16 aa, Artificial Sequence
  Description of Artificial Sequence Synthetic peptide
VIYSGGSTYYTDSVKG

SEQ ID NO: 140 - protein, 9 aa, Artificial Sequence
  Description of Artificial Sequence Synthetic peptide
DLHYYGMDV

SEQ ID NO: 141 - protein, 9 aa, Artificial Sequence
  Description of Artificial Sequence Synthetic peptide
DLAYYGMDV

SEQ ID NO: 142 - protein, 16 aa, Artificial Sequence
  Description of Artificial Sequence Synthetic peptide
VIYSGGTTYYADSVKG

SEQ ID NO: 143 - protein, 16 aa, Artificial Sequence
  Description of Artificial Sequence Synthetic peptide
VIYSGGSIFYADSVKG

SEQ ID NO: 144 - protein, 5 aa, Artificial Sequence
  Description of Artificial Sequence Synthetic peptide
SNYMC

SEQ ID NO: 145 - protein, 9 aa, Artificial Sequence
  Description of Artificial Sequence Synthetic peptide
DLGSYGMDV

SEQ ID NO: 146 - protein, 9 aa, Artificial Sequence
  Description of Artificial Sequence Synthetic peptide
DLPYYGMDV

SEQ ID NO: 147 - protein, 9 aa, Artificial Sequence
  Description of Artificial Sequence Synthetic peptide
DLTVYGMDV

SEQ ID NO: 148 - protein, 16 aa, Artificial Sequence
  Description of Artificial Sequence Synthetic peptide
LIYSGGSTYYADSVKG

SEQ ID NO: 149 - protein, 9 aa, Artificial Sequence
  Description of Artificial Sequence Synthetic peptide
DLVVWGMDV

SEQ ID NO: 150 - protein, 16 aa, Artificial Sequence
  Description of Artificial Sequence Synthetic peptide
VLYSGGSTYYADSVKG

SEQ ID NO: 151 - protein, 16 aa, Artificial Sequence
  Description of Artificial Sequence Synthetic peptide
IIYSGGTTYYADSVKG

SEQ ID NO: 152 - protein, 9 aa, Artificial Sequence
  Description of Artificial Sequence Synthetic peptide
DVVVWGMDV

SEQ ID NO: 153 - protein, 9 aa, Artificial Sequence
  Description of Artificial Sequence Synthetic peptide
DLIMYGMDV

SEQ ID NO: 154 - protein, 9 aa, Artificial Sequence
  Description of Artificial Sequence Synthetic peptide
DLQDYGMDV

SEQ ID NO: 155 - protein, 5 aa, Artificial Sequence
  Description of Artificial Sequence Synthetic peptide
RNYMN

SEQ ID NO: 156 - protein, 9 aa, Artificial Sequence
  Description of Artificial Sequence Synthetic peptide
DRVVYGMDV

SEQ ID NO: 157 - protein, 9 aa, Artificial Sequence
  Description of Artificial Sequence Synthetic peptide
DSPYYGMDV

SEQ ID NO: 158 - protein, 5 aa, Artificial Sequence
  Description of Artificial Sequence Synthetic peptide
NNYMS

SEQ ID NO: 159 - protein, 16 aa, Artificial Sequence
  Description of Artificial Sequence Synthetic peptide
IIYNDGSTYYADSVKG

SEQ ID NO: 160 - protein, 9 aa, Artificial Sequence
  Description of Artificial Sequence Synthetic peptide
DAVLTGMDV

SEQ ID NO: 161 - protein, 5 aa, Artificial Sequence
  Description of Artificial Sequence Synthetic peptide
TNYIS

SEQ ID NO: 162 - protein, 5 aa, Artificial Sequence
  Description of Artificial Sequence Synthetic peptide
GNYMC

SEQ ID NO: 163 - protein, 16 aa, Artificial Sequence
  Description of Artificial Sequence Synthetic peptide
VIFADGRAYYADSVRG

SEQ ID NO: 164 - protein, 9 aa, Artificial Sequence
  Description of Artificial Sequence Synthetic peptide
DMADYGMDV

SEQ ID NO: 165 - protein, 9 aa, Artificial Sequence
  Description of Artificial Sequence Synthetic peptide
DAASYGMDV

SEQ ID NO: 166 - protein, 16 aa, Artificial Sequence
  Description of Artificial Sequence Synthetic peptide
VIYSGGSTFYVDSVKG

SEQ ID NO: 167 - protein, 5 aa, Artificial Sequence
  Description of Artificial Sequence Synthetic peptide
SNYMR

SEQ ID NO: 168 - protein, 9 aa, Artificial Sequence
  Description of Artificial Sequence Synthetic peptide
DAQSYGMDD

SEQ ID NO: 169 - protein, 9 aa, Artificial Sequence
  Description of Artificial Sequence Synthetic peptide
DAQSYGMDV

SEQ ID NO: 170 - protein, 16 aa, Artificial Sequence
  Description of Artificial Sequence Synthetic peptide
VIYSGGSTYYADSAKG

SEQ ID NO: 171 - protein, 5 aa, Artificial Sequence
  Description of Artificial Sequence Synthetic peptide
SNYMI

SEQ ID NO: 172 - protein, 16 aa, Artificial Sequence
  Description of Artificial Sequence Synthetic peptide
VIYSGGSTYYADSVRG

SEQ ID NO: 173 - protein, 9 aa, Artificial Sequence
  Description of Artificial Sequence Synthetic peptide
DIVIYGMDV

SEQ ID NO: 174 - protein, 16 aa, Artificial Sequence
  Description of Artificial Sequence Synthetic peptide
VIYIGGSTYYADSVKG

SEQ ID NO: 175 - protein, 9 aa, Artificial Sequence
  Description of Artificial Sequence Synthetic peptide
DLQYRGMDV

SEQ ID NO: 176 - protein, 16 aa, Artificial Sequence
  Description of Artificial Sequence Synthetic peptide
IIYSGGSAFYTDSVKG

SEQ ID NO: 177 - protein, 9 aa, Artificial Sequence
  Description of Artificial Sequence Synthetic peptide
DLVVGGMDV

SEQ ID NO: 178 - protein, 9 aa, Artificial Sequence
  Description of Artificial Sequence Synthetic peptide
DNPMYGMDV

SEQ ID NO: 179 - protein, 5 aa, Artificial Sequence
  Description of Artificial Sequence Synthetic peptide
SHYMS

SEQ ID NO: 180 - protein, 16 aa, Artificial Sequence
  Description of Artificial Sequence Synthetic peptide
LIYGGHDTNYADSVKG

SEQ ID NO: 181 - protein, 9 aa, Artificial Sequence
  Description of Artificial Sequence Synthetic peptide
DRPLYGMDV

SEQ ID NO: 182 - protein, 9 aa, Artificial Sequence
  Description of Artificial Sequence Synthetic peptide
DRVVRGMDV

SEQ ID NO: 183 - protein, 16 aa, Artificial Sequence
  Description of Artificial Sequence Synthetic peptide
VIYSGGSTYYEDSVKG

SEQ ID NO: 184 - protein, 9 aa, Artificial Sequence
  Description of Artificial Sequence Synthetic peptide
DLSAYGMDV

SEQ ID NO: 185 - protein, 9 aa, Artificial Sequence
  Description of Artificial Sequence Synthetic peptide
DLIVYGMDV

SEQ ID NO: 186 - protein, 9 aa, Artificial Sequence
  Description of Artificial Sequence Synthetic peptide
DLMVRGMDV

SEQ ID NO: 187 - protein, 9 aa, Artificial Sequence
  Description of Artificial Sequence Synthetic peptide
DTDKYGMDV

SEQ ID NO: 188 - protein, 9 aa, Artificial Sequence
  Description of Artificial Sequence Synthetic peptide
DTDYYGMDV

SEQ ID NO: 189 - protein, 5 aa, Artificial Sequence
  Description of Artificial Sequence Synthetic peptide
GFYIH

SEQ ID NO: 190 - protein, 17 aa, Artificial Sequence
  Description of Artificial Sequence Synthetic peptide
RINPDSGATDYAQKFQG

SEQ ID NO: 191 - protein, 5 aa, Artificial Sequence
  Description of Artificial Sequence Synthetic peptide
GDLRD

SEQ ID NO: 192 - protein, 5 aa, Artificial Sequence
  Description of Artificial Sequence Synthetic peptide
GYYMH

SEQ ID NO: 193 - protein, 17 aa, Artificial Sequence
  Description of Artificial Sequence Synthetic peptide
RINPNSGGTNYAQKFQG

SEQ ID NO: 194 - protein, 5 aa, Artificial Sequence
  Description of Artificial Sequence Synthetic peptide
GHMDV

SEQ ID NO: 195 - protein, 5 aa, Artificial Sequence
  Description of Artificial Sequence Synthetic peptide
RNMDV

SEQ ID NO: 196 - protein, 7 aa, Artificial Sequence
  Description of Artificial Sequence Synthetic peptide
DAFGMDV

SEQ ID NO: 197 - protein, 5 aa, Artificial Sequence
  Description of Artificial Sequence Synthetic peptide
SYSMN

SEQ ID NO: 198 - protein, 17 aa, Artificial Sequence
  Description of Artificial Sequence Synthetic peptide
YIYRRDSSIFYADSVKG

SEQ ID NO: 199 - protein, 8 aa, Artificial Sequence
  Description of Artificial Sequence Synthetic peptide
EDWQSLDY

SEQ ID NO: 200 - protein, 8 aa, Artificial Sequence
  Description of Artificial Sequence Synthetic peptide
WDSRAFDI

SEQ ID NO: 201 - protein, 5 aa, Artificial Sequence
  Description of Artificial Sequence Synthetic peptide
TYWIG

SEQ ID NO: 202 - protein, 8 aa, Artificial Sequence
  Description of Artificial Sequence Synthetic peptide
YNSGWLDF

SEQ ID NO: 203 - protein, 5 aa, Artificial Sequence
  Description of Artificial Sequence Synthetic peptide
SYGMH

SEQ ID NO: 204 - protein, 17 aa, Artificial Sequence
  Description of Artificial Sequence Synthetic peptide
VIWFDERNRYYSDSVKG

SEQ ID NO: 205 - protein, 9 aa, Artificial Sequence
  Description of Artificial Sequence Synthetic peptide
ANNYFPFDY

SEQ ID NO: 206 - protein, 9 aa, Artificial Sequence
  Description of Artificial Sequence Synthetic peptide
DAQRYGMDV

SEQ ID NO: 207 - protein, 9 aa, Artificial Sequence
  Description of Artificial Sequence Synthetic peptide
DLGPGGMDV

SEQ ID NO: 208 - protein, 16 aa, Artificial Sequence
  Description of Artificial Sequence Synthetic peptide
VIYSGGSTYYADTVKG

SEQ ID NO: 209 - protein, 9 aa, Artificial Sequence
  Description of Artificial Sequence Synthetic peptide
DLSELGVDY

SEQ ID NO: 210 - protein, 9 aa, Artificial Sequence
  Description of Artificial Sequence Synthetic peptide
DLTIFGMDV

SEQ ID NO: 211 - protein, 17 aa, Artificial Sequence
  Description of Artificial Sequence Synthetic peptide
YISSISSYTNYADSVKG

SEQ ID NO: 212 - protein, 9 aa, Artificial Sequence
  Description of Artificial Sequence Synthetic peptide
DLVGGAFDI

SEQ ID NO: 213 - protein, 9 aa, Artificial Sequence
  Description of Artificial Sequence Synthetic peptide
DLVVRGVDI

SEQ ID NO: 214 - protein, 9 aa, Artificial Sequence
  Description of Artificial Sequence Synthetic peptide
DLVVSGMDV

SEQ ID NO: 215 - protein, 9 aa, Artificial Sequence
  Description of Artificial Sequence Synthetic peptide
DVPIYGMDV

SEQ ID NO: 216 - protein, 9 aa, Artificial Sequence
  Description of Artificial Sequence Synthetic peptide
DVVVYGMDV

SEQ ID NO: 217 - protein, 16 aa, Artificial Sequence
  Description of Artificial Sequence Synthetic peptide
VIYSGGSTFYSDSVKG

SEQ ID NO: 218 - protein, 9 aa, Artificial Sequence
  Description of Artificial Sequence Synthetic peptide
DWGEYYFDY

SEQ ID NO: 219 - protein, 9 aa, Artificial Sequence
  Description of Artificial Sequence Synthetic peptide
ELGVYGMDV

SEQ ID NO: 220 - protein, 9 aa, Artificial Sequence
  Description of Artificial Sequence Synthetic peptide
ELYYYGMDV

SEQ ID NO: 221 - protein, 9 aa, Artificial Sequence
  Description of Artificial Sequence Synthetic peptide
GYGDYYFDY

SEQ ID NO: 222 - protein, 9 aa, Artificial Sequence
  Description of Artificial Sequence Synthetic peptide
QDSGWAFDY

SEQ ID NO: 223 - protein, 16 aa, Artificial Sequence
  Description of Artificial Sequence Synthetic peptide
LIYSGGSTFYADSVKG

SEQ ID NO: 224 - protein, 9 aa, Artificial Sequence
  Description of Artificial Sequence Synthetic peptide
SLEYYGMDV

SEQ ID NO: 225 - protein, 5 aa, Artificial Sequence
  Description of Artificial Sequence Synthetic peptide
SNWIG

SEQ ID NO: 226 - protein, 9 aa, Artificial Sequence
  Description of Artificial Sequence Synthetic peptide
VGDGYPFDY

SEQ ID NO: 227 - protein, 6 aa, Artificial Sequence
  Description of Artificial Sequence Synthetic peptide
SSNWWS

SEQ ID NO: 228 - protein, 16 aa, Artificial Sequence
  Description of Artificial Sequence Synthetic peptide
EIYHSGSTNYNPSLKS

SEQ ID NO: 229 - protein, 9 aa, Artificial Sequence
  Description of Artificial Sequence Synthetic peptide
VPQADAFDI

SEQ ID NO: 230 - protein, 10 aa, Artificial Sequence
  Description of Artificial Sequence Synthetic peptide
APATYASFDY

SEQ ID NO: 231 - protein, 7 aa, Artificial Sequence
  Description of Artificial Sequence Synthetic peptide
SGDYYWS

SEQ ID NO: 232 - protein, 16 aa, Artificial Sequence
  Description of Artificial Sequence Synthetic peptide
YIYYSGSTYYNPSLKS

SEQ ID NO: 233 - protein, 10 aa, Artificial Sequence
  Description of Artificial Sequence Synthetic peptide
AQWLRGHHDY

SEQ ID NO: 234 - protein, 10 aa, Artificial Sequence
  Description of Artificial Sequence Synthetic peptide
DLDIVGAFDI

SEQ ID NO: 235 - protein, 10 aa, Artificial Sequence
  Description of Artificial Sequence Synthetic peptide
DLEILGGMDV

SEQ ID NO: 236 - protein, 10 aa, Artificial Sequence
  Description of Artificial Sequence Synthetic peptide
DLMAAGGMDV

SEQ ID NO: 237 - protein, 16 aa, Artificial Sequence
  Description of Artificial Sequence Synthetic peptide
VIYSGGSRYYADSVKG

SEQ ID NO: 238 - protein, 16 aa, Artificial Sequence
  Description of Artificial Sequence Synthetic peptide
VIYSGGSTYYADFVKG

SEQ ID NO: 239 - protein, 17 aa, Artificial Sequence
  Description of Artificial Sequence Synthetic peptide
WINPNSGGTNYAQKFQG

SEQ ID NO: 240 - protein, 10 aa, Artificial Sequence
  Description of Artificial Sequence Synthetic peptide
DLSNVVFFDS

SEQ ID NO: 241 - protein, 10 aa, Artificial Sequence
  Description of Artificial Sequence Synthetic peptide
DRWLRGDMDV

SEQ ID NO: 242 - protein, 5 aa, Artificial Sequence
  Description of Artificial Sequence Synthetic peptide
NAWMS

SEQ ID NO: 243 - protein, 19 aa, Artificial Sequence
  Description of Artificial Sequence Synthetic peptide
RIKTKTDGGTTDYAAPVKG

SEQ ID NO: 244 - protein, 10 aa, Artificial Sequence
  Description of Artificial Sequence Synthetic peptide
EWGYYDSLDY

SEQ ID NO: 245 - protein, 17 aa, Artificial Sequence
  Description of Artificial Sequence Synthetic peptide
YISSSGSTIYYADSVKG

SEQ ID NO: 246 - protein, 10 aa, Artificial Sequence
  Description of Artificial Sequence Synthetic peptide
GEWLRGGFDP

SEQ ID NO: 247 - protein, 17 aa, Artificial Sequence
  Description of Artificial Sequence Synthetic peptide
IIDPSGGSTSYAQKFQG

SEQ ID NO: 248 - protein, 10 aa, Artificial Sequence
  Description of Artificial Sequence Synthetic peptide
HDISPYYFDY

SEQ ID NO: 249 - protein, 10 aa, Artificial Sequence
  Description of Artificial Sequence Synthetic peptide
HENLYYGMDV

SEQ ID NO: 250 - protein, 7 aa, Artificial Sequence
  Description of Artificial Sequence Synthetic peptide
SSSYYWG

SEQ ID NO: 251 - protein, 16 aa, Artificial Sequence
  Description of Artificial Sequence Synthetic peptide
TFYYSRSTYYNPSLKS

SEQ ID NO: 252 - protein, 10 aa, Artificial Sequence
  Description of Artificial Sequence Synthetic peptide
LEWLRGHFDY

SEQ ID NO: 253 - protein, 10 aa, Artificial Sequence
  Description of Artificial Sequence Synthetic peptide
MWSGVTAFDI

SEQ ID NO: 254 - protein, 16 aa, Artificial Sequence
  Description of Artificial Sequence Synthetic peptide
SIYYSGSTYYNPSLKS

SEQ ID NO: 255 - protein, 10 aa, Artificial Sequence
  Description of Artificial Sequence Synthetic peptide
NEWLRGPFDY

SEQ ID NO: 256 - protein, 5 aa, Artificial Sequence
  Description of Artificial Sequence Synthetic peptide
SYDIN

SEQ ID NO: 257 - protein, 17 aa, Artificial Sequence
  Description of Artificial Sequence Synthetic peptide
WMNPNSGNTGYAQKFQG

SEQ ID NO: 258 - protein, 10 aa, Artificial Sequence
  Description of Artificial Sequence Synthetic peptide
NPGGSGQFDP

SEQ ID NO: 259 - protein, 10 aa, Artificial Sequence
  Description of Artificial Sequence Synthetic peptide
PVMSRDGMDV

SEQ ID NO: 260 - protein, 16 aa, Artificial Sequence
  Description of Artificial Sequence Synthetic peptide
VIYPGGTTYYADSVKG

SEQ ID NO: 261 - protein, 10 aa, Artificial Sequence
  Description of Artificial Sequence Synthetic peptide
QLPFGDYFDY

SEQ ID NO: 262 - protein, 5 aa, Artificial Sequence
  Description of Artificial Sequence Synthetic peptide
SNFMS

SEQ ID NO: 263 - protein, 10 aa, Artificial Sequence
  Description of Artificial Sequence Synthetic peptide
QRWRQGWFDP

SEQ ID NO: 264 - protein, 10 aa, Artificial Sequence
  Description of Artificial Sequence Synthetic peptide
REWLRGHVDV

SEQ ID NO: 265 - protein, 10 aa, Artificial Sequence
  Description of Artificial Sequence Synthetic peptide
RKWLRGAFDI

SEQ ID NO: 266 - protein, 5 aa, Artificial Sequence
  Description of Artificial Sequence Synthetic peptide
YYWIG

SEQ ID NO: 267 - protein, 10 aa, Artificial Sequence
  Description of Artificial Sequence Synthetic peptide
RSTTVGWLDY

SEQ ID NO: 268 - protein, 10 aa, Artificial Sequence
  Description of Artificial Sequence Synthetic peptide
RVYYYGWLDV

SEQ ID NO: 269 - protein, 5 aa, Artificial Sequence
  Description of Artificial Sequence Synthetic peptide
SYGIH

SEQ ID NO: 270 - protein, 17 aa, Artificial Sequence
  Description of Artificial Sequence Synthetic peptide
LISYDGSDKYYADPVKG

SEQ ID NO: 271 - protein, 10 aa, Artificial Sequence
  Description of Artificial Sequence Synthetic peptide
SSWYKLGFDP

SEQ ID NO: 272 - protein, 10 aa, Artificial Sequence
  Description of Artificial Sequence Synthetic peptide
TPWLRGAFDY

SEQ ID NO: 273 - protein, 5 aa, Artificial Sequence
  Description of Artificial Sequence Synthetic peptide
SYEMN

SEQ ID NO: 274 - protein, 10 aa, Artificial Sequence
  Description of Artificial Sequence Synthetic peptide
TQWLRGAFDI

SEQ ID NO: 275 - protein, 5 aa, Artificial Sequence
  Description of Artificial Sequence Synthetic peptide
VNYMT

SEQ ID NO: 276 - protein, 10 aa, Artificial Sequence
  Description of Artificial Sequence Synthetic peptide
VLPYGDYADF

SEQ ID NO: 277 - protein, 10 aa, Artificial Sequence
  Description of Artificial Sequence Synthetic peptide
VLPYGDYVDY

SEQ ID NO: 278 - protein, 5 aa, Artificial Sequence
  Description of Artificial Sequence Synthetic peptide
SNWIA

SEQ ID NO: 279 - protein, 17 aa, Artificial Sequence
  Description of Artificial Sequence Synthetic peptide
IIYPGDSDTTYSPSFQG

SEQ ID NO: 280 - protein, 11 aa, Artificial Sequence
  Description of Artificial Sequence Synthetic peptide
ALGHIGSGYDY

SEQ ID NO: 281 - protein, 5 aa, Artificial Sequence
  Description of Artificial Sequence Synthetic peptide
SHWIG

SEQ ID NO: 282 - protein, 11 aa, Artificial Sequence
  Description of Artificial Sequence Synthetic peptide
APSGYYNWFDP

SEQ ID NO: 283 - protein, 17 aa, Artificial Sequence
  Description of Artificial Sequence Synthetic peptide
IISYDGSNKYYADSVKG

SEQ ID NO: 284 - protein, 11 aa, Artificial Sequence
  Description of Artificial Sequence Synthetic peptide
AQSWLHWYFDL

SEQ ID NO: 285 - protein, 5 aa, Artificial Sequence
  Description of Artificial Sequence Synthetic peptide
HYAIS

SEQ ID NO: 286 - protein, 17 aa, Artificial Sequence
  Description of Artificial Sequence Synthetic peptide
RIIPMLDISNYAQKFKG

SEQ ID NO: 287 - protein, 11 aa, Artificial Sequence
  Description of Artificial Sequence Synthetic peptide
DHTILPKGMDV

SEQ ID NO: 288 - protein, 11 aa, Artificial Sequence
  Description of Artificial Sequence Synthetic peptide
DLRGSSGWYDI

SEQ ID NO: 289 - protein, 5 aa, Artificial Sequence
  Description of Artificial Sequence Synthetic peptide
SYAMH

SEQ ID NO: 290 - protein, 17 aa, Artificial Sequence
  Description of Artificial Sequence Synthetic peptide
VISSDGGNKYYADSVKG

SEQ ID NO: 291 - protein, 11 aa, Artificial Sequence
  Description of Artificial Sequence Synthetic peptide
DTLLLVDAFDI

SEQ ID NO: 292 - protein, 5 aa, Artificial Sequence
  Description of Artificial Sequence Synthetic peptide
DYQMS

SEQ ID NO: 293 - protein, 17 aa, Artificial Sequence
  Description of Artificial Sequence Synthetic peptide
YISSSSSYTNYADSVKG

SEQ ID NO: 294 - protein, 11 aa, Artificial Sequence
  Description of Artificial Sequence Synthetic peptide
DWGYSSPRFDY

SEQ ID NO: 295 - protein, 11 aa, Artificial Sequence
  Description of Artificial Sequence Synthetic peptide
HGNWANSDLDY

SEQ ID NO: 296 - protein, 11 aa, Artificial Sequence
  Description of Artificial Sequence Synthetic peptide
LPSSWYNWFDP

SEQ ID NO: 297 - protein, 5 aa, Artificial Sequence
  Description of Artificial Sequence Synthetic peptide
SDWIG

SEQ ID NO: 298 - protein, 11 aa, Artificial Sequence
  Description of Artificial Sequence Synthetic peptide
MLCGGDCPFDY

SEQ ID NO: 299 - protein, 11 aa, Artificial Sequence
  Description of Artificial Sequence Synthetic peptide
SIVTTNAGFDF

SEQ ID NO: 300 - protein, 11 aa, Artificial Sequence
  Description of Artificial Sequence Synthetic peptide
SSSGPHDAFDI

SEQ ID NO: 301 - protein, 11 aa, Artificial Sequence
  Description of Artificial Sequence Synthetic peptide
VLPLYGDYLDY

SEQ ID NO: 302 - protein, 5 aa, Artificial Sequence
  Description of Artificial Sequence Synthetic peptide
SYGIT

SEQ ID NO: 303 - protein, 17 aa, Artificial Sequence
  Description of Artificial Sequence Synthetic peptide
WISAYNGNTKYAQKLQG

SEQ ID NO: 304 - protein, 11 aa, Artificial Sequence
  Description of Artificial Sequence Synthetic peptide
VMGIAVAGTVV

SEQ ID NO: 305 - protein, 17 aa, Artificial Sequence
  Description of Artificial Sequence Synthetic peptide
AISSNGGSTYYANSVKG

SEQ ID NO: 306 - protein, 11 aa, Artificial Sequence
  Description of Artificial Sequence Synthetic peptide
VPDDLIWYFDL

SEQ ID NO: 307 - protein, 11 aa, Artificial Sequence
  Description of Artificial Sequence Synthetic peptide
VPDDLNWYFDL

SEQ ID NO: 308 - protein, 5 aa, Artificial Sequence
  Description of Artificial Sequence Synthetic peptide
SYGIS

SEQ ID NO: 309 - protein, 17 aa, Artificial Sequence
  Description of Artificial Sequence Synthetic peptide
WISAYNGNTNYAQKLQG

SEQ ID NO: 310 - protein, 11 aa, Artificial Sequence
  Description of Artificial Sequence Synthetic peptide
VVELGIGWFDP

SEQ ID NO: 311 - protein, 7 aa, Artificial Sequence
  Description of Artificial Sequence Synthetic peptide
STSFHWG

SEQ ID NO: 312 - protein, 16 aa, Artificial Sequence
  Description of Artificial Sequence Synthetic peptide
TISYSGRAYHNPSLKS

SEQ ID NO: 313 - protein, 11 aa, Artificial Sequence
  Description of Artificial Sequence Synthetic peptide
WNSHYYYGMHV

SEQ ID NO: 314 - protein, 5 aa, Artificial Sequence
  Description of Artificial Sequence Synthetic peptide
NYAMS

SEQ ID NO: 315 - protein, 17 aa, Artificial Sequence
  Description of Artificial Sequence Synthetic peptide
AISGSGGSTYYADSVKG

SEQ ID NO: 316 - protein, 12 aa, Artificial Sequence
  Description of Artificial Sequence Synthetic peptide
AIAAAGYWVFDY

SEQ ID NO: 317 - protein, 5 aa, Artificial Sequence
  Description of Artificial Sequence Synthetic peptide
KCVMS

SEQ ID NO: 318 - protein, 17 aa, Artificial Sequence
  Description of Artificial Sequence Synthetic peptide
SISDGGDNINDADSVKG

SEQ ID NO: 319 - protein, 12 aa, Artificial Sequence
  Description of Artificial Sequence Synthetic peptide
AKSGSDRHVFEI

SEQ ID NO: 320 - protein, 7 aa, Artificial Sequence
  Description of Artificial Sequence Synthetic peptide
SVDYYWS

SEQ ID NO: 321 - protein, 12 aa, Artificial Sequence
  Description of Artificial Sequence Synthetic peptide
DLRWGRGGGMDV

SEQ ID NO: 322 - protein, 17 aa, Artificial Sequence
  Description of Artificial Sequence Synthetic peptide
GISWNSGNIGYADSVKG

SEQ ID NO: 323 - protein, 12 aa, Artificial Sequence
  Description of Artificial Sequence Synthetic peptide
DSLGELLSGMDV

SEQ ID NO: 324 - protein, 17 aa, Artificial Sequence
  Description of Artificial Sequence Synthetic peptide
GISWNSGSIGYADSVKG

SEQ ID NO: 325 - protein, 12 aa, Artificial Sequence
  Description of Artificial Sequence Synthetic peptide
DSSAGHGDYFDY

SEQ ID NO: 326 - protein, 17 aa, Artificial Sequence
  Description of Artificial Sequence Synthetic peptide
GISWNSGGIAYADSVKG

SEQ ID NO: 327 - protein, 16 aa, Artificial Sequence
  Description of Artificial Sequence Synthetic peptide
VIYSGGTTYYTDSVKG

SEQ ID NO: 328 - protein, 12 aa, Artificial Sequence
  Description of Artificial Sequence Synthetic peptide
GDILTAPPPIDY

SEQ ID NO: 329 - protein, 7 aa, Artificial Sequence
  Description of Artificial Sequence Synthetic peptide
SNIVTWI

SEQ ID NO: 330 - protein, 18 aa, Artificial Sequence
  Description of Artificial Sequence Synthetic peptide
RTYYRSKWYNDYAVSVKS

SEQ ID NO: 331 - protein, 12 aa, Artificial Sequence
  Description of Artificial Sequence Synthetic peptide
GRFGGYFYGMDV

SEQ ID NO: 332 - protein, 12 aa, Artificial Sequence
  Description of Artificial Sequence Synthetic peptide
GRLGELLDAFDI

SEQ ID NO: 333 - protein, 5 aa, Artificial Sequence
  Description of Artificial Sequence Synthetic peptide
SYWMH

SEQ ID NO: 334 - protein, 17 aa, Artificial Sequence
  Description of Artificial Sequence Synthetic peptide
RINGDGSDTGYADSLRA

SEQ ID NO: 335 - protein, 12 aa, Artificial Sequence
  Description of Artificial Sequence Synthetic peptide
GVDYGRGAVLQH

SEQ ID NO: 336 - protein, 5 aa, Artificial Sequence
  Description of Artificial Sequence Synthetic peptide
DYWIG

SEQ ID NO: 337 - protein, 12 aa, Artificial Sequence
  Description of Artificial Sequence Synthetic peptide
HSLADPVHWFDP

SEQ ID NO: 338 - protein, 12 aa, Artificial Sequence
  Description of Artificial Sequence Synthetic peptide
LESIAAAGWADY

SEQ ID NO: 339 - protein, 17 aa, Artificial Sequence
  Description of Artificial Sequence Synthetic peptide
IINPGDSETIYSPSFQG

SEQ ID NO: 340 - protein, 12 aa, Artificial Sequence
  Description of Artificial Sequence Synthetic peptide
LGSGGSHNWFDP

SEQ ID NO: 341 - protein, 7 aa, Artificial Sequence
  Description of Artificial Sequence Synthetic peptide
SGDYYWN

SEQ ID NO: 342 - protein, 12 aa, Artificial Sequence
  Description of Artificial Sequence Synthetic peptide
SSPLVVTDAFDI

SEQ ID NO: 343 - protein, 12 aa, Artificial Sequence
  Description of Artificial Sequence Synthetic peptide
VGWGYDSEYFDL

SEQ ID NO: 344 - protein, 7 aa, Artificial Sequence
  Description of Artificial Sequence Synthetic peptide
SNSAAWN

SEQ ID NO: 345 - protein, 18 aa, Artificial Sequence
  Description of Artificial Sequence Synthetic peptide
RTYYRFKWYYDYALSLES

SEQ ID NO: 346 - protein, 12 aa, Artificial Sequence
  Description of Artificial Sequence Synthetic peptide
VSAPGPRGWFDP

SEQ ID NO: 347 - protein, 13 aa, Artificial Sequence
  Description of Artificial Sequence Synthetic peptide
ALEVNAFGDYFDY

SEQ ID NO: 348 - protein, 17 aa, Artificial Sequence
  Description of Artificial Sequence Synthetic peptide
IINPGGGSTSYAQKFQG

SEQ ID NO: 349 - protein, 13 aa, Artificial Sequence
  Description of Artificial Sequence Synthetic peptide
DAGYVPTTGGMDV

SEQ ID NO: 350 - protein, 5 aa, Artificial Sequence
  Description of Artificial Sequence Synthetic peptide
TYYWS

SEQ ID NO: 351 - protein, 16 aa, Artificial Sequence
  Description of Artificial Sequence Synthetic peptide
YIYNSGSTNYNPSLKS

SEQ ID NO: 352 - protein, 13 aa, Artificial Sequence
  Description of Artificial Sequence Synthetic peptide
DANLSGSFDALDI

SEQ ID NO: 353 - protein, 13 aa, Artificial Sequence
  Description of Artificial Sequence Synthetic peptide
DGGAVAETYGMDV

SEQ ID NO: 354 - protein, 5 aa, Artificial Sequence
  Description of Artificial Sequence Synthetic peptide
NHYMH

SEQ ID NO: 355 - protein, 13 aa, Artificial Sequence
  Description of Artificial Sequence Synthetic peptide
DRWFIPQSGYFDL

SEQ ID NO: 356 - protein, 13 aa, Artificial Sequence
  Description of Artificial Sequence Synthetic peptide
DSYYLPAMGPFDY

SEQ ID NO: 357 - protein, 13 aa, Artificial Sequence
  Description of Artificial Sequence Synthetic peptide
GAWGVPAASPSDP

SEQ ID NO: 358 - protein, 17 aa, Artificial Sequence
  Description of Artificial Sequence Synthetic peptide
IINPSGGSTSYAQKFQA

SEQ ID NO: 359 - protein, 13 aa, Artificial Sequence
  Description of Artificial Sequence Synthetic peptide
GGVVPAASSAFDI

SEQ ID NO: 360 - protein, 13 aa, Artificial Sequence
  Description of Artificial Sequence Synthetic peptide
GKWYSSPLEYFDY

SEQ ID NO: 361 - protein, 17 aa, Artificial Sequence
  Description of Artificial Sequence Synthetic peptide
AISWNSGSIDYADSVKG

SEQ ID NO: 362 - protein, 13 aa, Artificial Sequence
  Description of Artificial Sequence Synthetic peptide
GLLAEFVVPTLDY

SEQ ID NO: 363 - protein, 5 aa, Artificial Sequence
  Description of Artificial Sequence Synthetic peptide
SYWIS

SEQ ID NO: 364 - protein, 13 aa, Artificial Sequence
  Description of Artificial Sequence Synthetic peptide
GQQWLSNNWYFDL

SEQ ID NO: 365 - protein, 13 aa, Artificial Sequence
  Description of Artificial Sequence Synthetic peptide
HLDWNAPRGAFDI

SEQ ID NO: 366 - protein, 13 aa, Artificial Sequence
  Description of Artificial Sequence Synthetic peptide
LGHGDPGLRYFDL

SEQ ID NO: 367 - protein, 16 aa, Artificial Sequence
  Description of Artificial Sequence Synthetic peptide
EIFHSGSASYNPSLKS

SEQ ID NO: 368 - protein, 19 aa, Artificial Sequence
  Description of Artificial Sequence Synthetic peptide
RIKSKTDGGTTDYAAPVKG

SEQ ID NO: 369 - protein, 13 aa, Artificial Sequence
  Description of Artificial Sequence Synthetic peptide
NDVIQYYHYGMDV

SEQ ID NO: 370 - protein, 13 aa, Artificial Sequence
  Description of Artificial Sequence Synthetic peptide
NDVLQYYYYGMDV

SEQ ID NO: 371 - protein, 5 aa, Artificial Sequence
  Description of Artificial Sequence Synthetic peptide
DFAMS

SEQ ID NO: 372 - protein, 19 aa, Artificial Sequence
  Description of Artificial Sequence Synthetic peptide
FIRGTAYGGTTEYAASVKG

SEQ ID NO: 373 - protein, 13 aa, Artificial Sequence
  Description of Artificial Sequence Synthetic peptide
NHMTTVTWLGADI

SEQ ID NO: 374 - protein, 13 aa, Artificial Sequence
  Description of Artificial Sequence Synthetic peptide
PGSISLVRGVRDV

SEQ ID NO: 375 - protein, 13 aa, Artificial Sequence
  Description of Artificial Sequence Synthetic peptide
SDILQYYYYGMDV

SEQ ID NO: 376 - protein, 5 aa, Artificial Sequence
  Description of Artificial Sequence Synthetic peptide
NYGMH

SEQ ID NO: 377 - protein, 17 aa, Artificial Sequence
  Description of Artificial Sequence Synthetic peptide
GVSYDGSDKYYADSVKG

SEQ ID NO: 378 - protein, 13 aa, Artificial Sequence
  Description of Artificial Sequence Synthetic peptide
TVATHYYYYGMDV

SEQ ID NO: 379 - protein, 14 aa, Artificial Sequence
  Description of Artificial Sequence Synthetic peptide
AALYGDYEEGYFDY

SEQ ID NO: 380 - protein, 14 aa, Artificial Sequence
  Description of Artificial Sequence Synthetic peptide
AGYSYGYPEIYFDY

SEQ ID NO: 381 - protein, 14 aa, Artificial Sequence
  Description of Artificial Sequence Synthetic peptide
ALQPMDGGEYYFDY

SEQ ID NO: 382 - protein, 5 aa, Artificial Sequence
  Description of Artificial Sequence Synthetic peptide
DYAMY

SEQ ID NO: 383 - protein, 14 aa, Artificial Sequence
  Description of Artificial Sequence Synthetic peptide
DAGVTEYYYYGMDV

SEQ ID NO: 384 - protein, 14 aa, Artificial Sequence
  Description of Artificial Sequence Synthetic peptide
DIGFGELLSYGMDV

SEQ ID NO: 385 - protein, 14 aa, Artificial Sequence
  Description of Artificial Sequence Synthetic peptide
DIRKGDGFEFYFDY

SEQ ID NO: 386 - protein, 5 aa, Artificial Sequence
  Description of Artificial Sequence Synthetic peptide
SYAMS

SEQ ID NO: 387 - protein, 14 aa, Artificial Sequence
  Description of Artificial Sequence Synthetic peptide
DPMVRGPSFDYFDY

SEQ ID NO: 388 - protein, 5 aa, Artificial Sequence
  Description of Artificial Sequence Synthetic peptide
RYGMH

SEQ ID NO: 389 - protein, 17 aa, Artificial Sequence
  Description of Artificial Sequence Synthetic peptide
VISYDGSNKYYVDSVKG

SEQ ID NO: 390 - protein, 14 aa, Artificial Sequence
  Description of Artificial Sequence Synthetic peptide
DVPLGIAATYLFDY

SEQ ID NO: 391 - protein, 14 aa, Artificial Sequence
  Description of Artificial Sequence Synthetic peptide
EAGMGAAAGTAFDY

SEQ ID NO: 392 - protein, 17 aa, Artificial Sequence
  Description of Artificial Sequence Synthetic peptide
GISWNSGSIVYADSVKG

SEQ ID NO: 393 - protein, 14 aa, Artificial Sequence
  Description of Artificial Sequence Synthetic peptide
GHTAMHYYYYGMDV

SEQ ID NO: 394 - protein, 14 aa, Artificial Sequence
  Description of Artificial Sequence Synthetic peptide
HEGACSGGSCGIDY

SEQ ID NO: 395 - protein, 14 aa, Artificial Sequence
  Description of Artificial Sequence Synthetic peptide
NIYSYAYPQYYFDY

SEQ ID NO: 396 - protein, 14 aa, Artificial Sequence
  Description of Artificial Sequence Synthetic peptide
TVATHYYYYYGMDV

SEQ ID NO: 397 - protein, 15 aa, Artificial Sequence
  Description of Artificial Sequence Synthetic peptide
AGDSSGWAPLDAFDI

SEQ ID NO: 398 - protein, 15 aa, Artificial Sequence
  Description of Artificial Sequence Synthetic peptide
APIGYCTNGVCYFDY

SEQ ID NO: 399 - protein, 21 aa, Artificial Sequence
  Description of Artificial Sequence Synthetic peptide
RIIPILGIANFIANYAQKFQG

SEQ ID NO: 400 - protein, 15 aa, Artificial Sequence
  Description of Artificial Sequence Synthetic peptide
DDYSNYDYYYYGMDV

SEQ ID NO: 401 - protein, 15 aa, Artificial Sequence
  Description of Artificial Sequence Synthetic peptide
DLRDSSGYSFGAFDI

SEQ ID NO: 402 - protein, 17 aa, Artificial Sequence
  Description of Artificial Sequence Synthetic peptide
FISYDGSNKYYADSVKG

SEQ ID NO: 403 - protein, 15 aa, Artificial Sequence
  Description of Artificial Sequence Synthetic peptide
DMAVAGYYYYYGMDV

SEQ ID NO: 404 - protein, 17 aa, Artificial Sequence
  Description of Artificial Sequence Synthetic peptide
IINPSGGSRSYAQKFQG

SEQ ID NO: 405 - protein, 15 aa, Artificial Sequence
  Description of Artificial Sequence Synthetic peptide
DYDYVWGSYPNAFDI

SEQ ID NO: 406 - protein, 17 aa, Artificial Sequence
  Description of Artificial Sequence Synthetic peptide
GIIPMFGTANYAQKFQG

SEQ ID NO: 407 - protein, 15 aa, Artificial Sequence
  Description of Artificial Sequence Synthetic peptide
ERSVTKNLYYYGMDV

SEQ ID NO: 408 - protein, 15 aa, Artificial Sequence
  Description of Artificial Sequence Synthetic peptide
FPTYHDILTGYEVDY

SEQ ID NO: 409 - protein, 5 aa, Artificial Sequence
  Description of Artificial Sequence Synthetic peptide
NYAIS

SEQ ID NO: 410 - protein, 15 aa, Artificial Sequence
  Description of Artificial Sequence Synthetic peptide
GIGYSGSGSNDYFDY

SEQ ID NO: 411 - protein, 17 aa, Artificial Sequence
  Description of Artificial Sequence Synthetic peptide
VISYDGSNEYYADSVKG

SEQ ID NO: 412 - protein, 15 aa, Artificial Sequence
  Description of Artificial Sequence Synthetic peptide
GPWYSSGWYYQGFED

SEQ ID NO: 413 - protein, 17 aa, Artificial Sequence
  Description of Artificial Sequence Synthetic peptide
RIIPMFGIANYAQKFQG

SEQ ID NO: 414 - protein, 15 aa, Artificial Sequence
  Description of Artificial Sequence Synthetic peptide
HKYEYYDSSGYPFDY

SEQ ID NO: 415 - protein, 15 aa, Artificial Sequence
  Description of Artificial Sequence Synthetic peptide
LHRPYGDLQYNWFDP

SEQ ID NO: 416 - protein, 15 aa, Artificial Sequence
  Description of Artificial Sequence Synthetic peptide
PPNSSGANFRNAFDI

SEQ ID NO: 417 - protein, 15 aa, Artificial Sequence
  Description of Artificial Sequence Synthetic peptide
PPPTVTHYYYYGMDV

SEQ ID NO: 418 - protein, 16 aa, Artificial Sequence
  Description of Artificial Sequence Synthetic peptide
AGRTKRNYYYYYGMDV

SEQ ID NO: 419 - protein, 16 aa, Artificial Sequence
  Description of Artificial Sequence Synthetic peptide
DHRILSAGYYYYGMDV

SEQ ID NO: 420 - protein, 17 aa, Artificial Sequence
  Description of Artificial Sequence Synthetic peptide
GITWNSGSIGYADSVKG

SEQ ID NO: 421 - protein, 16 aa, Artificial Sequence
  Description of Artificial Sequence Synthetic peptide
DIGPYDFWSRSYGMDV

SEQ ID NO: 422 - protein, 5 aa, Artificial Sequence
  Description of Artificial Sequence Synthetic peptide
SYATH

SEQ ID NO: 423 - protein, 17 aa, Artificial Sequence
  Description of Artificial Sequence Synthetic peptide
VISSDGSKKYYADSVKG

SEQ ID NO: 424 - protein, 16 aa, Artificial Sequence
  Description of Artificial Sequence Synthetic peptide
DLVPWLVVKFHYGVDV

SEQ ID NO: 425 - protein, 16 aa, Artificial Sequence
  Description of Artificial Sequence Synthetic peptide
DRAVREGYNYYYGMDV

SEQ ID NO: 426 - protein, 5 aa, Artificial Sequence
  Description of Artificial Sequence Synthetic peptide
TYAMS

SEQ ID NO: 427 - protein, 17 aa, Artificial Sequence
  Description of Artificial Sequence Synthetic peptide
AISGSGGNTYYADSVKG

SEQ ID NO: 428 - protein, 16 aa, Artificial Sequence
  Description of Artificial Sequence Synthetic peptide
DRWRESSGWYPDAFDI

SEQ ID NO: 429 - protein, 16 aa, Artificial Sequence
  Description of Artificial Sequence Synthetic peptide
DVRYDSSGYYDIFRDY

SEQ ID NO: 430 - protein, 5 aa, Artificial Sequence
  Description of Artificial Sequence Synthetic peptide
NHAMY

SEQ ID NO: 431 - protein, 17 aa, Artificial Sequence
  Description of Artificial Sequence Synthetic peptide
VISYDGSKEYYADSVKG

SEQ ID NO: 432 - protein, 16 aa, Artificial Sequence
  Description of Artificial Sequence Synthetic peptide
EEGGSYFTHYYYGMDV

SEQ ID NO: 433 - protein, 16 aa, Artificial Sequence
  Description of Artificial Sequence Synthetic peptide
GGATYCSGGSCYSFDH

SEQ ID NO: 434 - protein, 16 aa, Artificial Sequence
  Description of Artificial Sequence Synthetic peptide
GGATYCSGGSCYSFDY

SEQ ID NO: 435 - protein, 17 aa, Artificial Sequence
  Description of Artificial Sequence Synthetic peptide
GISWNSGFMGYADSVKG

SEQ ID NO: 436 - protein, 16 aa, Artificial Sequence
  Description of Artificial Sequence Synthetic peptide
GLYQVRYKYYYYALDV

SEQ ID NO: 437 - protein, 16 aa, Artificial Sequence
  Description of Artificial Sequence Synthetic peptide
HNTIFGVLGSDYGMDV

SEQ ID NO: 438 - protein, 5 aa, Artificial Sequence
  Description of Artificial Sequence Synthetic peptide
SHWIS

SEQ ID NO: 439 - protein, 16 aa, Artificial Sequence
  Description of Artificial Sequence Synthetic peptide
HTLLGELSSPTNWFDP

SEQ ID NO: 440 - protein, 16 aa, Artificial Sequence
  Description of Artificial Sequence Synthetic peptide
RVRQWLVRPSWAAFDI

SEQ ID NO: 441 - protein, 16 aa, Artificial Sequence
  Description of Artificial Sequence Synthetic peptide
VDGLSSGSYLLPSIDY

SEQ ID NO: 442 - protein, 16 aa, Artificial Sequence
  Description of Artificial Sequence Synthetic peptide
VPYYYDSSGHRGGMDV

SEQ ID NO: 443 - protein, 17 aa, Artificial Sequence
  Description of Artificial Sequence Synthetic peptide
DRPDYDYVWGSLVPFDY

SEQ ID NO: 444 - protein, 17 aa, Artificial Sequence
  Description of Artificial Sequence Synthetic peptide
DYYASGSYSPEDYGMDV

SEQ ID NO: 445 - protein, 17 aa, Artificial Sequence
  Description of Artificial Sequence Synthetic peptide
GISWNSGRIGYADSVKG

SEQ ID NO: 446 - protein, 17 aa, Artificial Sequence
  Description of Artificial Sequence Synthetic peptide
EGTGDGYNLLIGGAFDI

SEQ ID NO: 447 - protein, 5 aa, Artificial Sequence
  Description of Artificial Sequence Synthetic peptide
TYGMH

SEQ ID NO: 448 - protein, 17 aa, Artificial Sequence
  Description of Artificial Sequence Synthetic peptide
GAFYYYGSGSYHYGMDV

SEQ ID NO: 449 - protein, 17 aa, Artificial Sequence
  Description of Artificial Sequence Synthetic peptide
PEWDYGDPLGYYYGMDV

SEQ ID NO: 450 - protein, 17 aa, Artificial Sequence
  Description of Artificial Sequence Synthetic peptide
SISSSSSYIYYADSVKG

SEQ ID NO: 451 - protein, 17 aa, Artificial Sequence
  Description of Artificial Sequence Synthetic peptide
VPAMEDGDYYYYYGMDV

SEQ ID NO: 452 - protein, 17 aa, Artificial Sequence
  Description of Artificial Sequence Synthetic peptide
YDFWSGQNTNYYYVLDV

SEQ ID NO: 453 - protein, 18 aa, Artificial Sequence
  Description of Artificial Sequence Synthetic peptide
DEDSGTLLPGFYYYGMDV

SEQ ID NO: 454 - protein, 17 aa, Artificial Sequence
  Description of Artificial Sequence Synthetic peptide
IINPSGGSTSYSQKFQG

SEQ ID NO: 455 - protein, 18 aa, Artificial Sequence
  Description of Artificial Sequence Synthetic peptide
DGIAAAGTEYYYYYGMDV

SEQ ID NO: 456 - protein, 18 aa, Artificial Sequence
  Description of Artificial Sequence Synthetic peptide
DGIAAGGTEYYYYYGMDV

SEQ ID NO: 457 - protein, 18 aa, Artificial Sequence
  Description of Artificial Sequence Synthetic peptide
DITFDWLGVWYYYYGMDV

SEQ ID NO: 458 - protein, 18 aa, Artificial Sequence
  Description of Artificial Sequence Synthetic peptide
EKAVAGPRPSYYYYGMDV

SEQ ID NO: 459 - protein, 7 aa, Artificial Sequence
  Description of Artificial Sequence Synthetic peptide
SGNYYWS

SEQ ID NO: 460 - protein, 16 aa, Artificial Sequence
  Description of Artificial Sequence Synthetic peptide
YIYYSGSTNYNPSLKS

SEQ ID NO: 461 - protein, 18 aa, Artificial Sequence
  Description of Artificial Sequence Synthetic peptide
ETYYYDSSGYYGSDAFDI

SEQ ID NO: 462 - protein, 17 aa, Artificial Sequence
  Description of Artificial Sequence Synthetic peptide
NIEQDGSEKYYVDSVKG

SEQ ID NO: 463 - protein, 18 aa, Artificial Sequence
  Description of Artificial Sequence Synthetic peptide
IYGYYDRSGYYYGEYFQH

SEQ ID NO: 464 - protein, 18 aa, Artificial Sequence
  Description of Artificial Sequence Synthetic peptide
LPFPYYYDSSGYYAAFDI

SEQ ID NO: 465 - protein, 19 aa, Artificial Sequence
  Description of Artificial Sequence Synthetic peptide
FIRGKAYGGTSEYAASVKG

SEQ ID NO: 466 - protein, 18 aa, Artificial Sequence
  Description of Artificial Sequence Synthetic peptide
NIALVVYGMRLDYYGMDV

SEQ ID NO: 467 - protein, 18 aa, Artificial Sequence
  Description of Artificial Sequence Synthetic peptide
RIVVVPAGPWFYYYGMDV

SEQ ID NO: 468 - protein, 17 aa, Artificial Sequence
  Description of Artificial Sequence Synthetic peptide
WINAGNGNTKYSQKFQG

SEQ ID NO: 469 - protein, 19 aa, Artificial Sequence
  Description of Artificial Sequence Synthetic peptide
DTGMRYSSGWYGDDYGMDV

SEQ ID NO: 470 - protein, 19 aa, Artificial Sequence
  Description of Artificial Sequence Synthetic peptide
ERRCGDCYEPHYYYYGMDV

SEQ ID NO: 471 - protein, 19 aa, Artificial Sequence
  Description of Artificial Sequence Synthetic peptide
VLADYGDYHVSLGYYGMDV

SEQ ID NO: 472 - protein, 19 aa, Artificial Sequence
  Description of Artificial Sequence Synthetic peptide
VLYYYDRSGYYSSESDFQH

SEQ ID NO: 473 - protein, 20 aa, Artificial Sequence
  Description of Artificial Sequence Synthetic peptide
AGGPLDGSGSYSQPEYYFDY

SEQ ID NO: 474 - protein, 20 aa, Artificial Sequence
  Description of Artificial Sequence Synthetic peptide
ATQRLYYYASGSFLPDAFDI

SEQ ID NO: 475 - protein, 20 aa, Artificial Sequence
  Description of Artificial Sequence Synthetic peptide
ATQRLYYYGSGSYLPDAFDI

SEQ ID NO: 476 - protein, 20 aa, Artificial Sequence
  Description of Artificial Sequence Synthetic peptide
DENRGYSSSWYDPEYYGMDV

SEQ ID NO: 477 - protein, 20 aa, Artificial Sequence
  Description of Artificial Sequence Synthetic peptide
DIGPEGGYSWRRGVYYGMDV

SEQ ID NO: 478 - protein, 20 aa, Artificial Sequence
  Description of Artificial Sequence Synthetic peptide
DISTYYGSGSYYDEDYGMDV

SEQ ID NO: 479 - protein, 20 aa, Artificial Sequence
  Description of Artificial Sequence Synthetic peptide
DVPTYYYDSSGWAEHYGMDV

SEQ ID NO: 480 - protein, 5 aa, Artificial Sequence
  Description of Artificial Sequence Synthetic peptide
SYSIT

SEQ ID NO: 481 - protein, 17 aa, Artificial Sequence
  Description of Artificial Sequence Synthetic peptide
RIIPILGIANFAQKFQG

SEQ ID NO: 482 - protein, 20 aa, Artificial Sequence
  Description of Artificial Sequence Synthetic peptide
ESGGHYYGSGSYYNSNWFDP

SEQ ID NO: 483 - protein, 20 aa, Artificial Sequence
  Description of Artificial Sequence Synthetic peptide
VGEGPTVAQDDYYYYYDMDV

SEQ ID NO: 484 - protein, 20 aa, Artificial Sequence
  Description of Artificial Sequence Synthetic peptide
VSFYYDSSGYYSANGNGMDV

SEQ ID NO: 485 - protein, 5 aa, Artificial Sequence
  Description of Artificial Sequence Synthetic peptide
DYGMS

SEQ ID NO: 486 - protein, 17 aa, Artificial Sequence
  Description of Artificial Sequence Synthetic peptide
GINWNGGNTGYADSVKG

SEQ ID NO: 487 - protein, 21 aa, Artificial Sequence
  Description of Artificial Sequence Synthetic peptide
AAEGKLRYFDWLFFADYGMDV

SEQ ID NO: 488 - protein, 21 aa, Artificial Sequence
  Description of Artificial Sequence Synthetic peptide
ANGYCSSTSCLDYYYYYGMDV

SEQ ID NO: 489 - protein, 21 aa, Artificial Sequence
  Description of Artificial Sequence Synthetic peptide
DKAGYCSSTSCYARELDAFDI

SEQ ID NO: 490 - protein, 17 aa, Artificial Sequence
  Description of Artificial Sequence Synthetic peptide
GIIPIFGTANYAQKFQD

SEQ ID NO: 491 - protein, 22 aa, Artificial Sequence
  Description of Artificial Sequence Synthetic peptide
DIAPHYYDILTGYYEGAWGFDY

SEQ ID NO: 492 - protein, 17 aa, Artificial Sequence
  Description of Artificial Sequence Synthetic peptide
VISSDGSNKYYADSVKG

SEQ ID NO: 493 - protein, 22 aa, Artificial Sequence
  Description of Artificial Sequence Synthetic peptide
DLGVVPAASRWDDYYYYYGMDV

SEQ ID NO: 494 - protein, 22 aa, Artificial Sequence
  Description of Artificial Sequence Synthetic peptide
DRENLSIFGVSQRLTRYYGMDV

SEQ ID NO: 495 - protein, 17 aa, Artificial Sequence
  Description of Artificial Sequence Synthetic peptide
GIIPIFGTANYAQKFKG

SEQ ID NO: 496 - protein, 22 aa, Artificial Sequence
  Description of Artificial Sequence Synthetic peptide
EEFDLVVVPAATTQYYYYGMDV

SEQ ID NO: 497 - protein, 7 aa, Artificial Sequence
  Description of Artificial Sequence Synthetic peptide
TSGVGVG

SEQ ID NO: 498 - protein, 16 aa, Artificial Sequence
  Description of Artificial Sequence Synthetic peptide
LIYWDDDKRYSPSLKS

SEQ ID NO: 499 - protein, 22 aa, Artificial Sequence
  Description of Artificial Sequence Synthetic peptide
SPDRRYYDILTGYSNLYWYFDL

SEQ ID NO: 500 - protein, 23 aa, Artificial Sequence
  Description of Artificial Sequence Synthetic peptide
ALYDSSGYYRPGRDFYYYYAMDV

SEQ ID NO: 501 - protein, 23 aa, Artificial Sequence
  Description of Artificial Sequence Synthetic peptide
DIKKLYYDILTGYYNDADYGMDV

SEQ ID NO: 502 - protein, 17 aa, Artificial Sequence
  Description of Artificial Sequence Synthetic peptide
GISWNSGVIGYADSVKG

SEQ ID NO: 503 - protein, 23 aa, Artificial Sequence
  Description of Artificial Sequence Synthetic peptide
DIKRFYYDILTGYYNDADYGMDV

SEQ ID NO: 504 - protein, 23 aa, Artificial Sequence
  Description of Artificial Sequence Synthetic peptide
DVSGGYYGSGGYYKYYYYYGMDV

SEQ ID NO: 505 - protein, 5 aa, Artificial Sequence
  Description of Artificial Sequence Synthetic peptide
DYYIH

SEQ ID NO: 506 - protein, 23 aa, Artificial Sequence
  Description of Artificial Sequence Synthetic peptide
EGGEWYDSSGYYSTWSYYYGMDV

SEQ ID NO: 507 - protein, 23 aa, Artificial Sequence
  Description of Artificial Sequence Synthetic peptide
EGGPNYYDSSGYYYDSYYYGMDV

SEQ ID NO: 508 - protein, 23 aa, Artificial Sequence
  Description of Artificial Sequence Synthetic peptide
EGGPNYYDSSGYYYDYYYYGMDV

SEQ ID NO: 509 - protein, 23 aa, Artificial Sequence
  Description of Artificial Sequence Synthetic peptide
HPPDYYGSGSYYNGGPGMGGMDV

SEQ ID NO: 510 - protein, 23 aa, Artificial Sequence
  Description of Artificial Sequence Synthetic peptide
VAERVHYDILTGYYPYYYYAMDV

SEQ ID NO: 511 - DNA, 66 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnngaaggaagtcctgtgcgag

SEQ ID NO: 512 - DNA, 66 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnngggaagtagtccttgacca

SEQ ID NO: 513 - DNA, 65 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnggggaagaagccctggac

SEQ ID NO: 514 - DNA, 68 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnntgggtggtacccagttatcaa

SEQ ID NO: 515 - DNA, 65 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnaagtagcccgtggccagg

SEQ ID NO: 516 - DNA, 58 bp, Artificial Sequence
  Description of Artificial Sequence Synthetic primer
acactctttccctacacgacgctcttccgatctggcctcagtgaaggtctcctgcaag

SEQ ID NO: 517 - DNA, 57 bp, Artificial Sequence
  Description of Artificial Sequence Synthetic primer
acactctttccctacacgacgctcttccgatctgtctggtcctacgctggtgaaccc

SEQ ID NO: 518 - DNA, 58 bp, Artificial Sequence
  Description of Artificial Sequence Synthetic primer
acactctttccctacacgacgctcttccgatctctggggggtccctgagactctcctg

SEQ ID NO: 519 - DNA, 58 bp, Artificial Sequence
  Description of Artificial Sequence Synthetic primer
acactctttccctacacgacgctcttccgatctcttcggagaccctgtccctcacctg

SEQ ID NO: 520 - DNA, 58 bp, Artificial Sequence
  Description of Artificial Sequence Synthetic primer
acactctttccctacacgacgctcttccgatctcggggagtctctgaagatctcctgt

SEQ ID NO: 521 - DNA, 58 bp, Artificial Sequence
  Description of Artificial Sequence Synthetic primer
acactctttccctacacgacgctcttccgatcttcgcagaccctctcactcacctgtg

SEQ ID NO: 522 - DNA, 24 bp, Artificial Sequence
  Description of Artificial Sequence Synthetic primer
caccagtgtggccttgttggcttg

SEQ ID NO: 523 - DNA, 24 bp, Artificial Sequence
  Description of Artificial Sequence Synthetic primer
gtttctcgtagtctgctttgctca

SEQ ID NO: 524 - DNA, 58 bp, Artificial Sequence
  Description of Artificial Sequence Synthetic primer
acactctttccctacacgacgctcttccgatctatgagggtccccgctcagctgctgg

SEQ ID NO: 525 - DNA, 58 bp, Artificial Sequence
  Description of Artificial Sequence Synthetic primer
acactctttccctacacgacgctcttccgatctatgaggctccccgctcagctgctgg

SEQ ID NO: 526 - DNA, 58 bp, Artificial Sequence
  Description of Artificial Sequence Synthetic primer
acactctttccctacacgacgctcttccgatctatgagggtccctgctcagctgctgg

SEQ ID NO: 527 - DNA, 58 bp, Artificial Sequence
  Description of Artificial Sequence Synthetic primer
acactctttccctacacgacgctcttccgatctatgaggctccctgctcagctgctgg

SEQ ID NO: 528 - DNA, 61 bp, Artificial Sequence
  Description of Artificial Sequence Synthetic primer
acactctttccctacacgacgctcttccgatctctcttcctcctgctactctggctcccag

SEQ ID NO: 529 - DNA, 58 bp, Artificial Sequence
  Description of Artificial Sequence Synthetic primer
acactctttccctacacgacgctcttccgatctatttctctgttgctctggatctctg

SEQ ID NO: 530 - DNA, 58 bp, Artificial Sequence
  Description of Artificial Sequence Synthetic primer
acactctttccctacacgacgctcttccgatctagctcctggggctcctgctgctctg

SEQ ID NO: 531 - DNA, 58 bp, Artificial Sequence
  Description of Artificial Sequence Synthetic primer
acactctttccctacacgacgctcttccgatctatgaggctccctgctcagctcttgg

SEQ ID NO: 532 - DNA, 57 bp, Artificial Sequence
  Description of Artificial Sequence Synthetic primer
acactctttccctacacgacgctcttccgatctatggggtcccaggttcacctcctc

SEQ ID NO: 533 - DNA, 57 bp, Artificial Sequence
  Description of Artificial Sequence Synthetic primer
acactctttccctacacgacgctcttccgatctatgttgccatcacaactcattggg

SEQ ID NO: 534 - DNA, 68 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnntttgatatccaccttggtccc

SEQ ID NO: 535 - DNA, 68 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnntttaatctccagtcgtgtccc

SEQ ID NO: 536 - DNA, 67 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnntgatctccagcttggtcccc

SEQ ID NO: 537 - DNA, 56 bp, Artificial Sequence
  Description of Artificial Sequence Synthetic primer
acactctttccctacacgacgctcttccgatctggtcctgggcccagtctgtgctg

SEQ ID NO: 538 - DNA, 56 bp, Artificial Sequence
  Description of Artificial Sequence Synthetic primer
acactctttccctacacgacgctcttccgatctggtcctgggcccagtctgccctg

SEQ ID NO: 539 - DNA, 56 bp, Artificial Sequence
  Description of Artificial Sequence Synthetic primer
acactctttccctacacgacgctcttccgatctgctctgtgacctcctatgagctg

SEQ ID NO: 540 - DNA, 56 bp, Artificial Sequence
  Description of Artificial Sequence Synthetic primer
acactctttccctacacgacgctcttccgatctggtctctctcgcagcctgtgctg

SEQ ID NO: 541 - DNA, 56 bp, Artificial Sequence
  Description of Artificial Sequence Synthetic primer
acactctttccctacacgacgctcttccgatctggtctctctcccagcctgtgctg

SEQ ID NO: 542 - DNA, 56 bp, Artificial Sequence
  Description of Artificial Sequence Synthetic primer
acactctttccctacacgacgctcttccgatctggtctctctcgcagcttgtgctg

SEQ ID NO: 543 - DNA, 56 bp, Artificial Sequence
  Description of Artificial Sequence Synthetic primer
acactctttccctacacgacgctcttccgatctggtctctctcccagcttgtgctg

SEQ ID NO: 544 - DNA, 56 bp, Artificial Sequence
  Description of Artificial Sequence Synthetic primer
acactctttccctacacgacgctcttccgatctgttcttgggccaattttatgctg

SEQ ID NO: 545 - DNA, 56 bp, Artificial Sequence
  Description of Artificial Sequence Synthetic primer
acactctttccctacacgacgctcttccgatctggtccaattcccaggctgtggtg

SEQ ID NO: 546 - DNA, 56 bp, Artificial Sequence
  Description of Artificial Sequence Synthetic primer
acactctttccctacacgacgctcttccgatctggtccaattctcaggctgtggtg

SEQ ID NO: 547 - DNA, 56 bp, Artificial Sequence
  Description of Artificial Sequence Synthetic primer
acactctttccctacacgacgctcttccgatctgagtggattctcagactgtggtg

SEQ ID NO: 548 - DNA, 56 bp, Artificial Sequence
  Description of Artificial Sequence Synthetic primer
acactctttccctacacgacgctcttccgatctggtcctgggcccagtctgtcgtg

SEQ ID NO: 549 - DNA, 56 bp, Artificial Sequence
  Description of Artificial Sequence Synthetic primer
acactctttccctacacgacgctcttccgatctagtgtcagtggtccaggcagggc

SEQ ID NO: 550 - DNA, 56 bp, Artificial Sequence
  Description of Artificial Sequence Synthetic primer
acactctttccctacacgacgctcttccgatctacaggatcctgggctcagtctgc

SEQ ID NO: 551 - DNA, 56 bp, Artificial Sequence
  Description of Artificial Sequence Synthetic primer
acactctttccctacacgacgctcttccgatctctcctatgagctgactcagccac

SEQ ID NO: 552 - DNA, 56 bp, Artificial Sequence
  Description of Artificial Sequence Synthetic primer
acactctttccctacacgacgctcttccgatcttcttctgagctgactcaggaccc

SEQ ID NO: 553 - DNA, 56 bp, Artificial Sequence
  Description of Artificial Sequence Synthetic primer
acactctttccctacacgacgctcttccgatctaggtctctgtgctctgcctgtgc

SEQ ID NO: 554 - DNA, 56 bp, Artificial Sequence
  Description of Artificial Sequence Synthetic primer
acactctttccctacacgacgctcttccgatctaggttccctctcgcagcctgtgc

SEQ ID NO: 555 - DNA, 67 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnaggacggtgaccttggtccc

SEQ ID NO: 556 - DNA, 67 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnaggacggtcagctgggtccc

SEQ ID NO: 557 - DNA, 67 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnaggacggtcaccttggtgcc

SEQ ID NO: 558 - DNA, 67 bp, Artificial Sequence
  Description of Artificial Sequence Synthetic primer
  modified_base (34)..(37): a, c, t, g, unknown or other
  modified_base (39)..(42): a, c, t, g, unknown or other
  modified_base (44)..(47): a, c, t, g, unknown or other
tgactggagttcagacgtgtgctcttccgatctnnnntnnnntnnnnaggacggtcagctgggtgcc

SEQ ID NO: 559 - DNA, 29 bp, Artificial Sequence
  Description of Artificial Sequence Synthetic primer
aatgatacggcgaccaccgagatctacac

SEQ ID NO: 560 - DNA, 32 bp, Artificial Sequence
  Description of Artificial Sequence Synthetic primer
acactctttccctacacgacgctcttccgatc

SEQ ID NO: 561 - DNA, 24 bp, Artificial Sequence
  Description of Artificial Sequence Synthetic primer
caagcagaagacggcatacgagat

SEQ ID NO: 562 - DNA, 30 bp, Artificial Sequence
  Description of Artificial Sequence Synthetic primer
gtgactggagttcagacgtgtgctcttccg

SEQ ID NO: 563 - DNA, 29 bp, Artificial Sequence
  Description of Artificial Sequence Synthetic primer
aatgatacggcgaccaccgagatctacac

SEQ ID NO: 564 - DNA, 33 bp, Artificial Sequence
  Description of Artificial Sequence Synthetic primer
acactctttccctacacgacgctcttccgatct

SEQ ID NO: 565 - DNA, 24 bp, Artificial Sequence
  Description of Artificial Sequence Synthetic primer
caagcagaagacggcatacgagat

SEQ ID NO: 566 - DNA, 34 bp, Artificial Sequence
  Description of Artificial Sequence Synthetic primer
gtgactggagttcagacgtgtgctcttccgatct

SEQ ID NO: 567 - DNA, 38 bp, Artificial Sequence
  Description of Artificial Sequence Synthetic primer
gggcccaggcggccgagctcgtgctgactcagccaccc

SEQ ID NO: 568 - DNA, 36 bp, Artificial Sequence
  Description of Artificial Sequence Synthetic primer
gggcccaggcggccgagctcgtgctgacgcagccgc

SEQ ID NO: 569 - DNA, 36 bp, Artificial Sequence
  Description of Artificial Sequence Synthetic primer
gggcccaggcggccgagctcgtcgtgacgcagccgc

SEQ ID NO: 570 - DNA, 37 bp, Artificial Sequence
  Description of Artificial Sequence Synthetic primer
gggcccaggcggccgagctcgtgttgacgcagccgcc

SEQ ID NO: 571 - DNA, 38 bp, Artificial Sequence
  Description of Artificial Sequence Synthetic primer
gggcccaggcggccgagctcgggctgactcagccaccc

SEQ ID NO: 572 - DNA, 38 bp, Artificial Sequence
  Description of Artificial Sequence Synthetic primer
gggcccaggcggccgagctcgccctgactcagcctcgc

SEQ ID NO: 573 - DNA, 38 bp, Artificial Sequence
  Description of Artificial Sequence Synthetic primer
gggcccaggcggccgagctcgccctgactcagcctgcc

SEQ ID NO: 574 - DNA, 38 bp, Artificial Sequence
  Description of Artificial Sequence Synthetic primer
gggcccaggcggccgagctcgccctgactcagcctccc

SEQ ID NO: 575 - DNA, 40 bp, Artificial Sequence
  Description of Artificial Sequence Synthetic primer
gggcccaggcggccgagctcgagctgactcagccaccctc

SEQ ID NO: 576 - DNA, 40 bp, Artificial Sequence
  Description of Artificial Sequence Synthetic primer
gggcccaggcggccgagctcgagctgacacagccaccctc

SEQ ID NO: 577 - DNA, 40 bp, Artificial Sequence
  Description of Artificial Sequence Synthetic primer
gggcccaggcggccgagctcgagctgactcagccacactc

SEQ ID NO: 578 - DNA, 40 bp, Artificial Sequence
  Description of Artificial Sequence Synthetic primer
gggcccaggcggccgagctcgagctgactcaggaccctgc

SEQ ID NO: 579 - DNA, 41 bp, Artificial Sequence
  Description of Artificial Sequence Synthetic primer
gggcccaggcggccgagctcgagctgacacagctaccctcg

SEQ ID NO: 580 - DNA, 38 bp, Artificial Sequence
  Description of Artificial Sequence Synthetic primer
gggcccaggcggccgagctcgagctgatgcagccaccc

SEQ ID NO: 581 - DNA, 41 bp, Artificial Sequence
  Description of Artificial Sequence Synthetic primer
gggcccaggcggccgagctcgagctgacacagccatcctca

SEQ ID NO: 582 - DNA, 41 bp, Artificial Sequence
  Description of Artificial Sequence Synthetic primer
gggcccaggcggccgagctcgagctgactcagccactctca

SEQ ID NO: 583 - DNA, 38 bp, Artificial Sequence
  Description of Artificial Sequence Synthetic primer
gggcccaggcggccgagctcgtgctgactcagcccccg

SEQ ID NO: 584 - DNA, 41 bp, Artificial Sequence
  Description of Artificial Sequence Synthetic primer
gggcccaggcggccgagctcgtgctgactcaatcatcctct

SEQ ID NO: 585 - DNA, 41 bp, Artificial Sequence
  Description of Artificial Sequence Synthetic primer
gggcccaggcggccgagctcgtgctgactcaatcgccctct

SEQ ID NO: 586 - DNA, 40 bp, Artificial Sequence
  Description of Artificial Sequence Synthetic primer
gggcccaggcggccgagctcgtgctgactcagccaccttc

SEQ ID NO: 587 - DNA, 40 bp, Artificial Sequence
  Description of Artificial Sequence Synthetic primer
gggcccaggcggccgagctcgtgctgactcagccaacctc

SEQ ID NO: 588 - DNA, 38 bp, Artificial Sequence
  Description of Artificial Sequence Synthetic primer
gggcccaggcggccgagctcgtgctgactcagccggct

SEQ ID NO: 589 - DNA, 40 bp, Artificial Sequence
  Description of Artificial Sequence Synthetic primer
gggcccaggcggccgagctcgtgctgactcagccgtcttc

SEQ ID NO: 590 - DNA, 41 bp, Artificial Sequence
  Description of Artificial Sequence Synthetic primer
gggcccaggcggccgagctcgtgctgactcagccatcttcc

SEQ ID NO: 591 - DNA, 40 bp, Artificial Sequence
  Description of Artificial Sequence Synthetic primer
gggcccaggcggccgagctcatgctgactcagccccactc

SEQ ID NO: 592 - DNA, 40 bp, Artificial Sequence
  Description of Artificial Sequence Synthetic primer
gggcccaggcggccgagctcgtggtgactcaggagccctc

SEQ ID NO: 593 - DNA, 40 bp, Artificial Sequence
  Description of Artificial Sequence Synthetic primer
gggcccaggcggccgagctcgtggtgacccaggagccatc

SEQ ID NO: 594 - DNA, 44 bp, Artificial Sequence
  Description of Artificial Sequence Synthetic primer
ggaagatctagaggaaccaccgcctaggacggtgaccttggtcc

SEQ ID NO: 595 - DNA, 44 bp, Artificial Sequence
  Description of Artificial Sequence Synthetic primer
ggaagatctagaggaaccaccgcctaggacggtcagcttggtcc

SEQ ID NO: 596 - DNA, 43 bp, Artificial Sequence
  Description of Artificial Sequence Synthetic primer
ggaagatctagaggaaccaccgccgaggacggtcaccttggtg

SEQ ID NO: 597 - DNA, 43 bp, Artificial Sequence
  Description of Artificial Sequence Synthetic primer
ggaagatctagaggaaccaccgccgaggacggtcagctgggtg

SEQ ID NO: 598 - DNA, 41 bp, Artificial Sequence
  Description of Artificial Sequence Synthetic primer
ggaagatctagaggaaccaccgccgagggcggtcagctggg

SEQ ID NO: 599 - DNA, 41 bp, Artificial Sequence
  Description of Artificial Sequence Synthetic primer
gggcccaggcggccgagctccagatgacccagtctccatct

SEQ ID NO: 600 - DNA, 41 bp, Artificial Sequence
  Description of Artificial Sequence Synthetic primer
gggcccaggcggccgagctccagttgacccagtctccatcc

SEQ ID NO: 601 - DNA, 41 bp, Artificial Sequence
  Description of Artificial Sequence Synthetic primer
gggcccaggcggccgagctccagatgacccagtctccatcc

SEQ ID NO: 602 - DNA, 41 bp, Artificial Sequence
  Description of Artificial Sequence Synthetic primer
gggcccaggcggccgagctccagatgacccagtctccttcc

SEQ ID NO: 603 - DNA, 40 bp, Artificial Sequence
  Description of Artificial Sequence Synthetic primer
gggcccaggcggccgagctccggatgacccagtctccatc

SEQ ID NO: 604 - DNA, 41 bp, Artificial Sequence
  Description of Artificial Sequence Synthetic primer
gggcccaggcggccgagctccggatgacccagtctccattc

SEQ ID NO: 605 - DNA, 41 bp, Artificial Sequence
  Description of Artificial Sequence Synthetic primer
gggcccaggcggccgagctctggatgacccagtctccatcc

SEQ ID NO: 606 - DNA, 41 bp, Artificial Sequence
  Description of Artificial Sequence Synthetic primer
gggcccaggcggccgagctcgtgatgacccagactccactc

SEQ ID NO: 607 - DNA, 41 bp, Artificial Sequence
  Description of Artificial Sequence Synthetic primer
gggcccaggcggccgagctcgtgatgactcagtctccactc

SEQ ID NO: 608 - DNA, 40 bp, Artificial Sequence
  Description of Artificial Sequence Synthetic primer
gggcccaggcggccgagctcgtgttgacacagtctccagc

SEQ ID NO: 609 - DNA, 40 bp, Artificial Sequence
  Description of Artificial Sequence Synthetic primer
gggcccaggcggccgagctcgtgatgacgcagtctccagc

SEQ ID NO: 610 - DNA, 39 bp, Artificial Sequence
  Description of Artificial Sequence Synthetic primer
gggcccaggcggccgagctcgtgttgacgcagtctccag

SEQ ID NO: 611 - DNA, 41 bp, Artificial Sequence
  Description of Artificial Sequence Synthetic primer
gggcccaggcggccgagctcgtaatgacacagtctccagcc

SEQ ID NO: 612 - DNA, 41 bp, Artificial Sequence
  Description of Artificial Sequence Synthetic primer
gggcccaggcggccgagctcgtgatgacccagtctccagac

SEQ ID NO: 613 - DNA, 39 bp, Artificial Sequence
  Description of Artificial Sequence Synthetic primer
gggcccaggcggccgagctcacactcacgcagtctccag

SEQ ID NO: 614 - DNA, 41 bp, Artificial Sequence
  Description of Artificial Sequence Synthetic primer
gggcccaggcggccgagctcgtgctgactcagtctccagac

SEQ ID NO: 615 - DNA, 42 bp, Artificial Sequence
  Description of Artificial Sequence Synthetic primer
ggaagatctagaggaaccacctttgatttccaccttggtccc

SEQ ID NO: 616 - DNA, 42 bp, Artificial Sequence
  Description of Artificial Sequence Synthetic primer
ggaagatctagaggaaccacctttgatctccagcttggtccc

SEQ ID NO: 617 - DNA, 42 bp, Artificial Sequence
  Description of Artificial Sequence Synthetic primer
ggaagatctagaggaaccacctttgatatccactttggtccc

SEQ ID NO: 618 - DNA, 42 bp, Artificial Sequence
  Description of Artificial Sequence Synthetic primer
ggaagatctagaggaaccacctttgatctccaccttggtccc

SEQ ID NO: 619 - DNA, 42 bp, Artificial Sequence
  Description of Artificial Sequence Synthetic primer
ggaagatctagaggaaccacctttaatctccagtcgtgtccc

SEQ ID NO: 620 - DNA, 74 bp, Artificial Sequence
  Description of Artificial Sequence Synthetic primer
ggtggttcctctagatcttcctcctctggtggcggtggctcgggcggtggtgggcaggttcagctggtgcagtc

SEQ ID NO: 621 - DNA, 72 bp, Artificial Sequence
  Description of Artificial Sequence Synthetic primer
ggtggttcctctagatcttcctcctctggtggcggtggctcgggcggtggtgggcaggtgcagctggtgcag

SEQ ID NO: 622 - DNA, 75 bp, Artificial Sequence
  Description of Artificial Sequence Synthetic primer
ggtggttcctctagatcttcctcctctggtggcggtggctcgggcggtggtgggcaggtccagctggtacagtct

SEQ ID NO: 623 - DNA, 74 bp, Artificial Sequence
  Description of Artificial Sequence Synthetic primer
ggtggttcctctagatcttcctcctctggtggcggtggctcgggcggtggtgggcaggtccagcttgtgcagtc

SEQ ID NO: 624 - DNA, 74 bp, Artificial Sequence
  Description of Artificial Sequence Synthetic primer
ggtggttcctctagatcttcctcctctggtggcggtggctcgggcggtggtgggcagatgcagctggtgcagtc

SEQ ID NO: 625 - DNA, 74 bp, Artificial Sequence
  Description of Artificial Sequence Synthetic primer
ggtggttcctctagatcttcctcctctggtggcggtggctcgggcggtggtgggcaaatgcagctggtgcagtc

SEQ ID NO: 626 - DNA, 74 bp, Artificial Sequence
  Description of Artificial Sequence Synthetic primer
ggtggttcctctagatcttcctcctctggtggcggtggctcgggcggtggtgggcaggtccagctggtgcaatc

SEQ ID NO: 627 - DNA, 72 bp, Artificial Sequence
  Description of Artificial Sequence Synthetic primer
ggtggttcctctagatcttcctcctctggtggcggtggctcgggcggtggtgggcaggtccagctggtgcag

SEQ ID NO: 628 - DNA, 74 bp, Artificial Sequence
  Description of Artificial Sequence Synthetic primer
ggtggttcctctagatcttcctcctctggtggcggtggctcgggcggtggtggggaggtccagctggtacagtc

SEQ ID NO: 629 - DNA, 75 bp, Artificial Sequence
  Description of Artificial Sequence Synthetic primer
ggtggttcctctagatcttcctcctctggtggcggtggctcgggcggtggtgggcaggtcaccttgaaggagtct

SEQ ID NO: 630 - DNA, 75 bp, Artificial Sequence
  Description of Artificial Sequence Synthetic primer
ggtggttcctctagatcttcctcctctggtggcggtggctcgggcggtggtgggcagatcaccttgaaggagtct

SEQ ID NO: 631 - DNA, 74 bp, Artificial Sequence
  Description of Artificial Sequence Synthetic primer
ggtggttcctctagatcttcctcctctggtggcggtggctcgggcggtggtgggcaggtcaccttgagggagtc

SEQ ID NO: 632 - DNA, 72 bp, Artificial Sequence
  Description of Artificial Sequence Synthetic primer
ggtggttcctctagatcttcctcctctggtggcggtggctcgggcggtggtgggcgggtcaccttgagggag

SEQ ID NO: 633 - DNA, 72 bp, Artificial Sequence
  Description of Artificial Sequence Synthetic primer
ggtggttcctctagatcttcctcctctggtggcggtggctcgggcggtggtgggcaggtgcagctggtggag

SEQ ID NO: 634 - DNA, 74 bp, Artificial Sequence
  Description of Artificial Sequence Synthetic primer
ggtggttcctctagatcttcctcctctggtggcggtggctcgggcggtggtgggcaggtgcagctgttggagtc

SEQ ID NO: 635 - DNA, 72 bp, Artificial Sequence
  Description of Artificial Sequence Synthetic primer
ggtggttcctctagatcttcctcctctggtggcggtggctcgggcggtggtggggaggtgcagctggtggag

SEQ ID NO: 636 - DNA, 74 bp, Artificial Sequence
  Description of Artificial Sequence Synthetic primer
ggtggttcctctagatcttcctcctctggtggcggtggctcgggcggtggtggggaggtgcatctggtggagtc

SEQ ID NO: 637 - DNA, 74 bp, Artificial Sequence
  Description of Artificial Sequence Synthetic primer
ggtggttcctctagatcttcctcctctggtggcggtggctcgggcggtggtggggaggtgcaactggtggagtc

SEQ ID NO: 638 - DNA, 74 bp, Artificial Sequence
  Description of Artificial Sequence Synthetic primer
ggtggttcctctagatcttcctcctctggtggcggtggctcgggcggtggtggggaggtgcagctgttggagtc

SEQ ID NO: 639 - DNA, 72 bp, Artificial Sequence
  Description of Artificial Sequence Synthetic primer
ggtggttcctctagatcttcctcctctggtggcggtggctcgggcggtggtgggcaggtgcagctggtggac

SEQ ID NO: 640 - DNA, 74 bp, Artificial Sequence
  Description of Artificial Sequence Synthetic primer
ggtggttcctctagatcttcctcctctggtggcggtggctcgggcggtggtgggcaggtacagctggtggagtc

SEQ ID NO: 641 - DNA, 74 bp, Artificial Sequence
  Description of Artificial Sequence Synthetic primer
ggtggttcctctagatcttcctcctctggtggcggtggctcgggcggtggtggggaagtgcagctggtggagtc

SEQ ID NO: 642 - DNA, 72 bp, Artificial Sequence
  Description of Artificial Sequence Synthetic primer
ggtggttcctctagatcttcctcctctggtggcggtggctcgggcggtggtgggcaggtgcagctgcaggag

SEQ ID NO: 643 - DNA, 74 bp, Artificial Sequence
  Description of Artificial Sequence Synthetic primer
ggtggttcctctagatcttcctcctctggtggcggtggctcgggcggtggtgggcaggtgcagctacaggagtc

SEQ ID NO: 644 - DNA, 74 bp, Artificial Sequence
  Description of Artificial Sequence Synthetic primer
ggtggttcctctagatcttcctcctctggtggcggtggctcgggcggtggtgggcaggtacagctgcaggagtc

SEQ ID NO: 645 - DNA, 72 bp, Artificial Sequence
  Description of Artificial Sequence Synthetic primer
ggtggttcctctagatcttcctcctctggtggcggtggctcgggcggtggtgggcagctgcagctgcaggag

SEQ ID NO: 646 - DNA, 74 bp, Artificial Sequence
  Description of Artificial Sequence Synthetic primer
ggtggttcctctagatcttcctcctctggtggcggtggctcgggcggtggtgggcaggtgcagctacagcagtg

SEQ ID NO: 647 - DNA, 75 bp, Artificial Sequence
  Description of Artificial Sequence Synthetic primer
ggtggttcctctagatcttcctcctctggtggcggtggctcgggcggtggtgggcaggtgcagctacaacagtgg

SEQ ID NO: 648 - DNA, 70 bp, Artificial Sequence
  Description of Artificial Sequence Synthetic primer
ggtggttcctctagatcttcctcctctggtggcggtggctcgggcggtggtgggcggctgcagctgcagg

SEQ ID NO: 649 - DNA, 74 bp, Artificial Sequence
  Description of Artificial Sequence Synthetic primer
ggtggttcctctagatcttcctcctctggtggcggtggctcgggcggtggtggggaagtgcagctggtgcagtc

SEQ ID NO: 650 - DNA, 72 bp, Artificial Sequence
  Description of Artificial Sequence Synthetic primer
ggtggttcctctagatcttcctcctctggtggcggtggctcgggcggtggtggggaggtgcagctggtgcag

SEQ ID NO: 651 - DNA, 74 bp, Artificial Sequence
  Description of Artificial Sequence Synthetic primer
ggtggttcctctagatcttcctcctctggtggcggtggctcgggcggtggtgggcaggtacagctgcagcagtc

SEQ ID NO: 652 - DNA, 74 bp, Artificial Sequence
  Description of Artificial Sequence Synthetic primer
ggtggttcctctagatcttcctcctctggtggcggtggctcgggcggtggtgggcaggtgcagctggtgcaatc

SEQ ID NO: 653 - DNA, 41 bp, Artificial Sequence
  Description of Artificial Sequence Synthetic primer
cctggccggcctggccactagttgaggagacggtgaccagg

SEQ ID NO: 654 - DNA, 42 bp, Artificial Sequence
  Description of Artificial Sequence Synthetic primer
cctggccggcctggccactagttgaggagacagtgaccaggg

SEQ ID NO: 655 - DNA, 43 bp, Artificial Sequence
  Description of Artificial Sequence Synthetic primer
cctggccggcctggccactagttgaagagacggtgaccattgt

SEQ ID NO: 656 - DNA, 41 bp, Artificial Sequence
  Description of Artificial Sequence Synthetic primer
cctggccggcctggccactagttgaggagacggtgaccgtg

SEQ ID NO: 657 - DNA, 24 bp, Artificial Sequence
  Description of Artificial Sequence Synthetic primer
ggtggttcctctagatcttcctcc

SEQ ID NO: 658 - DNA, 18 bp, Artificial Sequence
  Description of Artificial Sequence Synthetic primer
cctggccggcctggccac

SEQ ID NO: 659 - DNA, 17 bp, Artificial Sequence
  Description of Artificial Sequence Synthetic primer
gggcccaggcggccgag

SEQ ID NO: 660 - DNA, 21 bp, Artificial Sequence
  Description of Artificial Sequence Synthetic primer
ggaagatctagaggaaccacc

SEQ ID NO: 661 - DNA, 41 bp, Artificial Sequence
  Description of Artificial Sequence Synthetic primer
gaggaggaggaggaggaggcggggcccaggcggccgagctc

SEQ ID NO: 662 - DNA, 40 bp, Artificial Sequence
  Description of Artificial Sequence Synthetic primer
gaggaggaggaggaggagcctggccggcctggccactagt

SEQ ID NO: 663 - DNA, 19 bp, Artificial Sequence
  Description of Artificial Sequence Synthetic primer
ggcccaggcggcctttaca

SEQ ID NO: 664 - DNA, 20 bp, Artificial Sequence
  Description of Artificial Sequence Synthetic primer
ggccggcctggccaaagttg

SEQ ID NO: 665 - DNA, 32 bp, Artificial Sequence
  Description of Artificial Sequence Synthetic primer
gtgtctacgcatgggacagaaagagaatcagt

SEQ ID NO: 666 - DNA, 32 bp, Artificial Sequence
  Description of Artificial Sequence Synthetic primer
actgattctctttctgtcccatgcgtagacac

SEQ ID NO: 667 - DNA, 32 bp, Artificial Sequence
  Description of Artificial Sequence Synthetic primer
gtaactgtgtagcgtactatagtgtcctttat

SEQ ID NO: 668 - DNA, 32 bp, Artificial Sequence
  Description of Artificial Sequence Synthetic primer
ataaaggacactatagtacgctacacagttac

SEQ ID NO: 669 - DNA, 35 bp, Artificial Sequence
  Description of Artificial Sequence Synthetic primer
tgtgtagcggattatagtttcctttataattcagc

SEQ ID NO: 670 - DNA, 35 bp, Artificial Sequence
  Description of Artificial Sequence Synthetic primer
gctgaattataaaggaaactataatccgctacaca

SEQ ID NO: 671 - DNA, 26 bp, Artificial Sequence
  Description of Artificial Sequence Synthetic primer
ttcggggaagtgctgaacgctacccg

SEQ ID NO: 672 - DNA, 26 bp, Artificial Sequence
  Description of Artificial Sequence Synthetic primer
cgggtagcgttcagcacttccccgaa

SEQ ID NO: 673 - DNA, 24 bp, Artificial Sequence
  Description of Artificial Sequence Synthetic primer
gagatgaggtgattcaaatcgcgc

SEQ ID NO: 674 - DNA, 24 bp, Artificial Sequence
  Description of Artificial Sequence Synthetic primer
gcgcgatttgaatcacctcatctc

SEQ ID NO: 675 - DNA, 30 bp, Artificial Sequence
  Description of Artificial Sequence Synthetic primer
ggatgtgttatcgctagaaactctaacaac

SEQ ID NO: 676 - DNA, 30 bp, Artificial Sequence
  Description of Artificial Sequence Synthetic primer
gttgttagagtttctagcgataacacatcc

SEQ ID NO: 677 - DNA, 26 bp, Artificial Sequence
  Description of Artificial Sequence Synthetic primer
cattcggggaaatctttaacgctacc

SEQ ID NO: 678 - DNA, 26 bp, Artificial Sequence
  Description of Artificial Sequence Synthetic primer
ggtagcgttaaagatttccccgaatg

SEQ ID NO: 679 - DNA, 28 bp, Artificial Sequence
  Description of Artificial Sequence Synthetic primer
gggatgtgttatcagctggaactctaac

SEQ ID NO: 680 - DNA, 28 bp, Artificial Sequence
  Description of Artificial Sequence Synthetic primer
gttagagttccagctgataacacatccc

SEQ ID NO: 681 - DNA, 26 bp, Artificial Sequence
  Description of Artificial Sequence Synthetic primer
atttatcaggctagcagcacaccttg

SEQ ID NO: 682 - DNA, 26 bp, Artificial Sequence
  Description of Artificial Sequence Synthetic primer
caaggtgtgctgctagcctgataaat

SEQ ID NO: 683 - DNA, 28 bp, Artificial Sequence
  Description of Artificial Sequence Synthetic primer
caccttgcaatggtgccgaaggattcaa

SEQ ID NO: 684 - DNA, 28 bp, Artificial Sequence
  Description of Artificial Sequence Synthetic primer
ttgaatccttcggcaccattgcaaggtg

SEQ ID NO: 685 - protein, 20 aa, Artificial Sequence
  Description of Artificial Sequence Synthetic peptide
DENRGYSSRWYDPEYYGHDV

SEQ ID NO: 686 - protein, 20 aa, Artificial Sequence
  Description of Artificial Sequence Synthetic peptide
YYYYYGMDVWGQGTTVTVSS

SEQ ID NO: 687 - protein, 75 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCAR

SEQ ID NO: 688 - protein, 95 aa, Artificial Sequence
  Description of Artificial Sequence Synthetic polypeptide
AASGFTVSSNYMSWVRQAPGKGLEWVSVLYSGGSTFYADSVKGRFTISRDNSKNTLYLQMNSLRAEDTAVYYCTRDAQVYGMDVWGQGTTVTVSS

SEQ ID NO: 689 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCARDLAVYGMDVWGQGTTVTVSS

SEQ ID NO: 690 - protein, 95 aa, Artificial Sequence
  Description of Artificial Sequence Synthetic polypeptide
AASGITVSSNYMSWVRQAPGKGLEWVSVIYSGGSTYYADSVKGRITISRDNSKNTLYLQMNSLRAEDTAVYYCARDLAVYGMDVWGQGTTVTVSS

SEQ ID NO: 691 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCARDLVAYGMDVWGQGTTVTVSS

SEQ ID NO: 692 - protein, 95 aa, Artificial Sequence
  Description of Artificial Sequence Synthetic polypeptide
AASGLTVSSNYMSWVRQAPGKGLEWVSVIYAGGTTDYADSVKGRFTISRDNSKNTLYLEMNSLRAEDTAVYYCARDLVAYGMDVWGQGTTVTVSS

SEQ ID NO: 693 - protein, 95 aa, Artificial Sequence
  Description of Artificial Sequence Synthetic polypeptide
AASGVTVSSNYMSWVRQAPGKGLEWVSVIYSGGSTYYADSVKGRFTISRDNSKNTLYLQMNSLRAEDTAVYYCARDLVAYGMDVWGQGTTVTVSS

SEQ ID NO: 694 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYVQMNSLRAEDTAVYYCARDLVAYGMDVWGQGTTVTVSS

SEQ ID NO: 695 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCARDLYYYGMDVWGQGTTVTVSS

SEQ ID NO: 696 - protein, 95 aa, Artificial Sequence
  Description of Artificial Sequence Synthetic polypeptide
AASGLTVSSNYMSWVRQAPGKGLEWVSVIYSGGSTYYADSVKGRFTISRHNSKNTLYLQMNSLRAEDTAVYYCARDLYYYGMDVWGQGTTVTVSS

SEQ ID NO: 697 - protein, 95 aa, Artificial Sequence
  Description of Artificial Sequence Synthetic polypeptide
AASGFTVSSNYMTWVRQAPGKGLEWVSVIYSGGSTYYADSVKGRFTISRDNSKNTLYLQMNSLRAEDTAVYYCARDLVYYGMDVWGQGTTVTVSS

SEQ ID NO: 698 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCARDLVYYGMDVWGQGTTVTVSS

SEQ ID NO: 699 - protein, 95 aa, Artificial Sequence
  Description of Artificial Sequence Synthetic polypeptide
AASGFTVSSNYMSWVRQAPGKGLEWVSVIYSGGTTYYADSVKGRFTISRDNSKNTLYLQMNSLRAEDTAVYYCARDLDYYGMDVWGQGTTVTVSS

SEQ ID NO: 700 - protein, 95 aa, Artificial Sequence
  Description of Artificial Sequence Synthetic polypeptide
AASGFTVSSNYMSWVRQAPGKGLEWVSVIYSGGSTYYADSVKGRFTISRDNSKNTLYLQMNSLRAEDTAVYYCARDLDYYGMDVWGQGTTVTVSS

SEQ ID NO: 701 - protein, 8 aa, Artificial Sequence
  Description of Artificial Sequence Synthetic peptide
QQVNSYPT

SEQ ID NO: 702 - protein, 9 aa, Artificial Sequence
  Description of Artificial Sequence Synthetic peptide
QQAISFPYT

SEQ ID NO: 703 - protein, 13 aa, Artificial Sequence
  Description of Artificial Sequence Synthetic peptide
QSYDSSLSGSDVV

SEQ ID NO: 704 - protein, 9 aa, Artificial Sequence
  Description of Artificial Sequence Synthetic peptide
AAWDDSLRV

SEQ ID NO: 705 - protein, 11 aa, Artificial Sequence
  Description of Artificial Sequence Synthetic peptide
SAWDDSLDVYV

SEQ ID NO: 706 - protein, 9 aa, Artificial Sequence
  Description of Artificial Sequence Synthetic peptide
SSYTSSSRV

SEQ ID NO: 707 - protein, 10 aa, Artificial Sequence
  Description of Artificial Sequence Synthetic peptide
NSRDSNDNVV

SEQ ID NO: 708 - protein, 9 aa, Artificial Sequence
  Description of Artificial Sequence Synthetic peptide
QAWDSSTVV